(19) United States
(12) Patent Application Publication
(10) Pub. No.: US 2026/0078375 A1
(43) Pub. Date: Mar. 19, 2026

(54) RNAi Agents for Inhibiting Influenza A Viral Gene Expression, Compositions Thereof, and Methods of Use

(71) Applicant: Arrowhead Pharmaceuticals, Inc., Pasadena, CA (US)

(72) Inventors: Erik W. Bush, Verona, WI (US); Keivan Zandi, San Diego, CA (US); Zhao Xu, San Diego, CA (US); Tingting Yuan, Madison, WI (US); Alireza Saeidi, San Diego, CA (US); Casi Schienebeck, Deerfield, WI (US); Anthony Nicholas, Oregon, WI (US); Tao Pei, Middleton, WI (US)

(21) Appl. No.: 19/266,598

(22) Filed: Jul. 11, 2025

Related U.S. Application Data
(63) Continuation of application No. PCT/US2024/011462, filed on Jan. 12, 2024.
(60) Provisional application No. 63/479,859, filed on Jan. 13, 2023.
(60) Provisional application No. 63/488,062, filed on Mar. 2, 2023.
(60) Provisional application No. 63/499,065, filed on Apr. 28, 2023.
(60) Provisional application No. 63/596,336, filed on Nov. 6, 2023.

(51) Int. Cl. C12N 15/113; A61P 31/16

(52) U.S. Cl. CPC C12N 15/1131; A61P 31/16; C12N 2310/14; C12N 2310/313; C12N 2310/314; C12N 2310/321; C12N 2310/351

(57) ABSTRACT

Described are RNAi agents, compositions that include RNAi agents, and methods for inhibition of an influenza A viral genome. The influenza A viral (IAV) RNAi agents and RNAi agent conjugates disclosed herein inhibit the expression of an influenza A viral genome at targeted portions of the genome that are conserved across a variety of known influenza A viral genome variants, and are therefore capable of inhibiting expression of various influenza A virus strains. Pharmaceutical compositions that include one or more IAV RNAi agents, optionally with one or more additional therapeutics, are also described. Delivery of the described IAV RNAi agents to pulmonary cells, in vivo, provides for inhibition of influenza A viral genome expression, which can provide a therapeutic benefit to subjects, including human subjects, for the treatment of various diseases, disorders, and/or symptoms caused by influenza A viral infection.

### RELATED APPLICATIONS

This application is a continuation application of International Application No. PCT/US2024/011462, filed on Jan. 12, 2024, which claims the benefit of priority of U.S. Provisional Patent Applications Ser. No. 63/479,859, filed on Jan. 13, 2023, United States Provisional Patent Applications Serial No. 63,488,062, filed Mar. 2, 2023, U.S. Provisional Patent Applications Ser. No. 63/499,065, filed Apr. 28, 2023, and U.S. Provisional Patent Applications Ser. No. 63/596,336, filed Nov. 6, 2023, the contents of each of which are incorporated herein by reference in their entirety.

### FIELD OF THE INVENTION

The present disclosure relates to RNA interference (RNAi) agents, e.g., double stranded RNAi agents such as small interfering RNA (siRNA), for inhibition of influenza A (IAV) viral genome (or gene) expression, including compositions that include IAV RNAi agents, and methods of use thereof.

### SEQUENCE LISTING

This application contains a Sequence Listing which has been submitted in XML format and is hereby incorporated by reference in its entirety. The XML copy is named 30715-US1_SeqListing.xml, created Jul. 11, 2025, and is 6,282,366 bytes in size.

### BACKGROUND

Influenza, more commonly referred to as “Flu,” is a contagious respiratory illness caused by influenza viruses that infect the nose, throat, and sometimes the lungs. It can cause a broad spectrum of relatively mild to quite severe illness, and in some instances can lead to death. A 2018 study from the Centers for Disease Control and Prevention (CDC) concluded that on average, about 8 percent of the U.S. population gets sick from flu each season, with a range of between 3 percent and 11 percent, depending on the season.

Influenza virus is a member of the Orthomyxoviridae family and comes in four types: A, B, C and D. (Li, X., Gu, M., Zheng, Q. et al. Packaging signal of influenza A virus. Virol J 18, 36 (2021)). The influenza virus particle is composed of a viral envelope, matrix proteins and viral ribonucleocapsids (vRNPs). Influenza A and B virus genomes each comprise eight negative-sense, single-stranded viral RNA (vRNA) segments, which are: M1/M2, NS1, NA, NP, HA, PA, PB1, and PB2. (Bouvier N M, Palese P. The biology of influenza viruses. Vaccine. 2008 Sep. 12; 26 Suppl 4(Suppl 4):D49-53). Influenza C has a seven-segment genome. These viral segments encode the various proteins necessary to facilitate the influenza viral cycle of virus entry, viral RNA synthesis, viral protein synthesis, viral RNA packaging and assembly, and virus budding and release.

Flu has been reported to cause between 290,000 and 650,000 deaths per year. (https://www.cdc.gov/media/releases/2017/p1213-flu-death-estimate.html, last visited Mar. 1, 2023). Flu creates a huge economic burden and strain on the medical systems, with estimates of over $3 billion in annual direct medical costs in the United States alone. While flu vaccines that can be administered annually are widely available and can reduce illnesses and symptoms in many individuals, they do not prevent influenza-induced mortality in high-risk populations. Further, while currently there are 4 FDA-approved antiviral compounds that are indicated to be taken within 48 hours of symptom onset, they are not recommended for flu prevention. Moreover, while these products target either the NA vRNA segment (oseltamivir (Tamiflu®), zanamivir (Relenza®), and peramivir (Rapivab®) or the PA vRNA segment (baloxavir marboxil (Xofluza®)), strains of influenza that are resistant to the approved antiviral therapies are becoming more prevalent. Thus, there is an urgent need to develop novel and effective alternative therapies for influenza.

Influenza A viruses are divided into subtypes based on two proteins on the surface of the virus: hemagglutinin (H) and neuraminidase (N). (https://www.cdc.gov/flu/about/viruses/types.htm, Centers for Disease Control and Prevention.) There are 18 different hemagglutinin subtypes and 11 different neuraminidase subtypes (H1 through H18 and N1 through N11, respectively). While more than 130 influenza A subtype combinations have been identified in nature, primarily from wild birds, there are potentially many more influenza A subtype combinations given the propensity for virus “reassortment.” Reassortment is a process by which influenza viruses swap gene segments. Reassortment can occur when two influenza viruses infect a host at the same time and swap genetic information.

Influenza A viruses circulate and cause seasonal epidemics of disease. Currently known circulating viruses in humans include subtype A H1N1, subtype A H2N2, and subtype A H3N2. (Belser J A, Maines T R, Tumpey T M, Katz J M. Influenza A virus transmission: contributing factors and clinical implications. Expert Rev. Mol. Med. 12: e39.) In recent years, an increasing number of IAV subtypes have been detected in humans, including H5N1, H7N9, and H10N8. (Rejmanek D, Hosseini P R, Mazet J A, Daszak P, Goldstein T. Evolutionary Dynamics and Global Diversity of Influenza A Virus. J Virol. 2015 November; 89(21):10993-1001. doi: 10.1128/JVI.01573-15. Epub 2015 August 26. PMID: 26311890; PMCID: PMC4621101.)

Of note, the avian H5N1 virus often leads to severe cases when infecting humans. (Wang Y, Song T, Li K, Jin Y, Yue J, Ren H, Liang L. Different Subtypes of Influenza Viruses Target Different Human Proteins and Pathways Leading to Different Pathogenic Phenotypes. Biomed Res Int. 2019 Oct. 22; 2019:4794910. doi: 10.1155/2019/4794910. PMID: 31772934; PMCID: PMC6854240.) In 1996, for example, the highly pathogenic avian influenza (HPAI) H5N1 virus was first identified in domestic waterfowl in Southern China. In 1997, H5N1 poultry outbreaks occurred in China and Hong Kong with 18 associated human cases (6 deaths) in Hong Kong. This virus outbreak caused more than 860 human infections with a greater than 50% death rate. (https://www.cdc.gov/flu/avianflu/communication-resources/bird-flu-origin-infographic.html, Center for Disease Control and Prevention.) The first case of an avian influenza A (H5N1) virus in a person in the United States was reported on Apr. 28, 2022. Though current risk of H5N1 human infection remains low, people with job-related or recreational exposures to birds or infected mammals should take appropriate precautions to protect against bird flu H5N1. Right now, the H5N1 bird flu situation remains primarily an animal health issue. However, CDC is watching this situation closely and taking routine preparedness and prevention measures in case this virus changes to pose a greater human health risk.

### SUMMARY

There exists a need for novel RNA interference (RNAi) agents (termed RNAi agents, RNAi triggers, or triggers), e.g., double-stranded RNAi agents such as small interfering RNA (siRNA), that are able to selectively and efficiently inhibit the expression of influenza A viral genomes, including but not limited to selectively and efficiently inhibiting the influenza A mRNA expression and thus the replication of influenza A viral genome. Further, there exists a need for compositions of novel influenza A-genome-specific RNAi agents for use as a therapeutic or medicament for the treatment of influenza A and/or diseases or disorders (including alleviating symptoms) that can be mediated at least in part by a reduction in influenza A viral genome expression. While the concept of targeting influenza with an RNAi agent such as an siRNA, including the advantages of using this approach, has long been proposed (see, e.g., Sailen Bank, siRNA for Influenza Therapy, Viruses (2010) 2(7): 1448-1457), more than a decade later there still is no anti-influenza A siRNA drug that has received market authorization for treatment in humans.

The nucleotide sequences and chemical modifications of the influenza A virus (IAV) RNAi agents disclosed herein, as well as their combination with certain specific targeting ligands suitable for selectively and efficiently delivering the IAV RNAi agents to pulmonary cells in vivo, including delivery by inhalation administration, differ from those previously disclosed or known in the art and overcome the challenges and obstacles that others were unable to surmount. The IAV RNAi agents disclosed herein provide for highly potent and efficient in vivo inhibition of the expression of influenza A mRNA (or transcripts), and because of the conserved nature of the RNAi agent antisense strand sequences disclosed herein, are expected to effectively inhibit a vast majority of the thousands of known and unknown variants of influenza A mRNA (or transcripts).

In general, the present disclosure features IAV RNAi agents that are specific to influenza A viral genome and target a portion of the genome that is conserved across other influenza genomes, compositions that include IAV RNAi agents, and methods for inhibiting expression of an influenza A viral genome in vivo, using the IAV RNAi agents and compositions that include IAV RNAi agents described herein. The IAV RNAi agents described herein are able to selectively and efficiently decrease expression of influenza A viral genomes. The IAV RNAi agents can be used to inhibit expression of influenza A viral genomes of influenza A subtypes including but not limited to, H1N1, H2N2, H3N2, H5N1, H7N9, and H10N8.

The described IAV RNAi agents can be used in methods for therapeutic treatment (including potentially preventative or prophylactic treatment) of symptoms or diseases related to influenza A viral infection, including but not limited to infection of the nose, throat, lungs, and other parts of the respiratory system.

The described IAV RNAi agents can be used in methods for therapeutic treatment (including potentially preventative or prophylactic treatment) of symptoms or diseases related to influenza A viral infection, of influenza A subtypes including but not limited to, H1N1, H2N2, H3N2, H5N1, H7N9, and H10N8.

As the described IAV RNAi agents are designed to inhibit expression of influenza A viral genomes by targeting highly conserved genomic regions of the influenza A viral genome, the IAV RNAi agents can inhibit expression of multiple subtypes of the influenza A virus, including but not limited to, H1N1, H2N2, H3N2, H5N1, H7N9, and H10N8.

In one aspect, the disclosure features RNAi agents for inhibiting expression of influenza A viral genomes, wherein the RNAi agent includes a sense strand (also referred to as a passenger strand) and an antisense strand (also referred to as a guide strand). The sense strand and the antisense strand can be partially, substantially, or fully complementary to each other. The length of the RNAi agent sense strands described herein each can be 15 to 49 nucleotides in length. The length of the RNAi agent antisense strands described herein each can be 18 to 49 nucleotides in length. In some embodiments, the sense and antisense strands are independently 18 to 26 nucleotides in length. The sense and antisense strands can be either the same length or different lengths. In some embodiments, the sense and antisense strands are independently 21 to 26 nucleotides in length. In some embodiments, the sense and antisense strands are independently 21 to 24 nucleotides in length. In some embodiments, both the sense strand and the antisense strand are 21 nucleotides in length. In some embodiments, the antisense strands are independently 18, 19, 20, 21, 22, 23, 24, 25, 26, 27, 28, 29, or 30 nucleotides in length. In some embodiments, the sense strands are independently 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 nucleotides in length. The RNAi agents described herein, upon delivery to a cell expressing influenza A viral genomes such as a pulmonary cell, inhibit the expression of one or more influenza A viral genomes in vivo and/or in vitro through the RNA-induced silencing complex (RISC)-mediated cleavage of the viral RNA transcripts.

The IAV RNAi agents disclosed herein are designed to target influenza A viral genomes (see, e.g., SEQ ID NO:1-6) in a region that is anticipated to be conserved across a variety of different influenza viruses. In some embodiments, the IAV RNAi agents disclosed herein are designed to target a portion of an influenza A viral gene having the sequence of any of the sequences disclosed in Table 1A, Table 1B, Table 1C, Table 1D, Table 1E, or Table 1F.

In another aspect, the disclosure features compositions, including pharmaceutical compositions, that include one or more of the disclosed IAV RNAi agents that are able to selectively and efficiently decrease expression of an influenza A viral gene. The compositions that include one or more IAV RNAi agents described herein can be administered to a subject, such as a human or animal subject, for the treatment (including potential prophylactic treatment or inhibition) of symptoms and diseases associated influenza A viral infection, including but not limited to infection of the nose, throat, lungs, and other parts of the respiratory system.

Examples of IAV RNAi agent sense strands and antisense strands that can be used in an IAV RNAi agent are provided in Tables 3A, 3B, 3C, 3D, 3E, 3F, 4A, 4B, 4C, 4D, 4E, 4F, 5A, 5B, 5C, 5D, 5E, 5F, 6A, 6B, 6C, 6D, 6E, and 6F. Examples of IAV RNAi agent duplexes are provided in Tables 7A-1, 7A-2, 7A-3, 7A-4, 7A-5, 7A-6, 7B-1, 7B-2, 7B-3, 7B-4, 7B-5, 7B-6, 8A, 8B, 8C, 8D, 8E, 8F, 9A, 9B, 9C, 9D, 9E, 9F, and 10A, 10B, 10C, 10D, 10E, and 10F. Examples of 19-nucleotide core stretch sequences that may consist of or may be included in the sense strands and antisense strands of certain IAV RNAi agents disclosed herein, are provided in Table 2A, 2B, 2C, 2D, 2E, and 2F.

In another aspect, the disclosure features methods for delivering IAV RNAi agents to pulmonary epithelial cells in a subject, such as a mammal, in vivo. Also described herein are compositions for use in such methods.

In some embodiments, disclosed herein are methods for delivering IAV RNAi agents to pulmonary cells (epithelial cells (including airway epithelial cells, alveolar type I and type II pneumocytes), mesenchymal cells (including smooth muscle cells and fibroblasts), immune cells (including macrophages and mast cells) and endothelial cells) to a subject in vivo. In some embodiments, the subject is a human subject.

The methods disclosed herein include the administration of one or more IAV RNAi agents to a subject, e.g., a human or animal subject, by any suitable means known in the art. The pharmaceutical compositions disclosed herein that include one or more IAV RNAi agents can be administered in a number of ways depending upon whether local or systemic treatment is desired. Administration can be, but is not limited to, for example, intravenous, intraarterial, subcutaneous, intraperitoneal, subdermal (e.g., via an implanted device), and intraparenchymal administration. In some embodiments, the pharmaceutical compositions described herein are administered by inhalation (such as dry powder inhalation or aerosol inhalation), intranasal administration, intratracheal administration, or oropharyngeal aspiration administration.

In some embodiments, it is desired that the IAV RNAi agents described herein inhibit the expression of influenza A viral genomes in the pulmonary epithelium, for which the administration is by inhalation (e.g., by an inhaler device, such as a metered-dose inhaler, or a nebulizer such as a jet or vibrating mesh nebulizer, or a soft mist inhaler).

In some embodiments, the described IAV RNAi agents are able to selectively and efficiently inhibit expression of an influenza A viral genome. The IAV RNAi agents described herein can be used to inhibit expression of an influenza A viral genome such as, including but not limited to, H1N1, H2N2, H3N2, H5N1, H7N9, and H10N8.

The IAV RNAi agents can be delivered to target cells or tissues using any oligonucleotide delivery technology known in the art. In some embodiments, an IAV RNAi agent is delivered to cells or tissues by covalently linking the RNAi agent to a targeting group. In some embodiments, the targeting group can include a cell receptor ligand, such as an integrin targeting ligand. Integrins are a family of transmembrane receptors that facilitate cell-extracellular matrix (ECM) adhesion. In particular, integrin alpha-v-beta-6 (αvβ6) is an epithelial-specific integrin that is known to be a receptor for ECM proteins and the TGF-beta latency-associated peptide (LAP), and is expressed in various cells and tissues. Integrin αvβ6 is known to be highly upregulated in injured pulmonary epithelium. In some embodiments, the IAV RNAi agents described herein are linked to an integrin targeting ligand that has affinity for integrin αvβ6. As referred to herein, an “αvβ6 integrin targeting ligand” is a compound that has affinity for integrin αvβ6, which can be utilized as a ligand to facilitate the targeting and delivery of an RNAi agent to which it is attached to the desired cells and/or tissues (i.e., to cells expressing integrin αvβ6, such as pulmonary cells). In some embodiments, multiple αvβ6 integrin targeting ligands or clusters of αvβ6 integrin targeting ligands are linked to an IAV RNAi agent. In some embodiments, the IAV RNAi agent-αvβ6 integrin targeting ligand conjugates are selectively internalized by lung epithelial cells, either through receptor-mediated endocytosis or by other means.

Examples of targeting groups useful for delivering IAV RNAi agents that include αvβ6 integrin targeting ligands are disclosed, for example, in International Patent Application Publication No. WO 2018/085415 and International Patent Application Publication No. WO 2019/089765, each to Arrowhead Pharmaceuticals, Inc., the contents of each of which are incorporated by reference herein in their entirety.

A targeting group can be linked to the 3′ or 5′ end of a sense strand or an antisense strand of an IAV RNAi agent. In some embodiments, a targeting group is linked to the 3′ or 5′ end of the sense strand. In some embodiments, a targeting group is linked to the 5′ end of the sense strand. In some embodiments, a targeting group is linked internally to a nucleotide on the sense strand and/or the antisense strand of the RNAi agent. In some embodiments, a targeting group is linked to the RNAi agent via a linker.

In another aspect, the disclosure features compositions that include one or more IAV RNAi agents that have the duplex structures disclosed in Tables 7A-1, 7A-2, 7A-3, 7A-4, 7A-5, 7A-6, 7B-1, 7B-2, 7B-3, 7B-4, 7B-5, 7B-6, 8A, 8B, 8C, 8D, 8E, 8F, 9A, 9B, 9C, 9D, 9E, 9F, and 10A, 10B, 10C, 10D, 10E, and 10F.

The use of IAV RNAi agents provides methods for therapeutic (including prophylactic) treatment of diseases or disorders related to influenza A viral infection, including but not limited to infection of the nose, throat, lungs, and other parts of the respiratory system caused by the influenza A viral genome. The IAV RNAi agents disclosed herein can be used to treat various respiratory diseases and injury related to influenza infection. In some embodiments, the IAV RNAi agents disclosed herein can be used to treat or prevent a pulmonary inflammatory disease or condition caused by influenza infection. Use of the described IAV RNAi agents described herein can be used for therapeutic (including prophylactic) treatment of diseases or disorders related to influenza A viral infection caused by influenza A virus subtypes such as, including but not limited to, H1N1, H2N2, H3N2, H5N1, H7N9, and H10N8.

### Definitions

As used herein, the terms “oligonucleotide” and “polynucleotide” mean a polymer of linked nucleosides each of which can be independently modified or unmodified.

As used herein, an “RNAi agent” (also referred to as an “RNAi trigger”) means a chemical composition of matter that contains an RNA or RNA-like (e.g., chemically modified RNA) oligonucleotide molecule that is capable of degrading RNA or inhibiting (e.g., degrades or inhibits under appropriate conditions) translation of viral RNA (including all viral RNA and viral messenger RNA (mRNA) transcripts) of a target influenza virus in a sequence specific manner. As used herein, RNAi agents may operate through the RNA interference mechanism (i.e., inducing RNA interference through interaction with the RNA interference pathway machinery (RNA-induced silencing complex or RISC) of mammalian cells), or by any alternative mechanism(s) or pathway(s). While it is believed that RNAi agents, as that term is used herein, operate primarily through the RNA interference mechanism, the disclosed RNAi agents are not bound by or limited to any particular pathway or mechanism of action. RNAi agents disclosed herein are comprised of a sense strand and an antisense strand, and include, but are not limited to: small (or short) interfering RNAs (siRNAs), double stranded RNAs (dsRNA), micro RNAs (miRNAs), short hairpin RNAs (shRNA), and dicer substrates. The antisense strand of the RNAi agents described herein is at least partially complementary to the RNA being targeted (e.g., influenza A viral mRNA). RNAi agents can include one or more modified nucleotides and/or one or more non-phosphodiester linkages.

As used herein, the terms “silence,” “reduce,” “inhibit,” “down-regulate,” or “knockdown” when referring to expression of a given gene or a viral genome, mean that the expression of the viral gene or genome (including viral genomic RNA or subgenomic RNA), as measured by the level of RNA transcribed from the viral gene or genome, the number of viral genomes, or the level of polypeptide, protein, or protein subunit translated from the viral RNA in a cell, group of cells, tissue, organ, or subject in which the viral gene or genome is transcribed, is reduced when the cell, group of cells, tissue, organ, or subject is treated with the RNAi agents described herein as compared to a second cell, group of cells, tissue, organ, or subject that has not or have not been so treated.

As used herein, the terms “sequence” and “nucleotide sequence” mean a succession or order of nucleobases or nucleotides, described with a succession of letters using standard nomenclature.

As used herein, a “base,” “nucleotide base,” or “nucleobase,” is a heterocyclic pyrimidine or purine compound that is a component of a nucleotide, and includes the primary purine bases adenine and guanine, and the primary pyrimidine bases cytosine, thymine, and uracil. A nucleobase may further be modified to include, without limitation, universal bases, hydrophobic bases, promiscuous bases, size-expanded bases, and fluorinated bases. (See, e.g., Modified Nucleosides in Biochemistry, Biotechnology and Medicine, Herdewijn, P. ed. Wiley-VCH, 2008). The synthesis of such modified nucleobases (including phosphoramidite compounds that include modified nucleobases) is known in the art.

As used herein, and unless otherwise indicated, the term “complementary,” when used to describe a first nucleobase or nucleotide sequence (e.g., RNAi agent sense strand or targeted RNA) in relation to a second nucleobase or nucleotide sequence (e.g., RNAi agent antisense strand or a single-stranded antisense oligonucleotide), means the ability of an oligonucleotide or polynucleotide including the first nucleotide sequence to hybridize (form base pair hydrogen bonds under mammalian physiological conditions (or otherwise suitable in vivo or in vitro conditions)) and form a duplex or double helical structure under certain standard conditions with an oligonucleotide that includes the second nucleotide sequence. The person of ordinary skill in the art would be able to select the set of conditions most appropriate for a hybridization test. Complementary sequences include Watson-Crick base pairs or non-Watson-Crick base pairs and include natural or modified nucleotides or nucleotide mimics, at least to the extent that the above hybridization requirements are fulfilled. Sequence identity or complementarity is independent of modification. For example, a and Af, as defined herein, are complementary to U (or T) and identical to A for the purposes of determining identity or complementarity.

As used herein, “perfectly complementary” or “fully complementary” means that in a hybridized pair of nucleobase or nucleotide sequence molecules, all (100%) of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, “partially complementary” means that in a hybridized pair of nucleobase or nucleotide sequence molecules, at least 70%, but not all, of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, “substantially complementary” means that in a hybridized pair of nucleobase or nucleotide sequence molecules, at least 85%, but not all, of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, the terms “complementary,” “fully complementary,” “partially complementary,” and “substantially complementary” are used with respect to the nucleobase or nucleotide matching between the sense strand and the antisense strand of an RNAi agent, or between the antisense strand of an RNAi agent and a sequence of a Influenza RNA, such as a Influenza A viral genome RNA.

As used herein, the term “substantially identical” or “substantial identity,” as applied to a nucleic acid sequence means the nucleotide sequence (or a portion of a nucleotide sequence) has at least about 85% sequence identity or more, e.g., at least 90%, at least 95%, or at least 99% identity, compared to a reference sequence. Percentage of sequence identity is determined by comparing two optimally aligned sequences over a comparison window. The percentage is calculated by determining the number of positions at which the same type of nucleic acid base occurs in both sequences to yield the number of matched positions, dividing the number of matched positions by the total number of positions in the window of comparison and multiplying the result by 100 to yield the percentage of sequence identity. The inventions disclosed herein encompass nucleotide sequences substantially identical to those disclosed herein.

As used herein, the terms “treat,” “treatment,” and the like, mean the methods or steps taken to provide relief from or alleviation of the number, severity, and/or frequency of one or more symptoms of a disease in a subject. As used herein, “treat” and “treatment” may include the prevention, management, prophylactic treatment, and/or inhibition or reduction of the number, severity, and/or frequency of one or more symptoms of a disease in a subject.

As used herein, the phrase “introducing into a cell,” when referring to an RNAi agent, means functionally delivering the RNAi agent into a cell. The phrase “functional delivery,” means delivering the RNAi agent to the cell in a manner that enables the RNAi agent to have the expected biological activity, e.g., sequence-specific inhibition of gene or viral genome expression.

Unless stated otherwise, use of the symbol

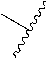

as used herein means that any group or groups may be linked thereto that is in accordance with the scope of the inventions described herein.

As used herein, the term “isomers” refers to compounds that have identical molecular formulae, but that differ in the nature or the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images are termed “enantiomers,” or sometimes optical isomers. A carbon atom bonded to four non-identical substituents is termed a “chiral center.”

As used herein, unless specifically identified in a structure as having a particular conformation, for each structure in which asymmetric centers are present and thus give rise to enantiomers, diastereomers, or other stereoisomeric configurations, each structure disclosed herein is intended to represent all such possible isomers, including their optically pure and racemic forms. For example, the structures disclosed herein are intended to cover mixtures of diastereomers as well as single stereoisomers.

As used in a claim herein, the phrase “consisting of” excludes any element, step, or ingredient not specified in the claim. When used in a claim herein, the phrase “consisting essentially of” limits the scope of a claim to the specified materials or steps and those that do not materially affect the basic and novel characteristic(s) of the claimed invention.

The person of ordinary skill in the art would readily understand and appreciate that the compounds and compositions disclosed herein may have certain atoms (e.g., N, O, or S atoms) in a protonated or deprotonated state, depending upon the environment in which the compound or composition is placed. Accordingly, as used herein, the structures disclosed herein envisage that certain functional groups, such as, for example, OH, SH, or NH, may be protonated or deprotonated. The disclosure herein is intended to cover the disclosed compounds and compositions regardless of their state of protonation based on the environment (such as pH), as would be readily understood by the person of ordinary skill in the art. Correspondingly, compounds described herein with labile protons or basic atoms should also be understood to represent salt forms of the corresponding compound. Compounds described herein may be in a free acid, free base, or salt form. Pharmaceutically acceptable salts of the compounds described herein should be understood to be within the scope of the invention.

As used herein, the term “linked” or “conjugated” when referring to the connection between two compounds or molecules means that two compounds or molecules are joined by a covalent bond. Unless stated, the terms “linked” and “conjugated” as used herein may refer to the connection between a first compound and a second compound either with or without any intervening atoms or groups of atoms.

As used herein, the term “including” is used to herein mean, and is used interchangeably with, the phrase “including but not limited to.” The term “or” is used herein to mean, and is used interchangeably with, the term “and/or,” unless the context clearly indicates otherwise.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other objects, features, aspects, and advantages of the invention will be apparent from the following detailed description, accompanying figures, and from the claims.

### BRIEF DESCRIPTION OF THE DRAWINGS

FIG. 1. Chemical structure representation of the tridentate αvβ6 epithelial cell targeting ligand referred to herein as Tri-SM6.1-αvβ6-(TA14).

FIG. 2. Immunohistochemistry (IHC) staining for anti-hemagglutinin (anti-HA) of mice administered with saline (no IAV RNAi agent) and with RNAi agent AC002564, as more fully described in Example 4.

FIG. 3. Bar graph showing lung viral load of CA07 H1N1 of mice infected with PBS or CA07, and subsequently administered saline, IAV RNAi agents, or Oseltamivir, as more fully described in Example 32.

FIG. 4. Bar graph showing percent of total inflammation of mice lungs infected with PBS or CA07, and subsequently administered saline, IAV RNAi agents, or Oseltamivir, as more fully described in Example 32.

FIG. 5. Histology of inflammation of mice lungs infected with PBS or CA07, and subsequently administered saline, IAV RNAi agents, or Oseltamivir, as more fully described in Example 32.

FIG. 6A. Lung viral load of mice test animals dosed with IAV RNAi agents, prior to and after infection with H5N1, as more fully described in Example 36.

FIG. 6B. Body weight of mice test animals dosed with IAV RNAi agents, prior to and after infection with H5N1, as more fully described in Example 36.

FIG. 6C. Clinical scores of mice test animals dosed with IAV RNAi agents, prior to and after infection with H5N1, as more fully described in Example 36.

FIG. 7A. Body weight of mice test animals dosed with IAV RNAi agents, prior to infection with H5N1, as more fully described in Example 37.

FIG. 7B. Body weight of mice test animals dosed with IAV RNAi agents, after infection with H5N1, as more fully described in Example 37.

FIG. 7C. Clinical scores of mice test animals dosed with IAV RNAi agents, prior to and after infection with H5N1, as more fully described in Example 37.

FIG. 7D. Survival index of the test animals treated IAV RNAi agents, prior to and after infection with H5N1, as more fully described in Example 37.

FIG. 7E. Lung viral load of mice test animals dosed with IAV RNAi agents, prior to and after infection with H5N1, as more fully described in Example 37.

FIG. 8. Lung weight and lung weight of total body weight, of test animals administered IAV RNAi agents, as more fully described in Example 38.

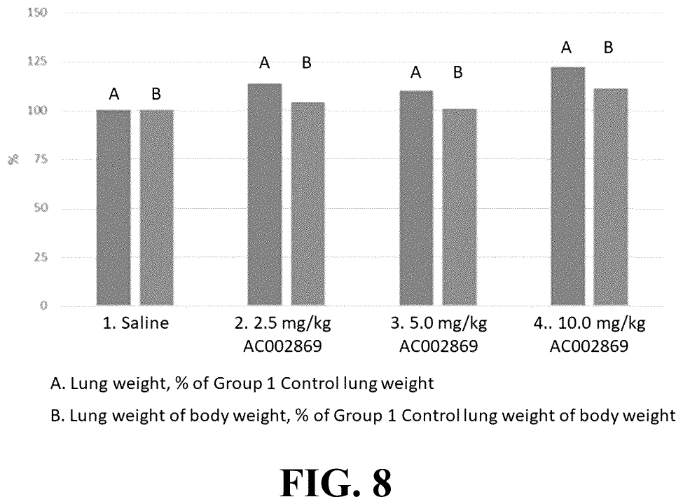

### DETAILED DESCRIPTION

### RNAi Agents

Described herein are RNAi agents for inhibiting expression of an influenza A viral gene transcript or genome (referred to herein as IAV RNAi agents or IAV RNAi triggers). Each IAV RNAi agent disclosed herein comprises a sense strand and an antisense strand. The sense strand can be 15 to 49 nucleotides in length, and the antisense strand can be 18 to 49 nucleotides in length. The sense and antisense strands can be either the same length or they can be different lengths. In some embodiments, the sense and antisense strands are each independently 18 to 27 nucleotides in length. In some embodiments, both the sense and antisense strands are each 19-26 nucleotides in length. In some embodiments, the sense and antisense strands are each 21-24 nucleotides in length. In some embodiments, the sense and antisense strands are each independently 19-21 nucleotides in length. In some embodiments, the sense strand is about 19 nucleotides in length while the antisense strand is about 21 nucleotides in length. In some embodiments, the sense strand is about 21 nucleotides in length while the antisense strand is about 23 nucleotides in length. In some embodiments, a sense strand is 23 nucleotides in length and an antisense strand is 21 nucleotides in length. In some embodiments, both the sense and antisense strands are each 21 nucleotides in length. In some embodiments, the RNAi agent sense strands are 15, 16, 17, 18, 19, 20, 21, 22, 23, 24, 25, 26, 27, 28, 29, 30, 31, 32, 33, 34, 35, 36, 37, 38, or 39 nucleotides in length. In some embodiments, the RNAi agent antisense strands are 18, 19, 20, 21, 22, 23, 24, 25, 26, 27, 28, 29, 30, 31, 32, 33, 34, 35, 36, 37, 38, or 39 nucleotides in length. In some embodiments, a double-stranded RNAi agent has a duplex length of about 16, 17, 18, 19, 20, 21, 22, 23 or 24 nucleotides.

Examples of nucleotide sequences used in forming IAV RNAi agents are provided in Tables 2A, 2B, 2C, 2D, 2E, 2F, 3A, 3B, 3C, 3D, 3E, 3F, 4A, 4B, 4C, 4D, 4E, 4F, 5A, 5B, 5C, 5D, 5E, 5F, 6A, 6B, 6C, 6D, 6E, 6F, 10A, 10B, 10C, 10D, 10E, and 10F. Examples of RNAi agent duplexes, that include the sense strand and antisense strand sequences in Tables 2A, 2B, 2C, 2D, 2E, 2F, 3A, 3B, 3C, 3D, 3E, 3F, 4A, 4B, 4C, 4D, 4E, 4F, 5A, 5B, 5C, 5D, 5E, 5F, 6A, 6B, 6C, 6D, 6E, and 6F are shown in Tables 7A-1, 7A-2, 7A-3, 7A-4, 7A-5, 7A-6, 7B-1, 7B-2, 7B-3, 7B-4, 7B-5, 7B-6, 8A, 8B, 8C, 8D, 8E, 8F, 9A, 9B, 9C, 9D, 9E, 9F, and 10A, 10B, 10C, 10D, 10E, and 10F.

In some embodiments, the region of perfect, substantial, or partial complementarity between the sense strand and the antisense strand is 15-26 (e.g., 15, 16, 17, 18, 19, 20, 21, 22, 23, 24, 25, or 26) nucleotides in length and occurs at or near the 5′ end of the antisense strand (e.g., this region may be separated from the 5′ end of the antisense strand by 0, 1, 2, 3, or 4 nucleotides that are not perfectly, substantially, or partially complementary).

A sense strand of the IAV RNAi agents described herein includes at least 15 consecutive nucleotides that have at least 85% identity to a core stretch sequence (also referred to herein as a “core stretch” or “core sequence”) of the same number of nucleotides in an influenza A viral genome RNA (including all viral RNA as well as viral mRNA). In some embodiments, a sense strand core stretch sequence is 100% (perfectly) complementary or at least about 85% (substantially) complementary to a core stretch sequence in the antisense strand, and thus the sense strand core stretch sequence is typically perfectly identical or at least about 85% identical to a nucleotide sequence of the same length (sometimes referred to, e.g., as a target sequence) present in the influenza A viral genome RNA target, which as noted elsewhere is a target sequence that is known to be conserved across a variety of influenza A viral genomes. In some embodiments, this sense strand core stretch is 15, 16, 17, 18, 19, 20, 21, 22, or 23 nucleotides in length. In some embodiments, this sense strand core stretch is 17 nucleotides in length. In some embodiments, this sense strand core stretch is 19 nucleotides in length.

An antisense strand of an IAV RNAi agent described herein includes at least 17 consecutive nucleotides that have at least 85% complementarity to a core stretch of the same number of nucleotides in an influenza A viral genome RNA or another influenza RNA being targeted, and at least 15 consecutive nucleotides that have at least 85% complementarity to a core stretch of the same number of nucleotides to a core stretch of the same number of nucleotides in the corresponding sense strand. In some embodiments, an antisense strand core stretch is 100% (perfectly) complementary or at least about 85% (substantially) complementary to a nucleotide sequence (e.g., target sequence) of the same length present in an influenza A viral genome RNA target. In some embodiments, this antisense strand core stretch is 15, 16, 17, 18, 19, 20, 21, 22, or 23 nucleotides in length. In some embodiments, this antisense strand core stretch is 19 nucleotides in length. In some embodiments, this antisense strand core stretch is 17 nucleotides in length. A sense strand core stretch sequence can be the same length as a corresponding antisense core sequence or it can be a different length.

The IAV RNAi agent sense and antisense strands anneal to form a duplex. A sense strand and an antisense strand of an IAV RNAi agent can be partially, substantially, or fully complementary to each other. Within the complementary duplex region, the sense strand core stretch sequence is at least 85% complementary or 100% complementary to the antisense core stretch sequence. In some embodiments, the sense strand core stretch sequence contains a sequence of at least 15, at least 16, at least 17, at least 18, at least 19, at least 20, at least 21, at least 22, or at least 23 nucleotides that is at least 85% or 100% complementary to a corresponding 15, 16, 17, 18, 19, 20, 21, 22, or 23 nucleotide sequence of the antisense strand core stretch sequence (i.e., the sense and antisense core stretch sequences of an IAV RNAi agent have a region of at least 15, at least 16, at least 17, at least 18, at least 19, at least 20, at least 21, at least 22, or at least 23 nucleotides that is at least 85% base paired or 100% base paired.)

In some embodiments, the antisense strand of an IAV RNAi agent disclosed herein differs by 0, 1, 2, or 3 nucleotides from any of the antisense strand sequences in Table 2A, 2B, 2C, 2D, 2E, 2F, 3A, 3B, 3C, 3D, 3E, or 3F. In some embodiments, the sense strand of an IAV RNAi agent disclosed herein differs by 0, 1, 2, or 3 nucleotides from any of the sense strand sequences in Table 2A, 2B, 2C, 2D, 2E, 2F, 4A, 4B, 4C, 4D, 4E, 4F, 5A, 5B, 5C, 5D, 5E, 5F, 6A, 6B, 6C, 6D, 6E, 6F, 10A, 10B, 10C, 10D, 10E, or 10F.

In some embodiments, the sense strand and/or the antisense strand can optionally and independently contain an additional 1, 2, 3, 4, 5, or 6 nucleotides (extension) at the 3′ end, the 5′ end, or both the 3′ and 5′ ends of the core stretch sequences. The antisense strand additional nucleotides, if present, may or may not be complementary to the corresponding sequence in an influenza A viral genome RNA. The sense strand additional nucleotides, if present, may or may not be identical to the corresponding sequence in an influenza A viral genome RNA. The antisense strand additional nucleotides, if present, may or may not be complementary to the corresponding sense strand's additional nucleotides, if present.

As used herein, an extension comprises 1, 2, 3, 4, 5, or 6 nucleotides at the 5′ and/or 3′ end of the sense strand core stretch sequence and/or antisense strand core stretch sequence. The extension nucleotides on a sense strand may or may not be complementary to nucleotides, either core stretch sequence nucleotides or extension nucleotides, in the corresponding antisense strand. Conversely, the extension nucleotides on an antisense strand may or may not be complementary to nucleotides, either core stretch nucleotides or extension nucleotides, in the corresponding sense strand. In some embodiments, both the sense strand and the antisense strand of an RNAi agent contain 3′ and 5′ extensions. In some embodiments, one or more of the 3′ extension nucleotides of one strand base pairs with one or more 5′ extension nucleotides of the other strand. In other embodiments, one or more of 3′ extension nucleotides of one strand do not base pair with one or more 5′ extension nucleotides of the other strand. In some embodiments, an IAV RNAi agent has an antisense strand having a 3′ extension and a sense strand having a 5′ extension. In some embodiments, the extension nucleotide(s) are unpaired and form an overhang. As used herein, an “overhang” refers to a stretch of one or more unpaired nucleotides located at a terminal end of either the sense strand or the antisense strand that does not form part of the hybridized or duplexed portion of an RNAi agent disclosed herein.

In some embodiments, an IAV RNAi agent comprises an antisense strand having a 3′ extension of 1, 2, 3, 4, 5, or 6 nucleotides in length. In other embodiments, an IAV RNAi agent comprises an antisense strand having a 3′ extension of 1, 2, or 3 nucleotides in length. In some embodiments, one or more of the antisense strand extension nucleotides comprise nucleotides that are complementary to the corresponding Influenza A viral genome RNA sequence. In some embodiments, one or more of the antisense strand extension nucleotides comprise nucleotides that are not complementary to the corresponding Influenza A viral genome RNA sequence.

In some embodiments, an IAV RNAi agent comprises a sense strand having a 3′ extension of 1, 2, 3, 4, or 5 nucleotides in length. In some embodiments, one or more of the sense strand extension nucleotides comprises adenosine, uracil, or thymidine nucleotides, AT dinucleotide, or nucleotides that correspond to or are the identical to nucleotides in an influenza A viral genome RNA sequence. In some embodiments, the 3′ sense strand extension includes or consists of one of the following sequences, but is not limited to: T, UT, TT, UU, UUT, TTT, or TTTT (each listed 5′ to 3′).

A sense strand can have a 3′ extension and/or a 5′ extension. In some embodiments, an IAV RNAi agent comprises a sense strand having a 5′ extension of 1, 2, 3, 4, 5, or 6 nucleotides in length. In some embodiments, one or more of the sense strand extension nucleotides comprise nucleotides that correspond to or are identical to nucleotides in an influenza A viral genome RNA sequence.

Examples of sequences used in forming IAV RNAi agents are provided in Tables 2A, 2B, 2C, 2D, 2E, 2F, 3A, 3B, 3C, 3D, 3E, 3F, 4A, 4B, 4C, 4D, 4E, 4F, 5A, 5B, 5C, 5D, 5E, 5F, 6A, 6B, 6C, 6D, 6E, 6F, 10A, 10B, 10C, 10D, 10E, and 10F. In some embodiments, an IAV RNAi agent antisense strand includes a sequence of any of the sequences in Tables 2A, 2B, 2C, 2D, 2E, 2F, 3A, 3B, 3C, 3D, 3E, 3F, 10A, 10B, 10C, 10D, 10E, or 10F. In certain embodiments, an IAV RNAi agent antisense strand comprises or consists of any one of the modified sequences in Table 3A, 3B, 3C, 3D, 3E, or 3F. In some embodiments, an IAV RNAi agent antisense strand includes the sequence of nucleotides (from 5′ end→3′ end) 1-17, 2-15, 2-17, 1-18, 2-18, 1-19, 2-19, 1-20, 2-20, 1-21, or 2-21, of any of the sequences in Tables 2A, 2B, 2C, 2D, 2E, 2F, 3A, 3B, 3C, 3D, 3E, or 3F. In some embodiments, an IAV RNAi agent sense strand includes the sequence of any of the sequences in Tables 2A, 2B, 2C, 2D, 2E, 2F, 4A, 4B, 4C, 4D, 4E, 4F, 5A, 5B, 5C, 5D, 5E, 5F, 6A, 6B, 6C, 6D, 6E, or 6F. In some embodiments, an IAV RNAi agent sense strand includes the sequence of nucleotides (from 5′ end→3′ end) 1-18, 1-19, 1-20, 1-21, 2-19, 2-20, 2-21, 3-20, 3-21, or 4-21 of any of the sequences in Tables 2A, 2B, 2C, 2D, 2E, 2F, 4A, 4B, 4C, 4D, 4E, 4F, 5A, 5B, 5C, 5D, 5E, 5F, 6A, 6B, 6C, 6D, 6E, or 6F. In certain embodiments, an IAV RNAi agent sense strand comprises or consists of a modified sequence of any one of the modified sequences in Table 4A, 4B, 4C, 4D, 4E, 4F, 5A, 5B, 5C, 5D, 5E, 5F, 6A, 6B, 6C, 6D, 6E, 6F, 10A, 10B, 10C, 10D, 10E, or 10F.

In some embodiments, the sense and antisense strands of the RNAi agents described herein contain the same number of nucleotides. In some embodiments, the sense and antisense strands of the RNAi agents described herein contain different numbers of nucleotides. In some embodiments, the sense strand 5′ end and the antisense strand 3′ end of an RNAi agent form a blunt end. In some embodiments, the sense strand 3′ end and the antisense strand 5′ end of an RNAi agent form a blunt end. In some embodiments, both ends of an RNAi agent form blunt ends. In some embodiments, neither end of an RNAi agent is blunt-ended. As used herein a “blunt end” refers to an end of a double stranded RNAi agent in which the terminal nucleotides of the two annealed strands are complementary (form a complementary base-pair).

In some embodiments, the sense strand 5′ end and the antisense strand 3′ end of an RNAi agent form a frayed end. In some embodiments, the sense strand 3′ end and the antisense strand 5′ end of an RNAi agent form a frayed end. In some embodiments, both ends of an RNAi agent form a frayed end. In some embodiments, neither end of an RNAi agent is a frayed end. As used herein a frayed end refers to an end of a double stranded RNAi agent in which the terminal nucleotides of the two annealed strands form a pair (i.e., do not form an overhang) but are not complementary (i.e. form a non-complementary pair). In some embodiments, one or more unpaired nucleotides at the end of one strand of a double stranded RNAi agent form an overhang. The unpaired nucleotides may be on the sense strand or the antisense strand, creating either 3′ or 5′ overhangs. In some embodiments, the RNAi agent contains: a blunt end and a frayed end, a blunt end and 5′ overhang end, a blunt end and a 3′ overhang end, a frayed end and a 5′ overhang end, a frayed end and a 3′ overhang end, two 5′ overhang ends, two 3′ overhang ends, a 5′ overhang end and a 3′ overhang end, two frayed ends, or two blunt ends. Typically, when present, overhangs are located at the 3′ terminal ends of the sense strand, the antisense strand, or both the sense strand and the antisense strand.

The IAV RNAi agents disclosed herein may also be comprised of one or more modified nucleotides. In some embodiments, substantially all of the nucleotides of the sense strand and substantially all of the nucleotides of the antisense strand of the IAV RNAi agent are modified nucleotides. The IAV RNAi agents disclosed herein may further be comprised of one or more modified internucleoside linkages, e.g., one or more phosphorothioate or phosphorodithioate linkages. In some embodiments, an IAV RNAi agent contains one or more modified nucleotides and one or more modified internucleoside linkages. In some embodiments, a 2′-modified nucleotide is combined with modified internucleoside linkage.

In some embodiments, an IAV RNAi agent is prepared or provided as a salt, mixed salt, or a free-acid. In some embodiments, an IAV RNAi agent is prepared as a pharmaceutically acceptable salt. In some embodiments, an IAV RNAi agent is prepared as a pharmaceutically acceptable sodium salt. Such forms that are well known in the art are within the scope of the inventions disclosed herein.

### Modified Nucleotides

Modified nucleotides, when used in various oligonucleotide constructs, can preserve activity of the compound in cells while at the same time increasing the serum stability of these compounds, and can also minimize the possibility of activating interferon activity in humans upon administration of the oligonucleotide construct.

In some embodiments, an IAV RNAi agent contains one or more modified nucleotides. As used herein, a “modified nucleotide” is a nucleotide other than a ribonucleotide (2′-hydroxyl nucleotide). In some embodiments, at least 50% (e.g., at least 60%, at least 70%, at least 80%, at least 90%, at least 95%, at least 97%, at least 98%, at least 99%, or 100%) of the nucleotides are modified nucleotides. As used herein, modified nucleotides can include, but are not limited to, deoxyribonucleotides, nucleotide mimics, abasic nucleotides, 2′-modified nucleotides, inverted nucleotides, modified nucleobase-comprising nucleotides, bridged nucleotides, peptide nucleic acids (PNAs), 2′,3′-seco nucleotide mimics (unlocked nucleobase analogues), locked nucleotides, 3′-O-methoxy (2′ internucleoside linked) nucleotides, 2′-F-Arabino nucleotides, 5′-Me, 2′-fluoro nucleotide, morpholino nucleotides, vinyl phosphonate deoxyribonucleotides, vinyl phosphonate-containing nucleotides, and cyclopropyl phosphonate-containing nucleotides. 2′-modified nucleotides (i.e., a nucleotide with a group other than a hydroxyl group at the 2′ position of the five-membered sugar ring) include, but are not limited to, 2′-O-methyl nucleotides (also referred to as 2′-methoxy nucleotides), 2′-fluoro nucleotides (also referred to herein as 2′-deoxy-2′-fluoro nucleotides), 2′-deoxy nucleotides, 2′-methoxyethyl (2′-O-2-methoxylethyl) nucleotides (also referred to as 2′-MOE), 2′-amino nucleotides, and 2′-alkyl nucleotides. It is not necessary for all positions in a given compound to be uniformly modified. Conversely, more than one modification can be incorporated in a single IAV RNAi agent or even in a single nucleotide thereof. The IAV RNAi agent sense strands and antisense strands can be synthesized and/or modified by methods known in the art. Modification at one nucleotide is independent of modification at another nucleotide.

Modified nucleobases include synthetic and natural nucleobases, such as 5-substituted pyrimidines, 6-azapyrimidines and N-2, N-6 and O-6 substituted purines, (e.g., 2-aminopropyladenine, 5-propynyluracil, or 5-propynylcytosine), 5-methylcytosine (5-me-C), 5-hydroxymethyl cytosine, inosine, xanthine, hypoxanthine, 2-aminoadenine, 6-alkyl (e.g., 6-methyl, 6-ethyl, 6-isopropyl, or 6-n-butyl) derivatives of adenine and guanine, 2-alkyl (e.g., 2-methyl, 2-ethyl, 2-isopropyl, or 2-n-butyl) and other alkyl derivatives of adenine and guanine, 2-thiouracil, 2-thiothymine, 2-thiocytosine, 5-halouracil, cytosine, 5-propynyl uracil, 5-propynyl cytosine, 6-azo uracil, 6-azo cytosine, 6-azo thymine, 5-uracil (pseudouracil), 4-thiouracil, 8-halo, 8-amino, 8-sulflydryl, 8-thioalkyl, 8-hydroxyl and other 8-substituted adenines and guanines, 5-halo (e.g., 5-bromo), 5-trifluoromethyl, and other 5-substituted uracils and cytosines, 7-methylguanine and 7-methyladenine, 8-azaguanine and 8-azaadenine, 7-deazaguanine, 7-deazaadenine, 3-deazaguanine, and 3-deazaadenine.

In some embodiments, the 5′ and/or 3′ end of the antisense strand can include abasic residues (Ab), which can also be referred to as an “abasic site” or “abasic nucleotide.” An abasic residue (Ab) is a nucleotide or nucleoside that lacks a nucleobase at the 1′ position of the sugar moiety. (See, e.g., U.S. Pat. No. 5,998,203). In some embodiments, an abasic residue can be placed internally in a nucleotide sequence. In some embodiments, Ab or AbAb can be added to the 3′ end of the antisense strand. In some embodiments, the 5′ end of the sense strand can include one or more additional abasic residues (e.g., (Ab) or (AbAb)). In some embodiments, UUAb, UAb, or Ab are added to the 3′ end of the sense strand. In some embodiments, an abasic (deoxyribose) residue can be replaced with a ribitol (abasic ribose) residue.

In some embodiments, all or substantially all of the nucleotides of an RNAi agent are modified nucleotides. As used herein, an RNAi agent wherein substantially all of the nucleotides present are modified nucleotides is an RNAi agent having four or fewer (i.e., 0, 1, 2, 3, or 4) nucleotides in both the sense strand and the antisense strand being ribonucleotides (i.e., unmodified). As used herein, a sense strand wherein substantially all of the nucleotides present are modified nucleotides is a sense strand having two or fewer (i.e., 0, 1, or 2) nucleotides in the sense strand being unmodified ribonucleotides. As used herein, an antisense sense strand wherein substantially all of the nucleotides present are modified nucleotides is an antisense strand having two or fewer (i.e., 0, 1, or 2) nucleotides in the antisense strand being unmodified ribonucleotides. In some embodiments, one or more nucleotides of an RNAi agent is an unmodified ribonucleotide. Chemical structures for certain modified nucleotides are set forth in Table 11 herein.

### Modified Internucleoside Linkages

In some embodiments, one or more nucleotides of an IAV RNAi agent are linked by non-standard linkages or backbones (i.e., modified internucleoside linkages or modified backbones). Modified internucleoside linkages or backbones include, but are not limited to, phosphorothioate groups (represented herein as a lower case “s”), chiral phosphorothioates, thiophosphates, phosphorodithioates, phosphotriesters, aminoalkyl-phosphotriesters, alkyl phosphonates (e.g., methyl phosphonates or 3′-alkylene phosphonates), chiral phosphonates, phosphinates, phosphoramidates (e.g., 3′-amino phosphoramidate, aminoalkylphosphoramidates, or thionophosphoramidates), thionoalkyl-phosphonates, thionoalkylphosphotriesters, morpholino linkages, boranophosphates having normal 3′-5′ linkages, 2′-5′ linked analogs of boranophosphates, or boranophosphates having inverted polarity wherein the adjacent pairs of nucleoside units are linked 3′-5′ to 5′-3′ or 2′-5′ to 5′-2′. In some embodiments, a modified internucleoside linkage or backbone lacks a phosphorus atom. Modified internucleoside linkages lacking a phosphorus atom include, but are not limited to, short chain alkyl or cycloalkyl inter-sugar linkages, mixed heteroatom and alkyl or cycloalkyl inter-sugar linkages, or one or more short chain heteroatomic or heterocyclic inter-sugar linkages. In some embodiments, modified internucleoside backbones include, but are not limited to, siloxane backbones, sulfide backbones, sulfoxide backbones, sulfone backbones, formacetyl and thioformacetyl backbones, methylene formacetyl and thioformacetyl backbones, alkene-containing backbones, sulfamate backbones, methyleneimino and methylenehydrazino backbones, sulfonate and sulfonamide backbones, amide backbones, and other backbones having mixed N, O, S, and CH₂ components.

In some embodiments, a sense strand of an IAV RNAi agent can contain 1, 2, 3, 4, 5, or 6 phosphorothioate linkages, an antisense strand of an IAV RNAi agent can contain 1, 2, 3, 4, 5, or 6 phosphorothioate linkages, or both the sense strand and the antisense strand independently can contain 1, 2, 3, 4, 5, or 6 phosphorothioate linkages. In some embodiments, a sense strand of an IAV RNAi agent can contain 1, 2, 3, or 4 phosphorothioate linkages, an antisense strand of an IAV RNAi agent can contain 1, 2, 3, or 4 phosphorothioate linkages, or both the sense strand and the antisense strand independently can contain 1, 2, 3, or 4 phosphorothioate linkages.

In some embodiments, an IAV RNAi agent sense strand contains at least two phosphorothioate internucleoside linkages. In some embodiments, the phosphorothioate internucleoside linkages are between the nucleotides at positions 1-3 from the 3′ end of the sense strand. In some embodiments, one phosphorothioate internucleoside linkage is at the 5′ end of the sense strand nucleotide sequence, and another phosphorothioate linkage is at the 3′ end of the sense strand nucleotide sequence. In some embodiments, two phosphorothioate internucleoside linkage are located at the 5′ end of the sense strand, and another phosphorothioate linkage is at the 3′ end of the sense strand. In some embodiments, the sense strand does not include any phosphorothioate internucleoside linkages between the nucleotides, but contains one, two, or three phosphorothioate linkages between the terminal nucleotides on both the 5′ and 3′ ends and the optionally present inverted abasic residue terminal caps. In some embodiments, the targeting ligand is linked to the sense strand via a phosphorothioate linkage.

In some embodiments, an IAV RNAi agent antisense strand contains four phosphorothioate internucleoside linkages. In some embodiments, the four phosphorothioate internucleoside linkages are between the nucleotides at positions 1-3 from the 5′ end of the antisense strand and between the nucleotides at positions 19-21, 20-22, 21-23, 22-24, 23-25, or 24-26 from the 5′ end. In some embodiments, three phosphorothioate internucleoside linkages are located between positions 1-4 from the 5′ end of the antisense strand, and a fourth phosphorothioate internucleoside linkage is located between positions 20-21 from the 5′ end of the antisense strand. In some embodiments, an IAV RNAi agent contains at least three or four phosphorothioate internucleoside linkages in the antisense strand.

### Capping Residues or Moieties

In some embodiments, the sense strand may include one or more capping residues or moieties, sometimes referred to in the art as a “cap,” a “terminal cap,” or a “capping residue.” As used herein, a “capping residue” is a non-nucleotide compound or other moiety that can be incorporated at one or more termini of a nucleotide sequence of an RNAi agent disclosed herein. A capping residue can provide the RNAi agent, in some instances, with certain beneficial properties, such as, for example, protection against exonuclease degradation. In some embodiments, inverted abasic residues (invAb) (also referred to in the art as “inverted abasic sites”) are added as capping residues (see Table 11). (See, e.g., F. Czauderna, Nucleic Acids Res., 2003, 31(11), 2705-16). Capping residues are generally known in the art, and include, for example, inverted abasic residues as well as carbon chains such as a terminal C₃H₇ (propyl), C₆H₁₃ (hexyl), or C₁₂H₂₅ (dodecyl) groups. In some embodiments, a capping residue is present at either the 5′ terminal end, the 3′ terminal end, or both the 5′ and 3′ terminal ends of the sense strand. In some embodiments, the 5′ end and/or the 3′ end of the sense strand may include more than one inverted abasic deoxyribose moiety as a capping residue.

In some embodiments, one or more inverted abasic residues (invAb) are added to the 3′ end of the sense strand. In some embodiments, one or more inverted abasic residues (invAb) are added to the 5′ end of the sense strand. In some embodiments, one or more inverted abasic residues or inverted abasic sites are inserted between the targeting ligand and the nucleotide sequence of the sense strand of the RNAi agent. In some embodiments, the inclusion of one or more inverted abasic residues or inverted abasic sites at or near the terminal end or terminal ends of the sense strand of an RNAi agent allows for enhanced activity or other desired properties of an RNAi agent.

In some embodiments, one or more inverted abasic residues (invAb) are added to the 5′ end of the sense strand. In some embodiments, one or more inverted abasic residues can be inserted between the targeting ligand and the nucleotide sequence of the sense strand of the RNAi agent. The inverted abasic residues may be linked via phosphate, phosphorothioate (e.g., shown herein as (invAb)s)), or other internucleoside linkages. In some embodiments, the inclusion of one or more inverted abasic residues at or near the terminal end or terminal ends of the sense strand of an RNAi agent may allow for enhanced activity or other desired properties of an RNAi agent. In some embodiments, an inverted abasic (deoxyribose) residue can be replaced with an inverted ribitol (abasic ribose) residue. In some embodiments, the 3′ end of the antisense strand core stretch sequence, or the 3′ end of the antisense strand sequence, may include an inverted abasic residue. The chemical structures for inverted abasic deoxyribose residues are shown in Table 11 below.

### IAV RNAi Agents

The IAV RNAi agents disclosed herein are designed to target specific positions on an influenza A viral genome (e.g., SEQ ID NO:1 (NC_026431.1), and SEQ ID NOS: 2-6)), and these specific targeted positions were selected in part because they also had sequences conserved across various other influenza genomes. As defined herein, an antisense strand sequence is designed to target an influenza A viral genome viral genome at a given position on the genome when the 5′ terminal nucleobase of the antisense strand is aligned with a position that is 21 nucleotides downstream (towards the 3′ end) from the position on the genome when base pairing to the gene or viral genome. For example, as illustrated in Tables 1A, 1B, 1C, 1D, 1E, 1F, 2A, 2B, 2C, 2D, 2E, and 2F herein, an antisense strand sequence designed to target an influenza A viral genome at position 150 requires that when base pairing to the genome, the 5′ terminal nucleobase of the antisense strand is aligned with position 170 of an influenza A viral genome.

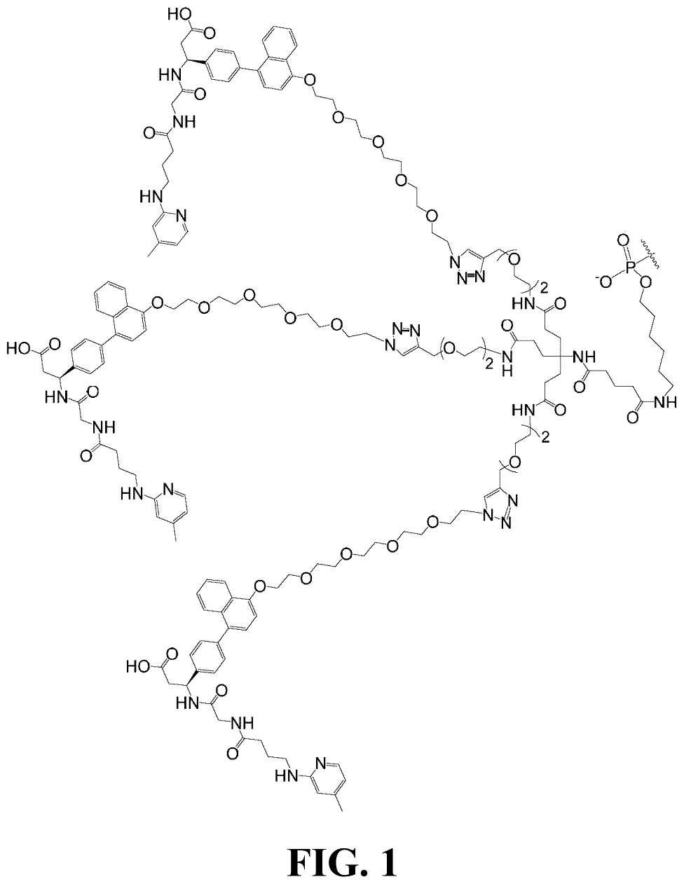

As provided herein, an IAV RNAi agent does not require that the nucleobase at position 1 (5′→3′) of the antisense strand be complementary to the viral genome, provided that there is at least 85% complementarity (e.g., at least 85, 86, 87, 88, 89, 90, 91, 92, 93, 94, 95, 96, 97, 98, 99, or 100% complementarity) of the antisense strand and the viral genome across a core stretch sequence of at least 17 consecutive nucleotides. For example, for an IAV RNAi agent disclosed herein that is designed to target position 150 of an influenza A viral genome viral genome, the 5′ terminal nucleobase of the antisense strand of the of the IAV RNAi agent must be aligned with position 170 of the respective genome; however, the 5′ terminal nucleobase of the antisense strand may be, but is not required to be, complementary to position 170 of an influenza A viral genome viral genome, provided that there is at least 85% complementarity (e.g., at least 85, 86, 87, 88, 89, 90, 91, 92, 93, 94, 95, 96, 97, 98, 99, or 100% complementarity) of the antisense strand and the viral genome transcript across a core stretch sequence of at least 17 consecutive nucleotides. As shown by, among other things, the various examples disclosed herein, the specific site of binding of the genome by the antisense strand of the IAV RNAi agent (e.g., whether the IAV RNAi agent is designed to target an influenza A viral genome viral genome at position 150, at position 429, at position 1217, or at some other position) is an important factor to the level of inhibition achieved by the IAV RNAi agent. (See, e.g., Kamola et al., The siRNA Non-seed Region and Its Target Sequences are Auxiliary Determinants of Off-Target Effects, PLOS Computational Biology, 11(12), FIG. 1 (2015)).

In some embodiments, the IAV RNAi agents disclosed herein target an influenza A viral genome viral genome at or near the positions of the respective influenza A viral genome sequences as shown in Table 1A, 1B, 1 C, 1D, 1E, and 1F. In some embodiments, the antisense strand of an IAV RNAi agent disclosed herein includes a core stretch sequence that is fully, substantially, or at least partially complementary to a target influenza A viral genome 19-mer sequence disclosed in Table 1A, 1B, 1C, 1D, 1E, and 1F.

TABLE 1A
Influenza A viral genome 19-mer Target Sequences (Targeting regions of
Influenza A virus (A/California/07/2009(H1N1)) segment 7 matrix protein 2
(M2) and matrix protein 1 (M1) genes (SEQ ID NO: 1))

| | Influenza A viral | | Targeted Viral |
|---|---|---|---|
| | genome 19-mer | Corresponding | Genome Position |
| SEQ ID | Target Sequences | Positions of Sequence | (as referred to |
| No. | (5′→3′) | on SEQ ID NO: 1 | herein) |
| 7 | AACCGAGGUCGAAACGUAC | 12-30 | 10 |
| 8 | GUACGUUCUUUCUAUCAUC | 27-45 | 25 |
| 9 | ACAAGACCAAUCUUGUCAC | 142-160 | 140 |
| 10 | AAUCUUGUCACCUCUGACU | 150-168 | 148 |
| 11 | UCUUGUCACCUCUGACUAA | 152-170 | 150 |
| 12 | CGAACAACAUGGAUAGAGC | 269-287 | 267 |
| 13 | CAACAUGGAUAGAGCAGUU | 273-291 | 271 |
| 14 | UCACUAAGCUAUUCAACUG | 346-364 | 344 |
| 15 | CAACUGGUGCACUUGCCAG | 359-377 | 357 |
| 16 | UGACCACAGAAGCUGCUUU | 413-431 | 411 |
| 17 | ACCAAUCCACUAAUCAGGC | 505-523 | 503 |
| 18 | CUAUUGGGACUCAUCCUAG | 653-671 | 651 |
| 19 | UGACGAUGGUCAUUUUGUC | 946-964 | 944 |
| 20 | GACGAUGGUCAUUUUGUCA | 947-965 | 945 |

TABLE 1B
Influenza A viral genome 19-mer Target Sequences (Targeting regions of
Influenza A virus (A/California/07/2009(H1N1)) segment 8 nuclear export
protein (NEP) and nonstructural protein 1 (NS1) genes (SEQ ID NO: 2))

| | Influenza A viral | | Targeted Viral |
|---|---|---|---|
| | genome 19-mer | Corresponding | Genome Position |
| SEQ ID | Target Sequences | Positions of Sequence | (as referred to |
| No. | (5′→3′) | on SEQ ID NO: 2 | herein) |
| 21 | GAUGCCCCAUUCCUUGAUC | 85-103 | 83 |
| 22 | AUGCCCCAUUCCUUGAUCG | 86-104 | 84 |
| 23 | UGCCCCAUUCCUUGAUCGG | 87-105 | 85 |
| 24 | GAAACAGCCACUCUUGUUG | 163-181 | 161 |
| 25 | AUCGUGGAAUGGAUCUUGA | 190-208 | 188 |
| 26 | UCUUGAAAGAGGAAUCCAG | 203-221 | 201 |
| 27 | GACAAUUGCAUCUGUACCU | 237-255 | 235 |
| 28 | GCAUCUGUACCUACUUCGC | 244-262 | 242 |
| 29 | GAGACUGGUUCAUGCUCAU | 299-317 | 297 |
| 30 | GACUGGUUCAUGCUCAUGC | 301-319 | 299 |
| 31 | UGAAAGCGAACUUCAGUGU | 389-407 | 387 |
| 32 | GAACUUCAGUGUAAUCUUU | 396-414 | 394 |
| 33 | CGAUUAGAGACCUUGAUAC | 418-436 | 416 |
| 34 | ACUACUAAGGGCUUUCACU | 435-453 | 433 |
| 35 | CUACUAAGGGCUUUCACUG | 436-454 | 434 |
| 36 | UCUCUUCCAGGACAUACUU | 493-511 | 491 |
| 37 | UGAGGAUGUCAAAAAUGCA | 513-531 | 511 |
| 38 | UGUCAAAAAUGCAGUUGGG | 519-537 | 517 |
| 39 | UGCAGUUGGGGUCCUCAUC | 528-546 | 526 |
| 40 | CAUCGGAGGACUUGAAUGG | 543-561 | 541 |
| 41 | AUCGGAGGACUUGAAUGGA | 544-562 | 542 |
| 42 | UCGGAGGACUUGAAUGGAA | 545-563 | 543 |
| 43 | GGAGGACUUGAAUGGAAUG | 547-565 | 545 |
| 44 | GAGGACUUGAAUGGAAUGG | 548-566 | 546 |
| 45 | UUGAAUGGAAUGGUAACAC | 554-572 | 552 |
| 46 | AUGGUAACACGGUUCGAGU | 563-581 | 561 |
| 47 | GUAACACGGUUCGAGUCUC | 566-584 | 564 |
| 48 | UACAGAGAUUCGCUUGGAG | 593-611 | 591 |
| 49 | GAUGAGAAUGGGAGACCUU | 619-637 | 617 |
| 50 | GAAUGGGAGACCUUCACUA | 624-642 | 622 |
| 51 | GACAGAGAAUAGUUUCGAA | 739-757 | 737 |
| 52 | UAUGCAAGCCUUACAACUA | 769-787 | 767 |
| 53 | AGAUAAGAGCUUUCUCGUU | 807-825 | 805 |
| 54 | CUUUCUCGUUUCAGCUUAU | 816-834 | 814 |

TABLE 1C
Influenza A viral genome 19-mer Target Sequences (Targeting regions of Influenza
A virus (A/California/07/2009(H1N1)) segment 2 polymerase PB1 (PB1) gene and
nonfunctional PB1-F2 protein (PB1-F2) gene (SEQ ID NO: 3))

| | | | Targeted Viral |
|---|---|---|---|
| | Influenza A viral genome 19-mer | Corresponding | Genome Position |
| SEQ ID | Target Sequences | Positions of Sequence | (as referred to |
| No. | (5′ → 3′) | on SEQ ID NO: 3 | herein) |
| 55 | GCCAUAAGCACCACAUUCC | 49-67 | 47 |
| 56 | ACCACAUUCCCUUAUACUG | 58-76 | 56 |
| 57 | ACAGAACACACCAAUACUC | 131-149 | 129 |
| 58 | AUGCCUUGAAACAAUGGAA | 318-336 | 316 |
| 59 | CCAACACCAUAGAAGUCUU | 431-449 | 429 |
| 60 | GUAAGAGACAACAUGACCA | 571-589 | 569 |
| 61 | GCAGAUUAGAGGUUUCGUA | 738-756 | 736 |
| 62 | GCAUUUGCGAAAAGCUUGA | 782-800 | 780 |
| 63 | CGAAAAGCUUGAACAGUCU | 789-807 | 787 |
| 64 | ACUGGGGACAACACUAAGU | 907-925 | 905 |
| 65 | CAAAAUGGCAAGACUAGGG | 1038-1056 | 1036 |
| 66 | AGUCCUGGGAUGAUGAUGG | 1210-1228 | 1208 |
| 67 | GGCAUGUUCAACAUGCUAA | 1228-1246 | 1226 |
| 68 | UCUCGAUACUGAAUCUUGG | 1262-1280 | 1260 |
| 69 | GGAGUGGACAGAUUCUACA | 1384-1402 | 1382 |
| 70 | AUUUGUGGCUAAUUUUAGC | 1500-1518 | 1498 |
| 71 | GUGAUAAAGAACAACAUGA | 1585-1603 | 1583 |
| 72 | GCUCUUCAAUUGUUCAUCA | 1639-1657 | 1637 |
| 73 | GAUGGAGGACCAAACUUAU | 1777-1795 | 1775 |
| 74 | AGGAUGAACAGAUGUACCA | 2051-2069 | 2049 |
| 75 | GGAAUUUCUAGCAUGGUGG | 2128-2146 | 2126 |
| 76 | UCAAGAAAGAAGAGUUCUC | 2204-2222 | 2202 |
| 77 | AGAUCAUGAAGAUCUGUUC | 2225-2243 | 2223 |
| 78 | AUCUGUUCCACCAUUGAAG | 2236-2254 | 2234 |
| 79 | CCACCAUUGAAGAACUCAG | 2243-2261 | 2241 |

TABLE 1D
Influenza A viral genome 19-mer Target Sequences (Targeting regions of Influenza
A virus (A/California/07/2009(H1N1)) segment 1 polymerase PB2 (PB2) gene (SEQ ID
NO: 4)

| | | | Targeted Viral |
|---|---|---|---|
| | Influenza A viral genome 19-mer | Corresponding | Genome Position |
| SEQ ID | Target Sequences | Positions of Sequence | (as referred to |
| No. | (5′ → 3′) | on SEQ ID NO: 4 | herein) |
| 80 | GAGAUACUCACUAAGACCA | 52-70 | 50 |
| 81 | UCGAAAGGUUGAAACAUGG | 365-383 | 363 |
| 82 | AUGGUGGCGUACAUGCUAG | 604-622 | 602 |
| 83 | GCUGCUAGAAACAUAGUAA | 784-802 | 782 |
| 84 | GACAAGCGGAUCAUCAGUC | 996-1014 | 994 |
| 85 | GAGGAUUGCAUGAUCAAGG | 1219-1237 | 1217 |
| 86 | GUGGUAGUGAGUAUUGACC | 1480-1498 | 1478 |
| 87 | AGGGAACGUACUAUUGUCU | 1524-1542 | 1522 |
| 88 | UGAUGUGGGAGAUCAAUGG | 1604-1622 | 1602 |
| 89 | UCGUAAGGACACUGUUCCA | 1784-1802 | 1782 |
| 90 | CAGUAUUCAAUUACAACAA | 1961-1979 | 1959 |
| 91 | GACUCUAGCAUACUUACUG | 2218-2236 | 2216 |

TABLE 1E
Influenza A viral genome 19-mer Target Sequences (Targeting regions of Influenza
A virus (A/California/07/2009(H1N1)) segment 5 nucleocapsid protein (NP) gene (SEQ ID
NO: 5))

| | | | Targeted Viral |
|---|---|---|---|
| | Influenza A viral genome 19-mer | Corresponding | Genome Position |
| SEQ ID | Target Sequences | Positions of Sequence | (as referred to |
| No. | (5′ → 3′) | on SEQ ID NO: 5 | herein) |
| 92 | GUCGGAAGAAUGAUUGGUG | 85-103 | 83 |
| 93 | GACUAAUCCAGAAUAGCAU | 164-182 | 162 |
| 94 | AGAGGAUGGUGCUUUCUGC | 191-209 | 189 |
| 95 | ACUCAUAUCAUGAUUUGGC | 400-418 | 398 |
| 96 | GAAUGAUGCCACAUAUCAG | 429-447 | 427 |
| 97 | UCUAAUGCAAGGUUCAACA | 495-513 | 493 |
| 98 | UCAGAAUGAUCAAACGUGG | 581-599 | 579 |
| 99 | CAAUGACCGAAAUUUCUGG | 603-621 | 601 |
| 100 | UGUGCAAUAUCCUCAAAGG | 665-683 | 663 |
| 101 | GGUCAGCACUCAUUCUGAG | 782-800 | 780 |
| 102 | AUCAGUUGCACAUAAAUCC | 804-822 | 802 |
| 103 | UCACAAGAGUCAAUUGGUG | 969-987 | 967 |
| 104 | GUAUCAAGUUUCAUAAGAG | 1027-1045 | 1025 |
| 105 | UGUGCAGCCUACAUUCUCA | 1221-1239 | 1219 |
| 106 | CUUCCUUUGACAUGAGUAA | 1430-1448 | 1428 |
| 107 | AAUGAAGGGUCUUAUUUCU | 1447-1465 | 1445 |

TABLE 1F
Influenza A viral genome 19-mer Target Sequences (Targeting regions of Influenza
A virus (A/California/07/2009(H1N1)) segment 3 polymerase PA (PA) gene (SEQ ID NO: 6))

| | | | Targeted Viral |
|---|---|---|---|
| | Influenza A viral genome 19-mer | Corresponding | Genome Position |
| SEQ ID | Target Sequences | Positions of Sequence | (as referred to |
| No. | (5′ → 3′) | on SEQ ID NO: 6 | herein) |
| 108 | CUUUGUGCGACAAUGCUUC | 9-27 | 7 |
| 109 | UAUUCGGAUUUCCAUUUCA | 142-160 | 140 |
| 110 | GGAAGAGACCGAAUCAUGG | 241-259 | 239 |
| 111 | AUCAAAACUAGGCUUUUCA | 511-529 | 509 |
| 112 | CUAUGGGAUUCCUUUCGUC | 559-577 | 557 |
| 113 | GGCGAAGAGACAAUUGAAG | 589-607 | 587 |
| 114 | CUAUGUAGAUGGAUUCGAG | 693-711 | 691 |
| 115 | CAAGCUUUCCCAAAUGUCA | 732-750 | 730 |
| 116 | ACUAUAUGAUGCAAUCAAA | 909-927 | 907 |
| 117 | CAAAGAUGUUGGAGACCUU | 1152-1170 | 1150 |
| 118 | GCUGGAUAGAACUUGAUGA | 1262-1280 | 1260 |
| 119 | CCCAAUGAUAAGCAAAUGU | 1449-1467 | 1447 |
| 120 | AGGAGUGCCUGAUUAAUGA | 2072-2090 | 2070 |

Influenza A virus (A/California/07/2009(H1N1) segment 7 matrix protein 2 (M2) and matrix protein 1 (M1) genes, complete cds (NC_026431.1) (SEQ ID NO:1), viral genome transcript (982 bases):

| | |
|---|---|
| 1 | atgagtcttc taaccgaggt cgaaacgtac gttctttcta |
| | tcatcccgtc aggccccctc |
| 61 | aaagccgaga tcgcgcagag actggaaagt gtctttgcag |
| | gaaagaacac agatcttgag |
| 121 | gctctcatgg aatggctaaa gacaagacca atcttgtcac |
| | ctctgactaa gggaatttta |
| 181 | ggatttgtgt tcacgctcac cgtgcccagt gagcgaggac |
| | tgcagcgtag acgctttgtc |
| 241 | caaaatgccc taaatgggaa tggggacccg aacaacatgg |
| | atagagcagt taaactatac |
| 301 | aagaagctca aaagagaaat aacgttccat ggggccaagg |
| | aggtgtcact aagctattca |
| 361 | actggtgcac ttgccagttg catgggcctc atatacaaca |
| | ggatgggaac agtgaccaca |
| 421 | gaagctgctt ttggtctagt gtgtgccact tgtgaacaga |
| | ttgctgattc acagcatcgg |
| 481 | tctcacagac agatggctac taccaccaat ccactaatca |
| | ggcatgaaaa cagaatggtg |
| 541 | ctggctagca ctacggcaaa ggctatggaa cagatggctg |
| | gatcgagtga acaggcagcg |
| 601 | gaggccatgg aggttgctaa tcagactagg cagatggtac |
| | atgcaatgag aactattggg |
| 661 | actcatccta gctccagtgc tggtctgaaa gatgaccttc |
| | ttgaaaattt gcaggcctac |
| 721 | cagaagcgaa tgggagtgca gatgcagcga ttcaagtgat |
| | cctctcgtca ttgcagcaaa |
| 781 | tatcattggg atcttgcacc tgatattgtg gattactgat |
| | cgtctttttt tcaaatgtat |
| 841 | ttatcgtcgc tttaaatacg gtttgaaaag agggccttct |
| | acggaaggag tgcctgagtc |
| 901 | catgagggaa gaatatcaac aggaacagca gagtgctgtg |
| | gatgttgacg atggtcattt |
| 961 | tgtcaacata gagctagagt aa |

While the influenza A M genomic segment includes both M1 and M2, as used herein, when referring to inhibiting expression of the influenza A M1 viral genomic segment it refers to an RNAi agent that targets the viral genome transcript anywhere in SEQ ID NO:1.

Influenza A virus (A/California/07/2009(H1N1)) segment 8 nuclear export protein (NEP) and nonstructural protein 1 (NS1) genes, complete cds (NC_026432.1) (SEQ ID NO:2), viral genome transcript (863 bases):

| | |
|---|---|
| 1 | atggactcca acaccatgtc aagctttcag gtagactgtt |
| | tcctttggca tatccgcaag |
| 61 | cgatttgcag acaatggatt gggtgatgcc ccattccttg |
| | atcggctccg ccgagatcaa |
| 121 | aagtccttaa aaggaagagg caacaccctt ggcctcgata |
| | tcgaaacagc cactcttgtt |
| 181 | gggaaacaaa tcgtggaatg gatcttgaaa gaggaatcca |
| | gcgagacact tagaatgaca |
| 241 | attgcatctg tacctacttc gcgctacctt tctgacatga |
| | ccctcgagga aatgtcacga |
| 301 | gactggttca tgctcatgcc taggcaaaag ataataggcc |
| | ctctttgcgt gcgattggac |
| 361 | caggcgatca tggaaaagaa catagtactg aaagcgaact |
| | tcagtgtaat ctttaaccga |
| 421 | ttagagacct tgatactact aagggctttc actgaggagg |
| | gagcaatagt tggagaaatt |
| 481 | tcaccattac cttctcttcc aggacatact tatgaggatg |
| | tcaaaaatgc agttggggtc |
| 541 | ctcatcggag gacttgaatg gaatggtaac acggttcgag |
| | tctctgaaaa tatacagaga |
| 601 | ttcgcttgga gaaactgtga tgagaatggg agaccttcac |
| | tacctccaga gcagaaatga |
| 661 | aaagtggcga gagcaattgg gacagaaatt tgaggaaata |
| | aggtggttaa ttgaagaaat |
| 721 | gcggcacaga ttgaaagcga cagagaatag tttcgaacaa |
| | ataacattta tgcaagcctt |
| 781 | acaactactg cttgaagtag aacaagagat aagagctttc |
| | tcgtttcagc ttatttaatg |
| 841 | ataaaaaaca cccttgtttc tac |

Influenza A virus (A/California/07/2009(H1N1)) segment 2 polymerase PB1 (PB1) gene, complete cds; and nonfunctional PB1-F2 protein (PB1-F2) gene, complete sequence (NC_026435.1) (SEQ ID NO:3), viral genome transcript (2274 bases):

| | |
|---|---|
| 1 | atggatgtca atccgactct acttttccta aaaattccag |
| | cgcaaaatgc cataagcacc |
| 61 | acattccctt atactggaga tcctccatac agccatggaa |
| | caggaacagg atacaccatg |
| 121 | gacacagtaa acagaacaca ccaatactca gaaaagggaa |
| | agtggacgac aaacacagag |
| 181 | actggtgcac cccagctcaa cccgattgat ggaccactac |
| | ctgaggataa tgaaccaagt |
| 241 | gggtatgcac aaacagactg tgttctagag gctatggctt |
| | tccttgaaga atcccaccca |
| 301 | ggaatatttg agaattcatg ccttgaaaca atggaagttg |
| | ttcaacaaac aagggtagat |
| 361 | aaactaactc aaggtcgcca gacttatgat tggacattaa |
| | acagaaatca accggcagca |
| 421 | actgcattgg ccaacaccat agaagtcttt agatcgaatg |
| | gcctaacagc taatgagtca |
| 481 | ggaaggctaa tagatttctt aaaggatgta atggaatcaa |
| | tgaacaaaga ggaaatagag |
| 541 | ataacaaccc actttcaaag aaaaaggaga gtaagagaca |
| | acatgaccaa gaagatggtc |
| 601 | acgcaaagaa caatagggaa gaaaaaacaa agactgaata |
| | agagaggcta tctaataaga |
| 661 | gcactgacat taaatacgat gaccaaagat gcagagagag |
| | gcaagttaaa aagaagggct |
| 721 | atcgcaacac ctgggatgca gattagaggt ttcgtatact |
| | ttgttgaaac tttagctagg |
| 781 | agcatttgcg aaaagcttga acagtctggg ctcccagtag |
| | ggggcaatga aaagaaggcc |
| 841 | aaactggcaa atgttgtgag aaagatgatg actaattcac |
| | aagacacaga gatttctttc |
| 901 | acaatcactg gggacaacac taagtggaat gaaaatcaaa |
| | atcctcgaat gttcctggcg |
| 961 | atgattacat atatcaccag aaatcaaccc gagtggttca |
| | gaaacatcct gagcatggca |
| 1021 | cccataatgt tctcaaacaa aatggcaaga ctagggaaag |
| | ggtacatgtt cgagagtaaa |
| 1081 | agaatgaaga ttcgaacaca aataccagca gaaatgctag |
| | caagcattga cctgaagtac |
| 1141 | ttcaatgaat caacaaagaa gaaaattgag aaaataaggc |
| | ctcttctaat agatggcaca |
| 1201 | gcatcactga gtcctgggat gatgatgggc atgttcaaca |
| | tgctaagtac ggtcttggga |
| 1261 | gtctcgatac tgaatcttgg acaaaagaaa tacaccaaga |
| | caatatactg gtgggatggg |
| 1321 | ctccaatcat ccgacgattt tgctctcata gtgaatgcac |
| | caaaccatga gggaatacaa |
| 1381 | gcaggagtgg acagattcta caggacctgc aagttagtgg |
| | gaatcaacat gagcaaaaag |
| 1441 | aagtcctata taaataagac agggacattt gaattcacaa |
| | gcttttttta tcgctatgga |
| 1501 | tttgtggcta attttagcat ggagctaccc agctttggag |
| | tgtctggagt aaatgaatca |
| 1561 | gctgacatga gtattggagt aacagtgata aagaacaaca |
| | tgataaacaa tgaccttgga |
| 1621 | cctgcaacgg cccagatggc tcttcaattg ttcatcaaag |
| | actacagata cacatatagg |
| 1681 | tgccataggg gagacacaca aattcagacg agaagatcat |
| | ttgagttaaa gaagctgtgg |
| 1741 | gatcaaaccc aatcaaaggt agggctatta gtatcagatg |
| | gaggaccaaa cttatacaat |
| 1801 | atacggaatc ttcacattcc tgaagtctgc ttaaaatggg |
| | agctaatgga tgatgattat |
| 1861 | cggggaagac tttgtaatcc cctgaatccc tttgtcagtc |
| | ataaagagat tgattctgta |
| 1921 | aacaatgctg tggtaatgcc agcccatggt ccagccaaaa |
| | gcatggaata tgatgccgtt |
| 1981 | gcaactacac attcctggat tcccaagagg aatcgttcta |
| | ttctcaacac aagccaaagg |
| 2041 | ggaattcttg aggatgaaca gatgtaccag aagtgctgca |
| | atctattcga gaaatttttc |
| 2101 | cctagcagtt catataggag accggttgga atttctagca |
| | tggtggaggc catggtgtct |
| 2161 | agggcccgga ttgatgccag ggtcgacttc gagtctggac |
| | ggatcaagaa agaagagttc |
| 2221 | tctgagatca tgaagatctg ttccaccatt gaagaactca |
| | gacggcaaaa ataa |

Influenza A virus (A/California/07/2009(H1N1)) segment 1 polymerase PB2 (PB2) gene, complete cds (NC_026438.1) (SEQ ID NO:4), viral genome transcript (2280 bases):

| | |
|---|---|
| 1 | atggagagaa taaaagaact gagagatcta atgtcgcagt |
| | cccgcactcg cgagatactc |
| 61 | actaagacca ctgtggacca tatggccata atcaaaaagt |
| | acacatcagg aaggcaagag |
| 121 | aagaaccccg cactcagaat gaagtggatg atggcaatga |
| | gatacccaat tacagcagac |
| 181 | aagagaataa tggacatgat tccagagagg aatgaacaag |
| | gacaaaccct ctggagcaaa |
| 241 | acaaacgatg ctggatcaga ccgagtgatg gtatcacctc |
| | tggccgtaac atggtggaat |
| 301 | aggaatggcc caacaacaag tacagttcat taccctaagg |
| | tatataaaac ttatttcgaa |
| 361 | aaggtcgaaa ggttgaaaca tggtaccttc ggccctgtcc |
| | acttcagaaa tcaagttaaa |
| 421 | ataaggagga gagttgatac aaaccctggc catgcagatc |
| | tcagtgccaa ggaggcacag |
| 481 | gatgtgatta tggaagttgt tttcccaaat gaagtggggg |
| | caagaatact gacatcagag |
| 541 | tcacagctgg caataacaaa agagaagaaa gaagagctcc |
| | aggattgtaa aattgctccc |
| 601 | ttgatggtgg cgtacatgct agaaagagaa ttggtccgta |
| | aaacaaggtt tctcccagta |
| 661 | gccggcggaa caggcagtgt ttatattgaa gtgttgcact |
| | taacccaagg gacgtgctgg |
| 721 | gagcagatgt acactccagg aggagaagtg agaaatgatg |
| | atgttgacca aagtttgatt |
| 781 | atcgctgcta gaaacatagt aagaagagca gcagtgtcag |
| | cagacccatt agcatctctc |
| 841 | ttggaaatgt gccacagcac acagattgga ggagtaagga |
| | tggtggacat ccttagacag |
| 901 | aatccaactg aggaacaagc cgtagacata tgcaaggcag |
| | caatagggtt gaggattagc |
| 961 | tcatctttca gttttggtgg gttcactttc aaaaggacaa |
| | gcggatcatc agtcaagaaa |
| 1021 | gaagaagaag tgctaacggg caacctccaa acactgaaaa |
| | taagagtaca tgaagggtat |
| 1081 | gaagaattca caatggttgg gagaagagca acagctattc |
| | tcagaaaggc aaccaggaga |
| 1141 | ttgatccagt tgatagtaag cgggagagac gagcagtcaa |
| | ttgctgaggc aataattgtg |
| 1201 | gccatggtat tctcacagga ggattgcatg atcaaggcag |
| | ttaggggcga tctgaacttt |
| 1261 | gtcaataggg caaaccagcg actgaacccc atgcaccaac |
| | tcttgaggca tttccaaaaa |
| 1321 | gatgcaaaag tgcttttcca gaactgggga attgaatcca |
| | tcgacaatgt gatgggaatg |
| 1381 | atcggaatac tgcccgacat gaccccaagc acggagatgt |
| | cgctgagagg gataagagtc |
| 1441 | agcaaaatgg gagtagatga atactccagc acggagagag |
| | tggtagtgag tattgaccga |
| 1501 | tttttaaggg ttagagatca aagagggaac gtactattgt |
| | ctcccgaaga agtcagtgaa |
| 1561 | acgcaaggaa ctgagaagtt gacaataact tattcgtcat |
| | caatgatgtg ggagatcaat |
| 1621 | ggccctgagt cagtgctagt caacacttat caatggataa |
| | tcaggaactg ggaaattgtg |
| 1681 | aaaattcaat ggtcacaaga tcccacaatg ttatacaaca |
| | aaatggaatt tgaaccattt |
| 1741 | cagtctcttg tccctaaggc aaccagaagc cggtacagtg |
| | gattcgtaag gacactgttc |
| 1801 | cagcaaatgc gggatgtgct tgggacattt gacactgtcc |
| | aaataataaa acttctcccc |
| 1861 | tttgctgctg ccccaccaga acagagtagg atgcaatttt |
| | cctcattgac tgtgaatgtg |
| 1921 | agaggatcag ggttgaggat actggtaaga ggcaattctc |
| | cagtattcaa ttacaacaag |
| 1981 | gcaaccaaac gacttacagt tcttggaaag gatgcaggtg |
| | cattgactga agatccagat |
| 2041 | gaaggcacat ctggggtgga gtctgctgtc ctgagaggat |
| | ttctcatttt gggcaaagaa |
| 2101 | gacaagagat atggcccagc attaagcatc aatgaactga |
| | gcaatcttgc aaaaggagag |
| 2161 | aaggctaatg tgctaattgg gcaaggggac gtagtgttgg |
| | taatgaaacg aaaacgggac |
| 2221 | tctagcatac ttactgacag ccagacagcg accaaaagaa |
| | ttcggatggc catcaattag |

Influenza A virus (A/California/07/2009(H1N1)) segment 5 nucleocapsid protein (NP) gene, complete cds (NC_026436.1) (SEQ ID NO:5), viral genome transcript (1497 bases):

| | |
|---|---|
| 1 | atggcgtctc aaggcaccaa acgatcatat gaacaaatgg |
| | agactggtgg ggagcgccag |
| 61 | gatgccacag aaatcagagc atctgtcgga agaatgattg |
| | gtggaatcgg gagattctac |
| 121 | atccaaatgt gcactgaact caaactcagt gattatgatg |
| | gacgactaat ccagaatagc |
| 181 | ataacaatag agaggatggt gctttctgct tttgatgaga |
| | gaagaaataa atacctagaa |
| 241 | gagcatccca gtgctgggaa ggaccctaag aaaacaggag |
| | gacccatata tagaagagta |
| 301 | gacggaaagt ggatgagaga actcatcctt tatgacaaag |
| | ragaaataag gagagtttgg |
| 361 | cgcctagcaa acaatggcga agatgcaaca gcaggtctta |
| | ctcatatcat gatttggcat |
| 421 | tccaacctga atgatgccac atatcagaga acaagagcgc |
| | ttgttcgcac cggaatggat |
| 481 | cccagaatgt gctctctaat gcaaggttca acacttccca |
| | gaaggtctgg tgccgcaggt |
| 541 | gctgcggtga aaggagttgg aacaatagca atggagttaa |
| | tcagaatgat caaacgtgga |
| 601 | atcaatgacc gaaatttctg gaggggtgaa aatggacgaa |
| | ggacaagggt tgcttatgaa |
| 661 | agaatgtgca atatcctcaa aggaaaattt caaacagctg |
| | cccagagggc aatgatggat |
| 721 | caagtaagag aaagtcgaaa cccaggaaac gctgagattg |
| | aagacctcat tttcctggca |
| 781 | cggtcagcac tcattctgag gggatcagtt gcacataaat |
| | cctgcctgcc tgcttgtgtg |
| 841 | tatgggcttg cagtagcaag tgggcatgac tttgaaaggg |
| | aagggtactc actggtcggg |
| 901 | atagacccat tcaaattact ccaaaacagc caagtggtca |
| | gcctgatgag accaaatgaa |
| 961 | aacccagctc acaagagtca attggtgtgg atggcatgcc |
| | actctgctgc atttgaagat |
| 1021 | ttaagagtat caagtttcat aagaggaaag aaagtgattc |
| | caagaggaaa gctttccaca |
| 1081 | agaggggtcc agattgcttc aaatgagaat gtggaaacca |
| | tggactccaa taccctggaa |
| 1141 | ctgagaagca gatactgggc cataaggacc aggagtggag |
| | gaaataccaa tcaacaaaag |
| 1201 | gcatccgcag gccagatcag tgtgcagcct acattctcag |
| | tgcagcggaa tctccctttt |
| 1261 | gaaagagcaa ccgttatggc agcattcagc gggaacaatg |
| | aaggacggac atccgacatg |
| 1321 | cgaacagaag ttataagaat gatggaaagt gcaaagccag |
| | aagatttgtc cttccagggg |
| 1381 | cggggagtct tcgagctctc ggacgaaaag gcaacgaacc |
| | cgatcgtgcc ttcctttgac |
| 1441 | atgagtaatg aagggtctta tttcttcgga gacaatgcag |
| | aggagtatga cagttga |

Influenza A virus (A/California/07/2009(H1N1)) segment 3 polymerase PA (PA) gene, complete cds (NC 026437.1) (SEQ ID NO:6), viral genome transcript (2151 bases):

| | |
|---|---|
| 1 | atggaagact ttgtgcgaca atgcttcaat ccaatgatcg |
| | tcgagcttgc ggraaaggca |
| 61 | atgaaagaat atggggaaga tccgaaaatc gaaactaaca |
| | agtttgctgc aatatgcaca |
| 121 | catttggaag tttgtttcat gtattcggat ttccatttca |
| | tcgacgaacg gggtgaatca |
| 181 | ataattgtag aatctggtga cccgaatgca ctattgaagc |
| | accgatttga gataattgaa |
| 241 | ggaagagacc gaatcatggc ctggacagtg gtgaacagta |
| | tatgtaacac aacaggggta |
| 301 | gagaagccta aatttcttcc tgatttgtat gattacaaag |
| | agaaccggtt cattgaaatt |
| 361 | ggagtaacac ggagggaagt ccacatatat tacctagaga |
| | aagccaacaa aataaaatct |
| 421 | gagaagacac acattcacat cttttcattc actggagagg |
| | agatggccac caaagcggac |
| 481 | tacacccttg acgaagagag cagggcaaga atcaaaacta |
| | ggcttttcac tataagacaa |
| 541 | gaaatggcca gtaggagtct atgggattcc tttcgtcagt |
| | ccgaaagagg cgaagagaca |
| 601 | attgaagaaa aatttgagat tacaggaact atgcgcaagc |
| | ttgccgacca aagtctccca |
| 661 | ccgaacttcc ccagccttga aaactttaga gcctatgtag |
| | atggattcga gccgaacggc |
| 721 | tgcattgagg gcaagctttc ccaaatgtca aaagaagtga |
| | acgccaaaat tgaaccattc |
| 781 | ttgaggacga caccacgccc cctcagattg cctgatgggc |
| | ctctttgcca tcagcggtca |
| 841 | aagttcctgc tgatggatgc tctgaaatta agtattgaag |
| | acccgagtca cgagggggag |
| 901 | ggaataccac tatatgatgc aatcaaatgc atgaagacat |
| | tctttggctg gaaagagcct |
| 961 | aacatagtca aaccacatga gaaaggcata aatcccaatt |
| | acctcatggc ttggaagcag |
| 1021 | gtgctagcag agctacagga cattgaaaat gaagagaaga |
| | tcccaaggac aaagaacatg |
| 1081 | aagagaacaa gccaattgaa gtgggcactc ggtgaaaata |
| | tggcaccaga aaaagtagac |
| 1141 | tttgatgact gcaaagatgt tggagacctt aaacagtatg |
| | acagtgatga gccagagccc |
| 1201 | agatctctag caagctgggt ccaaaatgaa ttcaataagg |
| | catgtgaatt gactgattca |
| 1261 | agctggatag aacttgatga aataggagaa gatgttgccc |
| | cgattgaaca tatcgcaagc |
| 1321 | atgaggagga actattttac agcagaagtg tcccactgca |
| | gggctactga atacataatg |
| 1381 | aagggagtgt acataaatac ggccttgctc aatgcatcct |
| | gtgcagccat ggatgacttt |
| 1441 | cagctgatcc caatgataag caaatgtagg accaaagaag |
| | gaagacggaa aacaaacctg |
| 1501 | tatgggttca ttataaaagg aaggtctcat ttgagaaatg |
| | atactgatgt ggtgaacttt |
| 1561 | gtaagtatgg agttctcact cactgacccg agactggagc |
| | cacacaaatg ggaaaaatac |
| 1621 | tgtgttcttg aaataggaga catgctcttg aggactgcga |
| | taggccaagt gtcgaggccc |
| 1681 | atgttcctat atgtgagaac caatggaacc tccaagatca |
| | agatgaaatg gggcatggaa |
| 1741 | atgaggcgct gccttcttca gtctcttcag cagattgaga |
| | gcatgattga ggccgagtct |
| 1801 | tctgtcaaag agaaagacat gaccaaggaa ttctttgaaa |
| | acaaatcgga aacatggcca |
| 1861 | atcggagagt cacccagggg agtggaggaa ggctctattg |
| | ggaaagtgtg caggacctta |
| 1921 | ctggcaaaat ctgtattcaa cagtctatat gcgtctccac |
| | aacttgaggg gttttcggct |
| 1981 | gaatctagaa aattgcttct cattgttcag gcacttaggg |
| | acaacctgga acctggaacc |
| 2041 | ttcgatcttg gggggctata tgaagcaatc gaggagtgcc |
| | tgattaatga tccctgggtt |
| 2101 | ttgcttaatg catcttggtt caactccttc ctcacacatg |
| | cactgaagta g |

In some embodiments, an IAV RNAi agent includes an antisense strand wherein position 19 of the antisense strand (5′≥3′) is capable of forming abase pair with position 1 of a 19-mer target sequence disclosed in Table 1A, 1B, 1C, 1D, 1E, or 1F. In some embodiments, an IAV RNAi agent includes an antisense strand wherein position 1 of the antisense strand (5′→3′) is capable of forming abase pair with position 19 of a 19-mer target sequence disclosed in Table 1A, 1B, 1C, 1D, 1E, or 1F.

In some embodiments, an IAV RNAi agent includes an antisense strand wherein position 2 of the antisense strand (5′→3′) is capable of forming a base pair with position 18 of a 19-mer target sequence disclosed in Table 1A, 1B, 1C, 1D, 1E, or 1F. In some embodiments, an IAV RNAi agent includes an antisense strand wherein positions 2 through 18 of the antisense strand (5′→3′) are capable of forming base pairs with each of the respective complementary bases located at positions 18 through 2 of the 19-mer target sequence disclosed in Table 1A, 1B, 1C, 1D, 1E, or 1F.

For the RNAi agents disclosed herein, the nucleotide at position 1 of the antisense strand (from 5′ end→3′ end) can be perfectly complementary to an influenza A viral genome or can be non-complementary to an influenza A viral genome being targeted. In some embodiments, the nucleotide at position 1 of the antisense strand (from 5′ end→3′ end) is a U, A, or dT. In some embodiments, the nucleotide at position 1 of the antisense strand (from 5′ end→3′ end) forms an A:U or U:A base pair with the sense strand.

In some embodiments, an IAV RNAi agent antisense strand comprises the sequence of nucleotides (from 5′ end→3′ end) 2-18 or 2-19 of any of the antisense strand sequences in Tables 2A, 2B, 2C, 2D, 2E, 2F, 3A, 3B, 3C, 3D, 3E, or 3F. In some embodiments, an IAV RNAi agent sense strand comprises the sequence of nucleotides (from 5′ end→3′ end) 1-17, 1-18, or 2-18 of any of the sense strand sequences in Table 2A, 2B, 2C, 2D, 2E, 2F, 4A, 4B, 4C, 4D, 4E, 4F, 5A, 5B, 5C, 5D, 5E, 5F, 6A, 6B, 6C, 6D, 6E, or 6F.

In some embodiments, an IAV RNAi agent is comprised of (i) an antisense strand comprising the sequence of nucleotides (from 5′ end→3′ end) 2-18 or 2-19 of any of the antisense strand sequences in Table 2 or Table 3, and (ii) a sense strand comprising the sequence of nucleotides (from 5′ end→3′ end) 1-17 or 1-18 of any of the sense strand sequences in Table 2A, 2B, 2C, 2D, 2E, 2F, 4A, 4B, 4C, 4D, 4E, 4F, 5A, 5B, 5C, 5D, 5E, 5F, 6A, 6B, 6C, 6D, 6E, or 6F.

In some embodiments, the IAV RNAi agents include core 19-mer nucleotide sequences shown in the following Tables 2A, 2B, 2C, 2D, 2E, and 2F.

TABLE 2A
IAV RNAi agent (targeting M1) Antisense Strand and Sense Strand Core Stretch Base Sequences
(N = any nucleobase)

| | | | | Corresponding | |
|---|---|---|---|---|---|
| | Antisense Strand Base Sequence | | Sense Strand Base Sequence | Positions of | Targeted |
| SEQ | (5′ → 3′) | SEQ | (5′ → 3′) | Identified | Viral |
| ID | (Shown as an Unmodified | ID | (Shown as an Unmodified | Sequence on | Genome |
| NO:. | Nucleotide Sequence) | NO:. | Nucleotide Sequence) | SEQ ID NO: 1 | Position |
| 121 | GUACGUUUCGACCUCGGUU | 638 | AACCGAGGUCGAAACGUAC | 12-30 | 10 |
| 122 | AUACGUUUCGACCUCGGUU | 639 | AACCGAGGUCGAAACGUAU | 12-30 | 10 |
| 123 | UUACGUUUCGACCUCGGUU | 640 | AACCGAGGUCGAAACGUAA | 12-30 | 10 |
| 124 | NUACGUUUCGACCUCGGUU | 641 | AACCGAGGUCGAAACGUAN | 12-30 | 10 |
| 125 | NUACGUUUCGACCUCGGUN | 642 | NACCGAGGUCGAAACGUAN | 12-30 | 10 |
| 126 | GAUGAUAGAAAGAACGUAC | 643 | GUACGUUCUUUCUAUCAUC | 27-45 | 25 |
| 127 | AAUGAUAGAAAGAACGUAC | 644 | GUACGUUCUUUCUAUCAUU | 27-45 | 25 |
| 128 | UAUGAUAGAAAGAACGUAC | 645 | GUACGUUCUUUCUAUCAUA | 27-45 | 25 |
| 129 | NAUGAUAGAAAGAACGUAC | 646 | GUACGUUCUUUCUAUCAUN | 27-45 | 25 |
| 130 | NAUGAUAGAAAGAACGUAN | 647 | NUACGUUCUUUCUAUCAUN | 27-45 | 25 |
| 131 | GUGACAAGAUUGGUCUUGU | 648 | ACAAGACCAAUCUUGUCAC | 142-160 | 140 |
| 132 | AUGACAAGAUUGGUCUUGU | 649 | ACAAGACCAAUCUUGUCAU | 142-160 | 140 |
| 133 | UUGACAAGAUUGGUCUUGU | 650 | ACAAGACCAAUCUUGUCAA | 142-160 | 140 |
| 134 | NUGACAAGAUUGGUCUUGU | 651 | ACAAGACCAAUCUUGUCAN | 142-160 | 140 |
| 135 | NUGACAAGAUUGGUCUUGN | 652 | NCAAGACCAAUCUUGUCAN | 142-160 | 140 |
| 136 | AGUCAGAGGUGACAAGAUU | 653 | AAUCUUGUCACCUCUGACU | 150-168 | 148 |
| 137 | UGUCAGAGGUGACAAGAUU | 654 | AAUCUUGUCACCUCUGACA | 150-168 | 148 |
| 138 | NGUCAGAGGUGACAAGAUU | 655 | AAUCUUGUCACCUCUGACN | 150-168 | 148 |
| 139 | NGUCAGAGGUGACAAGAUN | 656 | NAUCUUGUCACCUCUGACN | 150-168 | 148 |
| 140 | UUAGUCAGAGGUGACAAGA | 657 | UCUUGUCACCUCUGACUAA | 152-170 | 150 |
| 141 | AUAGUCAGAGGUGACAAGA | 658 | UCUUGUCACCUCUGACUAU | 152-170 | 150 |
| 142 | NUAGUCAGAGGUGACAAGA | 659 | UCUUGUCACCUCUGACUAN | 152-170 | 150 |
| 143 | NUAGUCAGAGGUGACAAGN | 660 | NCUUGUCACCUCUGACUAN | 152-170 | 150 |
| 144 | GCUCUAUCCAUGUUGUUCG | 661 | CGAACAACAUGGAUAGAGC | 269-287 | 267 |
| 145 | ACUCUAUCCAUGUUGUUCG | 662 | CGAACAACAUGGAUAGAGU | 269-287 | 267 |
| 146 | UCUCUAUCCAUGUUGUUCG | 663 | CGAACAACAUGGAUAGAGA | 269-287 | 267 |
| 147 | NCUCUAUCCAUGUUGUUCG | 664 | CGAACAACAUGGAUAGAGN | 269-287 | 267 |
| 148 | NCUCUAUCCAUGUUGUUCN | 665 | NGAACAACAUGGAUAGAGN | 269-287 | 267 |
| 149 | AACUGCUCUAUCCAUGUUG | 666 | CAACAUGGAUAGAGCAGUU | 273-291 | 271 |
| 150 | UACUGCUCUAUCCAUGUUG | 667 | CAACAUGGAUAGAGCAGUA | 273-291 | 271 |
| 151 | NACUGCUCUAUCCAUGUUG | 668 | CAACAUGGAUAGAGCAGUN | 273-291 | 271 |
| 152 | NACUGCUCUAUCCAUGUUN | 669 | NAACAUGGAUAGAGCAGUN | 273-291 | 271 |
| 153 | CAGUUGAAUAGCUUAGUGA | 670 | UCACUAAGCUAUUCAACUG | 346-364 | 344 |
| 154 | AAGUUGAAUAGCUUAGUGA | 671 | UCACUAAGCUAUUCAACUU | 346-364 | 344 |
| 155 | UAGUUGAAUAGCUUAGUGA | 672 | UCACUAAGCUAUUCAACUA | 346-364 | 344 |
| 156 | NAGUUGAAUAGCUUAGUGA | 673 | UCACUAAGCUAUUCAACUN | 346-364 | 344 |
| 157 | NAGUUGAAUAGCUUAGUGN | 674 | NCACUAAGCUAUUCAACUN | 346-364 | 344 |
| 158 | CUGGCAAGUGCACCAGUUG | 675 | CAACUGGUGCACUUGCCAG | 359-377 | 357 |
| 159 | AUGGCAAGUGCACCAGUUG | 676 | CAACUGGUGCACUUGCCAU | 359-377 | 357 |
| 160 | UUGGCAAGUGCACCAGUUG | 677 | CAACUGGUGCACUUGCCAA | 359-377 | 357 |
| 161 | NUGGCAAGUGCACCAGUUG | 678 | CAACUGGUGCACUUGCCAN | 359-377 | 357 |
| 162 | NUGGCAAGUGCACCAGUUN | 679 | NAACUGGUGCACUUGCCAN | 359-377 | 357 |
| 163 | AAAGCAGCUUCUGUGGUCA | 680 | UGACCACAGAAGCUGCUUU | 413-431 | 411 |
| 164 | UAAGCAGCUUCUGUGGUCA | 681 | UGACCACAGAAGCUGCUUA | 413-431 | 411 |
| 165 | NAAGCAGCUUCUGUGGUCA | 682 | UGACCACAGAAGCUGCUUN | 413-431 | 411 |
| 166 | NAAGCAGCUUCUGUGGUCN | 683 | NGACCACAGAAGCUGCUUN | 413-431 | 411 |
| 167 | GCCUGAUUAGUGGAUUGGU | 684 | ACCAAUCCACUAAUCAGGC | 505-523 | 503 |
| 168 | ACCUGAUUAGUGGAUUGGU | 685 | ACCAAUCCACUAAUCAGGU | 505-523 | 503 |
| 169 | UCCUGAUUAGUGGAUUGGU | 686 | ACCAAUCCACUAAUCAGGA | 505-523 | 503 |
| 170 | NCCUGAUUAGUGGAUUGGU | 687 | ACCAAUCCACUAAUCAGGN | 505-523 | 503 |
| 171 | NCCUGAUUAGUGGAUUGGN | 688 | NCCAAUCCACUAAUCAGGN | 505-523 | 503 |
| 172 | CUAGGAUGAGUCCCAAUAG | 689 | CUAUUGGGACUCAUCCUAG | 653-671 | 651 |
| 173 | AUAGGAUGAGUCCCAAUAG | 690 | CUAUUGGGACUCAUCCUAU | 653-671 | 651 |
| 174 | UUAGGAUGAGUCCCAAUAG | 691 | CUAUUGGGACUCAUCCUAA | 653-671 | 651 |
| 175 | NUAGGAUGAGUCCCAAUAG | 692 | CUAUUGGGACUCAUCCUAN | 653-671 | 651 |
| 176 | NUAGGAUGAGUCCCAAUAN | 693 | NUAUUGGGACUCAUCCUAN | 653-671 | 651 |
| 177 | GACAAAAUGACCAUCGUCA | 694 | UGACGAUGGUCAUUUUGUC | 946-964 | 944 |
| 178 | AACAAAAUGACCAUCGUCA | 695 | UGACGAUGGUCAUUUUGUU | 946-964 | 944 |
| 179 | UACAAAAUGACCAUCGUCA | 696 | UGACGAUGGUCAUUUUGUA | 946-964 | 944 |
| 180 | NACAAAAUGACCAUCGUCA | 697 | UGACGAUGGUCAUUUUGUN | 946-964 | 944 |
| 181 | NACAAAAUGACCAUCGUCN | 698 | NGACGAUGGUCAUUUUGUN | 946-964 | 944 |
| 182 | UGACAAAAUGACCAUCGUC | 699 | GACGAUGGUCAUUUUGUCA | 947-965 | 945 |
| 183 | AGACAAAAUGACCAUCGUC | 700 | GACGAUGGUCAUUUUGUCU | 947-965 | 945 |
| 184 | NGACAAAAUGACCAUCGUC | 701 | GACGAUGGUCAUUUUGUCN | 947-965 | 945 |
| 185 | NGACAAAAUGACCAUCGUN | 702 | NACGAUGGUCAUUUUGUCN | 947-965 | 945 |

TABLE 2B
IAV RNAi agent (targeting NS1) Antisense Strand and Sense Strand Core Stretch Base Sequences
(N = any nucleobase)

| | | | | Corresponding | |
|---|---|---|---|---|---|
| | Antisense Strand Base Sequence | | Sense Strand Base Sequence | Positions of | Targeted |
| SEQ | (5′ → 3′) | SEQ | (5′ → 3′) | Identified | Viral |
| ID | (Shown as an Unmodified | ID | (Shown as an Unmodified | Sequence on | Genome |
| NO:. | Nucleotide Sequence) | NO:. | Nucleotide Sequence) | SEQ ID NO: 2 | Position |
| 186 | GAUCAAGGAAUGGGGCAUC | 703 | GAUGCCCCAUUCCUUGAUC | 85-103 | 83 |
| 187 | AAUCAAGGAAUGGGGCAUC | 704 | GAUGCCCCAUUCCUUGAUU | 85-103 | 83 |
| 188 | UAUCAAGGAAUGGGGCAUC | 705 | GAUGCCCCAUUCCUUGAUA | 85-103 | 83 |
| 189 | NAUCAAGGAAUGGGGCAUC | 706 | GAUGCCCCAUUCCUUGAUN | 85-103 | 83 |
| 190 | NAUCAAGGAAUGGGGCAUN | 707 | NAUGCCCCAUUCCUUGAUN | 85-103 | 83 |
| 191 | CGAUCAAGGAAUGGGGCAU | 708 | AUGCCCCAUUCCUUGAUCG | 86-104 | 84 |
| 192 | AGAUCAAGGAAUGGGGCAU | 709 | AUGCCCCAUUCCUUGAUCU | 86-104 | 84 |
| 193 | UGAUCAAGGAAUGGGGCAU | 710 | AUGCCCCAUUCCUUGAUCA | 86-104 | 84 |
| 194 | NGAUCAAGGAAUGGGGCAU | 711 | AUGCCCCAUUCCUUGAUCN | 86-104 | 84 |
| 195 | NGAUCAAGGAAUGGGGCAN | 712 | NUGCCCCAUUCCUUGAUCN | 86-104 | 84 |
| 196 | CCGAUCAAGGAAUGGGGCA | 713 | UGCCCCAUUCCUUGAUCGG | 87-105 | 85 |
| 197 | ACGAUCAAGGAAUGGGGCA | 714 | UGCCCCAUUCCUUGAUCGU | 87-105 | 85 |
| 198 | UCGAUCAAGGAAUGGGGCA | 715 | UGCCCCAUUCCUUGAUCGA | 87-105 | 85 |
| 199 | NCGAUCAAGGAAUGGGGCA | 716 | UGCCCCAUUCCUUGAUCGN | 87-105 | 85 |
| 200 | NCGAUCAAGGAAUGGGGCN | 717 | NGCCCCAUUCCUUGAUCGN | 87-105 | 85 |
| 201 | CAACAAGAGUGGCUGUUUC | 718 | GAAACAGCCACUCUUGUUG | 163-181 | 161 |
| 202 | AAACAAGAGUGGCUGUUUC | 719 | GAAACAGCCACUCUUGUUU | 163-181 | 161 |
| 203 | UAACAAGAGUGGCUGUUUC | 720 | GAAACAGCCACUCUUGUUA | 163-181 | 161 |
| 204 | NAACAAGAGUGGCUGUUUC | 721 | GAAACAGCCACUCUUGUUN | 163-181 | 161 |
| 205 | NAACAAGAGUGGCUGUUUN | 722 | NAAACAGCCACUCUUGUUN | 163-181 | 161 |
| 206 | UCAAGAUCCAUUCCACGAU | 723 | AUCGUGGAAUGGAUCUUGA | 190-208 | 188 |
| 207 | ACAAGAUCCAUUCCACGAU | 724 | AUCGUGGAAUGGAUCUUGU | 190-208 | 188 |
| 208 | NCAAGAUCCAUUCCACGAU | 725 | AUCGUGGAAUGGAUCUUGN | 190-208 | 188 |
| 209 | NCAAGAUCCAUUCCACGAN | 726 | NUCGUGGAAUGGAUCUUGN | 190-208 | 188 |
| 210 | CUGGAUUCCUCUUUCAAGA | 727 | UCUUGAAAGAGGAAUCCAG | 203-221 | 201 |
| 211 | AUGGAUUCCUCUUUCAAGA | 728 | UCUUGAAAGAGGAAUCCAU | 203-221 | 201 |
| 212 | UUGGAUUCCUCUUUCAAGA | 729 | UCUUGAAAGAGGAAUCCAA | 203-221 | 201 |
| 213 | NUGGAUUCCUCUUUCAAGA | 730 | UCUUGAAAGAGGAAUCCAN | 203-221 | 201 |
| 214 | NUGGAUUCCUCUUUCAAGN | 731 | NCUUGAAAGAGGAAUCCAN | 203-221 | 201 |
| 215 | AGGUACAGAUGCAAUUGUC | 732 | GACAAUUGCAUCUGUACCU | 237-255 | 235 |
| 216 | UGGUACAGAUGCAAUUGUC | 733 | GACAAUUGCAUCUGUACCA | 237-255 | 235 |
| 217 | NGGUACAGAUGCAAUUGUC | 734 | GACAAUUGCAUCUGUACCN | 237-255 | 235 |
| 218 | NGGUACAGAUGCAAUUGUN | 735 | NACAAUUGCAUCUGUACCN | 237-255 | 235 |
| 219 | GCGAAGUAGGUACAGAUGC | 736 | GCAUCUGUACCUACUUCGC | 244-262 | 242 |
| 220 | ACGAAGUAGGUACAGAUGC | 737 | GCAUCUGUACCUACUUCGU | 244-262 | 242 |
| 221 | UCGAAGUAGGUACAGAUGC | 738 | GCAUCUGUACCUACUUCGA | 244-262 | 242 |
| 222 | NCGAAGUAGGUACAGAUGC | 739 | GCAUCUGUACCUACUUCGN | 244-262 | 242 |
| 223 | NCGAAGUAGGUACAGAUGN | 740 | NCAUCUGUACCUACUUCGN | 244-262 | 242 |
| 224 | AUGAGCAUGAACCAGUCUC | 741 | GAGACUGGUUCAUGCUCAU | 299-317 | 297 |
| 225 | UUGAGCAUGAACCAGUCUC | 742 | GAGACUGGUUCAUGCUCAA | 299-317 | 297 |
| 226 | NUGAGCAUGAACCAGUCUC | 743 | GAGACUGGUUCAUGCUCAN | 299-317 | 297 |
| 227 | NUGAGCAUGAACCAGUCUN | 744 | NAGACUGGUUCAUGCUCAN | 299-317 | 297 |
| 228 | GCAUGAGCAUGAACCAGUC | 745 | GACUGGUUCAUGCUCAUGC | 301-319 | 299 |
| 229 | ACAUGAGCAUGAACCAGUC | 746 | GACUGGUUCAUGCUCAUGU | 301-319 | 299 |
| 230 | UCAUGAGCAUGAACCAGUC | 747 | GACUGGUUCAUGCUCAUGA | 301-319 | 299 |
| 231 | NCAUGAGCAUGAACCAGUC | 748 | GACUGGUUCAUGCUCAUGN | 301-319 | 299 |
| 232 | NCAUGAGCAUGAACCAGUN | 749 | NACUGGUUCAUGCUCAUGN | 301-319 | 299 |
| 233 | ACACUGAAGUUCGCUUUCA | 750 | UGAAAGCGAACUUCAGUGU | 389-407 | 387 |
| 234 | UCACUGAAGUUCGCUUUCA | 751 | UGAAAGCGAACUUCAGUGA | 389-407 | 387 |
| 235 | NCACUGAAGUUCGCUUUCA | 752 | UGAAAGCGAACUUCAGUGN | 389-407 | 387 |
| 236 | NCACUGAAGUUCGCUUUCN | 753 | NGAAAGCGAACUUCAGUGN | 389-407 | 387 |
| 237 | AAAGAUUACACUGAAGUUC | 754 | GAACUUCAGUGUAAUCUUU | 396-414 | 394 |
| 238 | UAAGAUUACACUGAAGUUC | 755 | GAACUUCAGUGUAAUCUUA | 396-414 | 394 |
| 239 | NAAGAUUACACUGAAGUUC | 756 | GAACUUCAGUGUAAUCUUN | 396-414 | 394 |
| 240 | NAAGAUUACACUGAAGUUN | 757 | NAACUUCAGUGUAAUCUUN | 396-414 | 394 |
| 241 | GUAUCAAGGUCUCUAAUCG | 758 | CGAUUAGAGACCUUGAUAC | 418-436 | 416 |
| 242 | AUAUCAAGGUCUCUAAUCG | 759 | CGAUUAGAGACCUUGAUAU | 418-436 | 416 |
| 243 | UUAUCAAGGUCUCUAAUCG | 760 | CGAUUAGAGACCUUGAUAA | 418-436 | 416 |
| 244 | NUAUCAAGGUCUCUAAUCG | 761 | CGAUUAGAGACCUUGAUAN | 418-436 | 416 |
| 245 | NUAUCAAGGUCUCUAAUCN | 762 | NGAUUAGAGACCUUGAUAN | 418-436 | 416 |
| 246 | AGUGAAAGCCCUUAGUAGU | 763 | ACUACUAAGGGCUUUCACU | 435-453 | 433 |
| 247 | UGUGAAAGCCCUUAGUAGU | 764 | ACUACUAAGGGCUUUCACA | 435-453 | 433 |
| 248 | NGUGAAAGCCCUUAGUAGU | 765 | ACUACUAAGGGCUUUCACN | 435-453 | 433 |
| 249 | NGUGAAAGCCCUUAGUAGN | 766 | NCUACUAAGGGCUUUCACN | 435-453 | 433 |
| 250 | CAGUGAAAGCCCUUAGUAG | 767 | CUACUAAGGGCUUUCACUG | 436-454 | 434 |
| 251 | AAGUGAAAGCCCUUAGUAG | 768 | CUACUAAGGGCUUUCACUU | 436-454 | 434 |
| 252 | UAGUGAAAGCCCUUAGUAG | 769 | CUACUAAGGGCUUUCACUA | 436-454 | 434 |
| 253 | NAGUGAAAGCCCUUAGUAG | 770 | CUACUAAGGGCUUUCACUN | 436-454 | 434 |
| 254 | NAGUGAAAGCCCUUAGUAN | 771 | NUACUAAGGGCUUUCACUN | 436-454 | 434 |
| 255 | AAGUAUGUCCUGGAAGAGA | 772 | UCUCUUCCAGGACAUACUU | 493-511 | 491 |
| 256 | UAGUAUGUCCUGGAAGAGA | 773 | UCUCUUCCAGGACAUACUA | 493-511 | 491 |
| 257 | NAGUAUGUCCUGGAAGAGA | 774 | UCUCUUCCAGGACAUACUN | 493-511 | 491 |
| 258 | NAGUAUGUCCUGGAAGAGN | 775 | NCUCUUCCAGGACAUACUN | 493-511 | 491 |
| 259 | UGCAUUUUUGACAUCCUCA | 776 | UGAGGAUGUCAAAAAUGCA | 513-531 | 511 |
| 260 | AGCAUUUUUGACAUCCUCA | 777 | UGAGGAUGUCAAAAAUGCU | 513-531 | 511 |
| 261 | NGCAUUUUUGACAUCCUCA | 778 | UGAGGAUGUCAAAAAUGCN | 513-531 | 511 |
| 262 | NGCAUUUUUGACAUCCUCN | 779 | NGAGGAUGUCAAAAAUGCN | 513-531 | 511 |
| 263 | CCCAACUGCAUUUUUGACA | 780 | UGUCAAAAAUGCAGUUGGG | 519-537 | 517 |
| 264 | ACCAACUGCAUUUUUGACA | 781 | UGUCAAAAAUGCAGUUGGU | 519-537 | 517 |
| 265 | UCCAACUGCAUUUUUGACA | 782 | UGUCAAAAAUGCAGUUGGA | 519-537 | 517 |
| 266 | NCCAACUGCAUUUUUGACA | 783 | UGUCAAAAAUGCAGUUGGN | 519-537 | 517 |
| 267 | NCCAACUGCAUUUUUGACN | 784 | NGUCAAAAAUGCAGUUGGN | 519-537 | 517 |
| 268 | GAUGAGGACCCCAACUGCA | 785 | UGCAGUUGGGGUCCUCAUC | 528-546 | 526 |
| 269 | AAUGAGGACCCCAACUGCA | 786 | UGCAGUUGGGGUCCUCAUU | 528-546 | 526 |
| 270 | UAUGAGGACCCCAACUGCA | 787 | UGCAGUUGGGGUCCUCAUA | 528-546 | 526 |
| 271 | NAUGAGGACCCCAACUGCA | 788 | UGCAGUUGGGGUCCUCAUN | 528-546 | 526 |
| 272 | NAUGAGGACCCCAACUGCN | 789 | NGCAGUUGGGGUCCUCAUN | 528-546 | 526 |
| 273 | CCAUUCAAGUCCUCCGAUG | 790 | CAUCGGAGGACUUGAAUGG | 543-561 | 541 |
| 274 | ACAUUCAAGUCCUCCGAUG | 791 | CAUCGGAGGACUUGAAUGU | 543-561 | 541 |
| 275 | UCAUUCAAGUCCUCCGAUG | 792 | CAUCGGAGGACUUGAAUGA | 543-561 | 541 |
| 276 | NCAUUCAAGUCCUCCGAUG | 793 | CAUCGGAGGACUUGAAUGN | 543-561 | 541 |
| 277 | NCAUUCAAGUCCUCCGAUN | 794 | NAUCGGAGGACUUGAAUGN | 543-561 | 541 |
| 278 | UCCAUUCAAGUCCUCCGAU | 795 | AUCGGAGGACUUGAAUGGA | 544-562 | 542 |
| 279 | ACCAUUCAAGUCCUCCGAU | 796 | AUCGGAGGACUUGAAUGGU | 544-562 | 542 |
| 280 | NCCAUUCAAGUCCUCCGAU | 797 | AUCGGAGGACUUGAAUGGN | 544-562 | 542 |
| 281 | NCCAUUCAAGUCCUCCGAN | 798 | NUCGGAGGACUUGAAUGGN | 544-562 | 542 |
| 282 | UUCCAUUCAAGUCCUCCGA | 799 | UCGGAGGACUUGAAUGGAA | 545-563 | 543 |
| 283 | AUCCAUUCAAGUCCUCCGA | 800 | UCGGAGGACUUGAAUGGAU | 545-563 | 543 |
| 284 | NUCCAUUCAAGUCCUCCGA | 801 | UCGGAGGACUUGAAUGGAN | 545-563 | 543 |
| 285 | NUCCAUUCAAGUCCUCCGN | 802 | NCGGAGGACUUGAAUGGAN | 545-563 | 543 |
| 286 | CAUUCCAUUCAAGUCCUCC | 803 | GGAGGACUUGAAUGGAAUG | 547-565 | 545 |
| 287 | AAUUCCAUUCAAGUCCUCC | 804 | GGAGGACUUGAAUGGAAUU | 547-565 | 545 |
| 288 | UAUUCCAUUCAAGUCCUCC | 805 | GGAGGACUUGAAUGGAAUA | 547-565 | 545 |
| 289 | NAUUCCAUUCAAGUCCUCC | 806 | GGAGGACUUGAAUGGAAUN | 547-565 | 545 |
| 290 | NAUUCCAUUCAAGUCCUCN | 807 | NGAGGACUUGAAUGGAAUN | 547-565 | 545 |
| 291 | CCAUUCCAUUCAAGUCCUC | 808 | GAGGACUUGAAUGGAAUGG | 548-566 | 546 |
| 292 | ACAUUCCAUUCAAGUCCUC | 809 | GAGGACUUGAAUGGAAUGU | 548-566 | 546 |
| 293 | UCAUUCCAUUCAAGUCCUC | 810 | GAGGACUUGAAUGGAAUGA | 548-566 | 546 |
| 294 | NCAUUCCAUUCAAGUCCUC | 811 | GAGGACUUGAAUGGAAUGN | 548-566 | 546 |
| 295 | NCAUUCCAUUCAAGUCCUN | 812 | NAGGACUUGAAUGGAAUGN | 548-566 | 546 |
| 296 | GUGUUACCAUUCCAUUCAA | 813 | UUGAAUGGAAUGGUAACAC | 554-572 | 552 |
| 297 | AUGUUACCAUUCCAUUCAA | 814 | UUGAAUGGAAUGGUAACAU | 554-572 | 552 |
| 298 | UUGUUACCAUUCCAUUCAA | 815 | UUGAAUGGAAUGGUAACAA | 554-572 | 552 |
| 299 | NUGUUACCAUUCCAUUCAA | 816 | UUGAAUGGAAUGGUAACAN | 554-572 | 552 |
| 300 | NUGUUACCAUUCCAUUCAN | 817 | UUGAAUGGAAUGGUAACAN | 554-572 | 552 |
| 301 | ACUCGAACCGUGUUACCAU | 818 | AUGGUAACACGGUUCGAGU | 563-581 | 561 |
| 302 | UCUCGAACCGUGUUACCAU | 819 | AUGGUAACACGGUUCGAGA | 563-581 | 561 |
| 303 | NCUCGAACCGUGUUACCAU | 820 | AUGGUAACACGGUUCGAGN | 563-581 | 561 |
| 304 | NCUCGAACCGUGUUACCAN | 821 | NUGGUAACACGGUUCGAGN | 563-581 | 561 |
| 305 | GAGACUCGAACCGUGUUAC | 822 | GUAACACGGUUCGAGUCUC | 566-584 | 564 |
| 306 | AAGACUCGAACCGUGUUAC | 823 | GUAACACGGUUCGAGUCUU | 566-584 | 564 |
| 307 | UAGACUCGAACCGUGUUAC | 824 | GUAACACGGUUCGAGUCUA | 566-584 | 564 |
| 308 | NAGACUCGAACCGUGUUAC | 825 | GUAACACGGUUCGAGUCUN | 566-584 | 564 |
| 309 | NAGACUCGAACCGUGUUAN | 826 | NUAACACGGUUCGAGUCUN | 566-584 | 564 |
| 310 | CUCCAAGCGAAUCUCUGUA | 827 | UACAGAGAUUCGCUUGGAG | 593-611 | 591 |
| 311 | AUCCAAGCGAAUCUCUGUA | 828 | UACAGAGAUUCGCUUGGAU | 593-611 | 591 |
| 312 | UUCCAAGCGAAUCUCUGUA | 829 | UACAGAGAUUCGCUUGGAA | 593-611 | 591 |
| 313 | NUCCAAGCGAAUCUCUGUA | 830 | UACAGAGAUUCGCUUGGAN | 593-611 | 591 |
| 314 | NUCCAAGCGAAUCUCUGUN | 831 | NACAGAGAUUCGCUUGGAN | 593-611 | 591 |
| 315 | AAGGUCUCCCAUUCUCAUC | 832 | GAUGAGAAUGGGAGACCUU | 619-637 | 617 |
| 316 | UAGGUCUCCCAUUCUCAUC | 833 | GAUGAGAAUGGGAGACCUA | 619-637 | 617 |
| 317 | NAGGUCUCCCAUUCUCAUC | 834 | GAUGAGAAUGGGAGACCUN | 619-637 | 617 |
| 318 | NAGGUCUCCCAUUCUCAUN | 835 | NAUGAGAAUGGGAGACCUN | 619-637 | 617 |
| 319 | UAGUGAAGGUCUCCCAUUC | 836 | GAAUGGGAGACCUUCACUA | 624-642 | 622 |
| 320 | AAGUGAAGGUCUCCCAUUC | 837 | GAAUGGGAGACCUUCACUU | 624-642 | 622 |
| 321 | NAGUGAAGGUCUCCCAUUC | 838 | GAAUGGGAGACCUUCACUN | 624-642 | 622 |
| 322 | NAGUGAAGGUCUCCCAUUN | 839 | NAAUGGGAGACCUUCACUN | 624-642 | 622 |
| 323 | UUCGAAACUAUUCUCUGUC | 840 | GACAGAGAAUAGUUUCGAA | 739-757 | 737 |
| 324 | AUCGAAACUAUUCUCUGUC | 841 | GACAGAGAAUAGUUUCGAU | 739-757 | 737 |
| 325 | NUCGAAACUAUUCUCUGUC | 842 | GACAGAGAAUAGUUUCGAN | 739-757 | 737 |
| 326 | NUCGAAACUAUUCUCUGUN | 843 | NACAGAGAAUAGUUUCGAN | 739-757 | 737 |
| 327 | UAGUUGUAAGGCUUGCAUA | 844 | UAUGCAAGCCUUACAACUA | 769-787 | 767 |
| 328 | AAGUUGUAAGGCUUGCAUA | 845 | UAUGCAAGCCUUACAACUU | 769-787 | 767 |
| 329 | NAGUUGUAAGGCUUGCAUA | 846 | UAUGCAAGCCUUACAACUN | 769-787 | 767 |
| 330 | NAGUUGUAAGGCUUGCAUN | 847 | NAUGCAAGCCUUACAACUN | 769-787 | 767 |
| 331 | AACGAGAAAGCUCUUAUCU | 848 | AGAUAAGAGCUUUCUCGUU | 807-825 | 805 |
| 332 | UACGAGAAAGCUCUUAUCU | 849 | AGAUAAGAGCUUUCUCGUA | 807-825 | 805 |
| 333 | NACGAGAAAGCUCUUAUCU | 850 | AGAUAAGAGCUUUCUCGUN | 807-825 | 805 |
| 334 | NACGAGAAAGCUCUUAUCN | 851 | NGAUAAGAGCUUUCUCGUN | 807-825 | 805 |
| 335 | AUAAGCUGAAACGAGAAAG | 852 | CUUUCUCGUUUCAGCUUAU | 816-834 | 814 |
| 336 | UUAAGCUGAAACGAGAAAG | 853 | CUUUCUCGUUUCAGCUUAA | 816-834 | 814 |
| 337 | NUAAGCUGAAACGAGAAAG | 854 | CUUUCUCGUUUCAGCUUAN | 816-834 | 814 |
| 338 | NUAAGCUGAAACGAGAAAN | 855 | NUUUCUCGUUUCAGCUUAN | 816-834 | 814 |

TABLE 2C
IAV RNAi agent (targeting PB1) Antisense Strand and Sense Strand Core Stretch Base Sequences
(N = any nucleobase)

| | | | | Corresponding | |
|---|---|---|---|---|---|
| | Antisense Strand Base Sequence | | Sense Strand Base Sequence | Positions of | Targeted |
| SEQ | (5′ → 3′) | SEQ | (5′ → 3′) | Identified | Viral |
| ID | (Shown as an Unmodified | ID | (Shown as an Unmodified | Sequence on | Genome |
| NO:. | Nucleotide Sequence) | NO:. | Nucleotide Sequence) | SEQ ID NO: 3 | Position |
| 339 | GGAAUGUGGUGCUUAUGGC | 856 | GCCAUAAGCACCACAUUCC | 49-67 | 47 |
| 340 | AGAAUGUGGUGCUUAUGGC | 857 | GCCAUAAGCACCACAUUCU | 49-67 | 47 |
| 341 | UGAAUGUGGUGCUUAUGGC | 858 | GCCAUAAGCACCACAUUCA | 49-67 | 47 |
| 342 | NGAAUGUGGUGCUUAUGGC | 859 | GCCAUAAGCACCACAUUCN | 49-67 | 47 |
| 343 | NGAAUGUGGUGCUUAUGGN | 860 | NCCAUAAGCACCACAUUCN | 49-67 | 47 |
| 344 | CAGUAUAAGGGAAUGUGGU | 861 | ACCACAUUCCCUUAUACUG | 58-76 | 56 |
| 345 | AAGUAUAAGGGAAUGUGGU | 862 | ACCACAUUCCCUUAUACUU | 58-76 | 56 |
| 346 | UAGUAUAAGGGAAUGUGGU | 863 | ACCACAUUCCCUUAUACUA | 58-76 | 56 |
| 347 | NAGUAUAAGGGAAUGUGGU | 864 | ACCACAUUCCCUUAUACUN | 58-76 | 56 |
| 348 | NAGUAUAAGGGAAUGUGGN | 865 | NCCACAUUCCCUUAUACUN | 58-76 | 56 |
| 349 | GAGUAUUGGUGUGUUCUGU | 866 | ACAGAACACACCAAUACUC | 131-149 | 129 |
| 350 | AAGUAUUGGUGUGUUCUGU | 867 | ACAGAACACACCAAUACUU | 131-149 | 129 |
| 351 | UAGUAUUGGUGUGUUCUGU | 868 | ACAGAACACACCAAUACUA | 131-149 | 129 |
| 352 | NAGUAUUGGUGUGUUCUGU | 869 | ACAGAACACACCAAUACUN | 131-149 | 129 |
| 353 | NAGUAUUGGUGUGUUCUGN | 870 | NCAGAACACACCAAUACUN | 131-149 | 129 |
| 354 | UUCCAUUGUUUCAAGGCAU | 871 | AUGCCUUGAAACAAUGGAA | 318-336 | 316 |
| 355 | AUCCAUUGUUUCAAGGCAU | 872 | AUGCCUUGAAACAAUGGAU | 318-336 | 316 |
| 356 | NUCCAUUGUUUCAAGGCAU | 873 | AUGCCUUGAAACAAUGGAN | 318-336 | 316 |
| 357 | NUCCAUUGUUUCAAGGCAN | 874 | NUGCCUUGAAACAAUGGAN | 318-336 | 316 |
| 358 | AAGACUUCUAUGGUGUUGG | 875 | CCAACACCAUAGAAGUCUU | 431-449 | 429 |
| 359 | UAGACUUCUAUGGUGUUGG | 876 | CCAACACCAUAGAAGUCUA | 431-449 | 429 |
| 360 | NAGACUUCUAUGGUGUUGG | 877 | CCAACACCAUAGAAGUCUN | 431-449 | 429 |
| 361 | NAGACUUCUAUGGUGUUGN | 878 | NCAACACCAUAGAAGUCUN | 431-449 | 429 |
| 362 | UGGUCAUGUUGUCUCUUAC | 879 | GUAAGAGACAACAUGACCA | 571-589 | 569 |
| 363 | AGGUCAUGUUGUCUCUUAC | 880 | GUAAGAGACAACAUGACCU | 571-589 | 569 |
| 364 | NGGUCAUGUUGUCUCUUAC | 881 | GUAAGAGACAACAUGACCN | 571-589 | 569 |
| 365 | NGGUCAUGUUGUCUCUUAN | 882 | NUAAGAGACAACAUGACCN | 571-589 | 569 |
| 366 | UACGAAACCUCUAAUCUGC | 883 | GCAGAUUAGAGGUUUCGUA | 738-756 | 736 |
| 367 | AACGAAACCUCUAAUCUGC | 884 | GCAGAUUAGAGGUUUCGUU | 738-756 | 736 |
| 368 | NACGAAACCUCUAAUCUGC | 885 | GCAGAUUAGAGGUUUCGUN | 738-756 | 736 |
| 369 | NACGAAACCUCUAAUCUGN | 886 | NCAGAUUAGAGGUUUCGUN | 738-756 | 736 |
| 370 | UCAAGCUUUUCGCAAAUGC | 887 | GCAUUUGCGAAAAGCUUGA | 782-800 | 780 |
| 371 | ACAAGCUUUUCGCAAAUGC | 888 | GCAUUUGCGAAAAGCUUGU | 782-800 | 780 |
| 372 | NCAAGCUUUUCGCAAAUGC | 889 | GCAUUUGCGAAAAGCUUGN | 782-800 | 780 |
| 373 | NCAAGCUUUUCGCAAAUGN | 890 | NCAUUUGCGAAAAGCUUGN | 782-800 | 780 |
| 374 | AGACUGUUCAAGCUUUUCG | 891 | CGAAAAGCUUGAACAGUCU | 789-807 | 787 |
| 375 | UGACUGUUCAAGCUUUUCG | 892 | CGAAAAGCUUGAACAGUCA | 789-807 | 787 |
| 376 | NGACUGUUCAAGCUUUUCG | 893 | CGAAAAGCUUGAACAGUCN | 789-807 | 787 |
| 377 | NGACUGUUCAAGCUUUUCN | 894 | NGAAAAGCUUGAACAGUCN | 789-807 | 787 |
| 378 | ACUUAGUGUUGUCCCCAGU | 895 | ACUGGGGACAACACUAAGU | 907-925 | 905 |
| 379 | UCUUAGUGUUGUCCCCAGU | 896 | ACUGGGGACAACACUAAGA | 907-925 | 905 |
| 380 | NCUUAGUGUUGUCCCCAGU | 897 | ACUGGGGACAACACUAAGN | 907-925 | 905 |
| 381 | NCUUAGUGUUGUCCCCAGN | 898 | NCUGGGGACAACACUAAGN | 907-925 | 905 |
| 382 | CCCUAGUCUUGCCAUUUUG | 899 | CAAAAUGGCAAGACUAGGG | 1038-1056 | 1036 |
| 383 | ACCUAGUCUUGCCAUUUUG | 900 | CAAAAUGGCAAGACUAGGU | 1038-1056 | 1036 |
| 384 | UCCUAGUCUUGCCAUUUUG | 901 | CAAAAUGGCAAGACUAGGA | 1038-1056 | 1036 |
| 385 | NCCUAGUCUUGCCAUUUUG | 902 | CAAAAUGGCAAGACUAGGN | 1038-1056 | 1036 |
| 386 | NCCUAGUCUUGCCAUUUUN | 903 | NAAAAUGGCAAGACUAGGN | 1038-1056 | 1036 |
| 387 | CCAUCAUCAUCCCAGGACU | 904 | AGUCCUGGGAUGAUGAUGG | 1210-1228 | 1208 |
| 388 | ACAUCAUCAUCCCAGGACU | 905 | AGUCCUGGGAUGAUGAUGU | 1210-1228 | 1208 |
| 389 | UCAUCAUCAUCCCAGGACU | 906 | AGUCCUGGGAUGAUGAUGA | 1210-1228 | 1208 |
| 390 | NCAUCAUCAUCCCAGGACU | 907 | AGUCCUGGGAUGAUGAUGN | 1210-1228 | 1208 |
| 391 | NCAUCAUCAUCCCAGGACN | 908 | AGUCCUGGGAUGAUGAUGN | 1210-1228 | 1208 |
| 392 | UUAGCAUGUUGAACAUGCC | 909 | GGCAUGUUCAACAUGCUAA | 1228-1246 | 1226 |
| 393 | AUAGCAUGUUGAACAUGCC | 910 | GGCAUGUUCAACAUGCUAU | 1228-1246 | 1226 |
| 394 | NUAGCAUGUUGAACAUGCC | 911 | GGCAUGUUCAACAUGCUAN | 1228-1246 | 1226 |
| 395 | NUAGCAUGUUGAACAUGEN | 912 | NGCAUGUUCAACAUGCUAN | 1228-1246 | 1226 |
| 396 | CCAAGAUUCAGUAUCGAGA | 913 | UCUCGAUACUGAAUCUUGG | 1262-1280 | 1260 |
| 397 | ACAAGAUUCAGUAUCGAGA | 914 | UCUCGAUACUGAAUCUUGU | 1262-1280 | 1260 |
| 398 | UCAAGAUUCAGUAUCGAGA | 915 | UCUCGAUACUGAAUCUUGA | 1262-1280 | 1260 |
| 399 | NCAAGAUUCAGUAUCGAGA | 916 | UCUCGAUACUGAAUCUUGN | 1262-1280 | 1260 |
| 400 | NCAAGAUUCAGUAUCGAGN | 917 | NCUCGAUACUGAAUCUUGN | 1262-1280 | 1260 |
| 401 | UGUAGAAUCUGUCCACUCC | 918 | GGAGUGGACAGAUUCUACA | 1384-1402 | 1382 |
| 402 | AGUAGAAUCUGUCCACUCC | 919 | GGAGUGGACAGAUUCUACU | 1384-1402 | 1382 |
| 403 | NGUAGAAUCUGUCCACUCC | 920 | GGAGUGGACAGAUUCUACN | 1384-1402 | 1382 |
| 404 | NGUAGAAUCUGUCCACUCN | 921 | NGAGUGGACAGAUUCUACN | 1384-1402 | 1382 |
| 405 | GCUAAAAUUAGCCACAAAU | 922 | AUUUGUGGCUAAUUUUAGC | 1500-1518 | 1498 |
| 406 | ACUAAAAUUAGCCACAAAU | 923 | AUUUGUGGCUAAUUUUAGU | 1500-1518 | 1498 |
| 407 | UCUAAAAUUAGCCACAAAU | 924 | AUUUGUGGCUAAUUUUAGA | 1500-1518 | 1498 |
| 408 | NCUAAAAUUAGCCACAAAU | 925 | AUUUGUGGCUAAUUUUAGN | 1500-1518 | 1498 |
| 409 | NCUAAAAUUAGCCACAAAN | 926 | NUUUGUGGCUAAUUUUAGN | 1500-1518 | 1498 |
| 410 | UCAUGUUGUUCUUUAUCAC | 927 | GUGAUAAAGAACAACAUGA | 1585-1603 | 1583 |
| 411 | ACAUGUUGUUCUUUAUCAC | 928 | GUGAUAAAGAACAACAUGU | 1585-1603 | 1583 |
| 412 | NCAUGUUGUUCUUUAUCAC | 929 | GUGAUAAAGAACAACAUGN | 1585-1603 | 1583 |
| 413 | NCAUGUUGUUCUUUAUCAN | 930 | NUGAUAAAGAACAACAUGN | 1585-1603 | 1583 |
| 414 | UGAUGAACAAUUGAAGAGC | 931 | GCUCUUCAAUUGUUCAUCA | 1639-1657 | 1637 |
| 415 | AGAUGAACAAUUGAAGAGC | 932 | GCUCUUCAAUUGUUCAUCU | 1639-1657 | 1637 |
| 416 | NGAUGAACAAUUGAAGAGC | 933 | GCUCUUCAAUUGUUCAUCN | 1639-1657 | 1637 |
| 417 | NGAUGAACAAUUGAAGAGN | 934 | NCUCUUCAAUUGUUCAUCN | 1639-1657 | 1637 |
| 418 | AUAAGUUUGGUCCUCCAUC | 935 | GAUGGAGGACCAAACUUAU | 1777-1795 | 1775 |
| 419 | UUAAGUUUGGUCCUCCAUC | 936 | GAUGGAGGACCAAACUUAA | 1777-1795 | 1775 |
| 420 | NUAAGUUUGGUCCUCCAUC | 937 | GAUGGAGGACCAAACUUAN | 1777-1795 | 1775 |
| 421 | NUAAGUUUGGUCCUCCAUN | 938 | NAUGGAGGACCAAACUUAN | 1777-1795 | 1775 |
| 422 | UGGUACAUCUGUUCAUCCU | 939 | AGGAUGAACAGAUGUACCA | 2051-2069 | 2049 |
| 423 | AGGUACAUCUGUUCAUCCU | 940 | AGGAUGAACAGAUGUACCU | 2051-2069 | 2049 |
| 424 | NGGUACAUCUGUUCAUCCU | 941 | AGGAUGAACAGAUGUACCN | 2051-2069 | 2049 |
| 425 | NGGUACAUCUGUUCAUCCN | 942 | NGGAUGAACAGAUGUACCN | 2051-2069 | 2049 |
| 426 | CCACCAUGCUAGAAAUUCC | 943 | GGAAUUUCUAGCAUGGUGG | 2128-2146 | 2126 |
| 427 | ACACCAUGCUAGAAAUUCC | 944 | GGAAUUUCUAGCAUGGUGU | 2128-2146 | 2126 |
| 428 | UCACCAUGCUAGAAAUUCC | 945 | GGAAUUUCUAGCAUGGUGA | 2128-2146 | 2126 |
| 429 | NCACCAUGCUAGAAAUUCC | 946 | GGAAUUUCUAGCAUGGUGN | 2128-2146 | 2126 |
| 430 | NCACCAUGCUAGAAAUUCN | 947 | NGAAUUUCUAGCAUGGUGN | 2128-2146 | 2126 |
| 431 | GAGAACUCUUCUUUCUUGA | 948 | UCAAGAAAGAAGAGUUCUC | 2204-2222 | 2202 |
| 432 | AAGAACUCUUCUUUCUUGA | 949 | UCAAGAAAGAAGAGUUCUU | 2204-2222 | 2202 |
| 433 | UAGAACUCUUCUUUCUUGA | 950 | UCAAGAAAGAAGAGUUCUA | 2204-2222 | 2202 |
| 434 | NAGAACUCUUCUUUCUUGA | 951 | UCAAGAAAGAAGAGUUCUN | 2204-2222 | 2202 |
| 435 | NAGAACUCUUCUUUCUUGN | 952 | NCAAGAAAGAAGAGUUCUN | 2204-2222 | 2202 |
| 436 | GAACAGAUCUUCAUGAUCU | 953 | AGAUCAUGAAGAUCUGUUC | 2225-2243 | 2223 |
| 437 | AAACAGAUCUUCAUGAUCU | 954 | AGAUCAUGAAGAUCUGUUU | 2225-2243 | 2223 |
| 438 | UAACAGAUCUUCAUGAUCU | 955 | AGAUCAUGAAGAUCUGUUA | 2225-2243 | 2223 |
| 439 | NAACAGAUCUUCAUGAUCU | 956 | AGAUCAUGAAGAUCUGUUN | 2225-2243 | 2223 |
| 440 | NAACAGAUCUUCAUGAUCN | 957 | NGAUCAUGAAGAUCUGUUN | 2225-2243 | 2223 |
| 441 | CUUCAAUGGUGGAACAGAU | 958 | AUCUGUUCCACCAUUGAAG | 2236-2254 | 2234 |
| 442 | AUUCAAUGGUGGAACAGAU | 959 | AUCUGUUCCACCAUUGAAU | 2236-2254 | 2234 |
| 443 | UUUCAAUGGUGGAACAGAU | 960 | AUCUGUUCCACCAUUGAAA | 2236-2254 | 2234 |
| 444 | NUUCAAUGGUGGAACAGAU | 961 | AUCUGUUCCACCAUUGAAN | 2236-2254 | 2234 |
| 445 | NUUCAAUGGUGGAACAGAN | 962 | NUCUGUUCCACCAUUGAAN | 2236-2254 | 2234 |
| 446 | CUGAGUUCUUCAAUGGUGG | 963 | CCACCAUUGAAGAACUCAG | 2243-2261 | 2241 |
| 447 | AUGAGUUCUUCAAUGGUGG | 964 | CCACCAUUGAAGAACUCAU | 2243-2261 | 2241 |
| 448 | UUGAGUUCUUCAAUGGUGG | 965 | CCACCAUUGAAGAACUCAA | 2243-2261 | 2241 |
| 449 | NUGAGUUCUUCAAUGGUGG | 966 | CCACCAUUGAAGAACUCAN | 2243-2261 | 2241 |
| 450 | NUGAGUUCUUCAAUGGUGG | 967 | NCACCAUUGAAGAACUCAN | 2243-2261 | 2241 |

TABLE 2D
IAV RNAi agent (targeting PB1) Antisense Strand and Sense Strand Core Stretch Base Sequences
(N = any nucleobase)

| | | | | Corresponding | |
|---|---|---|---|---|---|
| | Antisense Strand Base Sequence | | Sense Strand Base Sequence | Positions of | Targeted |
| SEQ | (5′ → 3′) | SEQ | (5′ → 3′) | Identified | Viral |
| ID | (Shown as an Unmodified | ID | (Shown as an Unmodified | Sequence on | Genome |
| NO:. | Nucleotide Sequence) | NO:. | Nucleotide Sequence) | SEQ ID NO: 4 | Position |
| 451 | UGGUCUUAGUGAGUAUCUC | 968 | GAGAUACUCACUAAGACCA | 52-70 | 50 |
| 452 | AGGUCUUAGUGAGUAUCUC | 969 | GAGAUACUCACUAAGACCU | 52-70 | 50 |
| 453 | NGGUCUUAGUGAGUAUCUC | 970 | GAGAUACUCACUAAGACCN | 52-70 | 50 |
| 454 | NGGUCUUAGUGAGUAUCUN | 971 | NAGAUACUCACUAAGACCN | 52-70 | 50 |
| 455 | CCAUGUUUCAACCUUUCGA | 972 | UCGAAAGGUUGAAACAUGG | 365-383 | 363 |
| 456 | ACAUGUUUCAACCUUUCGA | 973 | UCGAAAGGUUGAAACAUGU | 365-383 | 363 |
| 457 | UCAUGUUUCAACCUUUCGA | 974 | UCGAAAGGUUGAAACAUGA | 365-383 | 363 |
| 458 | NCAUGUUUCAACCUUUCGA | 975 | UCGAAAGGUUGAAACAUGN | 365-383 | 363 |
| 459 | NCAUGUUUCAACCUUUCGN | 976 | UCGAAAGGUUGAAACAUGN | 365-383 | 363 |
| 460 | CUAGCAUGUACGCCACCAU | 977 | AUGGUGGCGUACAUGCUAG | 604-622 | 602 |
| 461 | AUAGCAUGUACGCCACCAU | 978 | AUGGUGGCGUACAUGCUAU | 604-622 | 602 |
| 462 | UUAGCAUGUACGCCACCAU | 979 | AUGGUGGCGUACAUGCUAA | 604-622 | 602 |
| 463 | NUAGCAUGUACGCCACCAU | 980 | AUGGUGGCGUACAUGCUAN | 604-622 | 602 |
| 464 | NUAGCAUGUACGCCACCAN | 981 | NUGGUGGCGUACAUGCUAN | 604-622 | 602 |
| 465 | UUACUAUGUUUCUAGCAGC | 982 | GCUGCUAGAAACAUAGUAA | 784-802 | 782 |
| 466 | AUACUAUGUUUCUAGCAGC | 983 | GCUGCUAGAAACAUAGUAU | 784-802 | 782 |
| 467 | NUACUAUGUUUCUAGCAGC | 984 | GCUGCUAGAAACAUAGUAN | 784-802 | 782 |
| 468 | NUACUAUGUUUCUAGCAGN | 985 | NCUGCUAGAAACAUAGUAN | 784-802 | 782 |
| 469 | GACUGAUGAUCCGCUUGUC | 986 | GACAAGCGGAUCAUCAGUC | 996-1014 | 994 |
| 470 | AACUGAUGAUCCGCUUGUC | 987 | GACAAGCGGAUCAUCAGUU | 996-1014 | 994 |
| 471 | UACUGAUGAUCCGCUUGUC | 988 | GACAAGCGGAUCAUCAGUA | 996-1014 | 994 |
| 472 | NACUGAUGAUCCGCUUGUC | 989 | GACAAGCGGAUCAUCAGUN | 996-1014 | 994 |
| 473 | NACUGAUGAUCCGCUUGUN | 990 | NACAAGCGGAUCAUCAGUN | 996-1014 | 994 |
| 474 | CCUUGAUCAUGCAAUCCUC | 991 | GAGGAUUGCAUGAUCAAGG | 1219-1237 | 1217 |
| 475 | ACUUGAUCAUGCAAUCCUC | 992 | GAGGAUUGCAUGAUCAAGU | 1219-1237 | 1217 |
| 476 | UCUUGAUCAUGCAAUCCUC | 993 | GAGGAUUGCAUGAUCAAGA | 1219-1237 | 1217 |
| 477 | NCUUGAUCAUGCAAUCCUC | 994 | GAGGAUUGCAUGAUCAAGN | 1219-1237 | 1217 |
| 478 | NCUUGAUCAUGCAAUCCUN | 995 | NAGGAUUGCAUGAUCAAGN | 1219-1237 | 1217 |
| 479 | GGUCAAUACUCACUACCAC | 996 | GUGGUAGUGAGUAUUGACC | 1480-1498 | 1478 |
| 480 | AGUCAAUACUCACUACCAC | 997 | GUGGUAGUGAGUAUUGACU | 1480-1498 | 1478 |
| 481 | UGUCAAUACUCACUACCAC | 998 | GUGGUAGUGAGUAUUGACA | 1480-1498 | 1478 |
| 482 | NGUCAAUACUCACUACCAC | 999 | GUGGUAGUGAGUAUUGACN | 1480-1498 | 1478 |
| 483 | NGUCAAUACUCACUACCAN | 1000 | NUGGUAGUGAGUAUUGACN | 1480-1498 | 1478 |
| 484 | AGACAAUAGUACGUUCCCU | 1001 | AGGGAACGUACUAUUGUCU | 1524-1542 | 1522 |
| 485 | UGACAAUAGUACGUUCCCU | 1002 | AGGGAACGUACUAUUGUCA | 1524-1542 | 1522 |
| 486 | NGACAAUAGUACGUUCCCU | 1003 | AGGGAACGUACUAUUGUCN | 1524-1542 | 1522 |
| 487 | NGACAAUAGUACGUUCCCN | 1004 | NGGGAACGUACUAUUGUCN | 1524-1542 | 1522 |
| 488 | CCAUUGAUCUCCCACAUCA | 1005 | UGAUGUGGGAGAUCAAUGG | 1604-1622 | 1602 |
| 489 | ACAUUGAUCUCCCACAUCA | 1006 | UGAUGUGGGAGAUCAAUGU | 1604-1622 | 1602 |
| 490 | UCAUUGAUCUCCCACAUCA | 1007 | UGAUGUGGGAGAUCAAUGA | 1604-1622 | 1602 |
| 491 | NCAUUGAUCUCCCACAUCA | 1008 | UGAUGUGGGAGAUCAAUGN | 1604-1622 | 1602 |
| 492 | NCAUUGAUCUCCCACAUCN | 1009 | NGAUGUGGGAGAUCAAUGN | 1604-1622 | 1602 |
| 493 | UGGAACAGUGUCCUUACGA | 1010 | UCGUAAGGACACUGUUCCA | 1784-1802 | 1782 |
| 494 | AGGAACAGUGUCCUUACGA | 1011 | UCGUAAGGACACUGUUCCU | 1784-1802 | 1782 |
| 495 | NGGAACAGUGUCCUUACGA | 1012 | UCGUAAGGACACUGUUCCN | 1784-1802 | 1782 |
| 496 | NGGAACAGUGUCCUUACGN | 1013 | NCGUAAGGACACUGUUCCN | 1784-1802 | 1782 |
| 497 | UUGUUGUAAUUGAAUACUG | 1014 | CAGUAUUCAAUUACAACAA | 1961-1979 | 1959 |
| 498 | AUGUUGUAAUUGAAUACUG | 1015 | CAGUAUUCAAUUACAACAU | 1961-1979 | 1959 |
| 499 | NUGUUGUAAUUGAAUACUG | 1016 | CAGUAUUCAAUUACAACAN | 1961-1979 | 1959 |
| 500 | NUGUUGUAAUUGAAUACUN | 1017 | NAGUAUUCAAUUACAACAN | 1961-1979 | 1959 |
| 501 | CAGUAAGUAUGCUAGAGUC | 1018 | GACUCUAGCAUACUUACUG | 2218-2236 | 2216 |
| 502 | AAGUAAGUAUGCUAGAGUC | 1019 | GACUCUAGCAUACUUACUU | 2218-2236 | 2216 |
| 503 | UAGUAAGUAUGCUAGAGUC | 1020 | GACUCUAGCAUACUUACUA | 2218-2236 | 2216 |
| 504 | NAGUAAGUAUGCUAGAGUC | 1021 | GACUCUAGCAUACUUACUN | 2218-2236 | 2216 |
| 505 | NAGUAAGUAUGCUAGAGUN | 1022 | NACUCUAGCAUACUUACUN | 2218-2236 | 2216 |

TABLE 2E
IAV RNAi agent (targeting NP) Antisense Strand and Sense Strand Core Stretch Base Sequences
(N = any nucleobase)

| | | | | Corresponding | |
|---|---|---|---|---|---|
| | Antisense Strand Base Sequence | | Sense Strand Base Sequence | Positions of | Targeted |
| SEQ | (5′ → 3′) | SEQ | (5′ → 3′) | Identified | Viral |
| ID | (Shown as an Unmodified | ID | (Shown as an Unmodified | Sequence on | Genome |
| NO:. | Nucleotide Sequence) | NO:. | Nucleotide Sequence) | SEQ ID NO: 5 | Position |
| 506 | CACCAAUCAUUCUUCCGAC | 1023 | GUCGGAAGAAUGAUUGGUG | 85-103 | 83 |
| 507 | AACCAAUCAUUCUUCCGAC | 1024 | GUCGGAAGAAUGAUUGGUU | 85-103 | 83 |
| 508 | UACCAAUCAUUCUUCCGAC | 1025 | GUCGGAAGAAUGAUUGGUA | 85-103 | 83 |
| 509 | NACCAAUCAUUCUUCCGAC | 1026 | GUCGGAAGAAUGAUUGGUN | 85-103 | 83 |
| 510 | NACCAAUCAUUCUUCCGAN | 1027 | NUCGGAAGAAUGAUUGGUN | 85-103 | 83 |
| 511 | AUGCUAUUCUGGAUUAGUC | 1028 | GACUAAUCCAGAAUAGCAU | 164-182 | 162 |
| 512 | UUGCUAUUCUGGAUUAGUC | 1029 | GACUAAUCCAGAAUAGCAA | 164-182 | 162 |
| 513 | NUGCUAUUCUGGAUUAGUC | 1030 | GACUAAUCCAGAAUAGCAN | 164-182 | 162 |
| 514 | NUGCUAUUCUGGAUUAGUN | 1031 | NACUAAUCCAGAAUAGCAN | 164-182 | 162 |
| 515 | GCAGAAAGCACCAUCCUCU | 1032 | AGAGGAUGGUGCUUUCUGC | 191-209 | 189 |
| 516 | ACAGAAAGCACCAUCCUCU | 1033 | AGAGGAUGGUGCUUUCUGU | 191-209 | 189 |
| 517 | UCAGAAAGCACCAUCCUCU | 1034 | AGAGGAUGGUGCUUUCUGA | 191-209 | 189 |
| 518 | NCAGAAAGCACCAUCCUCU | 1035 | AGAGGAUGGUGCUUUCUGN | 191-209 | 189 |
| 519 | NCAGAAAGCACCAUCCUCN | 1036 | NGAGGAUGGUGCUUUCUGN | 191-209 | 189 |
| 520 | GCCAAAUCAUGAUAUGAGU | 1037 | ACUCAUAUCAUGAUUUGGC | 400-418 | 398 |
| 521 | ACCAAAUCAUGAUAUGAGU | 1038 | ACUCAUAUCAUGAUUUGGU | 400-418 | 398 |
| 522 | UCCAAAUCAUGAUAUGAGU | 1039 | ACUCAUAUCAUGAUUUGGA | 400-418 | 398 |
| 523 | NCCAAAUCAUGAUAUGAGU | 1040 | ACUCAUAUCAUGAUUUGGN | 400-418 | 398 |
| 524 | NCCAAAUCAUGAUAUGAGN | 1041 | NCUCAUAUCAUGAUUUGGN | 400-418 | 398 |
| 525 | CUGAUAUGUGGCAUCAUUC | 1042 | GAAUGAUGCCACAUAUCAG | 429-447 | 427 |
| 526 | AUGAUAUGUGGCAUCAUUC | 1043 | GAAUGAUGCCACAUAUCAU | 429-447 | 427 |
| 527 | UUGAUAUGUGGCAUCAUUC | 1044 | GAAUGAUGCCACAUAUCAA | 429-447 | 427 |
| 528 | NUGAUAUGUGGCAUCAUUC | 1045 | GAAUGAUGCCACAUAUCAN | 429-447 | 427 |
| 529 | NUGAUAUGUGGCAUCAUUN | 1046 | NAAUGAUGCCACAUAUCAN | 429-447 | 427 |
| 530 | UGUUGAACCUUGCAUUAGA | 1047 | UCUAAUGCAAGGUUCAACA | 495-513 | 493 |
| 531 | AGUUGAACCUUGCAUUAGA | 1048 | UCUAAUGCAAGGUUCAACU | 495-513 | 493 |
| 532 | NGUUGAACCUUGCAUUAGA | 1049 | UCUAAUGCAAGGUUCAACN | 495-513 | 493 |
| 533 | NGUUGAACCUUGCAUUAGN | 1050 | NCUAAUGCAAGGUUCAACN | 495-513 | 493 |
| 534 | CCACGUUUGAUCAUUCUGA | 1051 | UCAGAAUGAUCAAACGUGG | 581-599 | 579 |
| 535 | ACACGUUUGAUCAUUCUGA | 1052 | UCAGAAUGAUCAAACGUGU | 581-599 | 579 |
| 536 | UCACGUUUGAUCAUUCUGA | 1053 | UCAGAAUGAUCAAACGUGA | 581-599 | 579 |
| 537 | NCACGUUUGAUCAUUCUGA | 1054 | UCAGAAUGAUCAAACGUGN | 581-599 | 579 |
| 538 | NCACGUUUGAUCAUUCUGN | 1055 | NCAGAAUGAUCAAACGUGN | 581-599 | 579 |
| 539 | CCAGAAAUUUCGGUCAUUG | 1056 | CAAUGACCGAAAUUUCUGG | 603-621 | 601 |
| 540 | ACAGAAAUUUCGGUCAUUG | 1057 | CAAUGACCGAAAUUUCUGU | 603-621 | 601 |
| 541 | UCAGAAAUUUCGGUCAUUG | 1058 | CAAUGACCGAAAUUUCUGA | 603-621 | 601 |
| 542 | NCAGAAAUUUCGGUCAUUG | 1059 | CAAUGACCGAAAUUUCUGN | 603-621 | 601 |
| 543 | NCAGAAAUUUCGGUCAUUN | 1060 | NAAUGACCGAAAUUUCUGN | 603-621 | 601 |
| 544 | CCUUUGAGGAUAUUGCACA | 1061 | UGUGCAAUAUCCUCAAAGG | 665-683 | 663 |
| 545 | ACUUUGAGGAUAUUGCACA | 1062 | UGUGCAAUAUCCUCAAAGU | 665-683 | 663 |
| 546 | UCUUUGAGGAUAUUGCACA | 1063 | UGUGCAAUAUCCUCAAAGA | 665-683 | 663 |
| 547 | NCUUUGAGGAUAUUGCACA | 1064 | UGUGCAAUAUCCUCAAAGN | 665-683 | 663 |
| 548 | NCUUUGAGGAUAUUGCACN | 1065 | NGUGCAAUAUCCUCAAAGN | 665-683 | 663 |
| 549 | CUCAGAAUGAGUGCUGACC | 1066 | GGUCAGCACUCAUUCUGAG | 782-800 | 780 |
| 550 | AUCAGAAUGAGUGCUGACC | 1067 | GGUCAGCACUCAUUCUGAU | 782-800 | 780 |
| 551 | UUCAGAAUGAGUGCUGACC | 1068 | GGUCAGCACUCAUUCUGAA | 782-800 | 780 |
| 552 | NUCAGAAUGAGUGCUGACC | 1069 | GGUCAGCACUCAUUCUGAN | 782-800 | 780 |
| 553 | NUCAGAAUGAGUGCUGACN | 1070 | NGUCAGCACUCAUUCUGAN | 782-800 | 780 |
| 554 | GGAUUUAUGUGCAACUGAU | 1071 | AUCAGUUGCACAUAAAUCC | 804-822 | 802 |
| 555 | AGAUUUAUGUGCAACUGAU | 1072 | AUCAGUUGCACAUAAAUCU | 804-822 | 802 |
| 556 | UGAUUUAUGUGCAACUGAU | 1073 | AUCAGUUGCACAUAAAUCA | 804-822 | 802 |
| 557 | NGAUUUAUGUGCAACUGAU | 1074 | AUCAGUUGCACAUAAAUCN | 804-822 | 802 |
| 558 | NGAUUUAUGUGCAACUGAN | 1075 | NUCAGUUGCACAUAAAUCN | 804-822 | 802 |
| 559 | CACCAAUUGACUCUUGUGA | 1076 | UCACAAGAGUCAAUUGGUG | 969-987 | 967 |
| 560 | AACCAAUUGACUCUUGUGA | 1077 | UCACAAGAGUCAAUUGGUU | 969-987 | 967 |
| 561 | UACCAAUUGACUCUUGUGA | 1078 | UCACAAGAGUCAAUUGGUA | 969-987 | 967 |
| 562 | NACCAAUUGACUCUUGUGA | 1079 | UCACAAGAGUCAAUUGGUN | 969-987 | 967 |
| 563 | NACCAAUUGACUCUUGUGN | 1080 | NCACAAGAGUCAAUUGGUN | 969-987 | 967 |
| 564 | CUCUUAUGAAACUUGAUAC | 1081 | GUAUCAAGUUUCAUAAGAG | 1027-1045 | 1025 |
| 565 | AUCUUAUGAAACUUGAUAC | 1082 | GUAUCAAGUUUCAUAAGAU | 1027-1045 | 1025 |
| 566 | UUCUUAUGAAACUUGAUAC | 1083 | GUAUCAAGUUUCAUAAGAA | 1027-1045 | 1025 |
| 567 | NUCUUAUGAAACUUGAUAC | 1084 | GUAUCAAGUUUCAUAAGAN | 1027-1045 | 1025 |
| 568 | NUCUUAUGAAACUUGAUAN | 1085 | NUAUCAAGUUUCAUAAGAN | 1027-1045 | 1025 |
| 569 | UGAGAAUGUAGGCUGCACA | 1086 | UGUGCAGCCUACAUUCUCA | 1221-1239 | 1219 |
| 570 | AGAGAAUGUAGGCUGCACA | 1087 | UGUGCAGCCUACAUUCUCU | 1221-1239 | 1219 |
| 571 | NGAGAAUGUAGGCUGCACA | 1088 | UGUGCAGCCUACAUUCUCN | 1221-1239 | 1219 |
| 572 | NGAGAAUGUAGGCUGCACN | 1089 | NGUGCAGCCUACAUUCUCN | 1221-1239 | 1219 |
| 573 | UUACUCAUGUCAAAGGAAG | 1090 | CUUCCUUUGACAUGAGUAA | 1430-1448 | 1428 |
| 574 | AUACUCAUGUCAAAGGAAG | 1091 | CUUCCUUUGACAUGAGUAU | 1430-1448 | 1428 |
| 575 | NUACUCAUGUCAAAGGAAG | 1092 | CUUCCUUUGACAUGAGUAN | 1430-1448 | 1428 |
| 576 | NUACUCAUGUCAAAGGAAN | 1093 | NUUCCUUUGACAUGAGUAN | 1430-1448 | 1428 |
| 577 | AGAAAUAAGACCCUUCAUU | 1094 | AAUGAAGGGUCUUAUUUCU | 1447-1465 | 1445 |
| 578 | UGAAAUAAGACCCUUCAUU | 1095 | AAUGAAGGGUCUUAUUUCA | 1447-1465 | 1445 |
| 579 | NGAAAUAAGACCCUUCAUU | 1096 | AAUGAAGGGUCUUAUUUCN | 1447-1465 | 1445 |
| 580 | NGAAAUAAGACCCUUCAUN | 1097 | NAUGAAGGGUCUUAUUUCN | 1447-1465 | 1445 |

TABLE 2F
IAV RNAi agent (targeting PA) Antisense Strand and Sense Strand Core Stretch Base Sequences
(N = any nucleobase)

| | | | | Corresponding | |
|---|---|---|---|---|---|
| | Antisense Strand Base Sequence | | Sense Strand Base Sequence | Positions of | Targeted |
| SEQ | (5′ → 3′) | SEQ | (5′ → 3′) | Identified | Viral |
| ID | (Shown as an Unmodified | ID | (Shown as an Unmodified | Sequence on | Genome |
| NO:. | Nucleotide Sequence) | NO:. | Nucleotide Sequence) | SEQ ID NO: 6 | Position |
| 581 | GAAGCAUUGUCGCACAAAG | 1098 | CUUUGUGCGACAAUGCUUC | 9-27 | 7 |
| 582 | AAAGCAUUGUCGCACAAAG | 1099 | CUUUGUGCGACAAUGCUUU | 9-27 | 7 |
| 583 | UAAGCAUUGUCGCACAAAG | 1100 | CUUUGUGCGACAAUGCUUA | 9-27 | 7 |
| 584 | NAAGCAUUGUCGCACAAAG | 1101 | CUUUGUGCGACAAUGCUUN | 9-27 | 7 |
| 585 | NAAGCAUUGUCGCACAAAN | 1102 | NUUUGUGCGACAAUGCUUN | 9-27 | 7 |
| 586 | UGAAAUGGAAAUCCGAAUA | 1103 | UAUUCGGAUUUCCAUUUCA | 142-160 | 140 |
| 587 | AGAAAUGGAAAUCCGAAUA | 1104 | UAUUCGGAUUUCCAUUUCU | 142-160 | 140 |
| 588 | NGAAAUGGAAAUCCGAAUA | 1105 | UAUUCGGAUUUCCAUUUCN | 142-160 | 140 |
| 589 | NGAAAUGGAAAUCCGAAUN | 1106 | NAUUCGGAUUUCCAUUUCN | 142-160 | 140 |
| 590 | CCAUGAUUCGGUCUCUUCC | 1107 | GGAAGAGACCGAAUCAUGG | 241-259 | 239 |
| 591 | ACAUGAUUCGGUCUCUUCC | 1108 | GGAAGAGACCGAAUCAUGU | 241-259 | 239 |
| 592 | UCAUGAUUCGGUCUCUUCC | 1109 | GGAAGAGACCGAAUCAUGA | 241-259 | 239 |
| 593 | NCAUGAUUCGGUCUCUUCC | 1110 | GGAAGAGACCGAAUCAUGN | 241-259 | 239 |
| 594 | NCAUGAUUCGGUCUCUUCN | 1111 | NGAAGAGACCGAAUCAUGN | 241-259 | 239 |
| 595 | UGAAAAGCCUAGUUUUGAU | 1112 | AUCAAAACUAGGCUUUUCA | 511-529 | 509 |
| 596 | AGAAAAGCCUAGUUUUGAU | 1113 | AUCAAAACUAGGCUUUUCU | 511-529 | 509 |
| 597 | NGAAAAGCCUAGUUUUGAU | 1114 | AUCAAAACUAGGCUUUUCN | 511-529 | 509 |
| 598 | NGAAAAGCCUAGUUUUGAN | 1115 | NUCAAAACUAGGCUUUUCN | 511-529 | 509 |
| 599 | GACGAAAGGAAUCCCAUAG | 1116 | CUAUGGGAUUCCUUUCGUC | 559-577 | 557 |
| 600 | AACGAAAGGAAUCCCAUAG | 1117 | CUAUGGGAUUCCUUUCGUU | 559-577 | 557 |
| 601 | UACGAAAGGAAUCCCAUAG | 1118 | CUAUGGGAUUCCUUUCGUA | 559-577 | 557 |
| 602 | NACGAAAGGAAUCCCAUAG | 1119 | CUAUGGGAUUCCUUUCGUN | 559-577 | 557 |
| 503 | NACGAAAGGAAUCCCAUAN | 1120 | NUAUGGGAUUCCUUUCGUN | 559-577 | 557 |
| 604 | CUUCAAUUGUCUCUUCGCC | 1121 | GGCGAAGAGACAAUUGAAG | 589-607 | 587 |
| 605 | AUUCAAUUGUCUCUUCGCC | 1122 | GGCGAAGAGACAAUUGAAU | 589-607 | 587 |
| 606 | UUUCAAUUGUCUCUUCGCC | 1123 | GGCGAAGAGACAAUUGAAA | 589-607 | 587 |
| 607 | NUUCAAUUGUCUCUUCGCC | 1124 | GGCGAAGAGACAAUUGAAN | 589-607 | 587 |
| 608 | NUUCAAUUGUCUCUUCGCN | 1125 | NGCGAAGAGACAAUUGAAN | 589-607 | 587 |
| 609 | CUCGAAUCCAUCUACAUAG | 1126 | CUAUGUAGAUGGAUUCGAG | 693-711 | 691 |
| 610 | AUCGAAUCCAUCUACAUAG | 1127 | CUAUGUAGAUGGAUUCGAU | 693-711 | 691 |
| 611 | UUCGAAUCCAUCUACAUAG | 1128 | CUAUGUAGAUGGAUUCGAA | 693-711 | 691 |
| 612 | NUCGAAUCCAUCUACAUAG | 1129 | CUAUGUAGAUGGAUUCGAN | 693-711 | 691 |
| 613 | NUCGAAUCCAUCUACAUAN | 1130 | NUAUGUAGAUGGAUUCGAN | 693-711 | 691 |
| 614 | UGACAUUUGGGAAAGCUUG | 1131 | CAAGCUUUCCCAAAUGUCA | 732-750 | 730 |
| 615 | AGACAUUUGGGAAAGCUUG | 1132 | CAAGCUUUCCCAAAUGUCU | 732-750 | 730 |
| 616 | NGACAUUUGGGAAAGCUUG | 1133 | CAAGCUUUCCCAAAUGUCN | 732-750 | 730 |
| 617 | NGACAUUUGGGAAAGCUUN | 1134 | NAAGCUUUCCCAAAUGUCN | 732-750 | 730 |
| 618 | UUUGAUUGCAUCAUAUAGU | 1135 | ACUAUAUGAUGCAAUCAAA | 909-927 | 907 |
| 619 | AUUGAUUGCAUCAUAUAGU | 1136 | ACUAUAUGAUGCAAUCAAU | 909-927 | 907 |
| 620 | NUUGAUUGCAUCAUAUAGU | 1137 | ACUAUAUGAUGCAAUCAAN | 909-927 | 907 |
| 621 | NUUGAUUGCAUCAUAUAGN | 1138 | NCUAUAUGAUGCAAUCAAN | 909-927 | 907 |
| 622 | AAGGUCUCCAACAUCUUUG | 1139 | CAAAGAUGUUGGAGACCUU | 1152-1170 | 1150 |
| 623 | UAGGUCUCCAACAUCUUUG | 1140 | CAAAGAUGUUGGAGACCUA | 1152-1170 | 1150 |
| 624 | NAGGUCUCCAACAUCUUUG | 1141 | CAAAGAUGUUGGAGACCUN | 1152-1170 | 1150 |
| 625 | NAGGUCUCCAACAUCUUUN | 1142 | NAAAGAUGUUGGAGACCUN | 1152-1170 | 1150 |
| 626 | UCAUCAAGUUCUAUCCAGC | 1143 | GCUGGAUAGAACUUGAUGA | 1262-1280 | 1260 |
| 627 | ACAUCAAGUUCUAUCCAGC | 1144 | GCUGGAUAGAACUUGAUGU | 1262-1280 | 1260 |
| 628 | NCAUCAAGUUCUAUCCAGC | 1145 | GCUGGAUAGAACUUGAUGN | 1262-1280 | 1260 |
| 629 | NCAUCAAGUUCUAUCCAGN | 1146 | NCUGGAUAGAACUUGAUGN | 1262-1280 | 1260 |
| 630 | ACAUUUGCUUAUCAUUGGG | 1147 | CCCAAUGAUAAGCAAAUGU | 1449-1467 | 1447 |
| 631 | UCAUUUGCUUAUCAUUGGG | 1148 | CCCAAUGAUAAGCAAAUGA | 1449-1467 | 1447 |
| 632 | NCAUUUGCUUAUCAUUGGG | 1149 | CCCAAUGAUAAGCAAAUGN | 1449-1467 | 1447 |
| 633 | NCAUUUGCUUAUCAUUGGN | 1150 | NCCAAUGAUAAGCAAAUGN | 1449-1467 | 1447 |
| 634 | UCAUUAAUCAGGCACUCCU | 1151 | AGGAGUGCCUGAUUAAUGA | 2072-2090 | 2070 |
| 635 | ACAUUAAUCAGGCACUCCU | 1152 | AGGAGUGCCUGAUUAAUGU | 2072-2090 | 2070 |
| 636 | NCAUUAAUCAGGCACUCCU | 1153 | AGGAGUGCCUGAUUAAUGN | 2072-2090 | 2070 |
| 637 | NCAUUAAUCAGGCACUCCN | 1154 | NGGAGUGCCUGAUUAAUGN | 2072-2090 | 2070 |

The IAV RNAi agent sense strands and antisense strands that comprise or consist of the nucleotide sequences in Table 2A, 2B, 2C, 2D, 2E, and 2F can be modified nucleotides or unmodified nucleotides. In some embodiments, the IAV RNAi agents having the sense and antisense strand sequences that comprise or consist of any of the nucleotide sequences in Table 2A, 2B, 2C, 2D, 2E, and 2F are all or substantially all modified nucleotides.

In some embodiments, the antisense strand of an IAV RNAi agent disclosed herein differs by 0, 1, 2, or 3 nucleotides from any of the antisense strand sequences in Table 2A, 2B, 2C, 2D, 2E, and 2F. In some embodiments, the sense strand of an IAV RNAi agent disclosed herein differs by 0, 1, 2, or 3 nucleotides from any of the sense strand sequences in Table 2A, 2B, 2C, 2D, 2E, and 2F.

As used herein, each N listed in a sequence disclosed in Table 2A, 2B, 2C, 2D, 2E, and 2F may be independently selected from any and all nucleobases (including those found on both modified and unmodified nucleotides). In some embodiments, an N nucleotide listed in a sequence disclosed in Table 2A, 2B, 2C, 2D, 2E, and 2F has a nucleobase that is complementary to the N nucleotide at the corresponding position on the other strand. In some embodiments, an N nucleotide listed in a sequence disclosed in Table 2A, 2B, 2C, 2D, 2E, and 2F has a nucleobase that is not complementary to the N nucleotide at the corresponding position on the other strand. In some embodiments, an N nucleotide listed in a sequence disclosed in Table 2A, 2B, 2C, 2D, 2E, and 2F has a nucleobase that is the same as the N nucleotide at the corresponding position on the other strand. In some embodiments, an N nucleotide listed in a sequence disclosed in Table 2A, 2B, 2C, 2D, 2E, and 2F has a nucleobase that is different from the N nucleotide at the corresponding position on the other strand.

Certain modified IAV RNAi agent sense and antisense strands are provided in Table 3A, 3B, 3C, 3D, 3E, 3F, 4A, 4B, 4C, 4D, 4E, 4F, 5A, 5B, 5C, 5D, 5E, 5F, 6A, 6B, 6C, 6D, 6E, 6F, 10A, 10B, 10C, 10D, 10E, and 10F. Certain modified IAV RNAi agent antisense strands, as well as their underlying unmodified nucleobase sequences, are provided in Table 3A, 3B, 3C, 3D, 3E, 3F. Certain modified IAV RNAi agent sense strands, as well as their underlying unmodified nucleobase sequences, are provided in Tables 4A, 4B, 4C, 4D, 4E, 4F, 5A, 5B, 5C, 5D, 5E, 5F, 6A, 6B, 6C, 6D, 6E, and 6F. In forming IAV RNAi agents, each of the nucleotides in each of the underlying base sequences listed in Tables 3A, 3B, 3C, 3D, 3E, 3F, 4A, 4B, 4C, 4D, 4E, 4F, 5A, 5B, 5C, 5D, 5E, 5F, 6A, 6B, 6C, 6D, 6E, 6F, as well as in Tables 2A, 2B, 2C, 2D, 2E, 2F, above, can be a modified nucleotide.

The IAV RNAi agents described herein are formed by annealing an antisense strand with a sense strand. A sense strand containing a sequence listed in Table 2A, 2B, 2C, 2D, 2E, 2F, 4A, 4B, 4C, 4D, 4E, 4F, 5A, 5B, 5C, 5D, 5E, 5F, 6A, 6B, 6C, 6D, 6E, or 6F can be hybridized to any antisense strand containing a sequence listed in Table 2A, 2B, 2C, 2D, 2E, 2F, 3A, 3B, 3C, 3D, 3E, or 3F provided the two sequences have a region of at least 85% complementarity over a contiguous 16, 17, 18, 19, 20, or 21 nucleotide sequence.

In some embodiments, an IAV RNAi agent antisense strand comprises a nucleotide sequence of any of the sequences in Table 2A, 2B, 2C, 2D, 2E, 2F, 3A, 3B, 3C, 3D, 3E, or 3F.

In some embodiments, an IAV RNAi agent comprises or consists of a duplex having the nucleobase sequences of the sense strand and the antisense strand of any of the sequences in 2A, 2B, 2C, 2D, 2E, 2F, 3A, 3B, 3C, 3D, 3E, 3F, 4A, 4B, 4C, 4D, 4E, 4F, 5A, 5B, 5C, 5D, 5E, 5F, 6A, 6B, 6C, 6D, 6E, 6F, 10A, 10B, 10C, 10D, 10E, or 10F.

Examples of antisense strands containing modified nucleotides are provided in Table 3A, 3B, 3C, 3D, 3E, and 3F. Examples of sense strands containing modified nucleotides are provided in Tables 4A, 4B, 4C, 4D, 4E, 4F, 5A, 5B, 5C, 5D, 5E, 5F, 6A, 6B, 6C, 6D, 6E, and 6F.

As used in Tables 3A, 3B, 3C, 3D, 3E, 3F, 4A, 4B, 4C, 4D, 4E, 4F, 5A, 5B, 5C, 5D, 5E, 5F, 6A, 6B, 6C, 6D, 6E, 6F, 10A, 10B, 10C, 10D, 10E, and 10F, the following notations are used to indicate modified nucleotides, targeting groups, and linking groups:
A=adenosine-3′-phosphate
C=cytidine-3′-phosphate
G=guanosine-3′-phosphate
U=uridine-3′-phosphate
I=inosine-3′-phosphate
a=2′-O-methyladenosine-3′-phosphate
as =2′-O-methyladenosine-3′-phosphorothioate
c=2′-O-methylcytidine-3′-phosphate
cs=2′-O-methylcytidine-3′-phosphorothioate
g=2′-O-methylguanosine-3′-phosphate
gs=2′-O-methylguanosine-3′-phosphorothioate
i=2′-O-methylinosine-3′-phosphate
is=2′-O-methylinosine-3′-phosphorothioate
t=2′-O-methyl-5-methyluridine-3′-phosphate
ts=2′-O-methyl-5-methyluridine-3′-phosphorothioate
u=2′-O-methyluridine-3′-phosphate
us=2′-O-methyluridine-3′-phosphorothioate
Af=2′-fluoroadenosine-3′-phosphate
Afs=2′-fluoroadenosine-3′-phosporothioate
Cf=2′-fluorocytidine-3′-phosphate
Cfs=2′-fluorocytidine-3′-phosphorothioate
Gf=2′-fluoroguanosine-3′-phosphate
Gfs=2′-fluoroguanosine-3′-phosphorothioate
Tf=2′-fluoro-5′-methyluridine-3′-phosphate
Tfs=2′-fluoro-5′-methyluridine-3′-phosphorothioate
Uf=2′-fluorouridine-3′-phosphate
Ufs=2′-fluorouridine-3′-phosphorothioate
dT=2′-deoxythymidine-3′-phosphate
A_{UNA}=2′,3′-seco-adenosine-3′-phosphate
A_{UNA}s=2′,3′-seco-adenosine-3′-phosphorothioate
C_{UNA}=2′,3′-seco-cytidine-3′-phosphate
C_{UNA}s=2′,3′-seco-cytidine-3′-phosphorothioate
G_{UNA}=2′,3′-seco-guanosine-3′-phosphate
G_{UNA}s=2′,3′-seco-guanosine-3′-phosphorothioate
U_{UNA}=2′,3′-seco-uridine-3′-phosphate
U_{UNA}s=2′,3′-seco-uridine-3′-phosphorothioate
a_2N=see Table 11
a_2Ns=see Table 11
(invAb)=inverted abasic deoxyribonucleotide-5′-phosphate, see Table 11
(invAb)s=inverted abasic deoxyribonucleotide-5′-phosphorothioate, see Table 11
s=phosphorothioate linkage
p=terminal phosphate (as synthesized)
vpdN=vinyl phosphonate deoxyribonucleotide
cPrpa=5′-cyclopropyl phosphonate-2′-O-methyladenosine-3′-phosphate (see Table 11)
cPrpas=5′-cyclopropyl phosphonate-2′-O-methyladenosine-3′-phosphorothioate (see Table 11)
cPrpu=5′-cyclopropyl phosphonate-2′-O-methyluridine-3′-phosphate (see Table 11)
cPrpus=5′-cyclopropyl phosphonate-2′-O-methyluridine-3′-phosphorothioate (see Table 11)
(Alk-SS-C6)=see Table 11
(C6-SS-Alk)=see Table 11
(C6-SS-C6)=see Table 11
(6-SS-6)=see Table 11
(C6-SS-Alk-Me)=see Table 11
(NH2-C6)=see Table 11
(TriAlk14)=see Table 11
(TriAlk14)s=see Table 11
—C6-=see Table 11
—C6s-=see Table 11
-L6-C6-=see Table 11
-L6-C6s-=see Table 11
Alk-cyHex-=see Table 11
Alk-cyHexs-=see Table 11
(TA14)=see Table 11 (structure of (TriAlk14)s after conjugation)
(TA14)s=see Table 11 (structure of (TriAlk14)s after conjugation)

As the person of ordinary skill in the art would readily understand, unless otherwise indicated by the sequence (such as, for example, by a phosphorothioate linkage “s”), when present in an oligonucleotide, the nucleotide monomers are mutually linked by 5′-3′-phosphodiester bonds. As the person of ordinary skill in the art would clearly understand, the inclusion of a phosphorothioate linkage as shown in the modified nucleotide sequences disclosed herein replaces the phosphodiester linkage typically present in oligonucleotides. Further, the person of ordinary skill in the art would readily understand that the terminal nucleotide at the 3′ end of a given oligonucleotide sequence would typically have a hydroxyl (—OH) group at the respective 3′ position of the given monomer instead of a phosphate moiety ex vivo. Additionally, for the embodiments disclosed herein, when viewing the respective strand 5′→3′, the inverted abasic residues are inserted such that the 3′ position of the deoxyribose is linked at the 3′ end of the preceding monomer on the respective strand (see, e.g., Table 11). Moreover, as the person of ordinary skill would readily understand and appreciate, while the phosphorothioate chemical structures depicted herein typically show the anion on the sulfur atom, the inventions disclosed herein encompass all phosphorothioate tautomers (e.g., where the sulfur atom has a double-bond and the anion is on an oxygen atom). Unless expressly indicated otherwise herein, such understandings of the person of ordinary skill in the art are used when describing the IAV RNAi agents and compositions of IAV RNAi agents disclosed herein.

Certain examples of targeting groups and linking groups used with the IAV RNAi agents disclosed herein are included in the chemical structures provided below in Table 11. Each sense strand and/or antisense strand can have any targeting groups or linking groups listed herein, as well as other targeting or linking groups, conjugated to the 5′ and/or 3′ end of the sequence.

TABLE 3A
IAV RNAi agent (targeting M1) Antisense Strand Sequences

| | | SEQ | Underlying Base Sequence (5′ → 3′) | SEQ |
|---|---|---|---|---|
| | | ID | (Shown as an Unmodified Nucleotide | ID |
| AS Strand ID | Modified Antisense Strand (5′ → 3′) | NO. | Sequence) | NO. |
| AM15368-AS | usUfsasCfgUfuucgaCfcUfcGfgUfuasg | 1155 | UUACGUUUCGACCUCGGUUAG | 1590 |
| AM15370-AS | usAfsusGfaUfagaaaGfaAfcGfuAfcgsu | 1156 | UAUGAUAGAAAGAACGUACGU | 1591 |
| AM15372-AS | usUfsgsAfcAfagauuGfgUfcUfuGfucsu | 1157 | UUGACAAGAUUGGUCUUGUCU | 1592 |
| AM15374-AS | asGfsusCfaGfaggugAfcAfaGfaUfugsg | 1158 | AGUCAGAGGUGACAAGAUUGG | 1593 |
| AM15376-AS | usUfsasGfuCfagaggUfgAfcAfaGfausc | 1159 | UUAGUCAGAGGUGACAAGAUC | 1594 |
| AM15378-AS | usCfsusCfuAfuccauGfuUfgUfuCfggsg | 1160 | UCUCUAUCCAUGUUGUUCGGG | 1595 |
| AM15380-AS | asAfscsUfgCfucuauCfcAfuGfuUfgusc | 1161 | AACUGCUCUAUCCAUGUUGUC | 1596 |
| AM15382-AS | usAfsgsUfuGfaauagCfuUfaGfuGfacsa | 1162 | UAGUUGAAUAGCUUAGUGACA | 1597 |
| AM15384-AS | usUfsgsGfcAfagugcAfcCfaGfuUfgasc | 1163 | UUGGCAAGUGCACCAGUUGAC | 1598 |
| AM15386-AS | asAfsasGfcAfgcuucUfgUfgGfuCfacsu | 1164 | AAAGCAGCUUCUGUGGUCACU | 1599 |
| AM15388-AS | usCfscsUfgAfuuaguGfgAfuUfgGfugsg | 1165 | UCCUGAUUAGUGGAUUGGUGG | 1600 |
| AM15390-AS | usUfsasGfgAfugaguCfcCfaAfuAfgusc | 1166 | UUAGGAUGAGUCCCAAUAGUC | 1601 |
| AM15392-AS | usAfscsAfaAfaugacCfaUfcGfuCfaasc | 1167 | UACAAAAUGACCAUCGUCAAC | 1602 |
| AM15394-AS | usGfsasCfaAfaaugaCfcAfuCfgUfcasc | 1168 | UGACAAAAUGACCAUCGUCAC | 1603 |
| AM16677-AS | cPrpusUfsasCfgUfuucgaCfcUfcGfgUfuasg | 1169 | UUACGUUUCGACCUCGGUUAG | 1590 |
| AM16812-AS | cPrpusAfsusGfaUfagaaaGfaAfcGfuAfcgsu | 1170 | UAUGAUAGAAAGAACGUACGU | 1591 |
| AM17566-AS | cPrpusAfscsAfaAfaugacCfaUfcGfuCfaasc | 1171 | UACAAAAUGACCAUCGUCAAC | 1602 |
| AM17568-AS | cPrpusGfsasCfaAfaaugaCfcAfuCfgUfcasc | 1172 | UGACAAAAUGACCAUCGUCAC | 1603 |
| AM17928-AS | cPrpusUfsAfscguuucgaCfcUfcGfguuasg | 1173 | UUACGUUUCGACCUCGGUUAG | 1590 |
| AM17929-AS | cPrpusUfsasCfguuucgaCfcUfcGfguuasg | 1174 | UUACGUUUCGACCUCGGUUAG | 1590 |
| AM17930-AS | cPrpusUfsascGfuuucgaCfcUfcGfguuasg | 1175 | UUACGUUUCGACCUCGGUUAG | 1590 |
| AM17931-AS | cPrpusUfsascguUfucgaCfcUfcGfguuasg | 1176 | UUACGUUUCGACCUCGGUUAG | 1590 |
| AM17933-AS | cPrpusUfsascguuuCfgaCfcUfcGfguuasg | 1177 | UUACGUUUCGACCUCGGUUAG | 1590 |
| AM17934-AS | cPrpusUfsaCfgUfuucgaCfcUfcGfgUfuasg | 1178 | UUACGUUUCGACCUCGGUUAG | 1590 |
| AM18487-AS | cPrpusCfsusCfuAfuccauGfuUfgUfuCfggsg | 1179 | UCUCUAUCCAUGUUGUUCGGG | 1595 |
| AM18489-AS | cPrpasAfscsUfgCfucuauCfcAfuGfuUfgusc | 1180 | AACUGCUCUAUCCAUGUUGUC | 1596 |
| AM18520-AS | cPrpusAfsuGfaUfagaaaGfaAfcGfuAfcsgsu | 1181 | UAUGAUAGAAAGAACGUACGU | 1591 |
| AM18521-AS | cPrpusAfsuGfaUfagaaaGfaAfcGfuAfcgsu | 1182 | UAUGAUAGAAAGAACGUACGU | 1591 |
| AM18523-AS | cPrpusAfsUfsgauagaaaGfaAfcGfuacgsu | 1183 | UAUGAUAGAAAGAACGUACGU | 1591 |
| AM18524-AS | cPrpusAfsusgAfuagaaaGfaAfcGfuacgsu | 1184 | UAUGAUAGAAAGAACGUACGU | 1591 |
| AM18525-AS | cPrpusAfsusgauAfgaaaGfaAfcGfuacgsu | 1185 | UAUGAUAGAAAGAACGUACGU | 1591 |
| AM18526-AS | cPrpusAfsusgauagAfaaGfaAfcGfuacgsu | 1186 | UAUGAUAGAAAGAACGUACGU | 1591 |
| AM19907-AS | cPrpusAfsusGfauagaaaGfaAfcGfuacgsu | 1187 | UAUGAUAGAAAGAACGUACGU | 1591 |
| AM19908-AS | cPrpusAfsUfgauagaaaGfaAfcGfuacgsu | 1188 | UAUGAUAGAAAGAACGUACGU | 1591 |
| AM19909-AS | cPrpusAfsuGfauagaaaGfaAfcGfuacgsu | 1189 | UAUGAUAGAAAGAACGUACGU | 1591 |
| AM19910-AS | cPrpusAfsugAfuagaaaGfaAfcGfuacgsu | 1190 | UAUGAUAGAAAGAACGUACGU | 1591 |
| AM19911-AS | cPrpusAfsugauAfgaaaGfaAfcGfuacgsu | 1191 | UAUGAUAGAAAGAACGUACGU | 1591 |
| AM19912-AS | cPrpusAfsugauagAfaaGfaAfcGfuacgsu | 1192 | UAUGAUAGAAAGAACGUACGU | 1591 |

TABLE 3B
IAV RNAi agent (targeting NS1) Antisense Strand Sequences

| | | SEQ | Underlying Base Sequence (5′ → 3′) | SEQ |
|---|---|---|---|---|
| | | ID | (Shown as an Unmodified Nucleotide | ID |
| AS Strand ID | Modified Antisense Strand (5′ → 3′) | NO. | Sequence) | NO. |
| AM14463-AS | usAfsusCfaAfggaauGfgGfgCfaUfcasc | 1193 | UAUCAAGGAAUGGGGCAUCAC | 1604 |
| AM14465-AS | usGfsasUfcAfaggaaUfgGfgGfcAfucsa | 1194 | UGAUCAAGGAAUGGGGCAUCA | 1605 |
| AM14467-AS | usCfsgsAfuCfaaggaAfuGfgGfgCfausc | 1195 | UCGAUCAAGGAAUGGGGCAUC | 1606 |
| AM14469-AS | usGfscsAfuUfuuugaCfaUfcCfuCfausc | 1196 | UGCAUUUUUGACAUCCUCAUC | 1607 |
| AM14471-AS | usCfsasUfuCfaagucCfuCfcGfaUfgasg | 1197 | UCAUUCAAGUCCUCCGAUGAG | 1608 |
| AM14473-AS | usCfscsAfuUfcaaguCfcUfcCfgAfugsa | 1198 | UCCAUUCAAGUCCUCCGAUGA | 1609 |
| AM14475-AS | usUfscsCfaUfucaagUfcCfuCfcGfausg | 1199 | UUCCAUUCAAGUCCUCCGAUG | 1610 |
| AM14477-AS | usAfsusUfcCfauucaAfgUfcCfuCfcgsa | 1200 | UAUUCCAUUCAAGUCCUCCGA | 1611 |
| AM14479-AS | usCfsasUfuCfcauucAfaGfuCfcUfccsg | 1201 | UCAUUCCAUUCAAGUCCUCCG | 1612 |
| AM14481-AS | usUfscsCfaAfgcgaaUfcUfcUfgUfausc | 1202 | UUCCAAGCGAAUCUCUGUAUC | 1613 |
| AM14695-AS | usCfsasUfgAfgcaugAfaCfcAfgUfcusc | 1203 | UCAUGAGCAUGAACCAGUCUC | 1614 |
| AM14697-AS | asCfsasCfuGfaaguuCfgCfuUfuCfagsu | 1204 | ACACUGAAGUUCGCUUUCAGU | 1615 |
| AM14699-AS | asGfsusGfaAfagcccUfuAfgUfaGfuasc | 1205 | AGUGAAAGCCCUUAGUAGUAC | 1616 |
| AM14701-AS | usAfsgsUfgAfaagccCfuUfaGfuAfgusc | 1206 | UAGUGAAAGCCCUUAGUAGUC | 1617 |
| AM14703-AS | asAfsgsUfaUfguccuGfgAfaGfaGfaasg | 1207 | AAGUAUGUCCUGGAAGAGAAG | 1618 |
| AM14705-AS | usCfscsAfaCfugcauUfuUfuGfaCfausc | 1208 | UCCAACUGCAUUUUUGACAUC | 1619 |
| AM14707-AS | usAfsusGfaGfgacccCfaAfcUfgCfausc | 1209 | UAUGAGGACCCCAACUGCAUC | 1620 |
| AM14709-AS | usUfsgsUfuAfccauuCfcAfuUfcAfagsc | 1210 | UUGUUACCAUUCCAUUCAAGC | 1621 |
| AM14711-AS | usAfsgsAfcUfcgaacCfgUfgUfuAfccsa | 1211 | UAGACUCGAACCGUGUUACCA | 1622 |
| AM14713-AS | asAfsgsGfuCfucccaUfuCfuCfaUfcasc | 1212 | AAGGUCUCCCAUUCUCAUCAC | 1623 |
| AM14715-AS | usAfsgsUfuGfuaaggCfuUfgCfaUfaasc | 1213 | UAGUUGUAAGGCUUGCAUAAC | 1624 |
| AM14717-AS | asUfsasAfgCfugaaaCfgAfgAfaAfgcsu | 1214 | AUAAGCUGAAACGAGAAAGCU | 1625 |
| AM15163-AS | usAfsasCfaAfgagugGfcUfgUfuUfcgsa | 1215 | UAACAAGAGUGGCUGUUUCGA | 1626 |
| AM15165-AS | usCfsasAfgAfuccauUfcCfaCfgAfuusc | 1216 | UCAAGAUCCAUUCCACGAUUC | 1627 |
| AM15167-AS | usUfsgsGfaUfuccucUfuUfcAfaGfausc | 1217 | UUGGAUUCCUCUUUCAAGAUC | 1628 |
| AM15169-AS | asGfsgsUfaCfagaugCfaAfuUfgUfcasc | 1218 | AGGUACAGAUGCAAUUGUCAC | 1629 |
| AM15171-AS | usCfsgsAfaGfuagguAfcAfgAfuGfcasc | 1219 | UCGAAGUAGGUACAGAUGCAC | 1630 |
| AM15173-AS | asUfsgsAfgCfaugaaCfcAfgUfcUfcgsu | 1220 | AUGAGCAUGAACCAGUCUCGU | 1631 |
| AM15175-AS | asAfsasGfaUfuacacUfgAfaGfuUfcgsc | 1221 | AAAGAUUACACUGAAGUUCGC | 1632 |
| AM15177-AS | usUfsasUfcAfaggucUfcUfaAfuCfggsu | 1222 | UUAUCAAGGUCUCUAAUCGGU | 1633 |
| AM15179-AS | asCfsusCfgAfaccguGfuUfaCfcAfuusc | 1223 | ACUCGAACCGUGUUACCAUUC | 1634 |
| AM15181-AS | usAfsgsUfgAfaggucUfcCfcAfuUfcusc | 1224 | UAGUGAAGGUCUCCCAUUCUC | 1635 |
| AM15183-AS | usUfscsGfaAfacuauUfcUfcUfgUfcgsc | 1225 | UUCGAAACUAUUCUCUGUCGC | 1636 |
| AM15185-AS | asAfscsGfaGfaaagcUfcUfuAfuCfucsc | 1226 | AACGAGAAAGCUCUUAUCUCC | 1637 |
| AM15546-AS | usUfscsCfaAfgcgaaUfcUfcUfgUfausg | 1227 | UUCCAAGCGAAUCUCUGUAUG | 1638 |
| AM15547-AS | cPrpusUfscsCfaAfgcgaaUfcUfcUfgUfausc | 1228 | UUCCAAGCGAAUCUCUGUAUC | 1613 |
| AM15548-AS | cPrpusUfscCfaAfgcgaaUfcUfcUfgUfasusc | 1229 | UUCCAAGCGAAUCUCUGUAUC | 1613 |
| AM15549-AS | usUfsCfscaagcgaaUfcUfcUfguausc | 1230 | UUCCAAGCGAAUCUCUGUAUC | 1613 |
| AM15550-AS | usUfscsCfaagcgaaUfcUfcUfguausc | 1231 | UUCCAAGCGAAUCUCUGUAUC | 1613 |
| AM15710-AS | usGfscsAfuuuuugaCfaUfcCfucausc | 1232 | UGCAUUUUUGACAUCCUCAUC | 1607 |
| AM15711-AS | usGfscsauuUfuugaCfaUfcCfucausc | 1233 | UGCAUUUUUGACAUCCUCAUC | 1607 |
| AM15714-AS | cPrpusGfscsauuUfuugaCfaUfcCfucausc | 1234 | UGCAUUUUUGACAUCCUCAUC | 1607 |
| AM15716-AS | usUfsgsUfuaccauuCfcAfuUfcaagsc | 1235 | UUGUUACCAUUCCAUUCAAGC | 1621 |
| AM15717-AS | usUfsgsuuaCfcauuCfcAfuUfcaagsc | 1236 | UUGUUACCAUUCCAUUCAAGC | 1621 |
| AM15720-AS | usUfscsgAfaacuauUfcUfcUfgucgsc | 1237 | UUCGAAACUAUUCUCUGUCGC | 1636 |
| AM15721-AS | usUfscsgaaAfcuauUfcUfcUfgucgsc | 1238 | UUCGAAACUAUUCUCUGUCGC | 1636 |
| AM15725-AS | asUfsasAfgCfugaaaCfgAfgAfaAfgcsc | 1239 | AUAAGCUGAAACGAGAAAGCC | 1639 |
| AM15727-AS | asUfsAfsagcugaaaCfgAfgAfaagcsu | 1240 | AUAAGCUGAAACGAGAAAGCU | 1625 |
| AM15728-AS | asUfsasAfgcugaaaCfgAfgAfaagcsu | 1241 | AUAAGCUGAAACGAGAAAGCU | 1625 |
| AM15729-AS | cPrpasUfsasAfgCfugaaaCfgAfgAfaAfgcsu | 1242 | AUAAGCUGAAACGAGAAAGCU | 1625 |
| AM15730-AS | cPrpasUfsaAfgCfugaaaCfgAfgAfaAfgscsu | 1243 | AUAAGCUGAAACGAGAAAGCU | 1625 |
| AM15872-AS | cPrpusUfsgsUfuAfccauuCfcAfuUfcAfagsc | 1244 | UUGUUACCAUUCCAUUCAAGC | 1621 |
| AM15874-AS | cPrpusUfscsGfaAfacuauUfcUfcUfgUfcgsc | 1245 | UUCGAAACUAUUCUCUGUCGC | 1636 |
| AM15877-AS | cPrpusGfscsAfuUfuuugaCfaUfcCfuCfausc | 1246 | UGCAUUUUUGACAUCCUCAUC | 1607 |
| AM17578-AS | cPrpusAfsusCfaAfggaauGfgGfgCfaUfcasc | 1247 | UAUCAAGGAAUGGGGCAUCAC | 1604 |
| AM17580-AS | cPrpusGfsasUfcAfaggaaUfgGfgGfcAfucsa | 1248 | UGAUCAAGGAAUGGGGCAUCA | 1605 |
| AM17582-AS | cPrpusCfsgsAfuCfaaggaAfuGfgGfgCfausc | 1249 | UCGAUCAAGGAAUGGGGCAUC | 1606 |
| AM17584-AS | cPrpusCfsasUfuCfaagucCfuCfcGfaUfgasg | 1250 | UCAUUCAAGUCCUCCGAUGAG | 1608 |
| AM17586-AS | cPrpusCfscsAfuUfcaaguCfcUfcCfgAfugsa | 1251 | UCCAUUCAAGUCCUCCGAUGA | 1609 |
| AM17588-AS | cPrpusUfscsCfaUfucaagUfcCfuCfcGfausg | 1252 | UUCCAUUCAAGUCCUCCGAUG | 1610 |
| AM17590-AS | cPrpusAfsusUfcCfauucaAfgUfcCfuCfcgsa | 1253 | UAUUCCAUUCAAGUCCUCCGA | 1611 |
| AM17592-AS | cPrpusCfsasUfuCfcauucAfaGfuCfcUfccsg | 1254 | UCAUUCCAUUCAAGUCCUCCG | 1612 |
| AM17594-AS | cPrpasCfsasCfuGfaaguuCfgCfuUfuCfagsu | 1255 | ACACUGAAGUUCGCUUUCAGU | 1615 |
| AM17596-AS | cPrpasAfsgsUfaUfguccuGfgAfaGfaGfaasg | 1256 | AAGUAUGUCCUGGAAGAGAAG | 1618 |
| AM17598-AS | cPrpusCfscsAfaCfugcauUfuUfuGfaCfausc | 1257 | UCCAACUGCAUUUUUGACAUC | 1619 |
| AM17600-AS | cPrpasAfsgsGfuCfucccaUfuCfuCfaUfcasc | 1258 | AAGGUCUCCCAUUCUCAUCAC | 1623 |
| AM18492-AS | cPrpusUfsgsuuaCfcauuCfcAfuUfcaagsc | 1259 | UUGUUACCAUUCCAUUCAAGC | 1621 |
| AM18493-AS | cPrpusUfscsgaaAfcuauUfcUfcUfgucgsc | 1260 | UUCGAAACUAUUCUCUGUCGC | 1636 |
| AM19062-AS | cPrpusUfsgsGfaUfuccucUfuUfcAfaGfausc | 1261 | UUGGAUUCCUCUUUCAAGAUC | 1628 |
| AM19064-AS | cPrpasUfsgsAfgCfaugaaCfcAfgUfcUfcgsu | 1262 | AUGAGCAUGAACCAGUCUCGU | 1631 |
| AM19066-AS | cPrpasAfsasGfaUfuacacUfgAfaGfuUfcgsc | 1263 | AAAGAUUACACUGAAGUUCGC | 1632 |
| AM19068-AS | cPrpusUfsasUfcAfaggucUfcUfaAfuCfggsu | 1264 | UUAUCAAGGUCUCUAAUCGGU | 1633 |
| AM19070-AS | cPrpasGfsusGfaAfagcccUfuAfgUfaGfuasc | 1265 | AGUGAAAGCCCUUAGUAGUAC | 1616 |
| AM19072-AS | cPrpasCfsusCfgAfaccguGfuUfaCfcAfuusc | 1266 | ACUCGAACCGUGUUACCAUUC | 1634 |
| AM19074-AS | cPrpusAfsgsAfcUfcgaacCfgUfgUfuAfccsa | 1267 | UAGACUCGAACCGUGUUACCA | 1622 |
| AM19076-AS | cPrpusAfsgsUfuGfuaaggCfuUfgCfaUfaasc | 1268 | UAGUUGUAAGGCUUGCAUAAC | 1624 |
| AM19078-AS | cPrpasAfscsGfaGfaaagcUfcUfuAfuCfucsc | 1269 | AACGAGAAAGCUCUUAUCUCC | 1637 |

TABLE 3C
IAV RNAi agent (targeting PB1) Antisense Strand Sequences

| | | SEQ | Underlying Base Sequence (5′ → 3′) | SEQ |
|---|---|---|---|---|
| | | ID | (Shown as an Unmodified Nucleotide | ID |
| AS Strand ID | Modified Antisense Strand (5′ → 3′) | NO. | Sequence) | NO. |
| AM15187-AS | usAfsgsUfaUfaagggAfaUfgUfgGfugsc | 1270 | UAGUAUAAGGGAAUGUGGUGC | 1640 |
| AM15189-AS | usAfsgsUfaUfuggugUfgUfuCfuGfuusc | 1271 | UAGUAUUGGUGUGUUCUGUUC | 1641 |
| AM15191-AS | usUfscsCfaUfuguuuCfaAfgGfcAfugsa | 1272 | UUCCAUUGUUUCAAGGCAUGA | 1642 |
| AM15193-AS | asAfsgsAfcUfucuauGfgUfgUfuGfgcsc | 1273 | AAGACUUCUAUGGUGUUGGCC | 1643 |
| AM15195-AS | usGfsgsUfcAfuguugUfcUfcUfuAfcusc | 1274 | UGGUCAUGUUGUCUCUUACUC | 1644 |
| AM15197-AS | usCfsasAfgCfuuuucGfcAfaAfuGfcusc | 1275 | UCAAGCUUUUCGCAAAUGCUC | 1645 |
| AM15199-AS | asCfsusUfaGfuguugUfcCfcCfaGfugsa | 1276 | ACUUAGUGUUGUCCCCAGUGA | 1646 |
| AM15201-AS | usCfscsUfaGfucuugCfcAfuUfuUfgusc | 1277 | UCCUAGUCUUGCCAUUUUGUC | 1647 |
| AM15203-AS | usUfsasGfcAfuguugAfaCfaUfgCfccsa | 1278 | UUAGCAUGUUGAACAUGCCCA | 1648 |
| AM15205-AS | usGfsusAfgAfaucugUfcCfaCfuCfcusg | 1279 | UGUAGAAUCUGUCCACUCCUG | 1649 |
| AM15207-AS | usCfsusAfaAfauuagCfcAfcAfaAfucsc | 1280 | UCUAAAAUUAGCCACAAAUCC | 1650 |
| AM15209-AS | usGfsasUfgAfacaauUfgAfaGfaGfccsa | 1281 | UGAUGAACAAUUGAAGAGCCA | 1651 |
| AM15211-AS | asUfsasAfgUfuugguCfcUfcCfaUfcusg | 1282 | AUAAGUUUGGUCCUCCAUCUG | 1652 |
| AM15213-AS | usGfsgsUfaCfaucugUfuCfaUfcCfucsa | 1283 | UGGUACAUCUGUUCAUCCUCA | 1653 |
| AM15215-AS | usCfsasCfcAfugcuaGfaAfaUfuCfcasc | 1284 | UCACCAUGCUAGAAAUUCCAC | 1654 |
| AM15217-AS | usUfsusCfaAfuggugGfaAfcAfgAfucsu | 1285 | UUUCAAUGGUGGAACAGAUCU | 1655 |
| AM15219-AS | usGfsasAfuGfuggugCfuUfaUfgGfcasu | 1286 | UGAAUGUGGUGCUUAUGGCAU | 1656 |
| AM15221-AS | usAfscsGfaAfaccucUfaAfuCfuGfcasc | 1287 | UACGAAACCUCUAAUCUGCAC | 1657 |
| AM15223-AS | asGfsasCfuGfuucaaGfcUfuUfuCfgcsa | 1288 | AGACUGUUCAAGCUUUUCGCA | 1658 |
| AM15225-AS | usCfsasUfcAfucaucCfcAfgGfaCfucsa | 1289 | UCAUCAUCAUCCCAGGACUCA | 1659 |
| AM15227-AS | usCfsasAfgAfuucagUfaUfcGfaGfacsu | 1290 | UCAAGAUUCAGUAUCGAGACU | 1660 |
| AM15229-AS | usCfsasUfgUfuguucUfuUfaUfcAfcusg | 1291 | UCAUGUUGUUCUUUAUCACUG | 1661 |
| AM15231-AS | usAfsgsAfaCfucuucUfuUfcUfuGfausc | 1292 | UAGAACUCUUCUUUCUUGAUC | 1662 |
| AM15233-AS | usAfsasCfaGfaucuuCfaUfgAfuCfucsc | 1293 | UAACAGAUCUUCAUGAUCUCC | 1663 |
| AM15235-AS | usUfsgsAfgUfucuucAfaUfgGfuGfgasc | 1294 | UUGAGUUCUUCAAUGGUGGAC | 1664 |
| AM16046-AS | cPrpusUfscsCfaUfuguuuCfaAfgGfcAfugsa | 1295 | UUCCAUUGUUUCAAGGCAUGA | 1642 |
| AM16048-AS | cPrpasGfsasCfuGfuucaaGfcUfuUfuCfgcsa | 1296 | AGACUGUUCAAGCUUUUCGCA | 1658 |
| AM16050-AS | cPrpusAfscsGfaAfaccucUfaAfuCfuGfcasc | 1297 | UACGAAACCUCUAAUCUGCAC | 1657 |
| AM16683-AS | cPrpusUfsusCfaAfuggugGfaAfcAfgAfucsu | 1298 | UUUCAAUGGUGGAACAGAUCU | 1655 |
| AM17574-AS | cPrpusAfsasCfaGfaucuuCfaUfgAfuCfucsc | 1299 | UAACAGAUCUUCAUGAUCUCC | 1663 |
| AM18497-AS | cPrpusAfsgsUfaUfuggugUfgUfuCfuGfuusc | 1300 | UAGUAUUGGUGUGUUCUGUUC | 1641 |
| AM19084-AS | cPrpusGfsasUfgAfacaauUfgAfaGfaGfccsa | 1301 | UGAUGAACAAUUGAAGAGCCA | 1665 |

TABLE 3D
IAV RNAi agent (targeting PB2) Antisense Strand Sequences

| | | SEQ | Underlying Base Sequence (5′ → 3′) | SEQ |
|---|---|---|---|---|
| | | ID | (Shown as an Unmodified Nucleotide | ID |
| AS Strand ID | Modified Antisense Strand (5′ → 3′) | NO. | Sequence) | NO. |
| AM15253-AS | usGfsgsUfcUfuagugAfgUfaUfcUfcgsc | 1302 | UGGUCUUAGUGAGUAUCUCGC | 1665 |
| AM15255-AS | usCfsasUfgUfuucaaCfcUfuUfcGfacsc | 1303 | UCAUGUUUCAACCUUUCGACC | 1666 |
| AM15257-AS | usUfsasGfcAfuguacGfcCfaCfcAfucsa | 1304 | UUAGCAUGUACGCCACCAUCA | 1667 |
| AM15259-AS | usUfsasCfuAfuguuuCfuAfgCfaGfcgsa | 1305 | UUACUAUGUUUCUAGCAGCGA | 1668 |
| AM15261-AS | usAfscsUfgAfugaucCfgCfuUfgUfccsu | 1306 | UACUGAUGAUCCGCUUGUCCU | 1669 |
| AM15263-AS | usCfsusUfgAfucaugCfaAfuCfcUfccsu | 1307 | UCUUGAUCAUGCAAUCCUCCU | 1670 |
| AM15265-AS | usGfsusCfaAfuacucAfcUfaCfcAfcusc | 1308 | UGUCAAUACUCACUACCACUC | 1671 |
| AM15267-AS | asGfsasCfaAfuaguaCfgUfuCfcCfucsu | 1309 | AGACAAUAGUACGUUCCCUCU | 1672 |
| AM15269-AS | usCfsasUfuGfaucucCfcAfcAfuCfausc | 1310 | UCAUUGAUCUCCCACAUCAUC | 1673 |
| AM15271-AS | usGfsgsAfaCfaguguCfcUfuAfcGfaasc | 1311 | UGGAACAGUGUCCUUACGAAC | 1674 |
| AM15273-AS | usUfsgsUfuGfuaauuGfaAfuAfcUfggsa | 1312 | UUGUUGUAAUUGAAUACUGGA | 1675 |
| AM15275-AS | usAfsgsUfaAfguaugCfuAfgAfgUfccsc | 1313 | UAGUAAGUAUGCUAGAGUCCC | 1676 |
| AM16685-AS | cPrpusAfsgsUfaAfguaugCfuAfgAfgUfccsc | 1314 | UAGUAAGUAUGCUAGAGUCCC | 1676 |
| AM17367-AS | cPrpusGfsgsUfcUfuagugAfgUfaUfcUfcgsc | 1315 | UGGUCUUAGUGAGUAUCUCGC | 1665 |
| AM17369-AS | cPrpusCfsasUfgUfuucaaCfcUfuUfcGfacsc | 1316 | UCAUGUUUCAACCUUUCGACC | 1666 |
| AM17371-AS | cPrpusUfsasGfcAfuguacGfcCfaCfcAfucsa | 1317 | UUAGCAUGUACGCCACCAUCA | 1667 |
| AM17576-AS | cPrpusAfscsUfgAfugaucCfgCfuUfgUfccsu | 1318 | UACUGAUGAUCCGCUUGUCCU | 1669 |
| AM18499-AS | cPrpusUfsasCfuAfuguuuCfuAfgCfaGfcgsa | 1319 | UUACUAUGUUUCUAGCAGCGA | 1668 |
| AM19086-AS | cPrpusUfsgsUfuGfuaauuGfaAfuAfcUfggsa | 1320 | UUGUUGUAAUUGAAUACUGGA | 1675 |

TABLE 3E
IAV RNAi agent (targeting NP) Antisense Strand Sequences

| | | SEQ | Underlying Base Sequence (5′ → 3′) | SEQ |
|---|---|---|---|---|
| | | ID | (Shown as an Unmodified Nucleotide | ID |
| AS Strand ID | Modified Antisense Strand (5′ → 3′) | NO. | Sequence) | NO. |
| AM15336-AS | usAfscsCfaAfucauuCfuUfcCfgAfcasg | 1321 | UACCAAUCAUUCUUCCGACAG | 1677 |
| AM15338-AS | asUfsgsCfuAfuucugGfaUfuAfgUfcgsu | 1322 | AUGCUAUUCUGGAUUAGUCGU | 1678 |
| AM15340-AS | usCfsasGfaAfagcacCfaUfcCfuCfucsu | 1323 | UCAGAAAGCACCAUCCUCUCU | 1679 |
| AM15342-AS | usCfscsAfaAfucaugAfuAfuGfaGfuasc | 1324 | UCCAAAUCAUGAUAUGAGUAC | 1680 |
| AM15344-AS | usUfsgsAfuAfuguggCfaUfcAfuUfcasg | 1325 | UUGAUAUGUGGCAUCAUUCAG | 1681 |
| AM15346-AS | usGfsusUfgAfaccuuGfcAfuUfaGfagsc | 1326 | UGUUGAACCUUGCAUUAGAGC | 1682 |
| AM15348-AS | usCfsasCfgUfuugauCfaUfuCfuGfausc | 1327 | UCACGUUUGAUCAUUCUGAUC | 1683 |
| AM15350-AS | usCfsasGfaAfauuucGfgUfcAfuUfgasc | 1328 | UCAGAAAUUUCGGUCAUUGAC | 1684 |
| AM15352-AS | usCfsusUfuGfaggauAfuUfgCfaCfausc | 1329 | UCUUUGAGGAUAUUGCACAUC | 1685 |
| AM15354-AS | usUfscsAfgAfaugagUfgCfuGfaCfcgsu | 1330 | UUCAGAAUGAGUGCUGACCGU | 1686 |
| AM15356-AS | usGfsasUfuUfaugugCfaAfcUfgAfucsc | 1331 | UGAUUUAUGUGCAACUGAUCC | 1687 |
| AM15358-AS | usAfscsCfaAfuugacUfcUfuGfuGfagsc | 1332 | UACCAAUUGACUCUUGUGAGC | 1688 |
| AM15360-AS | usUfscsUfuAfugaaaCfuUfgAfuAfcusc | 1333 | UUCUUAUGAAACUUGAUACUC | 1689 |
| AM15362-AS | usGfsasGfaAfuguagGfcUfgCfaCfacsu | 1334 | UGAGAAUGUAGGCUGCACACU | 1690 |
| AM15364-AS | usUfsasCfuCfaugucAfaAfgGfaAfggsc | 1335 | UUACUCAUGUCAAAGGAAGGC | 1691 |
| AM15366-AS | asGfsasAfaUfaagacCfcUfuCfaUfuasc | 1336 | AGAAAUAAGACCCUUCAUUAC | 1692 |
| AM16687-AS | cPrpusUfsasCfuCfaugucAfaAfgGfaAfggsc | 1337 | UUACUCAUGUCAAAGGAAGGC | 1691 |
| AM17373-AS | cPrpusGfsusUfgAfaccuuGfcAfuUfaGfagsc | 1338 | UGUUGAACCUUGCAUUAGAGC | 1682 |
| AM17375-AS | cPrpasUfsgsCfuAfuucugGfaUfuAfgUfcgsu | 1339 | AUGCUAUUCUGGAUUAGUCGU | 1678 |
| AM17570-AS | cPrpusCfsasCfgUfuugauCfaUfuCfuGfausc | 1340 | UCACGUUUGAUCAUUCUGAUC | 1683 |
| AM18491-AS | cPrpusAfscsCfaAfucauuCfuUfcCfgAfcasg | 1341 | UACCAAUCAUUCUUCCGACAG | 1677 |
| AM19052-AS | cPrpusCfsasGfaAfagcacCfaUfcCfuCfucsu | 1342 | UCAGAAAGCACCAUCCUCUCU | 1679 |
| AM19054-AS | cPrpusUfsgsAfuAfuguggCfaUfcAfuUfcasg | 1343 | UUGAUAUGUGGCAUCAUUCAG | 1681 |
| AM19056-AS | cPrpusUfscsAfgAfaugagUfgCfuGfaCfcgsu | 1344 | UUCAGAAUGAGUGCUGACCGU | 1686 |
| AM19058-AS | cPrpusAfscsCfaAfuugacUfcUfuGfuGfagsc | 1345 | UACCAAUUGACUCUUGUGAGC | 1688 |
| AM19060-AS | cPrpasGfsasAfaUfaagacCfcUfuCfaUfuasc | 1346 | AGAAAUAAGACCCUUCAUUAC | 1692 |

TABLE 3F
IAV RNAi agent (targeting PA) Antisense Strand Sequences

| | | SEQ | Underlying Base Sequence (5′ → 3′) | SEQ |
|---|---|---|---|---|
| | | ID | (Shown as an Unmodified Nucleotide | ID |
| AS Strand ID | Modified Antisense Strand (5′ → 3′) | NO. | Sequence) | NO. |
| AM15298-AS | usAfsasGfcAfuugucGfcAfcAfaAfgusc | 1347 | UAAGCAUUGUCGCACAAAGUC | 1693 |
| AM15300-AS | usGfsasAfaUfggaaaUfcCfgAfaUfacsc | 1348 | UGAAAUGGAAAUCCGAAUACC | 1694 |
| AM15302-AS | usCfsasUfgAfuucggUfcUfcUfuCfcusu | 1349 | UCAUGAUUCGGUCUCUUCCUU | 1695 |
| AM15304-AS | usGfsasAfa AfgccuaGfuUfuUfgAfuusc | 1350 | UGAAAAGCCUAGUUUUGAUUC | 1696 |
| AM15306-AS | usAfscsGfaAfaggaaUfcCfcAfuAfgasc | 1351 | UACGAAAGGAAUCCCAUAGAC | 1697 |
| AM15308-AS | usUfsusCfaAfuugucUfcUfuCfgCfcusc | 1352 | UUUCAAUUGUCUCUUCGCCUC | 1698 |
| AM15310-AS | usUfscsGfaAfuccauCfuAfcAfuAfggsc | 1353 | UUCGAAUCCAUCUACAUAGGC | 1699 |
| AM15312-AS | usGfsasCfaUfuugggAfaAfgCfuUfgcsc | 1354 | UGACAUUUGGGAAAGCUUGCC | 1700 |
| AM15314-AS | usUfsusGfaUfugcauCfaUfaUfaGfugsg | 1355 | UUUGAUUGCAUCAUAUAGUGG | 1701 |
| AM15316-AS | asAfsgsGfuCfuccaaCfaUfcUfuUfgcsa | 1356 | AAGGUCUCCAACAUCUUUGCA | 1702 |
| AM15318-AS | usCfsasUfcAfaguucUfaUfcCfaGfcusu | 1357 | UCAUCAAGUUCUAUCCAGCUU | 1703 |
| AM15320-AS | asCfsasUfuUfgcuuaUfcAfuUfgGfgasu | 1358 | ACAUUUGCUUAUCAUUGGGAU | 1704 |
| AM15322-AS | usCfsasUfuAfaucagGfcAfcUfcCfucsg | 1359 | UCAUUAAUCAGGCACUCCUCG | 1705 |
| AM16679-AS | cPrpusAfsasGfcAfuugucGfcAfcAfaAfgusc | 1360 | UAAGCAUUGUCGCACAAAGUC | 1693 |
| AM16681-AS | cPrpusUfsusGfaUfugcauCfaUfaUfaGfugsg | 1361 | UUUGAUUGCAUCAUAUAGUGG | 1701 |
| AM16808-AS | cPrpusGfsasAfaAfgccuaGfuUfuUfgAfuusc | 1362 | UGAAAAGCCUAGUUUUGAUUC | 1696 |
| AM16810-AS | cPrpasAfsgsGfuCfuccaaCfaUfcUfuUfgcsa | 1363 | AAGGUCUCCAACAUCUUUGCA | 1702 |
| AM17572-AS | cPrpusCfsasUfuAfaucagGfcAfcUfcCfucsg | 1364 | UCAUUAAUCAGGCACUCCUCG | 1705 |
| AM18495-AS | cPrpusGfsasAfaUfggaaaUfcCfgAfaUfacsc | 1365 | UGAAAUGGAAAUCCGAAUACC | 1694 |
| AM19080-AS | cPrpusUfscsGfaAfuccauCfuAfcAfuAfggsc | 1366 | UUCGAAUCCAUCUACAUAGGC | 1699 |
| AM19082-AS | cPrpasCfsasUfuUfgcuuaUfcAfuUfgGfgasu | 1367 | ACAUUUGCUUAUCAUUGGGAU | 1704 |

TABLE 4A
IAV RNAi Agent (targeting M1) Sense Strand Sequences (Shown Without Linkers, Conjugates, or
Capping Moieties.)

| | | SEQ | Underlying Base Sequence (5′ → 3′) | SEQ |
|---|---|---|---|---|
| | | ID | (Shown as an Unmodified Nucleotide | ID |
| Strand ID | Modified Antisense Strand (5′ → 3′) | NO. | Sequence) | NO. |
| AM16676-SS-NL | csuaaccgaGfGfUfcgaaacguaa | 1368 | CUAACCGAGGUCGAAACGUAA | 1706 |
| AM16811-SS-NL | ascguacguUfCfUfuucuaucaua | 1369 | ACGUACGUUCUUUCUAUCAUA | 1707 |
| AM17565-SS-NL | gsuugacgaUfGfGfucauuuugua | 1370 | GUUGACGAUGGUCAUUUUGUA | 1708 |
| AM17567-SS-NL | gsugacgauGfGfUfcauuuuguca | 1371 | GUGACGAUGGUCAUUUUGUCA | 1709 |
| AM17855-SS-NL | csuaaccgaGfGfUfcgaaacguaa | 1372 | CUAACCGAGGUCGAAACGUAA | 1706 |
| AM17927-SS-NL | csuaaccgaGfgUfcGfaaacguaa | 1373 | CUAACCGAGGUCGAAACGUAA | 1706 |
| AM17932-SS-NL | csuaaccgaGfgUfcgaaacguaa | 1374 | CUAACCGAGGUCGAAACGUAA | 1706 |
| AM18486-SS-NL | csccgaacaAfCfAfuggauagaga | 1375 | CCCGAACAACAUGGAUAGAGA | 1710 |
| AM18488-SS-NL | gsacaacauGfGfAfuagaicaguu | 1376 | GACAACAUGGAUAGAICAGUU | 1711 |
| AM18519-SS-NL | a_2NscguacguUfCfUfuucuaucaua | 1377 | (A^{2N})CGUACGUUCUUUCUAUCAUA | 1770 |
| AM18522-SS-NL | ascguacguUfcUfuUfcuaucaua | 1378 | ACGUACGUUCUUUCUAUCAUA | 1707 |
| AM18527-SS-NL | ascguacguUfcUfUfucuaucaua | 1379 | ACGUACGUUCUUUCUAUCAUA | 1707 |

a_2N = 2-aminoadenosine nucleotide; I = hypoxanthine (inosine) nucleotide

TABLE 4B
IAV RNAi Agent (targeting NS1) Sense Strand Sequences (Shown Without Linkers, Conjugates, or
Capping Moieties.)

| | | SEQ | Underlying Base Sequence (5′ → 3′) | SEQ |
|---|---|---|---|---|
| | | ID | (Shown as an Unmodified Nucleotide | ID |
| Strand ID | Modified Antisense Strand (5′ → 3′) | NO. | Sequence) | NO. |
| AM15869-SS-NL | gsauacagaGfAfUfucgcuuigaa | 1380 | GAUACAGAGAUUCGCUUIGAA | 1712 |
| AM15870-SS-NL | asgcuuucuCfGfUfuucagcuuau | 1381 | AGCUUUCUCGUUUCAGCUUAU | 1713 |
| AM15871-SS-NL | gscuugaauGfGfAfaugguaacaa | 1382 | GCUUGAAUGGAAUGGUAACAA | 1714 |
| AM15873-SS-NL | gscgacagaGfAfAfuaguuucgaa | 1383 | GCGACAGAGAAUAGUUUCGAA | 1715 |
| AM15876-SS-NL | gsaugaggaUfGfUfcaaaaaugca | 1384 | GAUGAGGAUGUCAAAAAUGCA | 1716 |
| AM16806-SS-NL | gsaugaggaUfgUfcAfaaaaugca | 1385 | GAUGAGGAUGUCAAAAAUGCA | 1716 |
| AM17577-SS-NL | gsugaugccCfCfAfuuccuugaua | 1386 | GUGAUGCCCCAUUCCUUGAUA | 1717 |
| AM17579-SS-NL | usgaugcccCfAfUfuccuugauca | 1387 | UGAUGCCCCAUUCCUUGAUCA | 1718 |
| AM17581-SS-NL | gsaugccccAfUfUfccuuiaucga | 1388 | GAUGCCCCAUUCCUUIAUCGA | 1719 |
| AM17583-SS-NL | csucaucggAfGfGfacuugaauga | 1389 | CUCAUCGGAGGACUUGAAUGA | 1720 |
| AM17585-SS-NL | uscaucggaGfGfAfcuugaauiga | 1390 | UCAUCGGAGGACUUGAAUIGA | 1721 |
| AM17587-SS-NL | csaucggagGfAfCfuugaauggaa | 1391 | CAUCGGAGGACUUGAAUGGAA | 1722 |
| AM17589-SS-NL | uscggaggaCfUfUfgaauggaaua | 1392 | UCGGAGGACUUGAAUGGAAUA | 1723 |
| AM17591-SS-NL | csggaggacUfUfGfaaugiaauga | 1393 | CGGAGGACUUGAAUGIAAUGA | 1724 |
| AM17593-SS-NL | ascugaaagCfGfAfacuucaiugu | 1394 | ACUGAAAGCGAACUUCAIUGU | 1725 |
| AM17595-SS-NL | csuucucuuCfCfAfggacauacuu | 1395 | CUUCUCUUCCAGGACAUACUU | 1726 |
| AM17597-SS-NL | gsaugucaaAfAfAfugcaiuugga | 1396 | GAUGUCAAAAAUGCAIUUGGA | 1727 |
| AM17599-SS-NL | gsugaugagAfAfUfgggaiaccuu | 1397 | GUGAUGAGAAUGGGAIACCUU | 1728 |
| AM19061-SS-NL | gsaucuugaAfAfGfaggaauccaa | 1398 | GAUCUUGAAAGAGGAAUCCAA | 1729 |
| AM19063-SS-NL | ascgagacuGfGfUfucauicucau | 1399 | ACGAGACUGGUUCAUICUCAU | 1730 |
| AM19065-SS-NL | gscgaacuuCfAfGfuguaaucuuu | 1400 | GCGAACUUCAGUGUAAUCUUU | 1731 |
| AM19067-SS-NL | asccgauuaGfAfGfaccuugauaa | 1401 | ACCGAUUAGAGACCUUGAUAA | 1732 |
| AM19069-SS-NL | gsuacuacuAfAfGfggcuuucacu | 1402 | GUACUACUAAGGGCUUUCACU | 1733 |
| AM19071-SS-NL | gsaaugguaAfCfAfcgguuciagu | 1403 | GAAUGGUAACACGGUUCIAGU | 1734 |
| AM19073-SS-NL | usgguaacaCfGfGfuucgaiucua | 1404 | UGGUAACACGGUUCGAIUCUA | 1735 |
| AM19075-SS-NL | gsuuaugcaAfGfCfcuuacaacua | 1405 | GUUAUGCAAGCCUUACAACUA | 1736 |
| AM19077-SS-NL | gsgagauaaGfAfGfcuuucuciuu | 1406 | GGAGAUAAGAGCUUUCUCIUU | 1737 |

a_2N = 2-aminoadenosine nucleotide; I = hypoxanthine (inosine) nucleotide

TABLE 4C
IAV RNAi Agent (targeting PB1) Sense Strand Sequences
(Shown Without Linkers, Conjugates, or Capping Moieties.)

| | | | Underlying Base | |
|---|---|---|---|---|
| | | SEQ | Sequence (5′ → 3′) | SEQ |
| | Modified Sense | ID | (Shown as an Unmodified | ID |
| Strand ID | Strand (5′ → 3′) | NO. | Nucleotide Sequence) | NO. |
| AM16045-SS-NL | uscaugccuUfGfAfaacaauggaa | 1407 | UCAUGCCUUGAAACAAUGGAA | 1738 |
| AM16047-SS-NL | usgcgaaaaGfCfUfugaacaiucu | 1408 | UGCGAAAAGCUUGAACAIUCU | 1739 |
| AM16049-SS-NL | gsugcagauUfAfGfagguuucgua | 1409 | GUGCAGAUUAGAGGUUUCGUA | 1740 |
| AM16682-SS-NL | asgaucuguUfCfCfaccauugaaa | 1410 | AGAUCUGUUCCACCAUUGAAA | 1741 |
| AM17573-SS-NL | gsgagaucaUfGfAfagaucuguua | 1411 | GGAGAUCAUGAAGAUCUGUUA | 1742 |
| AM17856-SS-NL | asgaucuguUfCfCfaccauugaaa | 1412 | AGAUCUGUUCCACCAUUGAAA | 1741 |
| AM18496-SS-NL | gsaacagaaCfAfCfaccaauacua | 1413 | GAACAGAACACACCAAUACUA | 1743 |
| AM19083-SS-NL | usggcucuuCfAfAfuuguucauca | 1414 | UGGCUCUUCAAUUGUUCAUCA | 1744 |

a_2N = 2-aminoadenosine nucleotide;
I = hypoxanthine (inosine) nucleotide

TABLE 4D
IAV RNAi Agent (targeting PB2) Sense Strand Sequences
(Shown Without Linkers, Conjugates, or Capping Moieties.)

| | | | Underlying Base | |
|---|---|---|---|---|
| | | SEQ | Sequence (5′ → 3′) | SEQ |
| | Modified Sense | ID | (Shown as an Unmodified | ID |
| Strand ID | Strand (5′ → 3′) | NO. | Nucleotide Sequence) | NO. |
| AM16684-SS-NL | gsggacucuAfGfCfauacuuacua | 1415 | GGGACUCUAGCAUACUUACUA | 1745 |
| AM17366-SS-NL | gscgagauaCfUfCfacuaaiacca | 1416 | GCGAGAUACUCACUAAIACCA | 1746 |
| AM17368-SS-NL | gsgucgaaaGfGfUfugaaacauga | 1417 | GGUCGAAAGGUUGAAACAUGA | 1747 |
| AM17370-SS-NL | usgauggugGfCfGfuacaugcuaa | 1418 | UGAUGGUGGCGUACAUGCUAA | 1748 |
| AM17575-SS-NL | asggacaagCfGfGfaucaucagua | 1419 | AGGACAAGCGGAUCAUCAGUA | 1749 |
| AM17857-SS-NL | gsggacucuAfGfCfauacuuacua | 1420 | GGGACUCUAGCAUACUUACUA | 1745 |
| AM18498-SS-NL | uscgcugcuAfGfAfaacauaguaa | 1421 | UCGCUGCUAGAAACAUAGUAA | 1750 |
| AM19085-SS-NL | usccaguauUfCfAfauuacaacaa | 1422 | UCCAGUAUUCAAUUACAACAA | 1751 |

a_2N = 2-aminoadenosine nucleotide;
I = hypoxanthine (inosine) nucleotide

TABLE 4E
IAV RNAi Agent (targeting NP) Sense Strand Sequences
(Shown Without Linkers, Conjugates, or Capping Moieties.)

| | | | Underlying Base | |
|---|---|---|---|---|
| | | SEQ | Sequence (5′ → 3′) | SEQ |
| | Modified Sense | ID | (Shown as an Unmodified | ID |
| Strand ID | Strand (5′ → 3′) | NO. | Nucleotide Sequence) | NO. |
| AM16686-SS-NL | gsccuuccuUfUfGfacaugaguaa | 1423 | GCCUUCCUUUGACAUGAGUAA | 1752 |
| AM17372-SS-NL | gscucuaauGfCfAfagguucaaca | 1424 | GCUCUAAUGCAAGGUUCAACA | 1753 |
| AM17374-SS-NL | ascgacuaaUfCfCfagaauagcau | 1425 | ACGACUAAUCCAGAAUAGCAU | 1754 |
| AM17569-SS-NL | gsaucagaaUfGfAfucaaaciuga | 1426 | GAUCAGAAUGAUCAAACIUGA | 1755 |
| AM17858-SS-NL | gsccuuccuUfUfGfacaugaguaa | 1427 | GCCUUCCUUUGACAUGAGUAA | 1752 |
| AM18490-SS-NL | csugucggaAfGfAfaugauuggua | 1428 | CUGUCGGAAGAAUGAUUGGUA | 1756 |
| AM19051-SS-NL | asgagaggaUfGfGfugcuuucuga | 1429 | AGAGAGGAUGGUGCUUUCUGA | 1757 |
| AM19053-SS-NL | csugaaugaUfGfCfcacauaucaa | 1430 | CUGAAUGAUGCCACAUAUCAA | 1758 |
| AM19055-SS-NL | ascggucagCfAfCfucauucugaa | 1431 | ACGGUCAGCACUCAUUCUGAA | 1759 |
| AM19057-SS-NL | gscucacaaGfAfGfucaauuggua | 1432 | GCUCACAAGAGUCAAUUGGUA | 1760 |
| AM19059-SS-NL | gsuaaugaaGfGfGfucuuauuucu | 1433 | GUAAUGAAGGGUCUUAUUUCU | 1761 |

a_2N = 2-aminoadenosine nucleotide;
I = hypoxanthine (inosine) nucleotide

TABLE 4F
IAV RNAi Agent (targeting PA) Sense Strand Sequences
(Shown Without Linkers, Conjugates, or Capping Moieties.)

| | | | Underlying Base | |
|---|---|---|---|---|
| | | SEQ | Sequence (5′ → 3′) | SEQ |
| | Modified Sense | ID | (Shown as an Unmodified | ID |
| Strand ID | Strand (5′ → 3′) | NO. | Nucleotide Sequence) | NO. |
| AM16678-SS-NL | gsacuuuguGfCfGfacaaugcuua | 1434 | GACUUUGUGCGACAAUGCUUA | 1762 |
| AM16680-SS-NL | cscacuauaUfGfAfugcaaucaaa | 1435 | CCACUAUAUGAUGCAAUCAAA | 1763 |
| AM16807-SS-NL | gsa_2NaucaaaAfCfUfaggcuuuuca | 1436 | G(A^{2N})AUCAAAACUAGGCUUUUCA | 1771 |
| AM16809-SS-NL | usgcaaagaUfGfUfuggaiaccuu | 1437 | UGCAAAGAUGUUGGAIACCUU | 1765 |
| AM17571-SS-NL | csgaggaguGfCfCfugauuaauga | 1438 | CGAGGAGUGCCUGAUUAAUGA | 1766 |
| AM17859-SS-NL | gsacuuuguGfCfGfacaaugcuua | 1439 | GACUUUGUGCGACAAUGCUUA | 1762 |
| AM17860-SS-NL | cscacuauaUfGfAfugcaaucaaa | 1440 | CCACUAUAUGAUGCAAUCAAA | 1763 |
| AM18494-SS-NL | gsguauucgGfAfUfuuccauuuca | 1441 | GGUAUUCGGAUUUCCAUUUCA | 1767 |
| AM19079-SS-NL | gsccuauguAfGfAfuggauucgaa | 1442 | GCCUAUGUAGAUGGAUUCGAA | 1768 |
| AM19081-SS-NL | asucccaauGfa_2NUfaagcaaaugu | 1443 | AUCCCAAUG(A^{2N})UAAGCAAAUGU | 1772 |

a_2N = 2-aminoadenosine nucleotide;
I = hypoxanthine (inosine) nucleotide

TABLE 5A
IAV RNAi Agent (targeting M1) Sense Strand Sequences (Shown With (TriAlk14)
Linker or Targeting Ligand (see Table 11 for structure information.))

| | | | Underlying Base | |
|---|---|---|---|---|
| | | SEQ | Sequence (5′ → 3′) | SEQ |
| | | ID | (Shown as an Unmodified | ID |
| Strand ID | Modified Sense Strand (5′ → 3′) | NO. | Nucleotide Sequence) | NO. |
| AM16676-SS | (TriAlk14)csuaaccgaGfGfUfcgaaacguaas(invAb) | 1444 | CUAACCGAGGUCGAAACGUAA | 1706 |
| AM16811-SS | (TriAlk14)ascguacguUfCfUfuucuaucauas(invAb) | 1445 | ACGUACGUUCUUUCUAUCAUA | 1707 |
| AM17565-SS | (TriAlk14)gsuugacgaUfGfGfucauuuuguas(invAb) | 1446 | GUUGACGAUGGUCAUUUUGUA | 1708 |
| AM17567-SS | (TriAlk14)gsugacgauGfGfUfcauuuugucas(invAb) | 1447 | GUGACGAUGGUCAUUUUGUCA | 1709 |
| AM17855-SS | (NH2-C6)csuaaccgaGfGfUfcgaaacguaas(invAb) | 1448 | CUAACCGAGGUCGAAACGUAA | 1706 |
| AM17927-SS | (TriAlk14)csuaaccgaGfgUfcGfaaacguaas(invAb) | 1449 | CUAACCGAGGUCGAAACGUAA | 1706 |
| AM17932-SS | (TriAlk14)csuaaccgaGfgUfcgaaacguaas(invAb) | 1450 | CUAACCGAGGUCGAAACGUAA | 1706 |
| AM18486-SS | (TriAlk14)csccgaacaAfCfAfuggauagagas(invAb) | 1451 | CCCGAACAACAUGGAUAGAGA | 1710 |
| AM18488-SS | (TriAlk14)gsacaacauGfGfAfuagaicaguus(invAb) | 1452 | GACAACAUGGAUAGAICAGUU | 1711 |
| AM18519-SS | (TriAlk14)a_2NscguacguUfCfUfuucuaucauas(invAb) | 1453 | (A^{2N})CGUACGUUCUUUCUAUCAUA | 1770 |
| AM18522-SS | (TriAlk14)ascguacguUfcUfuUfcuaucauas(invAb) | 1454 | ACGUACGUUCUUUCUAUCAUA | 1707 |
| AM18527-SS | (TriAlk14)ascguacguUfcUfUfucuaucauas(invAb) | 1455 | ACGUACGUUCUUUCUAUCAUA | 1707 |

(A^{2N}) = 2-aminoadenosine nucleotide;
I = hypoxanthine (inosine) nucleotide

TABLE 5B
IAV RNAi Agent (targeting NS1) Sense Strand Sequences (Shown With (TriAlk14)
Linker or Targeting Ligand (see Table 11 for structure information.))

| | | | Underlying Base | |
|---|---|---|---|---|
| | | SEQ | Sequence (5′ → 3′) | SEQ |
| | | ID | (Shown as an Unmodified | ID |
| Strand ID | Modified Sense Strand (5′ → 3′) | NO. | Nucleotide Sequence) | NO. |
| AM15869-SS | (TriAlk14)gsauacagaGfAfUfucgcuuigaas(invAb) | 1456 | GAUACAGAGAUUCGCUUIGAA | 1712 |
| AM15870-SS | (TriAlk14)asgcuuucuCfGfUfuucagcuuaus(invAb) | 1457 | AGCUUUCUCGUUUCAGCUUAU | 1713 |
| AM15871-SS | (TriAlk14)gscuugaauGfGfAfaugguaacaas(invAb) | 1458 | GCUUGAAUGGAAUGGUAACAA | 1714 |
| AM15873-SS | (TriAlk14)gscgacagaGfAfAfuaguuucgaas(invAb) | 1459 | GCGACAGAGAAUAGUUUCGAA | 1715 |
| AM15876-SS | (TriAlk14)gsaugaggaUfGfUfcaaaaaugcas(invAb) | 1460 | GAUGAGGAUGUCAAAAAUGCA | 1716 |
| AM16806-SS | (TriAlk14)gsaugaggaUfgUfcAfaaaaugcas(invAb) | 1461 | GAUGAGGAUGUCAAAAAUGCA | 1716 |
| AM17577-SS | (TriAlk14)gsugaugccCfCfAfuuccuugauas(invAb) | 1462 | GUGAUGCCCCAUUCCUUGAUA | 1717 |
| AM17579-SS | (TriAlk14)usgaugcccCfAfUfuccuugaucas(invAb) | 1463 | UGAUGCCCCAUUCCUUGAUCA | 1718 |
| AM17581-SS | (TriAlk14)gsaugccccAfUfUfccuuiaucgas(invAb) | 1464 | GAUGCCCCAUUCCUUIAUCGA | 1719 |
| AM17583-SS | (TriAlk14)csucaucggAfGfGfacuugaaugas(invAb) | 1465 | CUCAUCGGAGGACUUGAAUGA | 1720 |
| AM17585-SS | (TriAlk14)uscaucggaGfGfAfcuugaauigas(invAb) | 1466 | UCAUCGGAGGACUUGAAUIGA | 1721 |
| AM17587-SS | (TriAlk14)csaucggagGfAfCfuugaauggaas(invAb) | 1467 | CAUCGGAGGACUUGAAUGGAA | 1722 |
| AM17589-SS | (TriAlk14)uscggaggaCfUfUfgaauggaauas(invAb) | 1468 | UCGGAGGACUUGAAUGGAAUA | 1723 |
| AM17591-SS | (TriAlk14)csggaggacUfUfGfaaugiaaugas(invAb) | 1469 | CGGAGGACUUGAAUGIAAUGA | 1724 |
| AM17593-SS | (TriAlk14)ascugaaagCfGfAfacuucaiugus(invAb) | 1470 | ACUGAAAGCGAACUUCAIUGU | 1725 |
| AM17595-SS | (TriAlk14)csuucucuuCfCfAfggacauacuus(invAb) | 1471 | CUUCUCUUCCAGGACAUACUU | 1726 |
| AM17597-SS | (TriAlk14)gsaugucaaAfAfAfugcaiuuggas(invAb) | 1472 | GAUGUCAAAAAUGCAIUUGGA | 1727 |
| AM17599-SS | (TriAlk14)gsugaugagAfAfUfgggaiaccuus(invAb) | 1473 | GUGAUGAGAAUGGGAIACCUU | 1728 |
| AM19061-SS | (TriAlk14)gsaucuugaAfAfGfaggaauccaas(invAb) | 1474 | GAUCUUGAAAGAGGAAUCCAA | 1729 |
| AM19063-SS | (TriAlk14)ascgagacuGfGfUfucauicucaus(invAb) | 1475 | ACGAGACUGGUUCAUICUCAU | 1730 |
| AM19065-SS | (TriAlk14)gscgaacuuCfAfGfuguaaucuuus(invAb) | 1476 | GCGAACUUCAGUGUAAUCUUU | 1731 |
| AM19067-SS | (TriAlk14)asccgauuaGfAfGfaccuugauaas(invAb) | 1477 | ACCGAUUAGAGACCUUGAUAA | 1732 |
| AM19069-SS | (TriAlk14)gsuacuacuAfAfGfggcuuucacus(invAb) | 1478 | GUACUACUAAGGGCUUUCACU | 1733 |
| AM19071-SS | (TriAlk14)gsaaugguaAfCfAfcgguuciagus(invAb) | 1479 | GAAUGGUAACACGGUUCIAGU | 1734 |
| AM19073-SS | (TriAlk14)usgguaacaCfGfGfuucgaiucuas(invAb) | 1480 | UGGUAACACGGUUCGAIUCUA | 1735 |
| AM19075-SS | (TriAlk14)gsuuaugcaAfGfCfcuuacaacuas(invAb) | 1481 | GUUAUGCAAGCCUUACAACUA | 1736 |
| AM19077-SS | (TriAlk14)gsgagauaaGfAfGfcuuucuciuus(invAb) | 1482 | GGAGAUAAGAGCUUUCUCIUU | 1737 |

(A^{2N}) = 2-aminoadenosine nucleotide;
I = hypoxanthine (inosine) nucleotide

TABLE 5C
IAV RNAi Agent (targeting PB1) Sense Strand Sequences (Shown With (TriAlk14)
Linker or Targeting Ligand (see Table 11 for structure information.))

| | | | Underlying Base | |
|---|---|---|---|---|
| | | SEQ | Sequence (5′ → 3′) | SEQ |
| | | ID | (Shown as an Unmodified | ID |
| Strand ID | Modified Sense Strand (5′ → 3′) | NO. | Nucleotide Sequence) | NO. |
| AM16045-SS | (TriAlk14)uscaugccuUfGfAfaacaauggaas(invAb) | 1483 | UCAUGCCUUGAAACAAUGGAA | 1738 |
| AM16047-SS | (TriAlk14)usgcgaaaaGfCfUfugaacaiucus(invAb) | 1484 | UGCGAAAAGCUUGAACAIUCU | 1739 |
| AM16049-SS | (TriAlk14)gsugcagauUfAfGfagguuucguas(invAb) | 1485 | GUGCAGAUUAGAGGUUUCGUA | 1740 |
| AM16682-SS | (TriAlk14)asgaucuguUfCfCfaccauugaaas(invAb) | 1486 | AGAUCUGUUCCACCAUUGAAA | 1741 |
| AM17573-SS | (TriAlk14)gsgagaucaUfGfAfagaucuguuas(invAb) | 1487 | GGAGAUCAUGAAGAUCUGUUA | 1742 |
| AM17856-SS | (NH2-C6)asgaucuguUfCfCfaccauugaaas(invAb) | 1488 | AGAUCUGUUCCACCAUUGAAA | 1741 |
| AM18496-SS | (TriAlk14)gsaacagaaCfAfCfaccaauacuas(invAb) | 1489 | GAACAGAACACACCAAUACUA | 1743 |
| AM19083-SS | (TriAlk14)usggcucuuCfAfAfuuguucaucas(invAb) | 1490 | UGGCUCUUCAAUUGUUCAUCA | 1744 |

(A^{2N}) = 2-aminoadenosine nucleotide;
I = hypoxanthine (inosine) nucleotide

TABLE 5D
IAV RNAi Agent (targeting PB2) Sense Strand Sequences (Shown With (TriAlk14)
Linker or Targeting Ligand (see Table 11 for structure information.))

| | | | Underlying Base | |
|---|---|---|---|---|
| | | SEQ | Sequence (5′ → 3′) | SEQ |
| | | ID | (Shown as an Unmodified | ID |
| Strand ID | Modified Sense Strand (5′ → 3′) | NO. | Nucleotide Sequence) | NO. |
| AM16684-SS | (TriAlk14)gsggacucuAfGfCfauacuuacuas(invAb) | 1491 | GGGACUCUAGCAUACUUACUA | 1745 |
| AM17366-SS | (TriAlk14)gscgagauaCfUfCfacuaaiaccas(invAb) | 1492 | GCGAGAUACUCACUAAIACCA | 1746 |
| AM17368-SS | (TriAlk14)gsgucgaaaGfGfUfugaaacaugas(invAb) | 1493 | GGUCGAAAGGUUGAAACAUGA | 1747 |
| AM17370-SS | (TriAlk14)usgauggugGfCfGfuacaugcuaas(invAb) | 1494 | UGAUGGUGGCGUACAUGCUAA | 1748 |
| AM17575-SS | (TriAlk14)asggacaagCfGfGfaucaucaguas(invAb) | 1495 | AGGACAAGCGGAUCAUCAGUA | 1749 |
| AM17857-SS | (NH2-C6)gsggacucuAfGfCfauacuuacuas(invAb) | 1496 | GGGACUCUAGCAUACUUACUA | 1745 |
| AM18498-SS | (TriAlk14)uscgcugcuAfGfAfaacauaguaas(invAb) | 1497 | UCGCUGCUAGAAACAUAGUAA | 1750 |
| AM19085-SS | (TriAlk14)usccaguauUfCfAfauuacaacaas(invAb) | 1498 | UCCAGUAUUCAAUUACAACAA | 1751 |

(A^{2N}) = 2-aminoadenosine nucleotide;
I = hypoxanthine (inosine) nucleotide

TABLE 5E
IAV RNAi Agent (targeting NP) Sense Strand Sequences (Shown With (TriAlk14)
Linker or Targeting Ligand (see Table 11 for structure information.))

| | | | Underlying Base | |
|---|---|---|---|---|
| | | SEQ | Sequence (5′ → 3′) | SEQ |
| | | ID | (Shown as an Unmodified | ID |
| Strand ID | Modified Sense Strand (5′ → 3′) | NO. | Nucleotide Sequence) | NO. |
| AM16686-SS | (TriAlk14)gsccuuccuUfUfGfacaugaguaas(invAb) | 1499 | GCCUUCCUUUGACAUGAGUAA | 1752 |
| AM17372-SS | (TriAlk14)gscucuaauGfCfAfagguucaacas(invAb) | 1500 | GCUCUAAUGCAAGGUUCAACA | 1753 |
| AM17374-SS | (TriAlk14)ascgacuaaUfCfCfagaauagcaus(invAb) | 1501 | ACGACUAAUCCAGAAUAGCAU | 1754 |
| AM17569-SS | (TriAlk14)gsaucagaaUfGfAfucaaaciugas(invAb) | 1502 | GAUCAGAAUGAUCAAACIUGA | 1755 |
| AM17858-SS | (NH2-C6)gsccuuccuUfUfGfacaugaguaas(invAb) | 1503 | GCCUUCCUUUGACAUGAGUAA | 1752 |
| AM18490-SS | (TriAlk14)csugucggaAfGfAfaugauugguas(invAb) | 1504 | CUGUCGGAAGAAUGAUUGGUA | 1756 |
| AM19051-SS | (TriAlk14)asgagaggaUfGfGfugcuuucugas(invAb) | 1505 | AGAGAGGAUGGUGCUUUCUGA | 1757 |
| AM19053-SS | (TriAlk14)csugaaugaUfGfCfcacauaucaas(invAb) | 1506 | CUGAAUGAUGCCACAUAUCAA | 1758 |
| AM19055-SS | (TriAlk14)ascggucagCfAfCfucauucugaas(invAb) | 1507 | ACGGUCAGCACUCAUUCUGAA | 1759 |
| AM19057-SS | (TriAlk14)gscucacaaGfAfGfucaauugguas(invAb) | 1508 | GCUCACAAGAGUCAAUUGGUA | 1760 |
| AM19059-SS | (TriAlk14)gsuaaugaaGfGfGfucuuauuucus(invAb) | 1509 | GUAAUGAAGGGUCUUAUUUCU | 1761 |

(A^{2N}) = 2-aminoadenosine nucleotide;
I = hypoxanthine (inosine) nucleotide

TABLE 5F
IAV RNAi Agent (targeting PA) Sense Strand Sequences (Shown With (TriAlk14)
Linker or Targeting Ligand (see Table 11 for structure information.))

| | | | Underlying Base | |
|---|---|---|---|---|
| | | SEQ | Sequence (5′ → 3′) | SEQ |
| | | ID | (Shown as an Unmodified | ID |
| Strand ID | Modified Sense Strand (5′ → 3′) | NO. | Nucleotide Sequence) | NO. |
| AM16678-SS | (TriAlk14)gsacuuuguGfCfGfacaaugcuuas(invAb) | 1510 | GACUUUGUGCGACAAUGCUUA | 1762 |
| AM16680-SS | (TriAlk14)cscacuauaUfGfAfugcaaucaaas(invAb) | 1511 | CCACUAUAUGAUGCAAUCAAA | 1763 |
| AM16807-SS | (TriAlk14)gsa_2NaucaaaAfCfUfaggcuuuucas(invAb) | 1512 | G(A^{2N})AUCAAAACUAGGCUUUUCA | 1771 |
| AM16809-SS | (TriAlk14)usgcaaagaUfGfUfuggaiaccuus(invAb) | 1513 | UGCAAAGAUGUUGGAIACCUU | 1765 |
| AM17571-SS | (TriAlk14)csgaggaguGfCfCfugauuaaugas(invAb) | 1514 | CGAGGAGUGCCUGAUUAAUGA | 1766 |
| AM17859-SS | (NH2-C6)gsacuuuguGfCfGfacaaugcuuas(invAb) | 1515 | GACUUUGUGCGACAAUGCUUA | 1762 |
| AM17860-SS | (NH2-C6)cscacuauaUfGfAfugcaaucaaas(invAb) | 1516 | CCACUAUAUGAUGCAAUCAAA | 1763 |
| AM18494-SS | (TriAlk14)gsguauucgGfAfUfuuccauuucas(invAb) | 1517 | GGUAUUCGGAUUUCCAUUUCA | 1767 |
| AM19079-SS | (TriAlk14)gsccuauguAfGfAfuggauucgaas(invAb) | 1518 | GCCUAUGUAGAUGGAUUCGAA | 1768 |
| AM19081-SS | (TriAlk14)asucccaauGfa_2NUfaagcaaaugus(invAb) | 1519 | AUCCCAAUG(A^{2N})UAAGCAAAUGU | 1772 |

(A^{2N}) = 2-aminoadenosine nucleotide;
I = hypoxanthine (inosine) nucleotide

TABLE 6A
IAV RNAi Agent (targeting M1) Sense Strand Sequences
(Shown with Targeting Ligand Conjugate. The structure of αvβ6-SM6.1 is shown
in Table 11, and the structure of Tri-SM6.1-αvβ6-TA14 is shown in FIG. 1.)

| | | | Corresponding |
|---|---|---|---|
| | | | Sense Strand |
| | | | AM Number |
| | | SEQ | Without Linker |
| | | ID | or Conjugate |
| Strand ID | Modified Sense Strand (5′ → 3′) | NO. | (See Table 4) |
| CS003268 | Tri-SM6.1-αvβ6-TA14-csuaaccgaGfGfUfcgaaacguaas(invAb) | 1520 | AM16676-SS-NL |
| CS003315 | Tri-SM6.1-αvβ6-TA14-ascguacguUfCfUfuucuaucauas(invAb) | 1521 | AM16811-SS-NL |
| CS003507 | Tri-SM6.1-αvβ6-TA14-gsuugacgaUfGfGfucauuuuguas(invAb) | 1522 | AM17565-SS-NL |
| CS003509 | Tri-SM6.1-αvβ6-TA14-gsugacgauGfGfUfcauuuugucas(invAb) | 1523 | AM17567-SS-NL |
| CS003633 | Tri-SM6.1-αvβ6-TA14-csuaaccgaGfgUfcGfaaacguaas(invAb) | 1524 | AM17927-SS-NL |
| CS003638 | Tri-SM6.1-αvβ6-TA14-csuaaccgaGfgUfcgaaacguaas(invAb) | 1525 | AM17932-SS-NL |
| CS003670 | Tri-SM6.1-αvβ6-TA14-csccgaacaAfCfAfuggauagagas(invAb) | 1526 | AM18486-SS-NL |
| CS003672 | Tri-SM6.1-αvβ6-TA14-gsacaacauGfGfAfuagaicaguus(invAb) | 1527 | AM18488-SS-NL |
| CS003699 | Tri-SM6.1-αvβ6-TA14-a_2NscguacguUfCfUfuucuaucauas(invAb) | 1528 | AM18519-SS-NL |
| CS003702 | Tri-SM6.1-αvβ6-TA14-ascguacguUfcUfuUfcuaucauas(invAb) | 1529 | AM18522-SS-NL |
| CS003707 | Tri-SM6.1-αvβ6-TA14-ascguacguUfcUfUfucuaucauas(invAb) | 1530 | AM18527-SS-NL |

TABLE 6B
IAV RNAi Agent (targeting NS1) Sense Strand Sequences
(Shown with Targeting Ligand Conjugate. The structure of αvβ6-SM6.1 is shown
in Table 11, and the structure of Tri-SM6.1-αvβ6-TA14 is shown in FIG. 1.)

| | | | Corresponding |
|---|---|---|---|
| | | | Sense Strand |
| | | | AM Number |
| | | SEQ | Without Linker |
| | | ID | or Conjugate |
| Strand ID | Modified Sense Strand (5′ → 3′) | NO. | (See Table 4) |
| CS002930 | Tri-SM6.1-αvβ6-TA14-gsauacagaGfAfUfucgcuuigaas(invAb) | 1531 | AM15869-SS-NL |
| CS002932 | Tri-SM6.1-αvβ6-TA14-asgcuuucuCfGfUfuucagcuuaus(invAb) | 1532 | AM15870-SS-NL |
| CS002934 | Tri-SM6.1-αvβ6-TA14-gscuugaauGfGfAfaugguaacaas(invAb) | 1533 | AM15871-SS-NL |
| CS002936 | Tri-SM6.1-αvβ6-TA14-gscgacagaGfAfAfuaguuucgaas(invAb) | 1534 | AM15873-SS-NL |
| CS002939 | Tri-SM6.1-αvβ6-TA14-gsaugaggaUfGfUfcaaaaaugcas(invAb) | 1535 | AM15876-SS-NL |
| CS003308 | Tri-SM6.1-αvβ6-TA14-gsaugaggaUfgUfcAfaaaaugcas(invAb) | 1536 | AM16806-SS-NL |
| CS003519 | Tri-SM6.1-αvβ6-TA14-gsugaugccCfCfAfuuccuugauas(invAb) | 1537 | AM17577-SS-NL |
| CS003521 | Tri-SM6.1-αvβ6-TA14-usgaugcccCfAfUfuccuugaucas(invAb) | 1538 | AM17579-SS-NL |
| CS003523 | Tri-SM6.1-αvβ6-TA14-gsaugccccAfUfUfccuuiaucgas(invAb) | 1539 | AM17581-SS-NL |
| CS003525 | Tri-SM6.1-αvβ6-TA14-csucaucggAfGfGfacuugaaugas(invAb) | 1540 | AM17583-SS-NL |
| CS003527 | Tri-SM6.1-αvβ6-TA14-uscaucggaGfGfAfcuugaauigas(invAb) | 1541 | AM17585-SS-NL |
| CS003529 | Tri-SM6.1-αvβ6-TA14-csaucggagGfAfCfuugaauggaas(invAb) | 1542 | AM17587-SS-NL |
| CS003531 | Tri-SM6.1-αvβ6-TA14-uscggaggaCfUfUfgaauggaauas(invAb) | 1543 | AM17589-SS-NL |
| CS003533 | Tri-SM6.1-αvβ6-TA14-csggaggacUfUfGfaaugiaaugas(invAb) | 1544 | AM17591-SS-NL |
| CS003535 | Tri-SM6.1-αvβ6-TA14-ascugaaagCfGfAfacuucaiugus(invAb) | 1545 | AM17593-SS-NL |
| CS003537 | Tri-SM6.1-αvβ6-TA14-csuucucuuCfCfAfggacauacuus(invAb) | 1546 | AM17595-SS-NL |
| CS003539 | Tri-SM6.1-αvβ6-TA14-gsaugucaaAfAfAfugcaiuuggas(invAb) | 1547 | AM17597-SS-NL |
| CS003541 | Tri-SM6.1-αvβ6-TA14-gsugaugagAfAfUfgggaiaccuus(invAb) | 1548 | AM17599-SS-NL |
| CS003925 | Tri-SM6.1-αvβ6-TA14-gsaucuugaAfAfGfaggaauccaas(invAb) | 1549 | AM19061-SS-NL |
| CS003927 | Tri-SM6.1-αvβ6-TA14-ascgagacuGfGfUfucauicucaus(invAb) | 1550 | AM19063-SS-NL |
| CS003929 | Tri-SM6.1-αvβ6-TA14-gscgaacuuCfAfGfuguaaucuuus(invAb) | 1551 | AM19065-SS-NL |
| CS003931 | Tri-SM6.1-αvβ6-TA14-asccgauuaGfAfGfaccuugauaas(invAb) | 1552 | AM19067-SS-NL |
| CS003933 | Tri-SM6.1-αvβ6-TA14-gsuacuacuAfAfGfggcuuucacus(invAb) | 1553 | AM19069-SS-NL |
| CS003935 | Tri-SM6.1-αvβ6-TA14-gsaaugguaAfCfAfcgguuciagus(invAb) | 1554 | AM19071-SS-NL |
| CS003937 | Tri-SM6.1-αvβ6-TA14-usgguaacaCfGfGfuucgaiucuas(invAb) | 1555 | AM19073-SS-NL |
| CS003939 | Tri-SM6.1-αvβ6-TA14-gsuuaugcaAfGfCfcuuacaacuas(invAb) | 1556 | AM19075-SS-NL |
| CS003941 | Tri-SM6.1-αvβ6-TA14-gsgagauaaGfAfGfcuuucuciuus(invAb) | 1557 | AM19077-SS-NL |

TABLE 6C
IAV RNAi Agent (targeting PB1) Sense Strand Sequences
(Shown with Targeting Ligand Conjugate. The structure of αvβ6-SM6.1 is shown
in Table 11, and the structure of Tri-SM6.1-αvβ6-TA14 is shown in FIG. 1.)

| | | | Corresponding |
|---|---|---|---|
| | | | Sense Strand |
| | | | AM Number |
| | | SEQ | Without Linker |
| | | ID | or Conjugate |
| Strand ID | Modified Sense Strand (5′ → 3′) | NO. | (See Table 4) |
| CS003004 | Tri-SM6.1-αvβ6-TA14-uscaugccuUfGfAfaacaauggaas(invAb) | 1558 | AM16045-SS-NL |
| CS003006 | Tri-SM6.1-αvβ6-TA14-usgcgaaaaGfCfUfugaacaiucus(invAb) | 1559 | AM16047-SS-NL |
| CS003008 | Tri-SM6.1-αvβ6-TA14-gsugcagauUfAfGfagguuucguas(invAb) | 1560 | AM16049-SS-NL |
| CS003274 | Tri-SM6.1-αvβ6-TA14-asgaucuguUfCfCfaccauugaaas(invAb) | 1561 | AM16682-SS-NL |
| CS003515 | Tri-SM6.1-αvβ6-TA14-gsgagaucaUfGfAfagaucuguuas(invAb) | 1562 | AM17573-SS-NL |
| CS003681 | Tri-SM6.1-αvβ6-TA14-gsaacagaaCfAfCfaccaauacuas(invAb) | 1563 | AM18496-SS-NL |
| CS003947 | Tri-SM6.1-αvβ6-TA14-usggcucuuCfAfAfuuguucaucas(invAb) | 1564 | AM19083-SS-NL |

TABLE 6D
IAV RNAi Agent (targeting PB2) Sense Strand Sequences
(Shown with Targeting Ligand Conjugate. The structure of αvβ6-SM6.1 is shown
in Table 11, and the structure of Tri-SM6.1-αvβ6-TA14 is shown in FIG. 1.)

| | | | Corresponding |
|---|---|---|---|
| | | | Sense Strand |
| | | | AM Number |
| | | SEQ | Without Linker |
| | | ID | or Conjugate |
| Strand ID | Modified Sense Strand (5′ → 3′) | NO. | (See Table 4) |
| CS003276 | Tri-SM6.1-αvβ6-TA14-gsggacucuAfGfCfauacuuacuas(invAb) | 1565 | AM16684-SS-NL |
| CS003407 | Tri-SM6.1-αvβ6-TA14-gscgagauaCfUfCfacuaaiaccas(invAb) | 1566 | AM17366-SS-NL |
| CS003409 | Tri-SM6.1-αvβ6-TA14-gsgucgaaaGfGfUfugaaacaugas(invAb) | 1567 | AM17368-SS-NL |
| CS003411 | Tri-SM6.1-αvβ6-TA14-usgauggugGfCfGfuacaugcuaas(invAb) | 1568 | AM17370-SS-NL |
| CS003517 | Tri-SM6.1-αvβ6-TA14-asggacaagCfGfGfaucaucaguas(invAb) | 1569 | AM17575-SS-NL |
| CS003683 | Tri-SM6.1-αvβ6-TA14-uscgcugcuAfGfAfaacauaguaas(invAb) | 1570 | AM18498-SS-NL |
| CS003949 | Tri-SM6.1-αvβ6-TA14-usccaguauUfCfAfauuacaacaas(invAb) | 1571 | AM19085-SS-NL |

TABLE 6E
IAV RNAi Agent (targeting NP) Sense Strand Sequences
(Shown with Targeting Ligand Conjugate. The structure of αvβ6-SM6.1 is shown
in Table 11, and the structure of Tri-SM6.1-αvβ6-TA14 is shown in FIG. 1.)

| | | | Corresponding |
|---|---|---|---|
| | | | Sense Strand |
| | | | AM Number |
| | | SEQ | Without Linker |
| | | ID | or Conjugate |
| Strand ID | Modified Sense Strand (5′ → 3′) | NO. | (See Table 4) |
| CS003278 | Tri-SM6.1-αvβ6-TA14-gsccuuccuUfUfGfacaugaguaas(invAb) | 1572 | AM16686-SS-NL |
| CS003413 | Tri-SM6.1-αvβ6-TA14-gscucuaauGfCfAfagguucaacas(invAb) | 1573 | AM17372-SS-NL |
| CS003415 | Tri-SM6.1-αvβ6-TA14-ascgacuaaUfCfCfagaauagcaus(invAb) | 1574 | AM17374-SS-NL |
| CS003511 | Tri-SM6.1-αvβ6-TA14-gsaucagaaUfGfAfucaaaciugas(invAb) | 1575 | AM17569-SS-NL |
| CS003674 | Tri-SM6.1-αvβ6-TA14-csugucggaAfGfAfaugauugguas(invAb) | 1576 | AM18490-SS-NL |
| CS003915 | Tri-SM6.1-αvβ6-TA14-asgagaggaUfGfGfugcuuucugas(invAb) | 1577 | AM19051-SS-NL |
| CS003917 | Tri-SM6.1-αvβ6-TA14-csugaaugaUfGfCfcacauaucaas(invAb) | 1578 | AM19053-SS-NL |
| CS003919 | Tri-SM6.1-αvβ6-TA14-ascggucagCfAfCfucauucugaas(invAb) | 1579 | AM19055-SS-NL |
| CS003921 | Tri-SM6.1-αvβ6-TA14-gscucacaaGfAfGfucaauugguas(invAb) | 1580 | AM19057-SS-NL |
| CS003923 | Tri-SM6.1-αvβ6-TA14-gsuaaugaaGfGfGfucuuauuucus(invAb) | 1581 | AM19059-SS-NL |

TABLE 6F
IAV RNAi Agent (targeting PA) Sense Strand Sequences (Shown with Targeting
Ligand Conjugate. The structure of αvß6-SM6.1 is shown in Table 11,
and the structure of Tri-SM6.1-αvß6-TA14 is shown in FIG. 1.)

| | | | Corresponding |
|---|---|---|---|
| | | | Sense Strand |
| | | | AM Number |
| | | | Without Linker |
| Strand | | SEQ | or Conjugate |
| ID | Modified Sense Strand (5′ → 3′) | ID NO. | (see Table 4) |
| CS003270 | Tri-SM6.1-αvß6-TA14-gsacuuuguGfCfGfacaaugcuuas(invAb) | 1582 | AM16678-SS-NL |
| CS003272 | Tri-SM6.1-αvß6-TA14-cscacuauaUfGfAfugcaaucaaas(invAb) | 1583 | AM16680-SS-NL |
| CS003311 | Tri-SM6.1-αvß6-TA14-gsa_2NaucaaaAfCfUfaggcuuuucas(invAb) | 1584 | AM16807-SS-NL |
| CS003313 | Tri-SM6.1-αvß6-TA14-usgcaaagaUfGfUfuggaiaccuus(invAb) | 1585 | AM16809-SS-NL |
| CS003513 | Tri-SM6.1-αvß6-TA14-csgaggaguGfCfCfugauuaaugas(invAb) | 1586 | AM17571-SS-NL |
| CS003679 | Tri-SM6.1-αvß6-TA14-gsguauucgGfAfUfuuccauuucas(invAb) | 1587 | AM18494-SS-NL |
| CS003943 | Tri-SM6.1-αvß6-TA14-gsccuauguAfGfAfuggauucgaas(invAb) | 1588 | AM19079-SS-NL |
| CS003945 | Tri-SM6.1-αvß6-TA14-asucccaauGfa_2NUfaagcaaaugus(invAb) | 1589 | AM19081-SS-NL |

The IAV RNAi agents disclosed herein are formed by annealing an antisense strand with a sense strand. A sense strand containing a sequence listed in Table 2A, 2B, 2C, 2D, 2E, 2F, 4A, 4B, 4C, 4D, 4E, 4F, 5A, 5B, 5C, 5D, 5E, 5F, 6A, 6B, 6C, 6D, 6E, or 6F can be hybridized to any antisense strand containing a sequence listed in Table 2A, 2B, 2C, 2D, 2E, 2F, 3A, 3B, 3C, 3D, 3E, or 3F, provided the two sequences have a region of at least 85% complementarity over a contiguous 16, 17, 18, 19, 20, or 21 nucleotide sequence.

As shown in Tables 5A, 5B, 5C, 5D, 5E, and 5F above, certain of the example IAV RNAi agent nucleotide sequences are shown to further include reactive linking groups at one or both of the 5′ terminal end and the 3′ terminal end of the sense strand. For example, many of the IAV RNAi agent sense strand sequences shown in Tables 5A, 5B, 5C, 5D, 5E, and 5F above have a (TriAlk14) linking group at the 5′ end of the nucleotide sequence. Other linking groups, such as an (NH2-C6) linking group or a (6-SS-6) or (C6-SS-C6) linking group, may be present as well or alternatively in certain embodiments. Such reactive linking groups are positioned to facilitate the linking of targeting ligands, targeting groups, and/or PK/PD modulators to the IAV RNAi agents disclosed herein. Linking or conjugation reactions are well known in the art and provide for formation of covalent linkages between two molecules or reactants. Suitable conjugation reactions for use in the scope of the inventions herein include, but are not limited to, amide coupling reaction, Michael addition reaction, hydrazone formation reaction, inverse-demand Diels-Alder cycloaddition reaction, oxime ligation, and Copper (I)-catalyzed or strain-promoted azide-alkyne cycloaddition reaction cycloaddition reaction.

In some embodiments, targeting ligands, such as the integrin targeting ligands shown in the examples and figures disclosed herein, can be synthesized as activated esters, such as tetrafluorophenyl (TFP) esters, which can be displaced by a reactive amino group (e.g., NH₂-C₆) to attach the targeting ligand to the IAV RNAi agents disclosed herein. In some embodiments, targeting ligands are synthesized as azides, which can be conjugated to a propargyl (e.g., TriAlk14) or DBCO group, for example, via Copper (I)-catalyzed or strain-promoted azide-alkyne cycloaddition reaction.

Additionally, certain of the nucleotide sequences can be synthesized with a dT nucleotide at the 3′ terminal end of the sense strand, followed by (3′→5′) a linker (e.g., C6-SS-C6). The linker can, in some embodiments, facilitate the linkage to additional components, such as, for example, a PK/PD modulator or one or more targeting ligands. As described herein, the disulfide bond of C6-SS-C6 is first reduced, removing the dT from the molecule, which can then facilitate the conjugation of the desired PK/PD modulator. The terminal dT nucleotide therefore is not a part of the fully conjugated construct.

In some embodiments, the antisense strand of an IAV RNAi agent disclosed herein differs by 0, 1, 2, or 3 nucleotides from any of the antisense strand sequences in Table 3A, 3B, 3C, 3D, 3E, 3F, 10A, 10B, 10C, 10D, 10E., or 10F. In some embodiments, the sense strand of an IAV RNAi agent disclosed herein differs by 0, 1, 2, or 3 nucleotides from any of the sense strand sequences in Table 4A, 4B, 4C, 4D, 4E, 4F, 5A, 5B, 5C, 5D, 5E, 5F, 6A, 6B, 6C, 6D, 6E, 6F, 10A, 10B, 10C, 10D, 10E, or 10F.

In some embodiments, an IAV RNAi agent antisense strand comprises a nucleotide sequence of any of the sequences in Table 2 or Table 3. In some embodiments, an IAV RNAi agent antisense strand comprises the sequence of nucleotides (from 5′ end→3′ end) 1-17, 2-17, 1-18, 2-18, 1-19, 2-19, 1-20, 2-20, 1-21, 2-21, 1-22, 2-22, 1-23, 2-23, 1-24, or 2-24 of any of the sequences in Table 2A, 2B, 2C, 2D, 2E, 2F, 3A, 3B, 3C, 3D, 3E, 3F, 10A, 10B, 10C, 10D, 10E, or 10F. In certain embodiments, an IAV RNAi agent antisense strand comprises or consists of a modified sequence of any one of the modified sequences in Table 3 or Table 10.

In some embodiments, an IAV RNAi agent sense strand comprises the nucleotide sequence of any of the sequences in Table 2 or Table 4. In some embodiments, an IAV RNAi agent sense strand comprises the sequence of nucleotides (from 5′ end→3′ end) 1-17, 2-17, 3-17, 4-17, 1-18, 2-18, 3-18, 4-18, 1-19, 2-19, 3-19, 4-19, 1-20, 2-20, 3-20, 4-20, 1-21, 2-21, 3-21, 4-21, 1-22, 2-22, 3-22, 4-22, 1-23, 2-23, 3-23, 4-23, 1-24, 2-24, 3-24, or 4-24, of any of the sequences in Table 2A, 2B, 2C, 2D, 2E, 2F, 4A, 4B, 4C, 4D, 4E, 4F, 5A, 5B, 5C, 5D, 5E, 5F, 6A, 6B, 6C, 6D, 6E, 6F, 10A, 10B, 10C, 10D, 10E, or 10F. In certain embodiments, an IAV RNAi agent sense strand comprises or consists of a modified sequence of any one of the modified sequences in Table 3A, 3B, 3C, 3D, 3E, 3F, 10A, 10B, 10C, 10D, 10E, or 10F.

For the RNAi agents disclosed herein, the nucleotide at position 1 of the antisense strand (from 5′ end→3′ end) can be perfectly complementary to an influenza A viral genome viral genome, or can be non-complementary to an influenza A viral genome viral genome. In some embodiments, the nucleotide at position 1 of the antisense strand (from 5′ end→3′ end) is a U, A, or dT (or a modified version of U, A or dT). In some embodiments, the nucleotide at position 1 of the antisense strand (from 5′ end→3′ end) forms an A:U or U:A base pair with the sense strand.

In some embodiments, an IAV RNAi agent antisense strand comprises the sequence of nucleotides (from 5′ end→3′ end) 2-18 or 2-19 of any of the antisense strand sequences in Table 2A, 2B, 2C, 2D, 2E, 2F, 3A, 3B, 3C, 3D, 3E, 3F, 10A, 10B, 10C, 10D, 10E, or 10F. In some embodiments, an influenza A viral genome RNAi sense strand comprises the sequence of nucleotides (from 5′ end→3′ end) 1-17 or 1-18 of any of the sense strand sequences in Table 2A, 2B, 2C, 2D, 2E, 2F, 4A, 4B, 4C, 4D, 4E, 4F, 5A, 5B, 5C, 5D, 5E, 5F, 6A, 6B, 6C, 6D, 6E, 6F, 10A, 10B, 10C, 10D, 10E, or 10F.

In some embodiments, an IAV RNAi agent includes (i) an antisense strand comprising the sequence of nucleotides (from 5′ end→3′ end) 2-18 or 2-19 of any of the antisense strand sequences in Table 2, Table 3, or Table 10, and (ii) a sense strand comprising the sequence of nucleotides (from 5′ end→3′ end) 1-17 or 1-18 of any of the sense strand sequences in 2A, 2B, 2C, 2D, 2E, 2F, 4A, 4B, 4C, 4D, 4E, 4F, 5A, 5B, 5C, 5D, 5E, 5F, 6A, 6B, 6C, 6D, 6E, 6F, 10A, 10B, 10C, 10D, 10E, or 10F.

A sense strand containing a sequence listed in Table 2 or Table 4 can be hybridized to any antisense strand containing a sequence listed in Table 2 or Table 3 provided the two sequences have a region of at least 85% complementarity over a contiguous 16, 17, 18, 19, 20, or 21 nucleotide sequence. In some embodiments, the IAV RNAi agent has a sense strand consisting of the modified sequence of any of the modified sequences in Table 4A, 4B, 4C, 4D, 4E, 4F, 5A, 5B, 5C, 5D, 5E, 5F, 6A, 6B, 6C, 6D, 6E, 6F, 10A, 10B, 10C, 10D, 10E, or 10F, and an antisense strand consisting of the modified sequence of any of the modified sequences in Table 3A, 3B, 3C, 3D, 3E, 3F, 10A, 10B, 10C, 10D, 10E, or 10F. Certain representative sequence pairings are exemplified by the Duplex ID Nos. shown in Tables 7A-1, 7A-2, 7A-3, 7A-4, 7A-5, 7A-6, 7B-1, 7B-2, 7B-3, 7B-4, 7B-5, 7B-6, 8A, 8B, 8C, 8D, 8E, 8F, 9A, 9B, 9C, 9D, 9E, and 9F.

In some embodiments, an IAV RNAi agent comprises, consists of, or consists essentially of a duplex represented by any one of the Duplex ID Nos. presented herein. In some embodiments, an IAV RNAi agent consists of any of the Duplex ID Nos. presented herein. In some embodiments, an IAV RNAi agent comprises the sense strand and antisense strand nucleotide sequences of any of the Duplex ID Nos. presented herein. In some embodiments, an IAV RNAi agent comprises the sense strand and antisense strand nucleotide sequences of any of the Duplex ID Nos. presented herein and a targeting group, linking group, and/or other non-nucleotide group wherein the targeting group, linking group, and/or other non-nucleotide group is covalently linked (i.e., conjugated) to the sense strand or the antisense strand. In some embodiments, an IAV RNAi agent includes the sense strand and antisense strand modified nucleotide sequences of any of the Duplex ID Nos. presented herein. In some embodiments, an IAV RNAi agent comprises the sense strand and antisense strand modified nucleotide sequences of any of the Duplex ID Nos. presented herein and a targeting group, linking group, and/or other non-nucleotide group, wherein the targeting group, linking group, and/or other non-nucleotide group is covalently linked to the sense strand or the antisense strand.

In some embodiments, an IAV RNAi agent comprises an antisense strand and a sense strand having the nucleotide sequences of any of the antisense strand/sense strand duplexes of Tables 2A, 2B, 2C, 2D, 2E, 2F, 7A-1, 7A-2, 7A-3, 7A-4, 7A-5, 7A-6, 7B-1, 7B-2, 7B-3, 7B-4, 7B-5, 7B-6, 8A, 8B, 8C, 8D, 8E, 8F, 9A, 9B, 9C, 9D, 9E, 9F, 10A, 10B, 10C, 10D, 10E, or 10F, and comprises a targeting group. In some embodiments, an IAV RNAi agent comprises an antisense strand and a sense strand having the nucleotide sequences of any of the antisense strand/sense strand duplexes of Tables 2A, 2B, 2C, 2D, 2E, 2F, 7A-1, 7A-2, 7A-3, 7A-4, 7A-5, 7A-6, 7B-1, 7B-2, 7B-3, 7B-4, 7B-5, 7B-6, 8A, 8B, 8C, 8D, 8E, 8F, 9A, 9B, 9C, 9D, 9E, 9F, 10A, 10B, 10C, 10D, 10E, or 10F, and comprises one or more αvβ6 integrin targeting ligands.

In some embodiments, an IAV RNAi agent comprises an antisense strand and a sense strand having the nucleotide sequences of any of the antisense strand/sense strand duplexes of Tables 2A, 2B, 2C, 2D, 2E, 2F, 7A-1, 7A-2, 7A-3, 7A-4, 7A-5, 7A-6, 7B-1, 7B-2, 7B-3, 7B-4, 7B-5, 7B-6, 8A, 8B, 8C, 8D, 8E, 8F, 9A, 9B, 9C, 9D, 9E, 9F, 10A, 10B, 10C, 10D, 10E, or 10F, and comprises a targeting group that is an integrin targeting ligand. In some embodiments, an IAV RNAi agent comprises an antisense strand and a sense strand having the nucleotide sequences of any of the antisense strand/sense strand duplexes of Tables 2A, 2B, 2C, 2D, 2E, 2F, 7A-1, 7A-2, 7A-3, 7A-4, 7A-5, 7A-6, 7B-1, 7B-2, 7B-3, 7B-4, 7B-5, 7B-6, 8A, 8B, 8C, 8D, 8E, 8F, 9A, 9B, 9C, 9D, 9E, 9F, 10A, 10B, 10C, 10D, 10E, or 10F, and comprises one or more αvβ6 integrin targeting ligands or clusters of αvβ6 integrin targeting ligands (e.g., a tridentate αvβ6 integrin targeting ligand).

In some embodiments, an IAV RNAi agent comprises an antisense strand and a sense strand having the modified nucleotide sequences of any of the antisense strand/sense strand duplexes of Tables 7A-1, 7A-2, 7A-3, 7A-4, 7A-5, 7A-6, 7B-1, 7B-2, 7B-3, 7B-4, 7B-5, 7B-6, 8A, 8B, 8C, 8D, 8E, 8F, 9A, 9B, 9C, 9D, 9E, 9F, 10A, 10B, 10C, 10D, 10E, and 10F.

In some embodiments, an IAV RNAi agent comprises an antisense strand and a sense strand having the modified nucleotide sequences of any of the antisense strand/sense strand duplexes of Tables 7A-1, 7A-2, 7A-3, 7A-4, 7A-5, 7A-6, 7B-1, 7B-2, 7B-3, 7B-4, 7B-5, 7B-6, 8A, 8B, 8C, 8D, 8E, 8F, 9A, 9B, 9C, 9D, 9E, 9F, 10A, 10B, 10C, 10D, 10E, and 10F, and comprises an integrin targeting ligand.

In some embodiments, an IAV RNAi agent comprises, consists of, or consists essentially of any of the duplexes of Tables 7A-1, 7A-2, 7A-3, 7A-4, 7A-5, 7A-6, 7B-1, 7B-2, 7B-3, 7B-4, 7B-5, 7B-6, 8A, 8B, 8C, 8D, 8E, 8F, 9A, 9B, 9C, 9D, 9E, 9F, 10A, 10B, 10C, 10D, 10E, and 10F.

TABLE 7A-1
IAV RNAi agent (targeting M1) Duplexes with Corresponding Sense and Antisense
Strand ID Numbers and Sequence ID numbers for the modified and unmodified
nucleotide sequences. (Shown without Linking Agents or Conjugates)

| | | AS | AS | | SS | SS |
|---|---|---|---|---|---|---|
| | | modified | unmodified | | modified | unmodified |
| | | SEQ ID | SEQ ID | | SEQ ID | SEQ ID |
| Duplex | AS ID | NO: | NO: | SS ID | NO: | NO: |
| AD11733 | AM16677-AS | 1169 | 1590 | AM16676-SS-NL | 1368 | 1706 |
| AD11839 | AM16812-AS | 1170 | 1591 | AM16811-SS-NL | 1369 | 1707 |
| AD12417 | AM17566-AS | 1171 | 1602 | AM17565-SS-NL | 1370 | 1708 |
| AD12418 | AM17568-AS | 1172 | 1603 | AM17567-SS-NL | 1371 | 1709 |
| AD12619 | AM16677-AS | 1169 | 1590 | AM17855-SS-NL | 1372 | 1706 |
| AD12660 | AM17928-AS | 1173 | 1590 | AM17927-SS-NL | 1373 | 1706 |
| AD12661 | AM17929-AS | 1174 | 1590 | AM17927-SS-NL | 1373 | 1706 |
| AD12662 | AM17930-AS | 1175 | 1590 | AM17927-SS-NL | 1373 | 1706 |
| AD12663 | AM17931-AS | 1176 | 1590 | AM17927-SS-NL | 1373 | 1706 |
| AD12664 | AM17928-AS | 1173 | 1590 | AM17932-SS-NL | 1374 | 1706 |
| AD12665 | AM17929-AS | 1174 | 1590 | AM17932-SS-NL | 1374 | 1706 |
| AD12666 | AM17930-AS | 1175 | 1590 | AM17932-SS-NL | 1374 | 1706 |
| AD12667 | AM17931-AS | 1176 | 1590 | AM17932-SS-NL | 1374 | 1706 |
| AD12668 | AM17933-AS | 1177 | 1590 | AM17932-SS-NL | 1374 | 1706 |
| AD12669 | AM17934-AS | 1178 | 1590 | AM16676-SS-NL | 1368 | 1706 |
| AD13042 | AM18487-AS | 1179 | 1595 | AM18486-SS-NL | 1375 | 1710 |
| AD13043 | AM18489-AS | 1180 | 1596 | AM18488-SS-NL | 1376 | 1711 |
| AD13056 | AM16812-AS | 1170 | 1591 | AM18519-SS-NL | 1377 | 1770 |
| AD13057 | AM18520-AS | 1181 | 1591 | AM16811-SS-NL | 1369 | 1707 |
| AD13058 | AM18521-AS | 1182 | 1591 | AM16811-SS-NL | 1369 | 1707 |
| AD13059 | AM18523-AS | 1183 | 1591 | AM18522-SS-NL | 1378 | 1707 |
| AD13060 | AM18524-AS | 1184 | 1591 | AM18522-SS-NL | 1378 | 1707 |
| AD13061 | AM18525-AS | 1185 | 1591 | AM18522-SS-NL | 1378 | 1707 |
| AD13062 | AM18526-AS | 1186 | 1591 | AM18522-SS-NL | 1378 | 1707 |
| AD13063 | AM18524-AS | 1184 | 1591 | AM18527-SS-NL | 1379 | 1707 |
| AD13064 | AM18525-AS | 1185 | 1591 | AM18527-SS-NL | 1379 | 1707 |
| AD13065 | AM18526-AS | 1186 | 1591 | AM18527-SS-NL | 1379 | 1707 |
| AD14065 | AM17930-AS | 1175 | 1590 | AM16676-SS-NL | 1368 | 1706 |
| AD14135 | AM18523-AS | 1183 | 1591 | AM16811-SS-NL | 1369 | 1707 |
| AD14136 | AM19907-AS | 1187 | 1591 | AM16811-SS-NL | 1369 | 1707 |
| AD14137 | AM18524-AS | 1184 | 1591 | AM16811-SS-NL | 1369 | 1707 |
| AD14138 | AM18525-AS | 1185 | 1591 | AM16811-SS-NL | 1369 | 1707 |
| AD14139 | AM18526-AS | 1186 | 1591 | AM16811-SS-NL | 1369 | 1707 |
| AD14140 | AM19908-AS | 1188 | 1591 | AM16811-SS-NL | 1369 | 1707 |
| AD14141 | AM19909-AS | 1189 | 1591 | AM16811-SS-NL | 1369 | 1707 |
| AD14142 | AM19910-AS | 1190 | 1591 | AM16811-SS-NL | 1369 | 1707 |
| AD14143 | AM19911-AS | 1191 | 1591 | AM16811-SS-NL | 1369 | 1707 |
| AD14144 | AM19912-AS | 1192 | 1591 | AM16811-SS-NL | 1369 | 1707 |

TABLE 7A-2
IAV RNAi agent (targeting NS1) Duplexes with Corresponding Sense and Antisense
Strand ID Numbers and Sequence ID numbers for the modified and unmodified
nucleotide sequences. (Shown without Linking Agents or Conjugates)

| | | AS | AS | | SS | SS |
|---|---|---|---|---|---|---|
| | | modified | unmodified | | modified | unmodified |
| | | SEQ ID | SEQ ID | | SEQ ID | SEQ ID |
| Duplex | AS ID | NO: | NO: | SS ID | NO: | NO: |
| AD11159 | AM15547-AS | 1228 | 1613 | AM15869-SS-NL | 1380 | 1712 |
| AD11160 | AM15729-AS | 1242 | 1625 | AM15870-SS-NL | 1381 | 1713 |
| AD11161 | AM15872-AS | 1244 | 1621 | AM15871-SS-NL | 1382 | 1714 |
| AD11162 | AM15874-AS | 1245 | 1636 | AM15873-SS-NL | 1383 | 1715 |
| AD11164 | AM15877-AS | 1246 | 1607 | AM15876-SS-NL | 1384 | 1716 |
| AD11835 | AM15714-AS | 1234 | 1607 | AM16806-SS-NL | 1385 | 1716 |
| AD11836 | AM15548-AS | 1229 | 1613 | AM15869-SS-NL | 1380 | 1712 |
| AD12423 | AM17578-AS | 1247 | 1604 | AM17577-SS-NL | 1386 | 1717 |
| AD12424 | AM17580-AS | 1248 | 1605 | AM17579-SS-NL | 1387 | 1718 |
| AD12425 | AM17582-AS | 1249 | 1606 | AM17581-SS-NL | 1388 | 1719 |
| AD12426 | AM17584-AS | 1250 | 1608 | AM17583-SS-NL | 1389 | 1720 |
| AD12427 | AM17586-AS | 1251 | 1609 | AM17585-SS-NL | 1390 | 1721 |
| AD12428 | AM17588-AS | 1252 | 1610 | AM17587-SS-NL | 1391 | 1722 |
| AD12429 | AM17590-AS | 1253 | 1611 | AM17589-SS-NL | 1392 | 1723 |
| AD12430 | AM17592-AS | 1254 | 1612 | AM17591-SS-NL | 1393 | 1724 |
| AD12431 | AM17594-AS | 1255 | 1615 | AM17593-SS-NL | 1394 | 1725 |
| AD12432 | AM17596-AS | 1256 | 1618 | AM17595-SS-NL | 1395 | 1726 |
| AD12433 | AM17598-AS | 1257 | 1619 | AM17597-SS-NL | 1396 | 1727 |
| AD12434 | AM17600-AS | 1258 | 1623 | AM17599-SS-NL | 1397 | 1728 |
| AD13045 | AM18492-AS | 1259 | 1621 | AM15871-SS-NL | 1382 | 1714 |
| AD13046 | AM18493-AS | 1260 | 1636 | AM15873-SS-NL | 1383 | 1715 |
| AD13047 | AM15730-AS | 1243 | 1625 | AM15870-SS-NL | 1381 | 1713 |
| AD13400 | AM19062-AS | 1261 | 1628 | AM19061-SS-NL | 1398 | 1729 |
| AD13401 | AM19064-AS | 1262 | 1631 | AM19063-SS-NL | 1399 | 1730 |
| AD13402 | AM19066-AS | 1263 | 1632 | AM19065-SS-NL | 1400 | 1731 |
| AD13403 | AM19068-AS | 1264 | 1633 | AM19067-SS-NL | 1401 | 1732 |
| AD13404 | AM19070-AS | 1265 | 1616 | AM19069-SS-NL | 1402 | 1733 |
| AD13405 | AM19072-AS | 1266 | 1634 | AM19071-SS-NL | 1403 | 1734 |
| AD13406 | AM19074-AS | 1267 | 1622 | AM19073-SS-NL | 1404 | 1735 |
| AD13407 | AM19076-AS | 1268 | 1624 | AM19075-SS-NL | 1405 | 1736 |
| AD13408 | AM19078-AS | 1269 | 1637 | AM19077-SS-NL | 1406 | 1737 |
| AD11159 | AM15547-AS | 1228 | 1613 | AM15869-SS-NL | 1380 | 1712 |
| AD11160 | AM15729-AS | 1242 | 1625 | AM15870-SS-NL | 1381 | 1713 |
| AD11161 | AM15872-AS | 1244 | 1621 | AM15871-SS-NL | 1382 | 1714 |
| AD11162 | AM15874-AS | 1245 | 1636 | AM15873-SS-NL | 1383 | 1715 |
| AD11164 | AM15877-AS | 1246 | 1607 | AM15876-SS-NL | 1384 | 1716 |
| AD11835 | AM15714-AS | 1234 | 1607 | AM16806-SS-NL | 1385 | 1716 |
| AD11836 | AM15548-AS | 1229 | 1613 | AM15869-SS-NL | 1380 | 1712 |
| AD12423 | AM17578-AS | 1247 | 1604 | AM17577-SS-NL | 1386 | 1717 |
| AD12424 | AM17580-AS | 1248 | 1605 | AM17579-SS-NL | 1387 | 1718 |
| AD12425 | AM17582-AS | 1249 | 1606 | AM17581-SS-NL | 1388 | 1719 |
| AD12426 | AM17584-AS | 1250 | 1608 | AM17583-SS-NL | 1389 | 1720 |
| AD12427 | AM17586-AS | 1251 | 1609 | AM17585-SS-NL | 1390 | 1721 |
| AD12428 | AM17588-AS | 1252 | 1610 | AM17587-SS-NL | 1391 | 1722 |

TABLE 7A-3
IAV RNAi agent (targeting PB1) Duplexes with Corresponding Sense and Antisense
Strand ID Numbers and Sequence ID numbers for the modified and unmodified
nucleotide sequences. (Shown without Linking Agents or Conjugates)

| | | AS | AS | | SS | SS |
|---|---|---|---|---|---|---|
| | | modified | unmodified | | modified | unmodified |
| | | SEQ ID | SEQ ID | | SEQ ID | SEQ ID |
| Duplex | AS ID | NO: | NO: | SS ID | NO: | NO: |
| AD11292 | AM16046-AS | 1295 | 1642 | AM16045-SS-NL | 1407 | 1738 |
| AD11293 | AM16048-AS | 1296 | 1658 | AM16047-SS-NL | 1408 | 1739 |
| AD11294 | AM16050-AS | 1297 | 1657 | AM16049-SS-NL | 1409 | 1740 |
| AD11736 | AM16683-AS | 1298 | 1655 | AM16682-SS-NL | 1410 | 1741 |
| AD12421 | AM17574-AS | 1299 | 1663 | AM17573-SS-NL | 1411 | 1742 |
| AD12620 | AM16683-AS | 1298 | 1655 | AM17856-SS-NL | 1412 | 1741 |
| AD13049 | AM18497-AS | 1300 | 1641 | AM18496-SS-NL | 1413 | 1743 |
| AD13411 | AM19084-AS | 1301 | 1665 | AM19083-SS-NL | 1414 | 1744 |

TABLE 7A-4
IAV RNAi agent (targeting PB2) Duplexes with Corresponding Sense and Antisense
Strand ID Numbers and Sequence ID numbers for the modified and unmodified
nucleotide sequences. (Shown without Linking Agents or Conjugates)

| | | AS | AS | | SS | SS |
|---|---|---|---|---|---|---|
| | | modified | unmodified | | modified | unmodified |
| | | SEQ ID | SEQ ID | | SEQ ID | SEQ ID |
| Duplex | AS ID | NO: | NO: | SS ID | NO: | NO: |
| AD11737 | AM16685-AS | 1314 | 1676 | AM16684-SS-NL | 1415 | 1745 |
| AD12249 | AM17367-AS | 1315 | 1665 | AM17366-SS-NL | 1416 | 1746 |
| AD12250 | AM17369-AS | 1316 | 1666 | AM17368-SS-NL | 1417 | 1747 |
| AD12251 | AM17371-AS | 1317 | 1667 | AM17370-SS-NL | 1418 | 1748 |
| AD12422 | AM17576-AS | 1318 | 1669 | AM17575-SS-NL | 1419 | 1749 |
| AD12621 | AM16685-AS | 1314 | 1676 | AM17857-SS-NL | 1420 | 1745 |
| AD13050 | AM18499-AS | 1319 | 1668 | AM18498-SS-NL | 1421 | 1750 |
| AD13412 | AM19086-AS | 1320 | 1675 | AM19085-SS-NL | 1422 | 1751 |

TABLE 7A-5
IAV RNAi agent (targeting NP) Duplexes with Corresponding Sense and Antisense
Strand ID Numbers and Sequence ID numbers for the modified and unmodified
nucleotide sequences. (Shown without Linking Agents or Conjugates)

| | | AS | AS | | SS | SS |
|---|---|---|---|---|---|---|
| | | modified | unmodified | | modified | unmodified |
| | | SEQ ID | SEQ ID | | SEQ ID | SEQ ID |
| Duplex | AS ID | NO: | NO: | SS ID | NO: | NO: |
| AD11738 | AM16687-AS | 1337 | 1691 | AM16686-SS-NL | 1423 | 1752 |
| AD12252 | AM17373-AS | 1338 | 1682 | AM17372-SS-NL | 1424 | 1753 |
| AD12253 | AM17375-AS | 1339 | 1678 | AM17374-SS-NL | 1425 | 1754 |
| AD12419 | AM17570-AS | 1340 | 1683 | AM17569-SS-NL | 1426 | 1755 |
| AD12622 | AM16687-AS | 1337 | 1691 | AM17858-SS-NL | 1427 | 1752 |
| AD13044 | AM18491-AS | 1341 | 1677 | AM18490-SS-NL | 1428 | 1756 |
| AD13395 | AM19052-AS | 1342 | 1679 | AM19051-SS-NL | 1429 | 1757 |
| AD13396 | AM19054-AS | 1343 | 1681 | AM19053-SS-NL | 1430 | 1758 |
| AD13397 | AM19056-AS | 1344 | 1686 | AM19055-SS-NL | 1431 | 1759 |
| AD13398 | AM19058-AS | 1345 | 1688 | AM19057-SS-NL | 1432 | 1760 |
| AD13399 | AM19060-AS | 1346 | 1692 | AM19059-SS-NL | 1433 | 1761 |

TABLE 7A-6
IAV RNAi agent (targeting PA) Duplexes with Corresponding Sense and Antisense
Strand ID Numbers and Sequence ID numbers for the modified and unmodified
nucleotide sequences. (Shown without Linking Agents or Conjugates)

| | | AS | AS | | SS | SS |
|---|---|---|---|---|---|---|
| | | modified | unmodified | | modified | unmodified |
| | | SEQ ID | SEQ ID | | SEQ ID | SEQ ID |
| Duplex | AS ID | NO: | NO: | SS ID | NO: | NO: |
| AD11734 | AM16679-AS | 1360 | 1693 | AM16678-SS-NL | 1434 | 1762 |
| AD11735 | AM16681-AS | 1361 | 1701 | AM16680-SS-NL | 1435 | 1763 |
| AD11837 | AM16808-AS | 1362 | 1696 | AM16807-SS-NL | 1436 | 1771 |
| AD11838 | AM16810-AS | 1363 | 1702 | AM16809-SS-NL | 1437 | 1765 |
| AD12420 | AM17572-AS | 1364 | 1705 | AM17571-SS-NL | 1438 | 1766 |
| AD12623 | AM16679-AS | 1360 | 1396 | AM17859-SS-NL | 1439 | 1762 |
| AD12624 | AM16681-AS | 1361 | 1701 | AM17860-SS-NL | 1440 | 1763 |
| AD13048 | AM18495-AS | 1365 | 1694 | AM18494-SS-NL | 1441 | 1767 |
| AD13409 | AM19080-AS | 1366 | 1699 | AM19079-SS-NL | 1442 | 1768 |
| AD13410 | AM19082-AS | 1367 | 1704 | AM19081-SS-NL | 1443 | 1772 |

TABLE 7B-1
IAV RNAi agent (targeting M1) Duplexes with Corresponding
Sense and Antisense Strand ID Numbers and Sequence ID numbers
for the modified and unmodified nucleotide sequences.

| | | AS | AS | | SS | SS |
|---|---|---|---|---|---|---|
| | | modified | unmodified | | modified | unmodified |
| | | SEQ ID | SEQ ID | | SEQ ID | SEQ ID |
| Duplex | AS ID | NO: | NO: | SS ID | NO: | NO: |
| AD11733 | AM16677-AS | 1169 | 1590 | AM16676-SS | 1444 | 1706 |
| AD11839 | AM16812-AS | 1170 | 1591 | AM16811-SS | 1445 | 1707 |
| AD12417 | AM17566-AS | 1171 | 1602 | AM17565-SS | 1446 | 1708 |
| AD12418 | AM17568-AS | 1172 | 1603 | AM17567-SS | 1447 | 1709 |
| AD12619 | AM16677-AS | 1169 | 1590 | AM17855-SS | 1448 | 1706 |
| AD12660 | AM17928-AS | 1173 | 1590 | AM17927-SS | 1449 | 1706 |
| AD12661 | AM17929-AS | 1174 | 1590 | AM17927-SS | 1449 | 1706 |
| AD12662 | AM17930-AS | 1175 | 1590 | AM17927-SS | 1449 | 1706 |
| AD12663 | AM17931-AS | 1176 | 1590 | AM17927-SS | 1449 | 1706 |
| AD12664 | AM17928-AS | 1173 | 1590 | AM17932-SS | 1450 | 1706 |
| AD12665 | AM17929-AS | 1174 | 1590 | AM17932-SS | 1450 | 1706 |
| AD12666 | AM17930-AS | 1175 | 1590 | AM17932-SS | 1450 | 1706 |
| AD12667 | AM17931-AS | 1176 | 1590 | AM17932-SS | 1450 | 1706 |
| AD12668 | AM17933-AS | 1177 | 1590 | AM17932-SS | 1450 | 1706 |
| AD12669 | AM17934-AS | 1178 | 1590 | AM16676-SS | 1444 | 1706 |
| AD13042 | AM18487-AS | 1179 | 1595 | AM18486-SS | 1451 | 1710 |
| AD13043 | AM18489-AS | 1180 | 1596 | AM18488-SS | 1452 | 1711 |
| AD13056 | AM16812-AS | 1170 | 1591 | AM18519-SS | 1453 | 1770 |
| AD13057 | AM18520-AS | 1181 | 1591 | AM16811-SS | 1445 | 1707 |
| AD13058 | AM18521-AS | 1182 | 1591 | AM16811-SS | 1445 | 1707 |
| AD13059 | AM18523-AS | 1183 | 1591 | AM18522-SS | 1454 | 1707 |
| AD13060 | AM18524-AS | 1184 | 1591 | AM18522-SS | 1454 | 1707 |
| AD13061 | AM18525-AS | 1185 | 1591 | AM18522-SS | 1454 | 1707 |
| AD13062 | AM18526-AS | 1186 | 1591 | AM18522-SS | 1454 | 1707 |
| AD13063 | AM18524-AS | 1184 | 1591 | AM18527-SS | 1455 | 1707 |
| AD13064 | AM18525-AS | 1185 | 1591 | AM18527-SS | 1455 | 1707 |
| AD13065 | AM18526-AS | 1186 | 1591 | AM18527-SS | 1455 | 1707 |
| AD14065 | AM17930-AS | 1175 | 1590 | AM16676-SS | 1444 | 1706 |
| AD14135 | AM18523-AS | 1183 | 1591 | AM16811-SS | 1445 | 1707 |
| AD14136 | AM19907-AS | 1187 | 1591 | AM16811-SS | 1445 | 1707 |
| AD14137 | AM18524-AS | 1184 | 1591 | AM16811-SS | 1445 | 1707 |
| AD14138 | AM18525-AS | 1185 | 1591 | AM16811-SS | 1445 | 1707 |
| AD14139 | AM18526-AS | 1186 | 1591 | AM16811-SS | 1445 | 1707 |
| AD14140 | AM19908-AS | 1188 | 1591 | AM16811-SS | 1445 | 1707 |
| AD14141 | AM19909-AS | 1189 | 1591 | AM16811-SS | 1445 | 1707 |
| AD14142 | AM19910-AS | 1190 | 1591 | AM16811-SS | 1445 | 1707 |
| AD14143 | AM19911-AS | 1191 | 1591 | AM16811-SS | 1445 | 1707 |
| AD14144 | AM19912-AS | 1192 | 1591 | AM16811-SS | 1445 | 1707 |

TABLE 7B-2
IAV RNAi agent (targeting NS1) Duplexes with Corresponding
Sense and Antisense Strand ID Numbers and Sequence ID numbers
for the modified and unmodified nucleotide sequences.

| | | AS | AS | | SS | SS |
|---|---|---|---|---|---|---|
| | | modified | unmodified | | modified | unmodified |
| | | SEQ ID | SEQ ID | | SEQ ID | SEQ ID |
| Duplex | AS ID | NO: | NO: | SS ID | NO: | NO: |
| AD11159 | AM15547-AS | 1228 | 1613 | AM15869-SS | 1456 | 1712 |
| AD11160 | AM15729-AS | 1242 | 1625 | AM15870-SS | 1457 | 1713 |
| AD11161 | AM15872-AS | 1244 | 1621 | AM15871-SS | 1458 | 1714 |
| AD11162 | AM15874-AS | 1245 | 1636 | AM15873-SS | 1459 | 1715 |
| AD11164 | AM15877-AS | 1246 | 1607 | AM15876-SS | 1460 | 1716 |
| AD11835 | AM15714-AS | 1234 | 1607 | AM16806-SS | 1461 | 1716 |
| AD11836 | AM15548-AS | 1229 | 1613 | AM15869-SS | 1456 | 1712 |
| AD12423 | AM17578-AS | 1247 | 1604 | AM17577-SS | 1462 | 1717 |
| AD12424 | AM17580-AS | 1248 | 1605 | AM17579-SS | 1463 | 1718 |
| AD12425 | AM17582-AS | 1249 | 1606 | AM17581-SS | 1464 | 1719 |
| AD12426 | AM17584-AS | 1250 | 1608 | AM17583-SS | 1465 | 1720 |
| AD12427 | AM17586-AS | 1251 | 1609 | AM17585-SS | 1466 | 1721 |
| AD12428 | AM17588-AS | 1252 | 1610 | AM17587-SS | 1467 | 1722 |
| AD12429 | AM17590-AS | 1253 | 1611 | AM17589-SS | 1468 | 1723 |
| AD12430 | AM17592-AS | 1254 | 1612 | AM17591-SS | 1469 | 1724 |
| AD12431 | AM17594-AS | 1255 | 1615 | AM17593-SS | 1470 | 1725 |
| AD12432 | AM17596-AS | 1256 | 1618 | AM17595-SS | 1471 | 1726 |
| AD12433 | AM17598-AS | 1257 | 1619 | AM17597-SS | 1472 | 1727 |
| AD12434 | AM17600-AS | 1258 | 1623 | AM17599-SS | 1473 | 1728 |
| AD13045 | AM18492-AS | 1259 | 1621 | AM15871-SS | 1458 | 1714 |
| AD13046 | AM18493-AS | 1260 | 1636 | AM15873-SS | 1459 | 1715 |
| AD13047 | AM15730-AS | 1243 | 1625 | AM15870-SS | 1457 | 1713 |
| AD13400 | AM19062-AS | 1261 | 1628 | AM19061-SS | 1474 | 1729 |
| AD13401 | AM19064-AS | 1262 | 1631 | AM19063-SS | 1475 | 1730 |
| AD13402 | AM19066-AS | 1263 | 1632 | AM19065-SS | 1476 | 1731 |
| AD13403 | AM19068-AS | 1264 | 1633 | AM19067-SS | 1477 | 1732 |
| AD13404 | AM19070-AS | 1265 | 1616 | AM19069-SS | 1478 | 1733 |
| AD13405 | AM19072-AS | 1266 | 1634 | AM19071-SS | 1479 | 1734 |
| AD13406 | AM19074-AS | 1267 | 1622 | AM19073-SS | 1480 | 1735 |
| AD13407 | AM19076-AS | 1268 | 1624 | AM19075-SS | 1481 | 1736 |
| AD13408 | AM19078-AS | 1269 | 1637 | AM19077-SS | 1482 | 1737 |

TABLE 7B-3
IAV RNAi agent (targeting PB1) Duplexes with Corresponding
Sense and Antisense Strand ID Numbers and Sequence ID numbers
for the modified and unmodified nucleotide sequences.

| | | AS | AS | | SS | SS |
|---|---|---|---|---|---|---|
| | | modified | unmodified | | modified | unmodified |
| | | SEQ ID | SEQ ID | | SEQ ID | SEQ ID |
| Duplex | AS ID | NO: | NO: | SS ID | NO: | NO: |
| AD11292 | AM16046-AS | 1295 | 1642 | AM16045-SS | 1483 | 1738 |
| AD11293 | AM16048-AS | 1296 | 1658 | AM16047-SS | 1484 | 1739 |
| AD11294 | AM16050-AS | 1297 | 1657 | AM16049-SS | 1485 | 1740 |
| AD11736 | AM16683-AS | 1298 | 1655 | AM16682-SS | 1486 | 1741 |
| AD12421 | AM17574-AS | 1299 | 1663 | AM17573-SS | 1487 | 1742 |
| AD12620 | AM16683-AS | 1298 | 1655 | AM17856-SS | 1488 | 1741 |
| AD13049 | AM18497-AS | 1300 | 1641 | AM18496-SS | 1489 | 1743 |
| AD13411 | AM19084-AS | 1301 | 1665 | AM19083-SS | 1490 | 1744 |

TABLE 7B-4
IAV RNAi agent (targeting PB2) Duplexes with Corresponding
Sense and Antisense Strand ID Numbers and Sequence ID numbers
for the modified and unmodified nucleotide sequences.

| | | AS | AS | | SS | SS |
|---|---|---|---|---|---|---|
| | | modified | unmodified | | modified | unmodified |
| | | SEQ ID | SEQ ID | | SEQ ID | SEQ ID |
| Duplex | AS ID | NO: | NO: | SS ID | NO: | NO: |
| AD11737 | AM16685-AS | 1314 | 1676 | AM16684-SS | 1491 | 1745 |
| AD12249 | AM17367-AS | 1315 | 1665 | AM17366-SS | 1492 | 1746 |
| AD12250 | AM17369-AS | 1316 | 1666 | AM17368-SS | 1493 | 1747 |
| AD12251 | AM17371-AS | 1317 | 1667 | AM17370-SS | 1494 | 1748 |
| AD12422 | AM17576-AS | 1318 | 1669 | AM17575-SS | 1495 | 1749 |
| AD12621 | AM16685-AS | 1314 | 1676 | AM17857-SS | 1496 | 1745 |
| AD13050 | AM18499-AS | 1319 | 1668 | AM18498-SS | 1497 | 1750 |
| AD13412 | AM19086-AS | 1320 | 1675 | AM19085-SS | 1498 | 1751 |

TABLE 7B-5
IAV RNAi agent (targeting NP) Duplexes with Corresponding Sense and Antisense Strand ID
Numbers and Sequence ID numbers for the modified and unmodified nucleotide sequences.

| | | AS | AS | | SS | SS |
|---|---|---|---|---|---|---|
| | | modified | unmodified | | modified | unmodified |
| | | SEQ ID | SEQ ID | | SEQ ID | SEQ ID |
| Duplex | AS ID | NO: | NO: | SS ID | NO: | NO: |
| AD11738 | AM16687-AS | 1337 | 1691 | AM16686-SS | 1499 | 1752 |
| AD12252 | AM17373-AS | 1338 | 1682 | AM17372-SS | 1500 | 1753 |
| AD12253 | AM17375-AS | 1339 | 1678 | AM17374-SS | 1501 | 1754 |
| AD12419 | AM17570-AS | 1340 | 1683 | AM17569-SS | 1502 | 1755 |
| AD12622 | AM16687-AS | 1337 | 1691 | AM17858-SS | 1503 | 1752 |
| AD13044 | AM18491-AS | 1341 | 1677 | AM18490-SS | 1504 | 1756 |
| AD13395 | AM19052-AS | 1342 | 1679 | AM19051-SS | 1505 | 1757 |
| AD13396 | AM19054-AS | 1343 | 1681 | AM19053-SS | 1506 | 1758 |
| AD13397 | AM19056-AS | 1344 | 1686 | AM19055-SS | 1507 | 1759 |
| AD13398 | AM19058-AS | 1345 | 1688 | AM19057-SS | 1508 | 1760 |
| AD13399 | AM19060-AS | 1346 | 1692 | AM19059-SS | 1509 | 1761 |

TABLE 7B-6
IAV RNAi agent (targeting PA) Duplexes with Corresponding Sense and Antisense Strand ID
Numbers and Sequence ID numbers for the modified and unmodified nucleotide sequences.

| | | AS | AS | | SS | SS |
|---|---|---|---|---|---|---|
| | | modified | unmodified | | modified | unmodified |
| | | SEQ ID | SEQ ID | | SEQ ID | SEQ ID |
| Duplex | AS ID | NO: | NO: | SS ID | NO: | NO: |
| AD11734 | AM16679-AS | 1360 | 1693 | AM16678-SS | 1510 | 1762 |
| AD11735 | AM16681-AS | 1361 | 1701 | AM16680-SS | 1511 | 1763 |
| AD11837 | AM16808-AS | 1362 | 1696 | AM16807-SS | 1512 | 1771 |
| AD11838 | AM16810-AS | 1363 | 1702 | AM16809-SS | 1513 | 1765 |
| AD12420 | AM17572-AS | 1364 | 1705 | AM17571-SS | 1514 | 1766 |
| AD12623 | AM16679-AS | 1360 | 1693 | AM17859-SS | 1515 | 1762 |
| AD12624 | AM16681-AS | 1361 | 1701 | AM17860-SS | 1516 | 1763 |
| AD13048 | AM18495-AS | 1365 | 1694 | AM18494-SS | 1517 | 1767 |
| AD13409 | AM19080-AS | 1366 | 1699 | AM19079-SS | 1518 | 1768 |
| AD13410 | AM19082-AS | 1367 | 1704 | AM19081-SS | 1519 | 1772 |

TABLE 8A
IAV RNAi agent Duplexes (targeting M1) with Corresponding Sense and
Antisense Strand ID Numbers and Sequence ID numbers for the modified and
unmodified nucleotide sequences. (Shown with Targeting Ligand Conjugates)

| | | AS | AS | | SS | SS |
|---|---|---|---|---|---|---|
| | | modified | unmodified | | modified | unmodified |
| | | SEQ ID | SEQ ID | | SEQ ID | SEQ ID |
| Duplex | AS ID | NO: | NO: | SS ID | NO: | NO: |
| AC002564 | AM16677-AS | 1169 | 1590 | CS003268 | 1520 | 1706 |
| AC002601 | AM16812-AS | 1170 | 1591 | CS003315 | 1521 | 1707 |
| AC002754 | AM17566-AS | 1171 | 1602 | CS003507 | 1522 | 1708 |
| AC002755 | AM17568-AS | 1172 | 1603 | CS003509 | 1523 | 1709 |
| AC002863 | AM17928-AS | 1173 | 1590 | CS003633 | 1524 | 1706 |
| AC002864 | AM17929-AS | 1174 | 1590 | CS003633 | 1524 | 1706 |
| AC002865 | AM17930-AS | 1175 | 1590 | CS003633 | 1524 | 1706 |
| AC002866 | AM17931-AS | 1176 | 1590 | CS003633 | 1524 | 1706 |
| AC002867 | AM17928-AS | 1173 | 1590 | CS003638 | 1525 | 1706 |
| AC002868 | AM17929-AS | 1174 | 1590 | CS003638 | 1525 | 1706 |
| AC002869 | AM17930-AS | 1175 | 1590 | CS003638 | 1525 | 1706 |
| AC002870 | AM17931-AS | 1176 | 1590 | CS003638 | 1525 | 1706 |
| AC002871 | AM17933-AS | 1177 | 1590 | CS003638 | 1525 | 1706 |
| AC002872 | AM17934-AS | 1178 | 1590 | CS003268 | 1520 | 1706 |
| AC002902 | AM18487-AS | 1179 | 1595 | CS003670 | 1526 | 1710 |
| AC002903 | AM18489-AS | 1180 | 1596 | CS003672 | 1527 | 1711 |
| AC002925 | AM16812-AS | 1170 | 1591 | CS003699 | 1528 | 1770 |
| AC002926 | AM18520-AS | 1181 | 1591 | CS003315 | 1521 | 1707 |
| AC002927 | AM18521-AS | 1182 | 1591 | CS003315 | 1521 | 1707 |
| AC002928 | AM18523-AS | 1183 | 1591 | CS003702 | 1529 | 1707 |
| AC002929 | AM18524-AS | 1184 | 1591 | CS003702 | 1529 | 1707 |
| AC002930 | AM18525-AS | 1185 | 1591 | CS003702 | 1529 | 1707 |
| AC002931 | AM18526-AS | 1186 | 1591 | CS003702 | 1529 | 1707 |
| AC002932 | AM18524-AS | 1184 | 1591 | CS003707 | 1530 | 1707 |
| AC002933 | AM18525-AS | 1185 | 1591 | CS003707 | 1530 | 1707 |
| AC002934 | AM18526-AS | 1186 | 1591 | CS003707 | 1530 | 1707 |
| AC003274 | AM17930-AS | 1175 | 1590 | CS003268 | 1520 | 1706 |
| AC003405 | AM18523-AS | 1183 | 1591 | CS003315 | 1521 | 1707 |
| AC003406 | AM19907-AS | 1187 | 1591 | CS003315 | 1521 | 1707 |
| AC003407 | AM18524-AS | 1184 | 1591 | CS003315 | 1521 | 1707 |
| AC003408 | AM18525-AS | 1185 | 1591 | CS003315 | 1521 | 1707 |
| AC003409 | AM18526-AS | 1186 | 1591 | CS003315 | 1521 | 1707 |
| AC003410 | AM19908-AS | 1188 | 1591 | CS003315 | 1521 | 1707 |
| AC003411 | AM19909-AS | 1189 | 1591 | CS003315 | 1521 | 1707 |
| AC003412 | AM19910-AS | 1190 | 1591 | CS003315 | 1521 | 1707 |
| AC003413 | AM19911-AS | 1191 | 1591 | CS003315 | 1521 | 1707 |
| AC003414 | AM19912-AS | 1192 | 1591 | CS003315 | 1521 | 1707 |

TABLE 8B
IAV RNAi agent Duplexes (targeting NS1) with Corresponding Sense and
Antisense Strand ID Numbers and Sequence ID numbers for the modified and unmodified
nucleotide sequences. (Shown with Targeting Ligand Conjugates)

| | | AS | AS | | SS | SS |
|---|---|---|---|---|---|---|
| | | modified | unmodified | | modified | unmodified |
| | | SEQ ID | SEQ ID | | SEQ ID | SEQ ID |
| Duplex | AS ID | NO: | NO: | SS ID | NO: | NO: |
| AC002317 | AM15547-AS | 1228 | 1613 | CS002930 | 1531 | 1712 |
| AC002318 | AM15729-AS | 1242 | 1625 | CS002932 | 1532 | 1713 |
| AC002319 | AM15872-AS | 1244 | 1621 | CS002934 | 1533 | 1714 |
| AC002320 | AM15874-AS | 1245 | 1636 | CS002936 | 1534 | 1715 |
| AC002322 | AM15877-AS | 1246 | 1607 | CS002939 | 1535 | 1716 |
| AC002597 | AM15714-AS | 1234 | 1607 | CS003308 | 1536 | 1716 |
| AC002598 | AM15548-AS | 1229 | 1613 | CS002930 | 1531 | 1712 |
| AC002760 | AM17578-AS | 1247 | 1604 | CS003519 | 1537 | 1717 |
| AC002761 | AM17580-AS | 1248 | 1605 | CS003521 | 1538 | 1718 |
| AC002762 | AM17582-AS | 1249 | 1606 | CS003523 | 1539 | 1719 |
| AC002763 | AM17584-AS | 1250 | 1608 | CS003525 | 1540 | 1720 |
| AC002764 | AM17586-AS | 1251 | 1609 | CS003527 | 1541 | 1721 |
| AC002765 | AM17588-AS | 1252 | 1610 | CS003529 | 1542 | 1722 |
| AC002766 | AM17590-AS | 1253 | 1611 | CS003531 | 1543 | 1723 |
| AC002767 | AM17592-AS | 1254 | 1612 | CS003533 | 1544 | 1724 |
| AC002768 | AM17594-AS | 1255 | 1615 | CS003535 | 1545 | 1725 |
| AC002769 | AM17596-AS | 1256 | 1618 | CS003537 | 1546 | 1726 |
| AC002770 | AM17598-AS | 1257 | 1619 | CS003539 | 1547 | 1727 |
| AC002771 | AM17600-AS | 1258 | 1623 | CS003541 | 1548 | 1728 |
| AC002905 | AM18492-AS | 1259 | 1621 | CS002934 | 1533 | 1714 |
| AC002906 | AM18493-AS | 1260 | 1636 | CS002936 | 1534 | 1715 |
| AC002907 | AM15730-AS | 1243 | 1625 | CS002932 | 1532 | 1713 |
| AC003112 | AM19062-AS | 1261 | 1628 | CS003925 | 1549 | 1729 |
| AC003113 | AM19064-AS | 1262 | 1631 | CS003927 | 1550 | 1730 |
| AC003114 | AM19066-AS | 1263 | 1632 | CS003929 | 1551 | 1731 |
| AC003115 | AM19068-AS | 1264 | 1633 | CS003931 | 1552 | 1732 |
| AC003116 | AM19070-AS | 1265 | 1616 | CS003933 | 1553 | 1733 |
| AC003117 | AM19072-AS | 1266 | 1634 | CS003935 | 1554 | 1734 |
| AC003118 | AM19074-AS | 1267 | 1622 | CS003937 | 1555 | 1735 |
| AC003119 | AM19076-AS | 1268 | 1624 | CS003939 | 1556 | 1736 |
| AC003120 | AM19078-AS | 1269 | 1637 | CS003941 | 1557 | 1737 |

TABLE 8C
IAV RNAi agent Duplexes (targeting PB1) with Corresponding Sense and
Antisense Strand ID Numbers and Sequence ID numbers for the modified and unmodified
nucleotide sequences. (Shown with Targeting Ligand Conjugates)

| | | AS | AS | | SS | SS |
|---|---|---|---|---|---|---|
| | | modified | unmodified | | modified | unmodified |
| | | SEQ ID | SEQ ID | | SEQ ID | SEQ ID |
| Duplex | AS ID | NO: | NO: | SS ID | NO: | NO: |
| AC002367 | AM16046-AS | 1295 | 1642 | CS003004 | 1558 | 1738 |
| AC002368 | AM16048-AS | 1296 | 1658 | CS003006 | 1559 | 1739 |
| AC002369 | AM16050-AS | 1297 | 1657 | CS003008 | 1560 | 1740 |
| AC002567 | AM16683-AS | 1298 | 1655 | CS003274 | 1561 | 1741 |
| AC002758 | AM17574-AS | 1299 | 1663 | CS003515 | 1562 | 1742 |
| AC002909 | AM18497-AS | 1300 | 1641 | CS003681 | 1563 | 1743 |
| AC003123 | AM19084-AS | 1301 | 1665 | CS003947 | 1564 | 1744 |

TABLE 8D
IAV RNAi agent Duplexes (targeting PB2) with Corresponding Sense and
Antisense Strand ID Numbers and Sequence ID numbers for the modified and unmodified
nucleotide sequences. (Shown with Targeting Ligand Conjugates)

| | | AS | AS | | SS | SS |
|---|---|---|---|---|---|---|
| | | modified | unmodified | | modified | unmodified |
| | | SEQ ID | SEQ ID | | SEQ ID | SEQ ID |
| Duplex | AS ID | NO: | NO: | SS ID | NO: | NO: |
| AC002568 | AM16685-AS | 1314 | 1676 | CS003276 | 1565 | 1745 |
| AC002682 | AM17367-AS | 1315 | 1665 | CS003407 | 1566 | 1746 |
| AC002683 | AM17369-AS | 1316 | 1666 | CS003409 | 1567 | 1747 |
| AC002684 | AM17371-AS | 1317 | 1667 | CS003411 | 1568 | 1748 |
| AC002759 | AM17576-AS | 1318 | 1669 | CS003517 | 1569 | 1749 |
| AC002910 | AM18499-AS | 1319 | 1668 | CS003683 | 1570 | 1750 |
| AC003124 | AM19086-AS | 1320 | 1675 | CS003949 | 1571 | 1751 |

TABLE 8E
IAV RNAi agent Duplexes (targeting NP) with Corresponding Sense and
Antisense Strand ID Numbers and Sequence ID numbers for the modified and unmodified
nucleotide sequences. (Shown with Targeting Ligand Conjugates)

| | | AS | AS | | SS | SS |
|---|---|---|---|---|---|---|
| | | modified | unmodified | | modified | unmodified |
| | | SEQ ID | SEQ ID | | SEQ ID | SEQ ID |
| Duplex | AS ID | NO: | NO: | SS ID | NO: | NO: |
| AC002569 | AM16687-AS | 1337 | 1691 | CS003278 | 1572 | 1752 |
| AC002685 | AM17373-AS | 1338 | 1682 | CS003413 | 1573 | 1753 |
| AC002686 | AM17375-AS | 1339 | 1678 | CS003415 | 1574 | 1754 |
| AC002756 | AM17570-AS | 1340 | 1683 | CS003511 | 1575 | 1755 |
| AC002904 | AM18491-AS | 1341 | 1677 | CS003674 | 1576 | 1756 |
| AC003107 | AM19052-AS | 1342 | 1679 | CS003915 | 1577 | 1757 |
| AC003108 | AM19054-AS | 1343 | 1681 | CS003917 | 1578 | 1758 |
| AC003109 | AM19056-AS | 1344 | 1686 | CS003919 | 1579 | 1759 |
| AC003110 | AM19058-AS | 1345 | 1688 | CS003921 | 1580 | 1760 |
| AC003111 | AM19060-AS | 1346 | 1692 | CS003923 | 1581 | 1761 |

TABLE 8F
IAV RNAi agent Duplexes (targeting PA) with Corresponding Sense and
Antisense Strand ID Numbers and Sequence ID numbers for the modified and unmodified
nucleotide sequences. (Shown with Targeting Ligand Conjugates)

| | | AS | AS | | SS | SS |
|---|---|---|---|---|---|---|
| | | modified | unmodified | | modified | unmodified |
| | | SEQ ID | SEQ ID | | SEQ ID | SEQ ID |
| Duplex | AS ID | NO: | NO: | SS ID | NO: | NO: |
| AC002565 | AM16679-AS | 1360 | 1693 | CS003270 | 1582 | 1762 |
| AC002566 | AM16681-AS | 1361 | 1701 | CS003272 | 1583 | 1763 |
| AC002599 | AM16808-AS | 1362 | 1696 | CS003311 | 1584 | 1771 |
| AC002600 | AM16810-AS | 1363 | 1702 | CS003313 | 1585 | 1765 |
| AC002757 | AM17572-AS | 1364 | 1705 | CS003513 | 1586 | 1766 |
| AC002908 | AM18495-AS | 1365 | 1694 | CS003679 | 1587 | 1767 |
| AC003121 | AM19080-AS | 1366 | 1699 | CS003943 | 1588 | 1768 |
| AC003122 | AM19082-AS | 1367 | 1704 | CS003945 | 1589 | 1772 |

TABLE 9A
RNAi Agent (targeting M1) Conjugate Duplex ID Numbers Referencing
Position Targeted on Influenza A viral Genome

| | | | | Targeted |
|---|---|---|---|---|
| | | | | Influenza A |
| | | | | viral genome |
| | | | Duplex ID | Viral Genome |
| Conjugate | | | (Pre- | Position (Of SEQ |
| Duplex ID | AS ID | SS ID | Conjugation) | ID NO: 1) |
| AC002564 | AM16677-AS | CS003268 | AD11733 | 10 |
| AC002601 | AM16812-AS | CS003315 | AD11839 | 25 |
| AC002754 | AM17566-AS | CS003507 | AD12417 | 944 |
| AC002755 | AM17568-AS | CS003509 | AD12418 | 945 |
| AC002863 | AM17928-AS | CS003633 | AD12660 | 10 |
| AC002864 | AM17929-AS | CS003633 | AD12661 | 10 |
| AC002865 | AM17930-AS | CS003633 | AD12662 | 10 |
| AC002866 | AM17931-AS | CS003633 | AD12663 | 10 |
| AC002867 | AM17928-AS | CS003638 | AD12664 | 10 |
| AC002868 | AM17929-AS | CS003638 | AD12665 | 10 |
| AC002869 | AM17930-AS | CS003638 | AD12666 | 10 |
| AC002870 | AM17931-AS | CS003638 | AD12667 | 10 |
| AC002871 | AM17933-AS | CS003638 | AD12668 | 10 |
| AC002872 | AM17934-AS | CS003268 | AD12669 | 10 |
| AC002902 | AM18487-AS | CS003670 | AD13042 | 267 |
| AC002903 | AM18489-AS | CS003672 | AD13043 | 271 |
| AC002925 | AM16812-AS | CS003699 | AD13056 | 25 |
| AC002926 | AM18520-AS | CS003315 | AD13057 | 25 |
| AC002927 | AM18521-AS | CS003315 | AD13058 | 25 |
| AC002928 | AM18523-AS | CS003702 | AD13059 | 25 |
| AC002929 | AM18524-AS | CS003702 | AD13060 | 25 |
| AC002930 | AM18525-AS | CS003702 | AD13061 | 25 |
| AC002931 | AM18526-AS | CS003702 | AD13062 | 25 |
| AC002932 | AM18524-AS | CS003707 | AD13063 | 25 |
| AC002933 | AM18525-AS | CS003707 | AD13064 | 25 |
| AC002934 | AM18526-AS | CS003707 | AD13065 | 25 |
| AC003274 | AM17930-AS | CS003268 | AD14065 | 10 |
| AC003405 | AM18523-AS | CS003315 | AD14135 | 25 |
| AC003406 | AM19907-AS | CS003315 | AD14136 | 25 |
| AC003407 | AM18524-AS | CS003315 | AD14137 | 25 |
| AC003408 | AM18525-AS | CS003315 | AD14138 | 25 |
| AC003409 | AM18526-AS | CS003315 | AD14139 | 25 |
| AC003410 | AM19908-AS | CS003315 | AD14140 | 25 |
| AC003411 | AM19909-AS | CS003315 | AD14141 | 25 |
| AC003412 | AM19910-AS | CS003315 | AD14142 | 25 |
| AC003413 | AM19911-AS | CS003315 | AD14143 | 25 |
| AC003414 | AM19912-AS | CS003315 | AD14144 | 25 |

TABLE 9B
RNAi Agent (targeting NS1) Conjugate Duplex ID Numbers Referencing
Position Targeted on Influenza A viral Genome

| | | | | Targeted |
|---|---|---|---|---|
| | | | | Influenza A |
| | | | | viral genome |
| | | | Duplex ID | Viral Genome |
| Conjugate | | | (Pre- | Position (Of SEQ |
| Duplex ID | AS ID | SS ID | Conjugation) | ID NO: 2) |
| AC002317 | AM15547-AS | CS002930 | AD11159 | 591 |
| AC002318 | AM15729-AS | CS002932 | AD11160 | 814 |
| AC002319 | AM15872-AS | CS002934 | AD11161 | 552 |
| AC002320 | AM15874-AS | CS002936 | AD11162 | 737 |
| AC002322 | AM15877-AS | CS002939 | AD11164 | 511 |
| AC002597 | AM15714-AS | CS003308 | AD11835 | 511 |
| AC002598 | AM15548-AS | CS002930 | AD11836 | 591 |
| AC002760 | AM17578-AS | CS003519 | AD12423 | 83 |
| AC002761 | AM17580-AS | CS003521 | AD12424 | 84 |
| AC002762 | AM17582-AS | CS003523 | AD12425 | 85 |
| AC002763 | AM17584-AS | CS003525 | AD12426 | 541 |
| AC002764 | AM17586-AS | CS003527 | AD12427 | 542 |
| AC002765 | AM17588-AS | CS003529 | AD12428 | 543 |
| AC002766 | AM17590-AS | CS003531 | AD12429 | 545 |
| AC002767 | AM17592-AS | CS003533 | AD12430 | 546 |
| AC002768 | AM17594-AS | CS003535 | AD12431 | 387 |
| AC002769 | AM17596-AS | CS003537 | AD12432 | 491 |
| AC002770 | AM17598-AS | CS003539 | AD12433 | 517 |
| AC002771 | AM17600-AS | CS003541 | AD12434 | 617 |
| AC002905 | AM18492-AS | CS002934 | AD13045 | 552 |
| AC002906 | AM18493-AS | CS002936 | AD13046 | 737 |
| AC002907 | AM15730-AS | CS002932 | AD13047 | 814 |
| AC003112 | AM19062-AS | CS003925 | AD13400 | 201 |
| AC003113 | AM19064-AS | CS003927 | AD13401 | 297 |
| AC003114 | AM19066-AS | CS003929 | AD13402 | 394 |
| AC003115 | AM19068-AS | CS003931 | AD13403 | 416 |
| AC003116 | AM19070-AS | CS003933 | AD13404 | 433 |
| AC003117 | AM19072-AS | CS003935 | AD13405 | 561 |
| AC003118 | AM19074-AS | CS003937 | AD13406 | 564 |
| AC003119 | AM19076-AS | CS003939 | AD13407 | 767 |
| AC003120 | AM19078-AS | CS003941 | AD13408 | 805 |

TABLE 9C
RNAi Agent (targeting PB1) Conjugate Duplex ID Numbers Referencing
Position Targeted on Influenza A viral Genome

| | | | | Targeted |
|---|---|---|---|---|
| | | | | Influenza A |
| | | | | viral genome |
| | | | Duplex ID | Viral Genome |
| Conjugate | | | (Pre- | Position (Of SEQ |
| Duplex ID | AS ID | SS ID | Conjugation) | ID NO: 3) |
| AC002367 | AM16046-AS | CS003004 | AD11292 | 316 |
| AC002368 | AM16048-AS | CS003006 | AD11293 | 787 |
| AC002369 | AM16050-AS | CS003008 | AD11294 | 736 |
| AC002567 | AM16683-AS | CS003274 | AD11736 | 2234 |
| AC002758 | AM17574-AS | CS003515 | AD12421 | 2223 |
| AC002909 | AM18497-AS | CS003681 | AD13049 | 129 |
| AC003123 | AM19084-AS | CS003947 | AD13411 | 1637 |

TABLE 9D
RNAi Agent (targeting PB2) Conjugate Duplex ID Numbers Referencing
Position Targeted on Influenza A viral Genome

| | | | | Targeted |
|---|---|---|---|---|
| | | | | Influenza |
| | | | | A viral |
| | | | | genome |
| | | | | Viral |
| | | | | Genome |
| | | | Duplex ID | Position |
| Conjugate | | | (Pre- | (Of SEQ |
| Duplex ID | AS ID | SS ID | Conjugation) | ID NO: 4) |
| AC002568 | AM16685-AS | CS003276 | AD11737 | 2216 |
| AC002682 | AM17367-AS | CS003407 | AD12249 | 50 |
| AC002683 | AM17369-AS | CS003409 | AD12250 | 363 |
| AC002684 | AM17371-AS | CS003411 | AD12251 | 602 |
| AC002759 | AM17576-AS | CS003517 | AD12422 | 994 |
| AC002910 | AM18499-AS | CS003683 | AD13050 | 782 |
| AC003124 | AM19086-AS | CS003949 | AD13412 | 1959 |

TABLE 9E
RNAi Agent (targeting NP) Conjugate Duplex ID Numbers Referencing
Position Targeted on Influenza A viral Genome

| | | | | Targeted |
|---|---|---|---|---|
| | | | | Influenza |
| | | | | A viral |
| | | | | genome |
| | | | | Viral |
| | | | | Genome |
| | | | Duplex ID | Position |
| Conjugate | | | (Pre- | (Of SEQ |
| Duplex ID | AS ID | SS ID | Conjugation) | ID NO: 5) |
| AC002569 | AM16687-AS | CS003278 | AD11738 | 1428 |
| AC002685 | AM17373-AS | CS003413 | AD12252 | 493 |
| AC002686 | AM17375-AS | CS003415 | AD12253 | 162 |
| AC002756 | AM17570-AS | CS003511 | AD12419 | 579 |
| AC002904 | AM18491-AS | CS003674 | AD13044 | 83 |
| AC003107 | AM19052-AS | CS003915 | AD13395 | 189 |
| AC003108 | AM19054-AS | CS003917 | AD13396 | 427 |
| AC003109 | AM19056-AS | CS003919 | AD13397 | 780 |
| AC003110 | AM19058-AS | CS003921 | AD13398 | 967 |
| AC003111 | AM19060-AS | CS003923 | AD13399 | 1445 |

TABLE 9F
RNAi Agent (targeting PA) Conjugate Duplex ID Numbers Referencing
Position Targeted on Influenza A viral Genome

| | | | | Targeted |
|---|---|---|---|---|
| | | | | Influenza |
| | | | | A viral |
| | | | | genome |
| | | | | Viral |
| | | | | Genome |
| | | | Duplex ID | Position |
| Conjugate | | | (Pre- | (Of SEQ |
| Duplex ID | AS ID | SS ID | Conjugation) | ID NO: 6) |
| AC002565 | AM16679-AS | CS003270 | AD11734 | 7 |
| AC002566 | AM16681-AS | CS003272 | AD11735 | 907 |
| AC002599 | AM16808-AS | CS003311 | AD11837 | 509 |
| AC002600 | AM16810-AS | CS003313 | AD11838 | 1150 |
| AC002757 | AM17572-AS | CS003513 | AD12420 | 2070 |
| AC002908 | AM18495-AS | CS003679 | AD13048 | 140 |
| AC003121 | AM19080-AS | CS003943 | AD13409 | 691 |
| AC003122 | AM19082-AS | CS003945 | AD13410 | 1447 |

TABLE 10A
RNAi Agent (targeting M1) Conjugate ID Numbers With Chemically
Modified Antisense and Sense Strands (including Linkers and Conjugates)

| | | | | SEQ |
|---|---|---|---|---|
| AC ID | Sense Strand (Fully Modified | SEQ | | ID |
| Number | with Conjugated Targeting Ligand) | ID NO: | Antisense Strand (5′ → 3′) | NO: |
| AC002564 | Tri-SM6.1-αvß6-TA14- | 1520 | cPrpusUfsasCfgUfuucgaCfcUfcGfgUfuasg | 1169 |
| | csuaaccgaGfGfUfcgaaacguaas(invAb) | | | |
| AC002601 | Tri-SM6.1-αvß6-TA14- | 1521 | cPrpusAfsusGfaUfagaaaGfaAfcGfuAfcgsu | 1170 |
| | ascguacguUfCfUfuucuaucauas(invAb) | | | |
| AC002754 | Tri-SM6.1-αvß6-TA14- | 1522 | cPrpusAfscsAfaAfaugacCfaUfcGfuCfaasc | 1171 |
| | gsuugacgaUfGfGfucauuuuguas(invAb) | | | |
| AC002755 | Tri-SM6.1-αvß6-TA14- | 1523 | cPrpusGfsasCfaAfaaugaCfcAfuCfgUfcasc | 1172 |
| | gsugacgauGfGfUfcauuuugucas(invAb) | | | |
| AC002863 | Tri-SM6.1-αvß6-TA14- | 1524 | cPrpusUfsAfscguuucgaCfcUfcGfguuasg | 1173 |
| | csuaaccgaGfgUfcGfaaacguaas(invAb) | | | |
| AC002864 | Tri-SM6.1-αvß6-TA14- | 1524 | cPrpusUfsasCfguuucgaCfcUfcGfguuasg | 1174 |
| | csuaaccgaGfgUfcGfaaacguaas(invAb) | | | |
| AC002865 | Tri-SM6.1-αvß6-TA14- | 1524 | cPrpusUfsascGfuuucgaCfcUfcGfguuasg | 1175 |
| | csuaaccgaGfgUfcGfaaacguaas(invAb) | | | |
| AC002866 | Tri-SM6.1-αvß6-TA14- | 1524 | cPrpusUfsascguUfucgaCfcUfcGfguuasg | 1176 |
| | csuaaccgaGfgUfcGfaaacguaas(invAb) | | | |
| AC002867 | Tri-SM6.1-αvß6-TA14- | 1525 | cPrpusUfsAfscguuucgaCfcUfcGfguuasg | 1173 |
| | csuaaccgaGfgUfcgaaacguaas(invAb) | | | |
| AC002868 | Tri-SM6.1-αvß6-TA14- | 1525 | cPrpusUfsasCfguuucgaCfcUfcGfguuasg | 1174 |
| | csuaaccgaGfgUfcgaaacguaas(invAb) | | | |
| AC002869 | Tri-SM6.1-αvß6-TA14- | 1525 | cPrpusUfsascGfuuucgaCfcUfcGfguuasg | 1175 |
| | csuaaccgaGfgUfcgaaacguaas(invAb) | | | |
| AC002870 | Tri-SM6.1-αvß6-TA14- | 1525 | cPrpusUfsascguUfucgaCfcUfcGfguuasg | 1176 |
| | csuaaccgaGfgUfcgaaacguaas(invAb) | | | |
| AC002871 | Tri-SM6.1-αvß6-TA14- | 1525 | cPrpusUfsascguuuCfgaCfcUfcGfguuasg | 1177 |
| | csuaaccgaGfgUfcgaaacguaas(invAb) | | | |
| AC002872 | Tri-SM6.1-αvß6-TA14- | 1520 | cPrpusUfsaCfgUfuucgaCfcUfcGfgUfuasg | 1178 |
| | csuaaccgaGfGfUfcgaaacguaas(invAb) | | | |
| AC002902 | Tri-SM6.1-αvß6-TA14- | 1526 | cPrpusCfsusCfuAfuccauGfuUfgUfuCfggsg | 1179 |
| | csccgaacaAfCfAfuggauagagas(invAb) | | | |
| AC002903 | Tri-SM6.1-αvß6-TA14- | 1527 | cPrpasAfscsUfgCfucuauCfcAfuGfuUfgusc | 1180 |
| | gsacaacauGfGfAfuagaicaguus(invAb) | | | |
| AC002925 | Tri-SM6.1-αvß6-TA14- | 1528 | cPrpusAfsusGfaUfagaaaGfaAfcGfuAfcgsu | 1170 |
| | a_2NscguacguUfCfUfuucuaucauas(invAb) | | | |
| AC002926 | Tri-SM6.1-αvß6-TA14- | 1521 | cPrpusAfsuGfaUfagaaaGfaAfcGfuAfcsgsu | 1181 |
| | ascguacguUfCfUfuucuaucauas(invAb) | | | |
| AC002927 | Tri-SM6.1-αvß6-TA14- | 1521 | cPrpusAfsuGfaUfagaaaGfaAfcGfuAfcgsu | 1182 |
| | ascguacguUfCfUfuucuaucauas(invAb) | | | |
| AC002928 | Tri-SM6.1-αvß6-TA14- | 1529 | cPrpusAfsUfsgauagaaaGfaAfcGfuacgsu | 1183 |
| | ascguacguUfcUfuUfcuaucauas(invAb) | | | |
| AC002929 | Tri-SM6.1-αvß6-TA14- | 1529 | cPrpusAfsusgAfuagaaaGfaAfcGfuacgsu | 1184 |
| | ascguacguUfcUfuUfcuaucauas(invAb) | | | |
| AC002930 | Tri-SM6.1-αvß6-TA14- | 1529 | cPrpusAfsusgauAfgaaaGfaAfcGfuacgsu | 1185 |
| | ascguacguUfcUfuUfcuaucauas(invAb) | | | |
| AC002931 | Tri-SM6.1-αvß6-TA14- | 1529 | cPrpusAfsusgauagAfaaGfaAfcGfuacgsu | 1186 |
| | ascguacguUfcUfuUfcuaucauas(invAb) | | | |
| AC002932 | Tri-SM6.1-αvß6-TA14- | 1530 | cPrpusAfsusgAfuagaaaGfaAfcGfuacgsu | 1184 |
| | ascguacguUfcUfUfucuaucauas(invAb) | | | |
| AC002933 | Tri-SM6.1-αvß6-TA14- | 1530 | cPrpusAfsusgauAfgaaaGfaAfcGfuacgsu | 1185 |
| | ascguacguUfcUfUfucuaucauas(invAb) | | | |
| AC002934 | Tri-SM6.1-αvß6-TA14- | 1530 | cPrpusAfsusgauagAfaaGfaAfcGfuacgsu | 1186 |
| | ascguacguUfcUfUfucuaucauas(invAb) | | | |
| AC003274 | Tri-SM6.1-αvß6-TA14- | 1520 | cPrpusUfsascGfuuucgaCfcUfcGfguuasg | 1175 |
| | csuaaccgaGfGfUfcgaaacguaas(invAb) | | | |
| AC003405 | Tri-SM6.1-αvß6-TA14- | 1521 | cPrpusAfsUfsgauagaaaGfaAfcGfuacgsu | 1183 |
| | ascguacguUfCfUfuucuaucauas(invAb) | | | |
| AC003406 | Tri-SM6.1-αvß6-TA14- | 1521 | cPrpusAfsusGfauagaaaGfaAfcGfuacgsu | 1187 |
| | ascguacguUfCfUfuucuaucauas(invAb) | | | |
| AC003407 | Tri-SM6.1-αvß6-TA14- | 1521 | cPrpusAfsusgAfuagaaaGfaAfcGfuacgsu | 1184 |
| | ascguacguUfCfUfuucuaucauas(invAb) | | | |
| AC003408 | Tri-SM6.1-αvß6-TA14- | 1521 | cPrpusAfsusgauAfgaaaGfaAfcGfuacgsu | 1185 |
| | ascguacguUfCfUfuucuaucauas(invAb) | | | |
| AC003409 | Tri-SM6.1-αvß6-TA14- | 1521 | cPrpusAfsusgauagAfaaGfaAfcGfuacgsu | 1186 |
| | ascguacguUfCfUfuucuaucauas(invAb) | | | |
| AC003410 | Tri-SM6.1-αvß6-TA14- | 1521 | cPrpusAfsUfgauagaaaGfaAfcGfuacgsu | 1188 |
| | ascguacguUfCfUfuucuaucauas(invAb) | | | |
| AC003411 | Tri-SM6.1-αvß6-TA14- | 1521 | cPrpusAfsuGfauagaaaGfaAfcGfuacgsu | 1189 |
| | ascguacguUfCfUfuucuaucauas(invAb) | | | |
| AC003412 | Tri-SM6.1-αvß6-TA14- | 1521 | cPrpusAfsugAfuagaaaGfaAfcGfuacgsu | 1190 |
| | ascguacguUfCfUfuucuaucauas(invAb) | | | |
| AC003413 | Tri-SM6.1-αvß6-TA14- | 1521 | cPrpusAfsugauAfgaaaGfaAfcGfuacgsu | 1191 |
| | ascguacguUfCfUfuucuaucauas(invAb) | | | |
| AC003414 | Tri-SM6.1-αvß6-TA14- | 1521 | cPrpusAfsugauagAfaaGfaAfcGfuacgsu | 1192 |
| | ascguacguUfCfUfuucuaucauas(invAb) | | | |

TABLE 10B
RNAi Agent (targeting NS1) Conjugate ID Numbers With Chemically Modified
Antisense and Sense Strands (including Linkers and Conjugates)

| | Sense Strand (Fully Modified | | | |
|---|---|---|---|---|
| AC ID | with Conjugated Targeting | SEQ | | SEQ |
| Number | Ligand) (5′ → 3′) | ID NO: | Antisense Strand (5′ → 3′) | ID NO: |
| AC002317 | Tri-SM6.1-αvß6-TA14- | 1531 | cPrpusUfscsCfaAfgcgaaUfcUfcUfgUfausc | 1228 |
| | gsauacagaGfAfUfucgcuuigaas(invAb) | | | |
| AC002318 | Tri-SM6.1-αvß6-TA14- | 1532 | cPrpasUfsasAfgCfugaaaCfgAfgAfaAfgcsu | 1242 |
| | asgcuuucuCfGfUfuucagcuuaus(invAb) | | | |
| AC002319 | Tri-SM6.1-αvß6-TA14- | 1533 | cPrpusUfsgsUfuAfccauuCfcAfuUfcAfagsc | 1244 |
| | gscuugaauGfGfAfaugguaacaas(invAb) | | | |
| AC002320 | Tri-SM6.1-αvß6-TA14- | 1534 | cPrpusUfscsGfaAfacuauUfcUfcUfgUfcgsc | 1245 |
| | gscgacagaGfAfAfuaguuucgaas(invAb) | | | |
| AC002322 | Tri-SM6.1-αvß6-TA14- | 1535 | cPrpusGfscsAfuUfuuugaCfaUfcCfuCfausc | 1246 |
| | gsaugaggaUfGfUfcaaaaaugcas(invAb) | | | |
| AC002597 | Tri-SM6.1-αvß6-TA14- | 1536 | cPrpusGfscsauuUfuugaCfaUfcCfucausc | 1234 |
| | gsaugaggaUfgUfcAfaaaaugcas(invAb) | | | |
| AC002598 | Tri-SM6.1-αvß6-TA14- | 1531 | cPrpusUfscCfaAfgcgaaUfcUfcUfgUfasusc | 1229 |
| | gsauacagaGfAfUfucgcuuigaas(invAb) | | | |
| AC002760 | Tri-SM6.1-αvß6-TA14- | 1537 | cPrpusAfsusCfaAfggaauGfgGfgCfaUfcasc | 1247 |
| | gsugaugccCfCfAfuuccuugauas(invAb) | | | |
| AC002761 | Tri-SM6.1-αvß6-TA14- | 1538 | cPrpusGfsasUfcAfaggaaUfgGfgGfcAfucsa | 1248 |
| | usgaugcccCfAfUfuccuugaucas(invAb) | | | |
| AC002762 | Tri-SM6.1-αvß6-TA14- | 1539 | cPrpusCfsgsAfuCfaaggaAfuGfgGfgCfausc | 1249 |
| | gsaugccccAfUfUfccuuiaucgas(invAb) | | | |
| AC002763 | Tri-SM6.1-αvß6-TA14- | 1540 | cPrpusCfsasUfuCfaagucCfuCfcGfaUfgasg | 1250 |
| | csucaucggAfGfGfacuugaaugas(invAb) | | | |
| AC002764 | Tri-SM6.1-αvß6-TA14- | 1541 | cPrpusCfscsAfuUfcaaguCfcUfcCfgAfugsa | 1251 |
| | uscaucggaGfGfAfcuugaauigas(invAb) | | | |
| AC002765 | Tri-SM6.1-αvß6-TA14- | 1542 | cPrpusUfscsCfaUfucaagUfcCfuCfcGfausg | 1252 |
| | csaucggagGfAfCfuugaauggaas(invAb) | | | |
| AC002766 | Tri-SM6.1-αvß6-TA14- | 1543 | cPrpusAfsusUfcCfauucaAfgUfcCfuCfcgsa | 1253 |
| | uscggaggaCfUfUfgaauggaauas(invAb) | | | |
| AC002767 | Tri-SM6.1-αvß6-TA14- | 1544 | cPrpusCfsasUfuCfcauucAfaGfuCfcUfccsg | 1254 |
| | csggaggacUfUfGfaaugiaaugas(invAb) | | | |
| AC002768 | Tri-SM6.1-αvß6-TA14- | 1545 | cPrpasCfsasCfuGfaaguuCfgCfuUfuCfagsu | 1255 |
| | ascugaaagCfGfAfacuucaiugus(invAb) | | | |
| AC002769 | Tri-SM6.1-αvß6-TA14- | 1546 | cPrpasAfsgsUfaUfguccuGfgAfaGfaGfaasg | 1256 |
| | csuucucuuCfCfAfggacauacuus(invAb) | | | |
| AC002770 | Tri-SM6.1-αvß6-TA14- | 1547 | cPrpusCfscsAfaCfugcauUfuUfuGfaCfausc | 1257 |
| | gsaugucaaAfAfAfugcaiuuggas(invAb) | | | |
| AC002771 | Tri-SM6.1-αvß6-TA14- | 1548 | cPrpasAfsgsGfuCfucccaUfuCfuCfaUfcasc | 1258 |
| | gsugaugagAfAfUfgggaiaccuus(invAb) | | | |
| AC002905 | Tri-SM6.1-αvß6-TA14- | 1533 | cPrpusUfsgsuuaCfcauuCfcAfuUfcaagsc | 1259 |
| | gscuugaauGfGfAfaugguaacaas(invAb) | | | |
| AC002906 | Tri-SM6.1-αvß6-TA14- | 1534 | cPrpusUfscsgaaAfcuauUfcUfcUfgucgsc | 1260 |
| | gscgacagaGfAfAfuaguuucgaas(invAb) | | | |
| AC002907 | Tri-SM6.1-αvß6-TA14- | 1532 | cPrpasUfsaAfgCfugaaaCfgAfgAfaAfgscsu | 1243 |
| | asgcuuucuCfGfUfuucagcuuaus(invAb) | | | |
| AC003112 | Tri-SM6.1-αvß6-TA14- | 1549 | cPrpusUfsgsGfaUfuccucUfuUfcAfaGfausc | 1261 |
| | gsaucuugaAfAfGfaggaauccaas(invAb) | | | |
| AC003113 | Tri-SM6.1-αvß6-TA14- | 1550 | cPrpasUfsgsAfgCfaugaaCfcAfgUfcUfcgsu | 1262 |
| | ascgagacuGfGfUfucauicucaus(invAb) | | | |
| AC003114 | Tri-SM6.1-αvß6-TA14- | 1551 | cPrpasAfsasGfaUfuacacUfgAfaGfuUfcgsc | 1263 |
| | gscgaacuuCfAfGfuguaaucuuus(invAb) | | | |
| AC003115 | Tri-SM6.1-αvß6-TA14- | 1552 | cPrpusUfsasUfcAfaggucUfcUfaAfuCfggsu | 1264 |
| | asccgauuaGfAfGfaccuugauaas(invAb) | | | |
| AC003116 | Tri-SM6.1-αvß6-TA14- | 1553 | cPrpasGfsusGfaAfagcccUfuAfgUfaGfuasc | 1265 |
| | gsuacuacuAfAfGfggcuuucacus(invAb) | | | |
| AC003117 | Tri-SM6.1-αvß6-TA14- | 1554 | cPrpasCfsusCfgAfaccguGfuUfaCfcAfuusc | 1266 |
| | gsaaugguaAfCfAfcgguuciagus(invAb) | | | |
| AC003118 | Tri-SM6.1-αvß6-TA14- | 1555 | cPrpusAfsgsAfcUfcgaacCfgUfgUfuAfccsa | 1267 |
| | usgguaacaCfGfGfuucgaiucuas(invAb) | | | |
| AC003119 | Tri-SM6.1-αvß6-TA14- | 1556 | cPrpusAfsgsUfuGfuaaggCfuUfgCfaUfaasc | 1268 |
| | gsuuaugcaAfGfCfcuuacaacuas(invAb) | | | |
| AC003120 | Tri-SM6.1-αvß6-TA14- | 1557 | cPrpasAfscsGfaGfaaagcUfcUfuAfuCfucsc | 1269 |
| | gsgagauaaGfAfGfcuuucuciuus(invAb) | | | |

TABLE 10C
RNAi Agent (targeting PB1) Conjugate ID Numbers With Chemically Modified Antisense
and Sense Strands (including Linkers and Conjugates)

| | Sense Strand (Fully Modified | | | |
|---|---|---|---|---|
| AC ID | with Conjugated Targeting | SEQ | | SEQ |
| Number | Ligand) (5′ → 3′) | ID NO: | Antisense Strand (5′ → 3′) | ID NO: |
| AC002367 | Tri-SM6.1-αvß6-TA14- | 1558 | cPrpusUfscsCfaUfuguuuCfaAfgGfcAfugsa | 1295 |
| | uscaugccuUfGfAfaacaauggaas(invAb) | | | |
| AC002368 | Tri-SM6.1-αvß6-TA14- | 1559 | cPrpasGfsasCfuGfuucaaGfcUfuUfuCfgcsa | 1296 |
| | usgcgaaaaGfCfUfugaacaiucus(invAb) | | | |
| AC002369 | Tri-SM6.1-αvß6-TA14- | 1560 | cPrpusAfscsGfaAfaccucUfaAfuCfuGfcasc | 1297 |
| | gsugcagauUfAfGfagguuucguas(invAb) | | | |
| AC002567 | Tri-SM6.1-αvß6-TA14- | 1561 | cPrpusUfsusCfaAfuggugGfaAfcAfgAfucsu | 1298 |
| | asgaucuguUfCfCfaccauugaaas(invAb) | | | |
| AC002758 | Tri-SM6.1-αvß6-TA14- | 1562 | cPrpusAfsasCfaGfaucuuCfaUfgAfuCfucsc | 1299 |
| | gsgagaucaUfGfAfagaucuguuas(invAb) | | | |
| AC002909 | Tri-SM6.1-αvß6-TA14- | 1563 | cPrpusAfsgsUfaUfuggugUfgUfuCfuGfuusc | 1300 |
| | gsaacagaaCfAfCfaccaauacuas(invAb) | | | |
| AC003123 | Tri-SM6.1-αvß6-TA14- | 1564 | cPrpusGfsasUfgAfacaauUfgAfaGfaGfccsa | 1301 |
| | usggcucuuCfAfAfuuguucaucas(invAb) | | | |

TABLE 10D
RNAi Agent (targeting PB2) Conjugate ID Numbers With Chemically Modified Antisense
and Sense Strands (including Linkers and Conjugates)

| | Sense Strand (Fully Modified | | | |
|---|---|---|---|---|
| AC ID | with Conjugated Targeting | SEQ | | SEQ |
| Number | Ligand) (5′ → 3′) | ID NO: | Antisense Strand (5′ → 3′) | ID NO: |
| AC002568 | Tri-SM6.1-αvß6-TA14- | 1565 | cPrpusAfsgsUfaAfguaugCfuAfgAfgUfccsc | 1314 |
| | gsggacucuAfGfCfauacuuacuas(invAb) | | | |
| AC002682 | Tri-SM6.1-αvß6-TA14- | 1566 | cPrpusGfsgsUfcUfuagugAfgUfaUfcUfcgsc | 1315 |
| | gscgagauaCfUfCfacuaaiaccas(invAb) | | | |
| AC002683 | Tri-SM6.1-αvß6-TA14- | 1567 | cPrpusCfsasUfgUfuucaaCfcUfuUfcGfacsc | 1316 |
| | gsgucgaaaGfGfUfugaaacaugas(invAb) | | | |
| AC002684 | Tri-SM6.1-αvß6-TA14- | 1568 | cPrpusUfsasGfcAfuguacGfcCfaCfcAfucsa | 1317 |
| | usgauggugGfCfGfuacaugcuaas(invAb) | | | |
| AC002759 | Tri-SM6.1-αvß6-TA14- | 1569 | cPrpusAfscsUfgAfugaucCfgCfuUfgUfccsu | 1318 |
| | asggacaagCfGfGfaucaucaguas(invAb) | | | |
| AC002910 | Tri-SM6.1-αvß6-TA14- | 1570 | cPrpusUfsasCfuAfuguuuCfuAfgCfaGfcgsa | 1319 |
| | uscgcugcuAfGfAfaacauaguaas(invAb) | | | |
| AC003124 | Tri-SM6.1-αvß6-TA14- | 1571 | cPrpusUfsgsUfuGfuaauuGfaAfuAfcUfggsa | 1320 |
| | usccaguauUfCfAfauuacaacaas(invAb) | | | |

TABLE 10E
RNAi Agent (targeting NP) Conjugate ID Numbers With Chemically Modified Antisense
and Sense Strands (including Linkers and Conjugates)

| | Sense Strand (Fully Modified | | | |
|---|---|---|---|---|
| ACID | with Conjugated Targeting | SEQ | | SEQ |
| Number | Ligand) (5′ → 3′) | ID NO: | Antisense Strand (5′ → 3′) | ID NO: |
| AC002569 | Tri-SM6.1-αvß6-TA14- | 1572 | cPrpusUfsasCfuCfaugucAfaAfgGfaAfggsc | 1337 |
| | gsccuuccuUfUfGfacaugaguaas(invAb) | | | |
| AC002685 | Tri-SM6.1-αvß6-TA14- | 1573 | cPrpusGfsusUfgAfaccuuGfcAfuUfaGfagsc | 1338 |
| | gscucuaauGfCfAfagguucaacas(invAb) | | | |
| AC002686 | Tri-SM6.1-αvß6-TA14- | 1574 | cPrpasUfsgsCfuAfuucugGfaUfuAfgUfcgsu | 1339 |
| | ascgacuaaUfCfCfagaauagcaus(invAb) | | | |
| AC002756 | Tri-SM6.1-αvß6-TA14- | 1575 | cPrpusCfsasCfgUfuugauCfaUfuCfuGfausc | 1340 |
| | gsaucagaaUfGfAfucaaaciugas(invAb) | | | |
| AC002904 | Tri-SM6.1-αvß6-TA14- | 1576 | cPrpusAfscsCfaAfucauuCfuUfcCfgAfcasg | 1341 |
| | csugucggaAfGfAfaugauugguas(invAb) | | | |
| AC003107 | Tri-SM6.1-αvß6-TA14- | 1577 | cPrpusCfsasGfaAfagcacCfaUfcCfuCfucsu | 1342 |
| | asgagaggaUfGfGfugcuuucugas(invAb) | | | |
| AC003108 | Tri-SM6.1-αvß6-TA14- | 1578 | cPrpusUfsgsAfuAfuguggCfaUfcAfuUfcasg | 1343 |
| | csugaaugaUfGfCfcacauaucaas(invAb) | | | |
| AC003109 | Tri-SM6.1-αvß6-TA14- | 1579 | cPrpusUfscsAfgAfaugagUfgCfuGfaCfcgsu | 1344 |
| | ascggucagCfAfCfucauucugaas(invAb) | | | |
| AC003110 | Tri-SM6.1-αvß6-TA14- | 1580 | cPrpusAfscsCfaAfuugacUfcUfuGfuGfagsc | 1345 |
| | gscucacaaGfAfGfucaauugguas(invAb) | | | |
| AC003111 | Tri-SM6.1-αvß6-TA14- | 1581 | cPrpasGfsasAfaUfaagacCfcUfuCfaUfuasc | 1346 |
| | gsuaaugaaGfGfGfucuuauuucus(invAb) | | | |

TABLE 10F
RNAi Agent (targeting PA) Conjugate ID Numbers With Chemically Modified Antisense
and Sense Strands (including Linkers and Conjugates)

| | Sense Strand (Fully Modified with | SEQ | | SEQ |
|---|---|---|---|---|
| ac id | Conjugating Targeting | ID | | ID |
| Number | Ligand) (5′ → 3′) | NO: | Antisense Strand (5′ → 3′) | NO: |
| AC002565 | Tri-SM6.1-αvß6-TA14- | 1582 | cPrpusAfsasGfcAfuugucGfcAfcAfaAfgusc | 1360 |
| | gsacuuuguGfCfGfacaaugcuuas(invAb) | | | |
| AC002566 | Tri-SM6.1-αvß6-TA14- | 1583 | cPrpusUfsusGfaUfugcauCfaUfaUfaGfugsg | 1361 |
| | cscacuauaUfGfAfugcaaucaaas(invAb) | | | |
| AC002599 | Tri-SM6.1-αvß6-TA14- | 1584 | cPrpusGfsasAfaAfgccuaGfuUfuUfgAfuusc | 1362 |
| | gsa_2NaucaaaAfCfUfaggcuuuucas(invAb) | | | |
| AC002600 | Tri-SM6.1-αvß6-TA14- | 1585 | cPrpasAfsgsGfuCfuccaaCfaUfcUfuUfgcsa | 1363 |
| | usgcaaagaUfGfUfuggaiaccuus(invAb) | | | |
| AC002757 | Tri-SM6.1-αvß6-TA14- | 1586 | cPrpusCfsasUfuAfaucagGfcAfcUfcCfucsg | 1364 |
| | csgaggaguGfCfCfugauuaaugas(invAb) | | | |
| AC002908 | Tri-SM6.1-αvß6-TA14- | 1587 | cPrpusGfsasAfaUfggaaaUfcCfgAfaUfacsc | 1365 |
| | gsguauucgGfAfUfuuccauuucas(invAb) | | | |
| AC003121 | Tri-SM6.1-αvß6-TA14- | 1588 | cPrpusUfscsGfaAfuccauCfuAfcAfuAfggsc | 1366 |
| | gsccuauguAfGfAfuggauucgaas(invAb) | | | |
| AC003122 | Tri-SM6.1-αvß6-TA14- | 1589 | cPrpasCfsasUfuUfgcuuaUfcAfuUfgGfgasu | 1367 |
| | asucccaauGfa_2NUfaagcaaaugus(invAb) | | | |

In some embodiments, an IAV RNAi agent is prepared or provided as a salt, mixed salt, or a free-acid. In some embodiments, an IAV RNAi agent is prepared or provided as a pharmaceutically acceptable salt. In some embodiments, an IAV RNAi agent is prepared or provided as a pharmaceutically acceptable sodium or potassium salt. The RNAi agents described herein, upon delivery to a cell expressing an influenza A viral genome viral genome, inhibit or knockdown expression of one or more influenza A viral genomes in vivo and/or in vitro.

### Targeting Groups, Linking Groups, Pharmacokinetic/Pharmacodynamic (PK/PD) Modulators, and Delivery Vehicles

In some embodiments, an IAV RNAi agent contains or is conjugated to one or more non-nucleotide groups including, but not limited to, a targeting group, a linking group, a pharmacokinetic/pharmacodynamic (PK/PD) modulator, a delivery polymer, or a delivery vehicle. The non-nucleotide group can enhance targeting, delivery, or attachment of the RNAi agent. The non-nucleotide group can be covalently linked to the 3′ and/or 5′ end of either the sense strand and/or the antisense strand. In some embodiments, an IAV RNAi agent contains a non-nucleotide group linked to the 3′ and/or 5′ end of the sense strand. In some embodiments, a non-nucleotide group is linked to the 5′ end of an IAV RNAi agent sense strand. A non-nucleotide group can be linked directly or indirectly to the RNAi agent via a linker/linking group. In some embodiments, a non-nucleotide group is linked to the RNAi agent via a labile, cleavable, or reversible bond or linker.

In some embodiments, a non-nucleotide group enhances the pharmacokinetic or biodistribution properties of an RNAi agent or conjugate to which it is attached to improve cell- or tissue-specific distribution and cell-specific uptake of the conjugate. In some embodiments, a non-nucleotide group enhances endocytosis of the RNAi agent.

Targeting groups or targeting moieties enhance the pharmacokinetic or biodistribution properties of a conjugate or RNAi agent to which they are attached to improve cell-specific (including, in some cases, organ specific) distribution and cell-specific (or organ specific) uptake of the conjugate or RNAi agent. A targeting group can be monovalent, divalent, trivalent, tetravalent, or have higher valency for the target to which it is directed. Representative targeting groups include, without limitation, compounds with affinity to cell surface molecule, cell receptor ligands, hapten, antibodies, monoclonal antibodies, antibody fragments, and antibody mimics with affinity to cell surface molecules. In some embodiments, a targeting group is linked to an RNAi agent using a linker, such as a PEG linker or one, two, or three abasic and/or ribitol (abasic ribose) residues, which in some instances can serve as linkers.

A targeting group, with or without a linker, can be attached to the 5′ or 3′ end of any of the sense and/or antisense strands disclosed in Tables 2A, 2B, 2C, 2D, 2E, 2F, 3A, 3B, 3C, 3D, 3E, 3F, 4A, 4B, 4C, 4D, 4E, 4F, 5A, 5B, 5C, 5D, 5E, 5F, 6A, 6B, 6C, 6D, 6E, 6F, 10A, 10B, 10C, 10D, 10E, and 10F. A linker, with or without a targeting group, can be attached to the 5′ or 3′ end of any of the sense and/or antisense strands disclosed in Tables 2A, 2B, 2C, 2D, 2E, 2F, 3A, 3B, 3C, 3D, 3E, 3F, 4A, 4B, 4C, 4D, 4E, 4F, 5A, 5B, 5C, 5D, 5E, 5F, 6A, 6B, 6C, 6D, 6E, 6F, 10A, 10B, 10C, 10D, 10E, and 10F.

The IAV RNAi agents described herein can be synthesized having a reactive group, such as an amino group (also referred to herein as an amine), at the 5′-terminus and/or the 3′-terminus. The reactive group can be used subsequently to attach a targeting moiety using methods typical in the art.

For example, in some embodiments, the IAV RNAi agents disclosed herein are synthesized having an NH₂-C₆ group at the 5′-terminus of the sense strand of the RNAi agent. The terminal amino group subsequently can be reacted to form a conjugate with, for example, a group that includes an αvβ6 integrin targeting ligand. In some embodiments, the IAV RNAi agents disclosed herein are synthesized having one or more alkyne groups at the 5′-terminus of the sense strand of the RNAi agent. The terminal alkyne group(s) can subsequently be reacted to form a conjugate with, for example, a group that includes an αvβ6 integrin targeting ligand.

In some embodiments, a targeting group comprises an integrin targeting ligand. In some embodiments, an integrin targeting ligand is an αvβ6 integrin targeting ligand. The use of an αvβ6 integrin targeting ligand facilitates cell-specific targeting to cells having αvβ6 on its respective surface, and binding of the integrin targeting ligand can facilitate entry of the therapeutic agent, such as an RNAi agent, to which it is linked, into cells such as epithelial cells, including pulmonary epithelial cells and renal epithelial cells. Integrin targeting ligands can be monomeric or monovalent (e.g., having a single integrin targeting moiety) or multimeric or multivalent (e.g., having multiple integrin targeting moieties). The targeting group can be attached to the 3′ and/or 5′ end of the RNAi oligonucleotide using methods known in the art. The preparation of targeting groups, such as αvβ6 integrin targeting ligands, is described, for example, in International Patent Application Publication No. WO 2018/085415 and in International Patent Application Publication No. WO 2019/089765, the contents of each of which are incorporated herein in its entirety.

In some embodiments, targeting groups are linked to the IAV RNAi agents without the use of an additional linker. In some embodiments, the targeting group is designed having a linker readily present to facilitate the linkage to an IAV RNAi agent. In some embodiments, when two or more RNAi agents are included in a composition, the two or more RNAi agents can be linked to their respective targeting groups using the same linkers. In some embodiments, when two or more RNAi agents are included in a composition, the two or more RNAi agents are linked to their respective targeting groups using different linkers.

In some embodiments, a linking group is conjugated to the RNAi agent. The linking group facilitates covalent linkage of the agent to a targeting group, pharmacokinetic modulator, delivery polymer, or delivery vehicle. The linking group can be linked to the 3′ and/or the 5′ end of the RNAi agent sense strand or antisense strand. In some embodiments, the linking group is linked to the RNAi agent sense strand. In some embodiments, the linking group is conjugated to the 5′ or 3′ end of an RNAi agent sense strand. In some embodiments, a linking group is conjugated to the 5′ end of an RNAi agent sense strand. Examples of linking groups, include but are not limited to: C6-SS-C6, 6-SS-6, reactive groups such a primary amines (e.g., NH2-C6) and alkynes, alkyl groups, abasic residues/nucleotides, amino acids, tri-alkyne functionalized groups, ribitol, and/or PEG groups. Examples of certain linking groups are provided in Table 11.

A linker or linking group is a connection between two atoms that links one chemical group (such as an RNAi agent) or segment of interest to another chemical group (such as a targeting group, pharmacokinetic modulator, or delivery polymer) or segment of interest via one or more covalent bonds. A labile linkage contains a labile bond. A linkage can optionally include a spacer that increases the distance between the two joined atoms. A spacer may further add flexibility and/or length to the linkage. Spacers include, but are not limited to, alkyl groups, alkenyl groups, alkynyl groups, aryl groups, aralkyl groups, aralkenyl groups, and aralkynyl groups; each of which can contain one or more heteroatoms, heterocycles, amino acids, nucleotides, and saccharides. Spacer groups are well known in the art and the preceding list is not meant to limit the scope of the description. In some embodiments, an IAV RNAi agent is conjugated to a polyethylene glycol (PEG) moiety, or to a hydrophobic group having 12 or more carbon atoms, such as a cholesterol or palmitoyl group.

In some embodiments, an IAV RNAi agent is linked to one or more pharmacokinetic/pharmacodynamic (PK/PD) modulators. PK/PD modulators can increase circulation time of the conjugated drug and/or increase the activity of the RNAi agent through improved cell receptor binding, improved cellular uptake, and/or other means. Various PK/PD modulators suitable for use with RNAi agents are known in the art. In some embodiments, the PK/PD modulatory can be cholesterol or cholesteryl derivatives, or in some circumstances a PK/PD modulator can be comprised of alkyl groups, alkenyl groups, alkynyl groups, aryl groups, aralkyl groups, aralkenyl groups, or aralkynyl groups, each of which may be linear, branched, cyclic, and/or substituted or unsubstituted. In some embodiments, the location of attachment for these moieties is at the 5′ or 3′ end of the sense strand, at the 2′ position of the ribose ring of any given nucleotide of the sense strand, and/or attached to the phosphate or phosphorothioate backbone at any position of the sense strand.

Any of the IAV RNAi agent nucleotide sequences listed in Tables 2A, 2B, 2C, 2D, 2E, 2F, 3A, 3B, 3C, 3D, 3E, 3F, 4A, 4B, 4C, 4D, 4E, 4F, 5A, 5B, 5C, 5D, 5E, 5F, 6A, 6B, 6C, 6D, 6E, 6F, 10A, 10B, 10C, 10D, 10E, and 10F, whether modified or unmodified, can contain 3′ and/or 5′ targeting group(s), linking group(s), and/or PK/PD modulator(s). Any of the IAV RNAi agent sequences listed in Tables 3A, 3B, 3C, 3D, 3E, 3F, 4A, 4B, 4C, 4D, 4E, 4F, 5A, 5B, 5C, 5D, 5E, 5F, 6A, 6B, 6C, 6D, 6E, 6F, 10A, 10B, 10C, 10D, 10E, and 10F, or are otherwise described herein, which contain a 3′ or 5′ targeting group, linking group, and/or PK/PD modulator can alternatively contain no 3′ or 5′ targeting group, linking group, or PK/PD modulator, or can contain a different 3′ or 5′ targeting group, linking group, or pharmacokinetic modulator including, but not limited to, those depicted in Table 11. Any of the IAV RNAi agent duplexes listed in Tables 7A-1, 7A-2, 7A-3, 7A-4, 7A-5, 7A-6, 7B-1, 7B-2, 7B-3, 7B-4, 7B-5, 7B-6, 8A, 8B, 8C, 8D, 8E, 8F, 9A, 9B, 9C, 9D, 9E, 9F, 10A, 10B, 10C, 10D, 10E, and 10F, whether modified or unmodified, can further comprise a targeting group or linking group, including, but not limited to, those depicted in Table 11, and the targeting group or linking group can be attached to the 3′ or 5′ terminus of either the sense strand or the antisense strand of the IAV RNAi agent duplex.

Examples of certain modified nucleotides, capping moieties, and linking groups are provided in Table 11.

TABLE 11
Structures Representing Various Modified Nucleotides, Capping Moieties, and
Linking Groups (wherein [symbol]
indicates the point of connection)

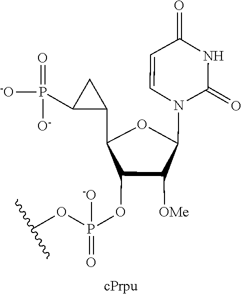

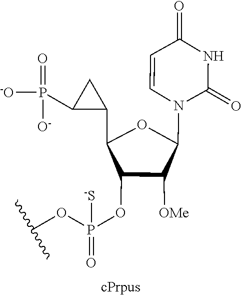

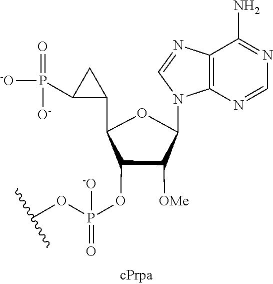

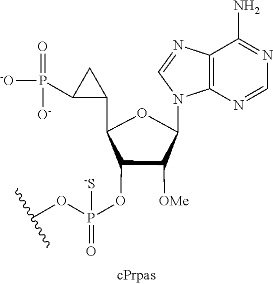

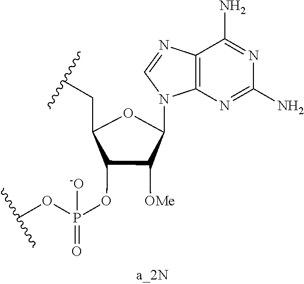

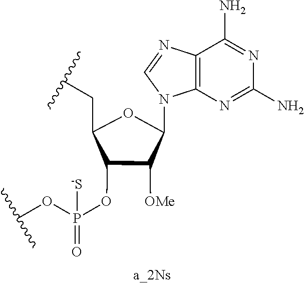

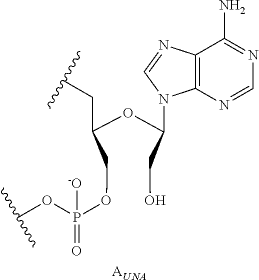

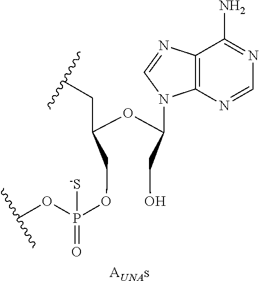

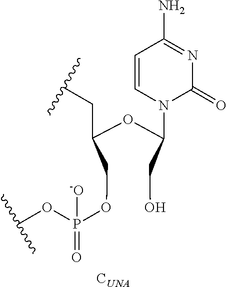

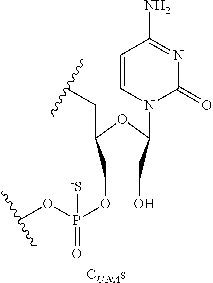

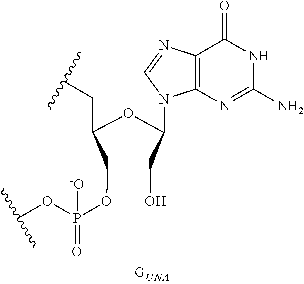

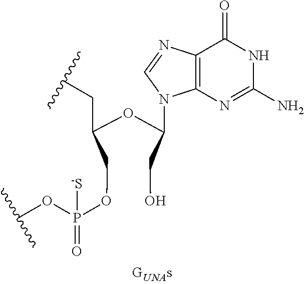

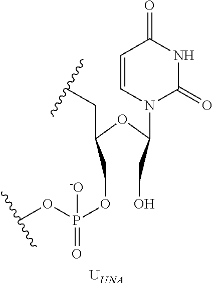

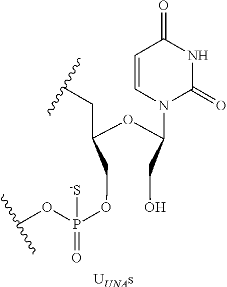

When positioned internally:
linkage towards 5′ end

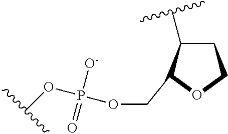

linkage towards 3′ end
(invAb)
When positioned internally:
linkage towards 5′ end

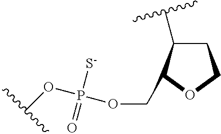

linkage towards 3′ end
(invAb)s
When positioned at the 3′ terminal end:
linkage towards 5′ end

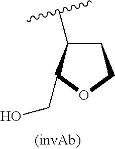

When positioned at the 3′ terminal end:
linkage towards 5′ end

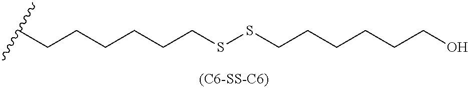 [SMILES: *CCCCCCSSCCCCCCO]

When positioned internally:

| | |
|---|---|
| linkage towards 5′ end | linkage towards 3′ end |

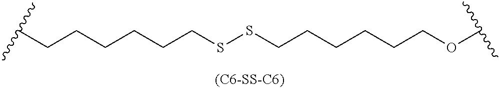 [SMILES: *CCCCCCSSCCCCCCO*]

When positioned at the 3′ terminal end:
linkage towards 5′ end

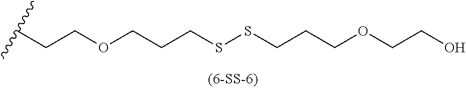 [SMILES: *CCOCCCSSCCCOCCO]

When positioned internally:

| | |
|---|---|
| linkage towards 5′ end | linkage towards 3′ end |

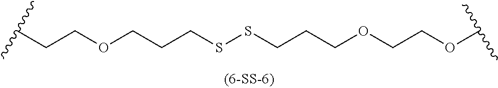 [SMILES: *CCOCCCSSCCCOCCO*]

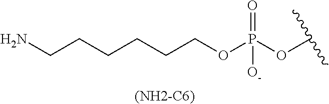

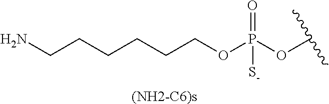

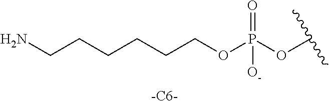

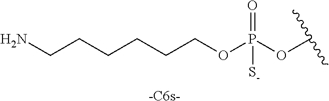

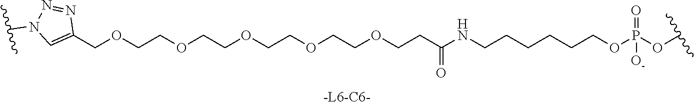

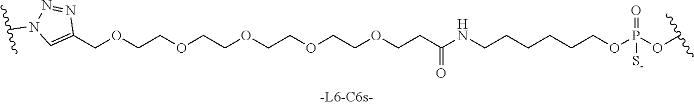

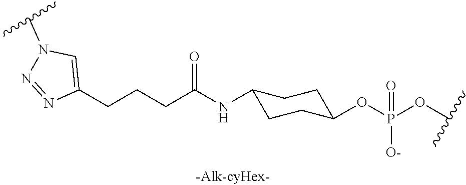 [SMILES: *OP(=O)([O-])OC1CCC(NC(=O)CCCc2cn(*)nn2)CC1]

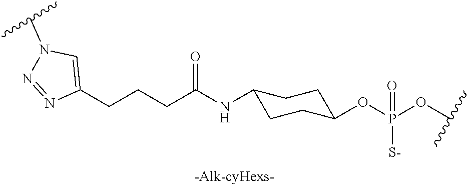 [SMILES: *OP(=O)([S-])OC1CCC(NC(=O)CCCc2cn(*)nn2)CC1]

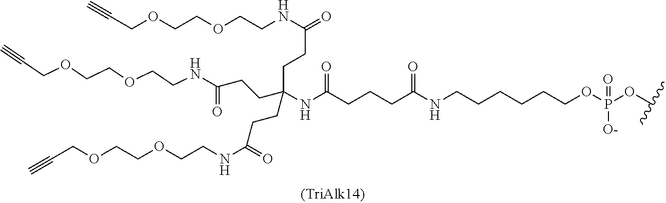 [SMILES: *OP(=O)([O-])OCCCCCCNC(=O)CCCC(=O)NC(CCC(=O)NCCOCCOCC#C)(CCC(=O)NCCOCCOCC#C)CCC(=O)NCCOCCOCC#C]

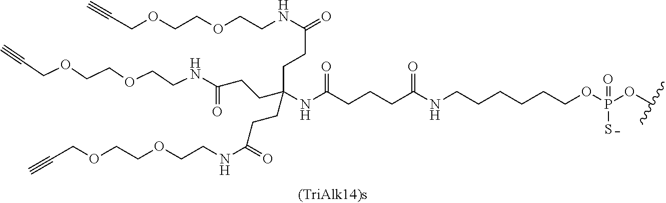 [SMILES: *OP(=O)([S-])OCCCCCCNC(=O)CCCC(=O)NC(CCC(=O)NCCOCCOCC#C)(CCC(=O)NCCOCCOCC#C)CCC(=O)NCCOCCOCC#C]

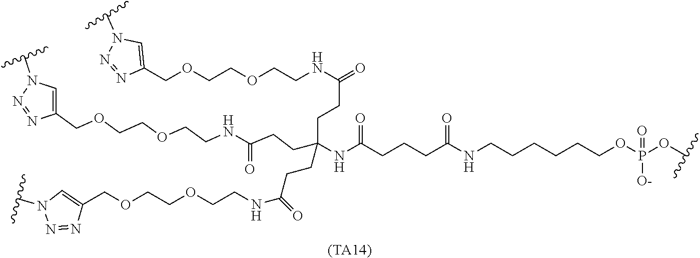

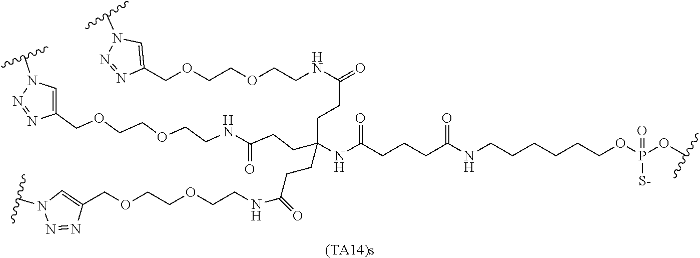 [SMILES: *OP(=O)([S-])OCCCCCCNC(=O)CCCC(=O)NC(CCC(=O)NCCOCCOCc1cn(*)nn1)(CCC(=O)NCCOCCOCc1cn(*)nn1)CCC(=O)NCCOCCOCc1cn(*)nn1 ; "(TA14)s"]

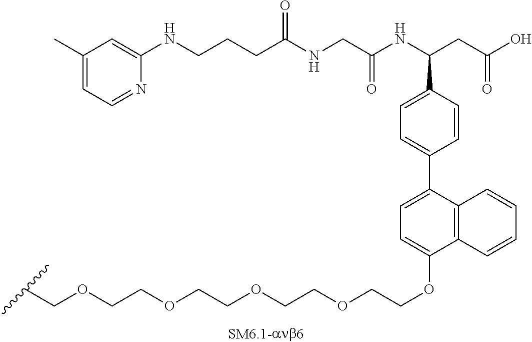 [SMILES: *COCCOCCOCCOCCOc1ccc(-c2ccc([C@H](CC(=O)O)NC(=O)CNC(=O)CCCNc3cc(C)ccn3)cc2)c2ccccc12]

Alternatively, other linking groups known in the art may be used. In many instances, linking groups can be commercially acquired or alternatively, are incorporated into commercially available nucleotide phosphoramidites. (See, e.g., International Patent Application Publication No. WO 2019/161213, which is incorporated herein by reference in its entirety).

In some embodiments, an IAV RNAi agent is delivered without being conjugated to a targeting ligand or pharmacokinetic/pharmacodynamic (PK/PD) modulator (referred to as being “naked” or a “naked RNAi agent”).

In some embodiments, an IAV RNAi agent is conjugated to a targeting group, a linking group, a PK modulator, and/or another non-nucleotide group to facilitate delivery of the IAV RNAi agent to the cell or tissue of choice, for example, to an epithelial cell in vivo. In some embodiments, an IAV RNAi agent is conjugated to a targeting group wherein the targeting group includes an integrin targeting ligand. In some embodiments, the integrin targeting ligand is an αvβ6 integrin targeting ligand. In some embodiments, a targeting group includes one or more αvβ6 integrin targeting ligands.

In some embodiments, a delivery vehicle may be used to deliver an RNAi agent to a cell or tissue. A delivery vehicle is a compound that improves delivery of the RNAi agent to a cell or tissue. A delivery vehicle can include, or consist of, but is not limited to: a polymer, such as an amphipathic polymer, a membrane active polymer, a peptide, a melittin peptide, a melittin-like peptide (MLP), a lipid, a reversibly modified polymer or peptide, or a reversibly modified membrane active polyamine.

In some embodiments, the RNAi agents can be combined with lipids, nanoparticles, polymers, liposomes, micelles, DPCs or other delivery systems available in the art for nucleic acid delivery. The RNAi agents can also be chemically conjugated to targeting groups, lipids (including, but not limited to cholesteryl and cholesteryl derivatives), encapsulating in nanoparticles, liposomes, micelles, conjugating to polymers or DPCs (see, for example WO 2000/053722, WO 2008/022309, WO 2011/104169, and WO 2012/083185, WO 2013/032829, WO 2013/158141, each of which is incorporated herein by reference), by iontophoresis, or by incorporation into other delivery vehicles or systems available in the art such as hydrogels, cyclodextrins, biodegradable nanocapsules, bioadhesive microspheres, or proteinaceous vectors. In some embodiments the RNAi agents can be conjugated to antibodies having affinity for pulmonary epithelial cells. In some embodiments, the RNAi agents can be linked to targeting ligands that have affinity for pulmonary epithelial cells or receptors present on pulmonary epithelial cells.

### Pharmaceutical Compositions and Formulations

The IAV RNAi agents disclosed herein can be prepared as pharmaceutical compositions or formulations (also referred to herein as “medicaments”). In some embodiments, pharmaceutical compositions include at least one IAV RNAi agent. These pharmaceutical compositions are particularly useful in the inhibition of the expression of influenza A viral genome RNA or an influenza RNA transcript in a target cell, a group of cells, a tissue, or an organism. The pharmaceutical compositions can be used to treat a subject having a disease, disorder, or condition that would benefit from reduction in the level of the target influenza virus mRNA or RNA transcript, or inhibition in expression of the target viral genome. The pharmaceutical compositions can be used to treat a subject at risk of developing a disease or disorder that would benefit from reduction of the level of the target RNA or the target viral genome. In one embodiment, the method includes administering an IAV RNAi agent linked to a targeting ligand as described herein, to a subject to be treated. In some embodiments, one or more pharmaceutically acceptable excipients (including vehicles, carriers, diluents, and/or delivery polymers) are added to the pharmaceutical compositions that include an IAV RNAi agent, thereby forming a pharmaceutical formulation or medicament suitable for in vivo delivery to a subject, including a human.

The pharmaceutical compositions that include an IAV RNAi agent and methods disclosed herein decrease the level of the target influenza A viral RNA in a cell, group of cells, group of cells, tissue, organ, or subject, including by administering to the subject a therapeutically effective amount of a herein described IAV RNAi agent, thereby inhibiting the expression of Influenza A viral genome RNA or another influenza RNA or RNA transcript in the subject. In some embodiments, the subject has been previously identified or diagnosed as having a disease or disorder related to influenza infection, including influenza A viral genome infection, such as of symptoms and diseases associated influenza A viral infection, including but not limited to infection of the nose, throat, lungs, and other parts of the respiratory system. In some embodiments, the subject has been previously diagnosed with having pulmonary inflammation or other pulmonary symptoms consistent with an influenza infection.

Embodiments of the present disclosure include pharmaceutical compositions for delivering an IAV RNAi agent to a pulmonary epithelial cell in vivo. Such pharmaceutical compositions can include, for example, an IAV RNAi agent conjugated to a targeting group that comprises an integrin targeting ligand. In some embodiments, the integrin targeting ligand is comprised of an αvβ6 integrin ligand.

In some embodiments, the described pharmaceutical compositions including an IAV RNAi agent are used for treating or managing clinical presentations in a subject that would benefit from the inhibition of expression of influenza A viral genome. In some embodiments, a therapeutically or prophylactically effective amount of one or more of pharmaceutical compositions is administered to a subject in need of such treatment. In some embodiments, administration of any of the disclosed IAV RNAi agents can be used to decrease the number, severity, and/or frequency of symptoms of a disease in a subject.

In some embodiments, the described IAV RNAi agents are optionally combined with one or more additional (i.e., second, third, etc.) therapeutics. A second therapeutic can be another IAV RNAi agent (e.g., an IAV RNAi agent that targets a different sequence within an influenza A viral genome viral genome). In some embodiments, a second therapeutic can be an RNAi agent that targets the influenza A viral genome or the genome of a different influenza virus. An additional therapeutic can also be a small molecule drug, antibody, antibody fragment, peptide, vaccine, and/or aptamer. The IAV RNAi agents, with or without the one or more additional therapeutics, can be combined with one or more excipients to form pharmaceutical compositions.

The described pharmaceutical compositions that include an IAV RNAi agent can be used to treat at least one symptom in a subject having a disease or disorder caused by an influenza virus infection. In some embodiments, the subject is administered a therapeutically effective amount of one or more pharmaceutical compositions that include an IAV RNAi agent thereby treating the symptom. In other embodiments, the subject is administered a prophylactically effective amount of one or more IAV RNAi agents, thereby preventing or inhibiting the at least one symptom by preventing the influenza virus from establishing itself and replicating in the cells of the organism.

The described pharmaceutical compositions that include an IAV RNAi agent can be used to treat at least one symptom in a subject having a disease or disorder caused by an influenza virus infection (including potentially preventative or prophylactic treatment). The influenza virus infection can be caused by influenza A subtypes including but not limited to, H1N1, H2N2, H3N2, H5N1, H7N9, and H10N8.

In some embodiments, one or more of the described IAV RNAi agents are administered to a mammal in a pharmaceutically acceptable carrier or diluent. In some embodiments, the mammal is a human.

The route of administration is the path by which an IAV RNAi agent is brought into contact with the body. In general, methods of administering drugs, oligonucleotides, and nucleic acids, for treatment of a mammal are well known in the art and can be applied to administration of the compositions described herein. The IAV RNAi agents disclosed herein can be administered via any suitable route in a preparation appropriately tailored to the particular route. Thus, in some embodiments, the herein described pharmaceutical compositions are administered via inhalation, intranasal administration, intratracheal administration, or oropharyngeal aspiration administration. In some embodiments, the pharmaceutical compositions can be administered by injection, for example, intravenously, intramuscularly, intracutaneously, subcutaneously, intraarticularly, intraocularly, or intraperitoneally, or topically.

The pharmaceutical compositions including an IAV RNAi agent described herein can be delivered to a cell, group of cells, tissue, or subject using oligonucleotide delivery technologies known in the art. In general, any suitable method recognized in the art for delivering a nucleic acid molecule (in vitro or in vivo) can be adapted for use with the compositions described herein. For example, delivery can be by local administration, (e.g., direct injection, implantation, or topical administering), systemic administration, or subcutaneous, intravenous, intraperitoneal, or parenteral routes, including intracranial (e.g., intraventricular, intraparenchymal and intrathecal), intramuscular, transdermal, airway (aerosol), nasal, oral, rectal, or topical (including buccal and sublingual) administration. In some embodiments, the compositions are administered via inhalation, intranasal administration, oropharyngeal aspiration administration, or intratracheal administration. For example, in some embodiments, it is desired that the IAV RNAi agents described herein inhibit the expression of an influenza A viral genome or the genome of another influenza virus in the pulmonary epithelium, for which administration via inhalation (e.g., by an inhaler device, such as a metered-dose inhaler, or a nebulizer such as a jet or vibrating mesh nebulizer, or a soft mist inhaler) is particularly suitable and advantageous

In some embodiments, the pharmaceutical compositions described herein comprise one or more pharmaceutically acceptable excipients. The pharmaceutical compositions described herein are formulated for administration to a subject.

As used herein, a pharmaceutical composition or medicament includes a pharmacologically effective amount of at least one of the described therapeutic compounds and one or more pharmaceutically acceptable excipients. Pharmaceutically acceptable excipients (excipients) are substances other than the Active Pharmaceutical Ingredient (API, therapeutic product, e.g., IAV RNAi agent) that are intentionally included in the drug delivery system. Excipients do not exert or are not intended to exert a therapeutic effect at the intended dosage. Excipients can act to a) aid in processing of the drug delivery system during manufacture, b) protect, support or enhance stability, bioavailability or patient acceptability of the API, c) assist in product identification, and/or d) enhance any other attribute of the overall safety, effectiveness, of delivery of the API during storage or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, detergents, dextran, dextrose, diluents, disintegrants, emulsifiers, extenders, fillers, flavors, glidants, humectants, lubricants, oils, polymers, preservatives, saline, salts, solvents, sugars, surfactants, suspending agents, sustained release matrices, sweeteners, thickening agents, tonicity agents, vehicles, water-repelling agents, and wetting agents.

Pharmaceutical compositions suitable for injectable use include sterile aqueous solutions (where water-soluble) or dispersions and sterile powders for the extemporaneous preparation of sterile injectable solutions or dispersion. For intravenous administration, suitable carriers include physiological saline, bacteriostatic water, Cremophor® EL™ (BASF, Parsippany, NJ) or phosphate buffered saline (PBS). It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof.

Formulations suitable for intra-articular administration can be in the form of a sterile aqueous preparation of the drug that can be in microcrystalline form, for example, in the form of an aqueous microcrystalline suspension. Liposomal formulations or biodegradable polymer systems can also be used to present the drug for both intra-articular and ophthalmic administration.

Formulations suitable for inhalation administration can be prepared by incorporating the active compound in the desired amount in an appropriate solvent, followed by sterile filtration. In general, formulations for inhalation administration are sterile solutions at physiological pH and have low viscosity (<5 cP). Salts may be added to the formulation to balance tonicity. In some cases, surfactants or co-solvents can be added to increase active compound solubility and improve aerosol characteristics. In some cases, excipients can be added to control viscosity in order to ensure size and distribution of nebulized droplets.

In some embodiments, pharmaceutical formulations that include the IAV RNAi agents disclosed herein suitable for inhalation administration can be prepared in water for injection (sterile water), or an aqueous sodium phosphate buffer (for example, the IAV RNAi agent formulated in 0.5 mM sodium phosphate monobasic, 0.5 mM sodium phosphate dibasic, in water).

The active compounds can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in U.S. Pat. No. 4,522,811.

The IAV RNAi agents can be formulated in compositions in dosage unit form for ease of administration and uniformity of dosage. Dosage unit form refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the therapeutic effect to be achieved, and the limitations inherent in the art of compounding such an active compound for the treatment of individuals.

A pharmaceutical composition can contain other additional components commonly found in pharmaceutical compositions. Such additional components include, but are not limited to: anti-pruritics, astringents, local anesthetics, or anti-inflammatory agents (e.g., antihistamine, diphenhydramine, etc.). It is also envisioned that cells, tissues, or isolated organs that express or comprise the herein defined RNAi agents may be used as “pharmaceutical compositions.” As used herein, “pharmacologically effective amount,” “therapeutically effective amount,” or simply “effective amount” refers to that amount of an RNAi agent to produce a pharmacological, therapeutic, or preventive result.

In some embodiments, the methods disclosed herein further comprise the step of administering a second therapeutic or treatment in addition to administering an RNAi agent disclosed herein. In some embodiments, the second therapeutic is another IAV RNAi agent (e.g., an IAV RNAi agent that targets a different sequence within the influenza A viral genome target). In other embodiments, the second therapeutic can be a small molecule drug, an antibody, an antibody fragment, a peptide, a vaccine, and/or an aptamer.

In some embodiments, described herein are compositions that include a combination or cocktail of at least two IAV RNAi agents having different sequences. In some embodiments, the two or more IAV RNAi agents are each separately and independently linked to targeting groups. In some embodiments, the two or more IAV RNAi agents are each linked to targeting groups that include or consist of integrin targeting ligands. In some embodiments, the two or more IAV RNAi agents are each linked to targeting groups that include or consist of αvβ6 integrin targeting ligands.

Described herein are compositions for delivery of IAV RNAi agents to pulmonary epithelial cells.

Generally, an effective amount of an IAV RNAi agent disclosed herein will be in the range of from about 0.0001 to about 30 mg/kg of body weight/deposited dose, e.g., from about 0.001 to about 5 mg/kg of body weight/deposited dose. In some embodiments, an effective amount of an IAV RNAi agent will be in the range of from about 0.01 mg/kg to about 3.0 mg/kg of body weight per deposited dose. In some embodiments, an effective amount of an IAV RNAi agent will be in the range of from about 0.03 mg/kg to about 2.0 mg/kg of body weight per deposited dose. In some embodiments, an effective amount of an IAV RNAi agent will be in the range of from about 0.01 to about 1.0 mg/kg of deposited dose per body weight. In some embodiments, an effective amount of an IAV RNAi agent will be in the range of from about 0.50 to about 1.0 mg/kg of deposited dose per body weight. The amount administered will also likely depend on such variables as the overall health status of the patient, the relative biological efficacy of the compound delivered, the formulation of the drug, the presence and types of excipients in the formulation, and the route of administration. Also, it is to be understood that the initial dosage administered can be increased beyond the above upper level to rapidly achieve the desired blood-level or tissue level, or the initial dosage can be smaller than the optimum. In some embodiments, a dose is administered daily. In some embodiments, a dose is administered weekly. In further embodiments, a dose is administered bi-weekly, tri-weekly, once monthly, or once quarterly (i.e., once every three months).

For treatment of disease or for formation of a medicament or composition for treatment of a disease, the pharmaceutical compositions described herein including an IAV RNAi agent can be combined with an excipient or with a second therapeutic agent or treatment including, but not limited to: a second or other RNAi agent, a small molecule drug, an antibody, an antibody fragment, a peptide, a vaccine and/or an aptamer.

The described IAV RNAi agents, when added to pharmaceutically acceptable excipients or adjuvants, can be packaged into kits, containers, packs, or dispensers. The pharmaceutical compositions described herein can be packaged in dry powder or aerosol inhalers, other metered-dose inhalers, nebulizers, pre-filled syringes, or vials.

### Methods of Treatment and Inhibition of Influenza A Viral Genomes

The IAV RNAi agents disclosed herein can be used to treat a subject (e.g., a human or other mammal) having a disease or disorder that would benefit from administration of the RNAi agent. In some embodiments, the RNAi agents disclosed herein can be used to treat a subject (e.g., a human) that would benefit from a reduction and/or inhibition in expression of influenza A viral genome mRNA and/or viral transcripts, or a reduction and/or inhibition of another influenza virus that is infecting the subject.

In some embodiments, the RNAi agents disclosed herein can be used to treat a subject (e.g., a human) having a disease or disorder caused by an influenza virus infection, including but not limited to, pulmonary inflammation or symptoms and diseases associated influenza A viral infection, including but not limited to infection of the nose, throat, lungs, and other parts of the respiratory system. Treatment of a subject can include therapeutic and/or prophylactic treatment. The subject is administered a therapeutically effective amount of any one or more IAV RNAi agents described herein. The subject can be a human, patient, or human patient. The subject may be an adult, adolescent, child, or infant. Administration of a pharmaceutical composition described herein can be to a human being or animal.

In certain embodiments, the present disclosure provides methods for treatment of diseases, disorders, conditions, or pathological states mediated at least in part by influenza A viral genome viral genome expression, in a patient in need thereof, wherein the methods include administering to the patient any of the IAV RNAi agents described herein.

In some embodiments, the IAV RNAi agents are used to treat or manage a clinical presentation or pathological state in a subject, wherein the clinical presentation or pathological state is caused by an influenza virus infection. The subject is administered a therapeutically effective amount of one or more of the IAV RNAi agents or IAV RNAi agent-containing compositions described herein. In some embodiments, the method comprises administering a composition comprising an IAV RNAi agent described herein to a subject to be treated.

In a further aspect, the disclosure features methods of treatment (including prophylactic or preventative treatment) of diseases or symptoms that may be addressed by a reduction in influenza mRNA or RNA transcripts, including for example a reduction in influenza A viral genome mRNA or RNA transcripts, the methods comprising administering to a subject in need thereof an IAV RNAi agent that includes an antisense strand comprising the sequence of any of the sequences in Table 2A, 2B, 2C, 2D, 2E, 2F, 3A, 3B, 3C, 3D, 3E, 3F, 10A, 10B, 10C, 10D, 10E, or 10F. Also described herein are compositions for use in such methods.

In another aspect, the disclosure provides methods for the treatment (including prophylactic treatment) of a pathological state (such as a condition or disease) caused by an influenza virus infection, wherein the methods include administering to a subject a therapeutically effective amount of an RNAi agent that includes an antisense strand comprising the sequence of any of the sequences in Table 2A, 2B, 2C, 2D, 2E, 2F, 3A, 3B, 3C, 3D, 3E, 3F, 10A, 10B, 10C, 10D, 10E, or 10F.

In some embodiments, methods for inhibiting expression of an influenza A viral genome viral genome are disclosed herein, wherein the methods include administering to a cell an RNAi agent that includes an antisense strand comprising the sequence of any of the sequences in Table 2A, 2B, 2C, 2D, 2E, 2F, 3A, 3B, 3C, 3D, 3E, 3F, 10A, 10B, 10C, 10D, 10E, or 10F.

In some embodiments, methods for the treatment (including prophylactic treatment) of a pathological state mediated at least in part by influenza A viral RNA are disclosed herein, wherein the methods include administering to a subject a therapeutically effective amount of an RNAi agent that includes a sense strand comprising the sequence of any of the sequences in Table 2A, 2B, 2C, 2D, 2E, 2F, 4A, 4B, 4C, 4D, 4E, 4F, 5A, 5B, 5C, 5D, 5E, 5F, 6A, 6B, 6C, 6D, 6E, 6F, 10A, 10B, 10C, 10D, 10E, or 10F.

In some embodiments, methods for inhibiting expression of a Influenza A viral genome viral genome are disclosed herein, wherein the methods comprise administering to a cell an RNAi agent that includes a sense strand comprising the sequence of any of the sequences in Table 2A, 2B, 2C, 2D, 2E, 2F, 4A, 4B, 4C, 4D, 4E, 4F, 5A, 5B, 5C, 5D, 5E, 5F, 6A, 6B, 6C, 6D, 6E, 6F, 10A, 10B, 10C, 10D, 10E, or 10F.

In some embodiments, methods for the treatment (including prophylactic treatment) of a pathological state mediated at least in part by Influenza A viral genome viral RNA are disclosed herein, wherein the methods include administering to a subject a therapeutically effective amount of an RNAi agent that includes a sense strand comprising the sequence of any of the sequences in Table 4A, 4B, 4C, 4D, 4E, 4F, 5A, 5B, 5C, 5D, 5E, 5F, 6A, 6B, 6C, 6D, 6E, 6F, 10A, 10B, 10C, 10D, 10E, or 10F, and an antisense strand comprising the sequence of any of the sequences in Table 3A, 3B, 3C, 3D, 3E, 3F, 10A, 10B, 10C, 10D, 10E, or 10F.

In some embodiments, methods for inhibiting expression of an influenza A viral genome are disclosed herein, wherein the methods include administering to a cell an RNAi agent that includes a sense strand comprising the sequence of any of the sequences in Table 4A, 4B, 4C, 4D, 4E, 4F, 5A, 5B, 5C, 5D, 5E, 5F, 6A, 6B, 6C, 6D, 6E, 6F, 10A, 10B, 10C, TOD, 10E, or 10F, and an antisense strand comprising the sequence of any of the sequences in Table 3A, 3B, 3C, 3D, 3E, 3F, 10A, 10B, 10C, 10D, 10E, or 10F.

In some embodiments, methods of inhibiting expression of an influenza A viral genome are disclosed herein, wherein the methods include administering to a subject an IAV RNAi agent that includes a sense strand consisting of the nucleobase sequence of any of the sequences in Table 4A, 4B, 4C, 4D, 4E, 4F, 5A, 5B, 5C, 5D, 5E, 5F, 6A, 6B, 6C, 6D, 6E, 6F, 10A, 10B, 10C, 10D, 10E, or 10F, and the antisense strand consisting of the nucleobase sequence of any of the sequences in Table 3A, 3B, 3C, 3D, 3E, 3F, 10A, 10B, 10C, 10D, 10E, or 10F. In other embodiments, disclosed herein are methods of inhibiting expression of an influenza A viral genome, wherein the methods include administering to a subject an IAV RNAi agent that includes a sense strand consisting of the modified sequence of any of the modified sequences in Table 4A, 4B, 4C, 4D, 4E, 4F, 5A, 5B, 5C, 5D, 5E, 5F, 6A, 6B, 6C, 6D, 6E, 6F, 10A, 10B, 10C, 10D, 10E, or 10F, and the antisense strand consisting of the modified sequence of any of the modified sequences in Table 3A, 3B, 3C, 3D, 3E, 3F, 10A, 10B, 10C, 10D, 10E, or 10F.

In some embodiments, methods for inhibiting expression of an influenza A viral genome in a cell are disclosed herein, wherein the methods include administering one or more IAV RNAi agents comprising a duplex structure of one of the duplexes set forth in Tables 7A-1, 7A-2, 7A-3, 7A-4, 7A-5, 7A-6, 7B-1, 7B-2, 7B-3, 7B-4, 7B-5, 7B-6, 8A, 8B, 8C, 8D, 8E, 8F, 9A, 9B, 9C, 9D, 9E, and 9F.

In some embodiments, methods for inhibiting expression of an influenza A viral genome are disclosed herein, wherein the methods include administering one or more IAV RNAi agents directed to Influenza A virus (A/California/07/2009(H1N1)) segment 7 matrix protein 2 (M2) and matrix protein 1 (M1) genes (referred to herein as M1). In some embodiments, methods for inhibiting expression of an influenza A viral genome are disclosed herein, wherein the methods include administering to a subject an IAV RNAi agent that includes a sense strand consisting of the nucleobase sequence of any of the sequences in Table 4A, 5A, 6A, or 10A, and the antisense strand consisting of the nucleobase sequence of any of the sequences in Table 3A or 10A. In other embodiments, disclosed herein are methods of inhibiting expression of an influenza A viral genome, wherein the methods include administering to a subject an IAV RNAi agent that includes a sense strand consisting of the modified sequence of any of the modified sequences in Table 4A, 5A, 6A, or 10A, and the antisense strand consisting of the modified sequence of any of the modified sequences in Table 3A or 10A.

In some embodiments, methods for inhibiting expression of an influenza A viral genome are disclosed herein, wherein the methods include administering one or more IAV RNAi agents directed to Influenza A virus (A/California/07/2009(H1N1)) segment 8 nuclear export protein (NEP) and nonstructural protein 1 (NS1) genes. In some embodiments, methods for inhibiting expression of an influenza A viral genome are disclosed herein, wherein the methods include administering to a subject an IAV RNAi agent that includes a sense strand consisting of the nucleobase sequence of any of the sequences in Table 4B, 5B, 6B, or 10B, and the antisense strand consisting of the nucleobase sequence of any of the sequences in Table 3B or 10B. In other embodiments, disclosed herein are methods of inhibiting expression of an influenza A viral genome, wherein the methods include administering to a subject an IAV RNAi agent that includes a sense strand consisting of the modified sequence of any of the modified sequences in Table 4B, 5B, 6B, or 10B, and the antisense strand consisting of the modified sequence of any of the modified sequences in Table 3B or 10B.

In some embodiments, methods for inhibiting expression of an influenza A viral genome are disclosed herein, wherein the methods include administering one or more IAV RNAi agents directed to Influenza A virus (A/California/07/2009(H1N1)) segment 2 polymerase PB1 (PB1) gene and nonfunctional PB1-F2 protein (PB1-F2) gene. In some embodiments, methods for inhibiting expression of an influenza A viral genome are disclosed herein, wherein the methods include administering to a subject an IAV RNAi agent that includes a sense strand consisting of the nucleobase sequence of any of the sequences in Table 4C, 5C, 6C, or 10C, and the antisense strand consisting of the nucleobase sequence of any of the sequences in Table 3C or 10C. In other embodiments, disclosed herein are methods of inhibiting expression of an influenza A viral genome, wherein the methods include administering to a subject an IAV RNAi agent that includes a sense strand consisting of the modified sequence of any of the modified sequences in Table 4C, 5C, 6C, or 10C, and the antisense strand consisting of the modified sequence of any of the modified sequences in Table 3C or 10C.

In some embodiments, methods for inhibiting expression of an influenza A viral genome are disclosed herein, wherein the methods include administering one or more IAV RNAi agents directed to Influenza A virus (A/California/07/2009(H1N1)) segment 1 polymerase PB2 (PB2) gene. In some embodiments, methods for inhibiting expression of an influenza A viral genome are disclosed herein, wherein the methods include administering to a subject an IAV RNAi agent that includes a sense strand consisting of the nucleobase sequence of any of the sequences in Table 4D, 5D, 6D, or 10D, and the antisense strand consisting of the nucleobase sequence of any of the sequences in Table 3D or TOD. In other embodiments, disclosed herein are methods of inhibiting expression of an influenza A viral genome, wherein the methods include administering to a subject an IAV RNAi agent that includes a sense strand consisting of the modified sequence of any of the modified sequences in Table 4D, 5D, 6D, or 10D, and the antisense strand consisting of the modified sequence of any of the modified sequences in Table 3D or 10D.

In some embodiments, methods for inhibiting expression of an influenza A viral genome are disclosed herein, wherein the methods include administering one or more IAV RNAi agents directed to Influenza A virus (A/California/07/2009(H1N1)) segment 5 nucleocapsid protein (NP) gene. In some embodiments, methods for inhibiting expression of an influenza A viral genome are disclosed herein, wherein the methods include administering to a subject an IAV RNAi agent that includes a sense strand consisting of the nucleobase sequence of any of the sequences in Table 4E, 5E, 6E, or 10E, and the antisense strand consisting of the nucleobase sequence of any of the sequences in Table 3E or 10E. In other embodiments, disclosed herein are methods of inhibiting expression of an influenza A viral genome, wherein the methods include administering to a subject an IAV RNAi agent that includes a sense strand consisting of the modified sequence of any of the modified sequences in Table 4E, 5E, 6E, or 10E, and the antisense strand consisting of the modified sequence of any of the modified sequences in Table 3E or 10E.

In some embodiments, methods for inhibiting expression of an influenza A viral genome are disclosed herein, wherein the methods include administering one or more IAV RNAi agents directed to Influenza A virus (A/California/07/2009(H1N1)) segment 3 polymerase PA (PA) gene. In some embodiments, methods for inhibiting expression of an influenza A viral genome are disclosed herein, wherein the methods include administering to a subject an IAV RNAi agent that includes a sense strand consisting of the nucleobase sequence of any of the sequences in Table 4F, 5F, 6F, or 10F, and the antisense strand consisting of the nucleobase sequence of any of the sequences in Table 3F or 10F. In other embodiments, disclosed herein are methods of inhibiting expression of an influenza A viral genome, wherein the methods include administering to a subject an IAV RNAi agent that includes a sense strand consisting of the modified sequence of any of the modified sequences in Table 4F, 5F, 6F, or 10F, and the antisense strand consisting of the modified sequence of any of the modified sequences in Table 3F or 10F.

In some embodiments, methods for inhibiting expression of an influenza A viral genome are disclosed herein, wherein the methods include administering one or more IAV RNAi agents directed to a single influenza A viral gene selected from the group consisting of: M1 (which includes M2), NEP, NS1, PB1, PB1-F2, PB2, NP, and PA.

In some embodiments, methods for inhibiting expression of an influenza A viral genome are disclosed herein, wherein the methods include administering one or more IAV RNAi agents directed to a combination of two or more influenza A viral genomes selected from the group consisting of: M1 (which includes M2), NEP, NS1, PB1, PB1-F2, PB2, NP, and PA.

In some embodiments, the influenza A viral RNA level in certain epithelial cells of subject to whom a described IAV RNAi agent is administered is reduced by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or greater than 99%, relative to the subject prior to being administered the IAV RNAi agent or to a subject not receiving the IAV RNAi agent. In some embodiments, the influenza A viral subgenomic RNA levels in certain epithelial cells of a subject to whom a described IAV RNAi agent is administered is reduced by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or greater than 99%, relative to the subject prior to being administered the IAV RNAi agent or to a subject not receiving the IAV RNAi agent. The viral RNA transcript level, mRNA level, and/or subgenomic RNA level in the subject may be reduced in a cell, group of cells, and/or tissue of the subject. In some embodiments, the influenza mRNA levels in certain epithelial cells subject to whom a described IAV RNAi agent has been administered is reduced by at least about 30%, 35%, 40%, 45%, 50%, 55%, 60%, 65%, 70%, 75%, 80%, 85%, 90%, 95%, or 98% relative to the subject prior to being administered the IAV RNAi agent or to a subject not receiving the IAV RNAi agent.

Reductions in viral RNA can be assessed by any methods known in the art and are collectively referred to herein as a decrease in, reduction of, or inhibition of influenza A viral genome. The Examples set forth herein illustrate known methods for assessing inhibition of Influenza A viral genome viral RNA.

### Cells, Tissues, Organs, and Non-Human Organisms

Cells, tissues, organs, and non-human organisms that include at least one of the IAV RNAi agents described herein are contemplated. The cell, tissue, organ, or non-human organism is made by delivering the RNAi agent to the cell, tissue, organ, or non-human organism.

### Additional Illustrative Embodiments

Provided here are certain additional illustrative embodiments of the disclosed technology. These embodiments are illustrative only and do not limit the scope of the present disclosure or of the claims attached hereto.
1. An RNAi agent for inhibiting expression of an influenza A viral genome, comprising:
   an antisense strand comprising at least 17 contiguous nucleotides differing by 0 or 1 nucleotides from any one of the sequences provided in Table 2A, 2B, 2C, 2D, 2E, 2F, 3A, 3B, 3C, 3D, 3E, or 3F; and
   a sense strand comprising a nucleotide sequence that is at least partially complementary to the antisense strand.
2. The RNAi agent of embodiment 1, wherein the antisense strand comprises nucleotides 2-18 of any one of the sequences provided in Table 2A, 2B, 2C, 2D, 2E, 2F, 3A, 3B, 3C, 3D, 3E, or 3F.
3. The RNAi agent of embodiment 1 or embodiment 2, wherein the sense strand comprises a nucleotide sequence of at least 17 contiguous nucleotides differing by 0 or 1 nucleotides from any one of the sequences provided in Table 2A, 2B, 2C, 2D, 2E, 2F, 4A, 4B, 4C, 4D, 4E, or 4F, and wherein the sense strand has a region of at least 85% complementarity over the 17 contiguous nucleotides to the antisense strand.
4. The RNAi agent of any one of embodiments 1-3, wherein at least one nucleotide of the IAV RNAi agent is a modified nucleotide or includes a modified internucleoside linkage.
5. The RNAi agent of any one of embodiments 1-4, wherein all or substantially all of the nucleotides are modified nucleotides.
6. The RNAi agent of any one of embodiments 4-5, wherein the modified nucleotide is selected from the group consisting of: 2′-O-methyl nucleotide, 2′-fluoro nucleotide, 2′-deoxy nucleotide, 2′,3′-seco nucleotide mimic, locked nucleotide, 2′-F-arabino nucleotide, 2′-methoxyethyl nucleotide, abasic nucleotide, ribitol, inverted nucleotide, inverted 2′-O-methyl nucleotide, inverted 2′-deoxy nucleotide, 2′-amino-modified nucleotide, 2′-alkyl-modified nucleotide, morpholino nucleotide, vinyl phosphonate-containing nucleotide, cyclopropyl phosphonate-containing nucleotide, and 3′-O-methyl nucleotide.
7. The RNAi agent of embodiment 5, wherein all or substantially all of the nucleotides are modified with 2′-O-methyl nucleotides, 2′-fluoro nucleotides, or combinations thereof.
8. The RNAi agent of any one of embodiments 1-7, wherein the antisense strand comprises the nucleotide sequence of any one of the modified sequences provided in Table 3A, 3B, 3C, 3D, 3E, and 3F.
9. The RNAi agent of any one of embodiments 1-8, wherein the sense strand comprises the nucleotide sequence of any one of the modified sequences provided in Table 4A, 4B, 4C, 4D. 4E, and 4F.
10. The RNAi agent of embodiment 1, wherein the antisense strand comprises the nucleotide sequence of any one of the modified sequences provided in Table 3A, 3B, 3C, 3D, 3E, and 3F, and the sense strand comprises the nucleotide sequence of any one of the modified sequences provided in Table 4A, 4B, 4C, 4D, 4E, and 4F.
11. The RNAi agent of any one of embodiments 1-10, wherein the sense strand is between 18 and 30 nucleotides in length, and the antisense strand is between 18 and 30 nucleotides in length.
12. The RNAi agent of embodiment 11, wherein the sense strand and the antisense strand are each between 18 and 27 nucleotides in length.
13. The RNAi agent of embodiment 12, wherein the sense strand and the antisense strand are each between 18 and 24 nucleotides in length.
14. The RNAi agent of embodiment 13, wherein the sense strand and the antisense strand are each 21 nucleotides in length.
15. The RNAi agent of embodiment 14, wherein the RNAi agent has two blunt ends.
16. The RNAi agent of any one of embodiments 1-15, wherein the sense strand comprises one or two terminal caps.
17. The RNAi agent of any one of embodiments 1-16, wherein the sense strand comprises one or two inverted abasic residues.
18. The RNAi agent of embodiment 1, wherein the RNAi agent is comprised of a sense strand and an antisense strand that form a duplex having the structure of any one of the duplexes in Table 7A-1, 7A-2, 7A-3, 7A-4, 7A-5, 7A-6, 7B-1, 7B-2, 7B-3, 7B-4, 7B-5, 7B-6, 8A, 8B, 8C, 8D, 8E, 8F, 9A, 9B, 9C, 9D, 9E, 9F, 10A, 10B, 10C, 10D, 10E, or 10F.
19. The RNAi agent of embodiment 18, wherein all or substantially all of the nucleotides are modified nucleotides.
20. The RNAi agent of embodiment 1, comprising an antisense strand that consists of, consists essentially of, or comprises a nucleotide sequence that differs by 0 or 1 nucleotides from the following nucleotide sequences (5′→3′):

(SEQ ID NO: 1590)
UUACGUUUCGACCUCGGUUAG.

21. The RNAi agent of embodiment 20, wherein the sense strand consists of, consists essentially of, or comprises a nucleotide sequence that differs by 0 or 1 nucleotides from the following nucleotide sequences 5′→3′):

(SEQ ID NO: 1706)
CUAACCGAGGUCGAAACGUAA.

22. The RNAi agent of embodiment 20 or 21, wherein all or substantially all of the nucleotides are modified nucleotides.
23. The RNAi agent of embodiment 1, comprising an antisense strand that comprises, consists of, or consists essentially of a modified nucleotide sequence that differs by 0 or 1 nucleotides from one of the following nucleotide sequences (5′→3′):

(SEQ ID NO: 1176)
cPrpusUfsascguUfucgaCfcUfcGfguuasg;
or
(SEQ ID NO: 1175)
cPrpusUfsascGfuuucgaCfcUfcGfguuasg;

wherein a represents 2′-O-methyl adenosine, c represents 2′-O-methyl cytidine, g represents 2′-O-methyl guanosine, and u represents 2′-O-methyl uridine; Af represents 2′-fluoro adenosine, Cf represents 2′-fluoro cytidine, Gf represents 2′-fluoro guanosine, and Uf represents 2′-fluoro uridine; cPrpu represents a 5′-cyclopropyl phosphonate-2′-O-methyl uridine; s represents a phosphorothioate linkage; and wherein all or substantially all of the nucleotides on the sense strand are modified nucleotides.
24. The RNAi agent of embodiment 1, wherein the sense strand comprises, consists of, or consists essentially of a modified nucleotide sequence that differs by 0 or 1 nucleotides from one of the following nucleotide sequences (5′→3′):

(SEQ ID NO: 1373)
csuaaccgaGfgUfcGfaaacguaa;
or
(SEQ ID NO: 1374)
csuaaccgaGfgUfcgaaacguaa;

wherein a represents 2′-O-methyl adenosine, c represents 2′-O-methyl cytidine, g represents 2′-O-methyl guanosine, and u represents 2′-O-methyl uridine; Af represents 2′-fluoro adenosine, Cf represents 2′-fluoro cytidine, Gf represents 2′-fluoro guanosine, and Uf represents 2′-fluoro uridine; cPrpu represents a 5′-cyclopropyl phosphonate-2′-O-methyl uridine; s represents a phosphorothioate linkage; and wherein all or substantially all of the nucleotides on the antisense strand are modified nucleotides.
25. The RNAi agent of any one of embodiments 20-24, wherein the sense strand further includes inverted abasic residues at the 3′ terminal end of the nucleotide sequence, at the 5′ end of the nucleotide sequence, or at both.
26. The RNAi agent of any one of embodiments 1-25, wherein the RNAi agent is linked to a targeting ligand.
27. The RNAi agent of embodiment 26, wherein the targeting ligand has affinity for a cell receptor expressed on an epithelial cell.
28. The RNAi agent of embodiment 27, wherein the targeting ligand comprises an integrin targeting ligand.
29. The RNAi agent of embodiment 28, wherein the integrin targeting ligand is an αvβ6 integrin targeting ligand.
30. The RNAi agent of embodiment 29, wherein the targeting ligand comprises the structure:

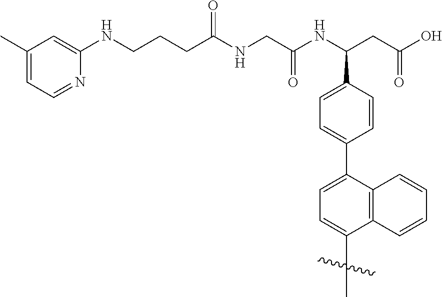 [SMILES: *c1ccc(-c2ccc([C@H](CC(=O)O)NC(=O)CNC(=O)CCCNc3cc(C)ccn3)cc2)c2ccccc12]

or a pharmaceutically acceptable salt thereof, or

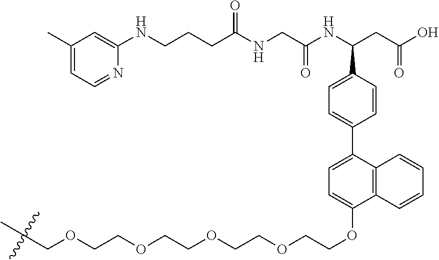 [SMILES: *COCCOCCOCCOCCOc1ccc(-c2ccc([C@H](CC(=O)O)NC(=O)CNC(=O)CCCNc3cc(C)ccn3)cc2)c2ccccc12]

or a pharmaceutically acceptable salt thereof,
wherein [symbol] indicates the point of connection to the RNAi agent.
31. The RNAi agent of any one of embodiments 26-29, wherein the targeting ligand has a structure selected from the group consisting of:

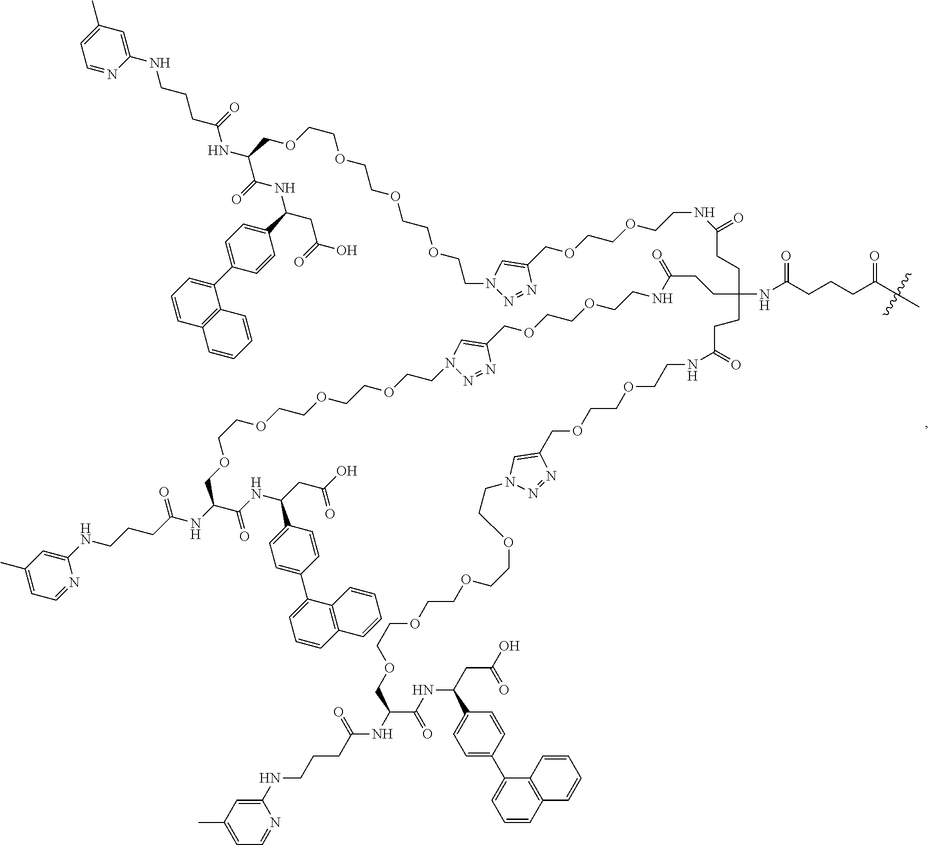 [SMILES: *C(=O)CCCC(=O)NC(CCC(=O)NCCOCCOCc1cn(CCOCCOCCOCCOC[C@H](NC(=O)CCCNc2cc(C)ccn2)C(=O)N[C@@H](CC(=O)O)c2ccc(-c3cccc4ccccc34)cc2)nn1)(CCC(=O)NCCOCCOCc1cn(CCOCCOCCOCCOC[C@H](NC(=O)CCCNc2cc(C)ccn2)C(=O)N[C@@H](CC(=O)O)c2ccc(-c3cccc4ccccc34)cc2)nn1)CCC(=O)NCCOCCOCc1cn(CCOCCOCCOCCOC[C@H](NC(=O)CCCNc2cc(C)ccn2)C(=O)N[C@@H](CC(=O)O)c2ccc(-c3cccc4ccccc34)cc2)nn1]

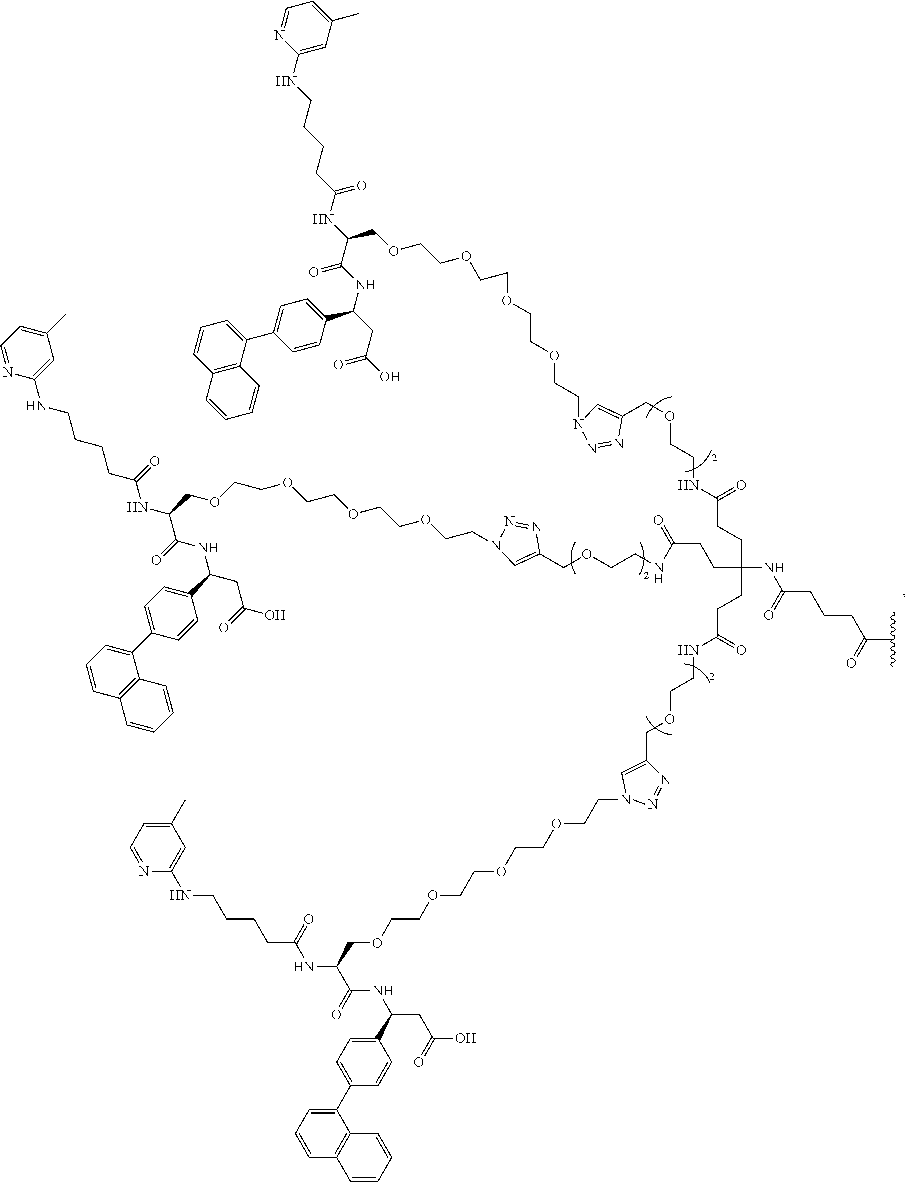

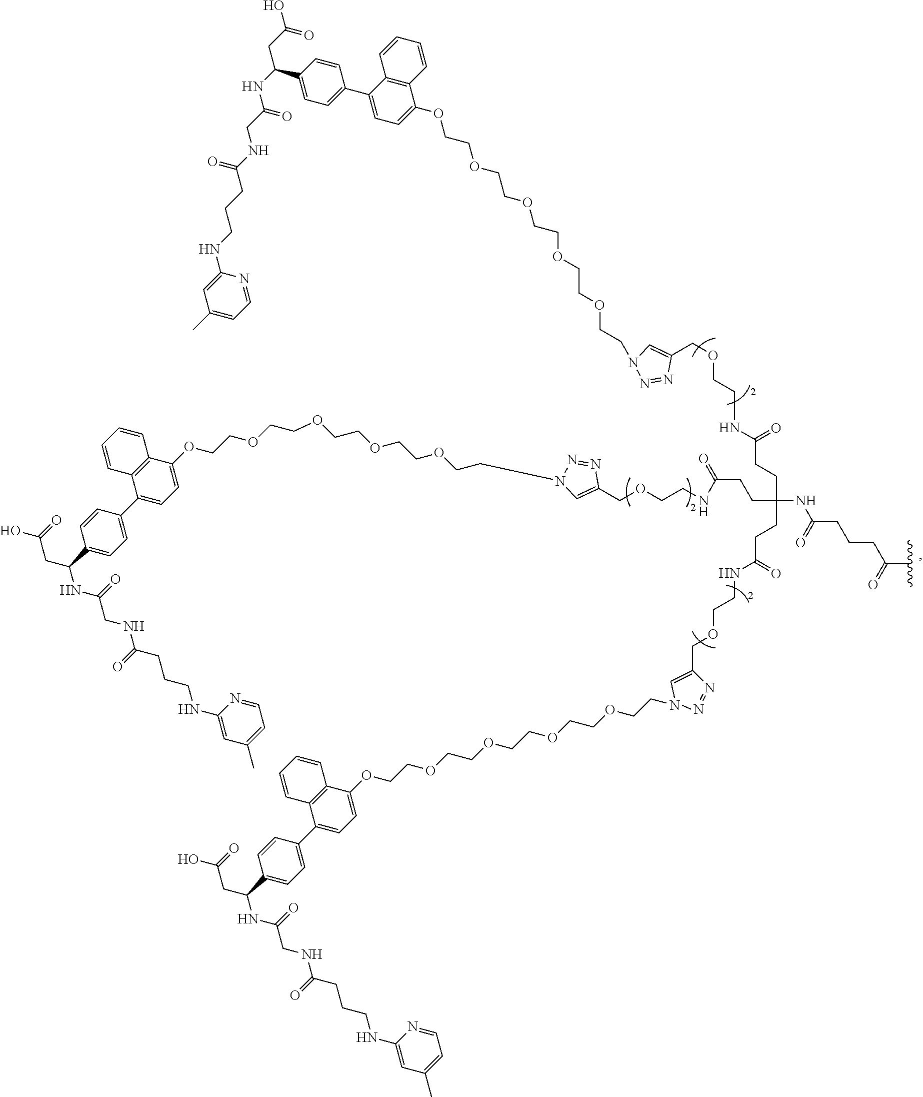

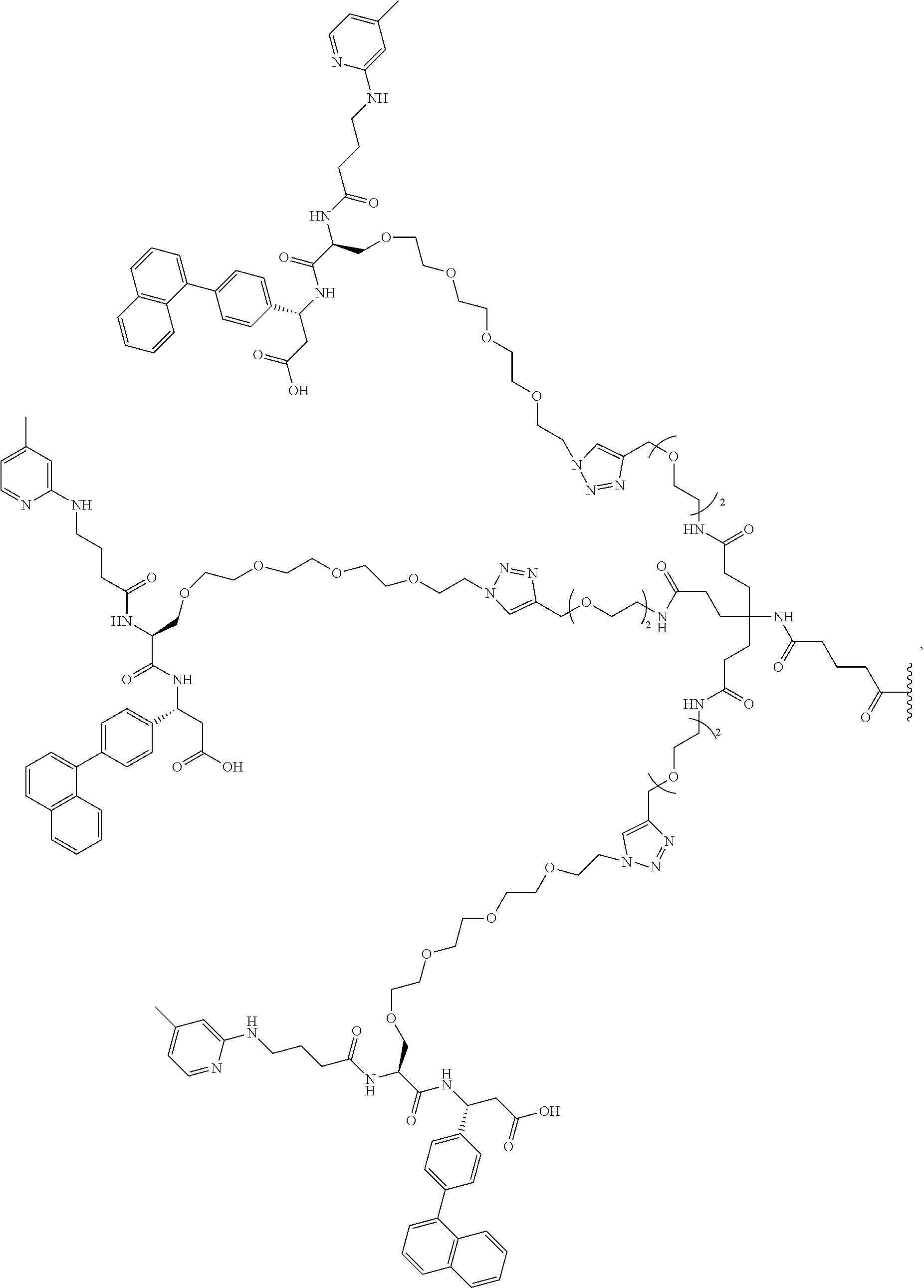

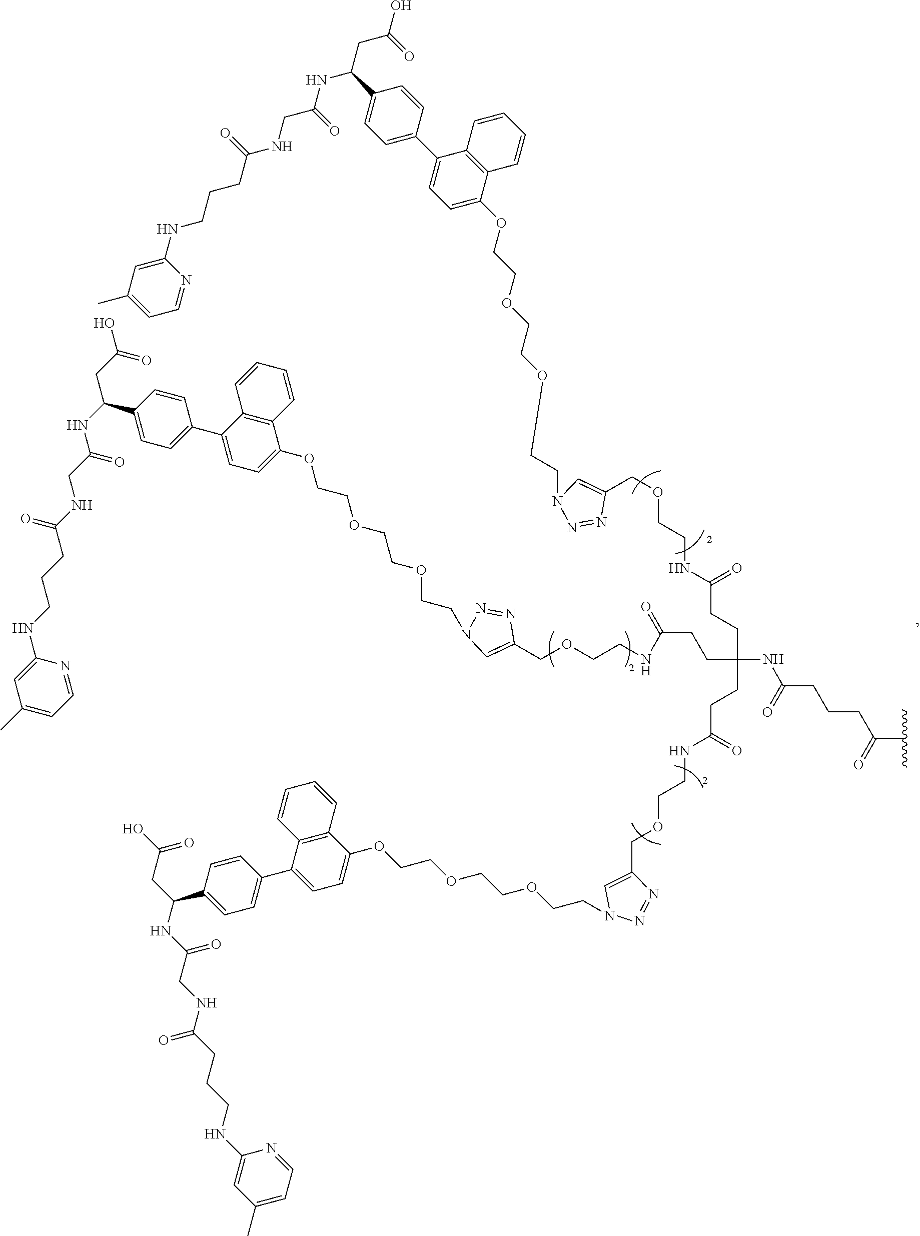

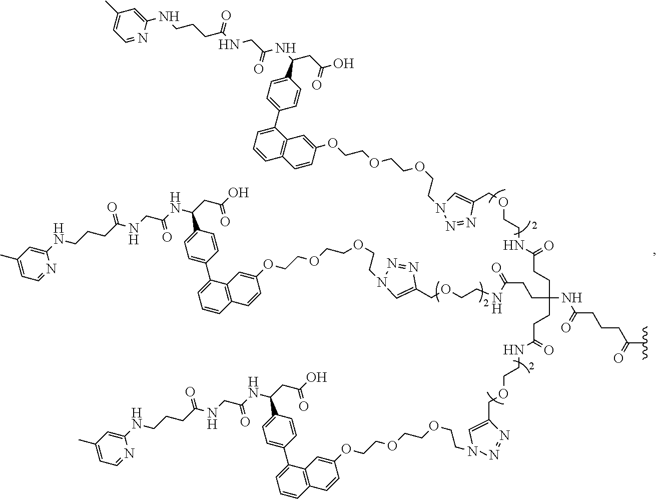

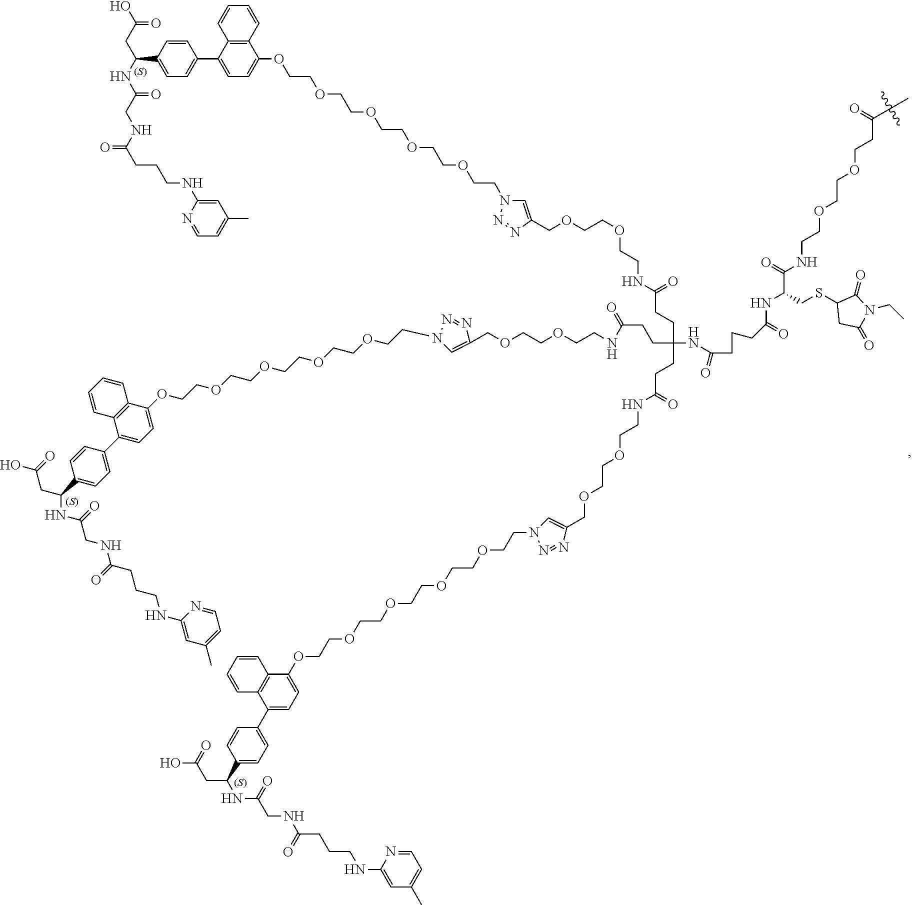

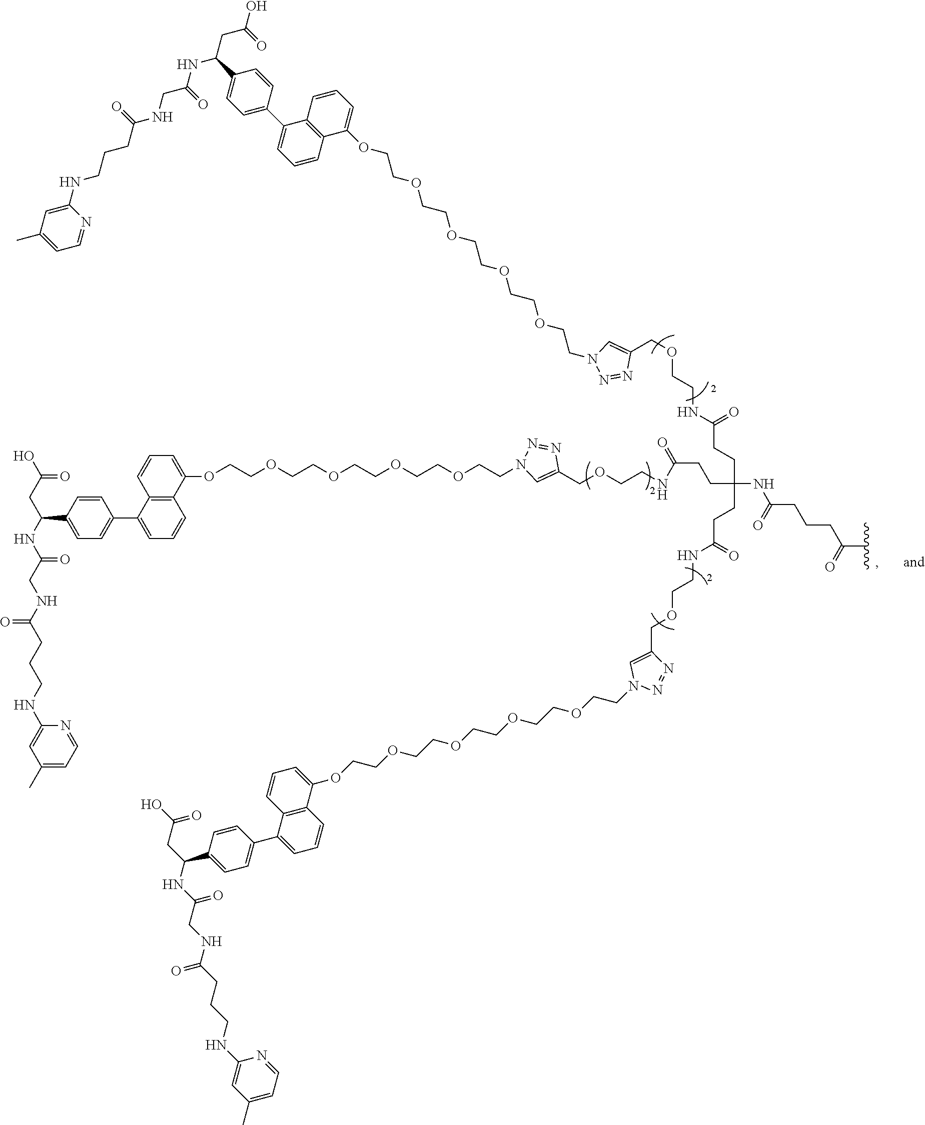

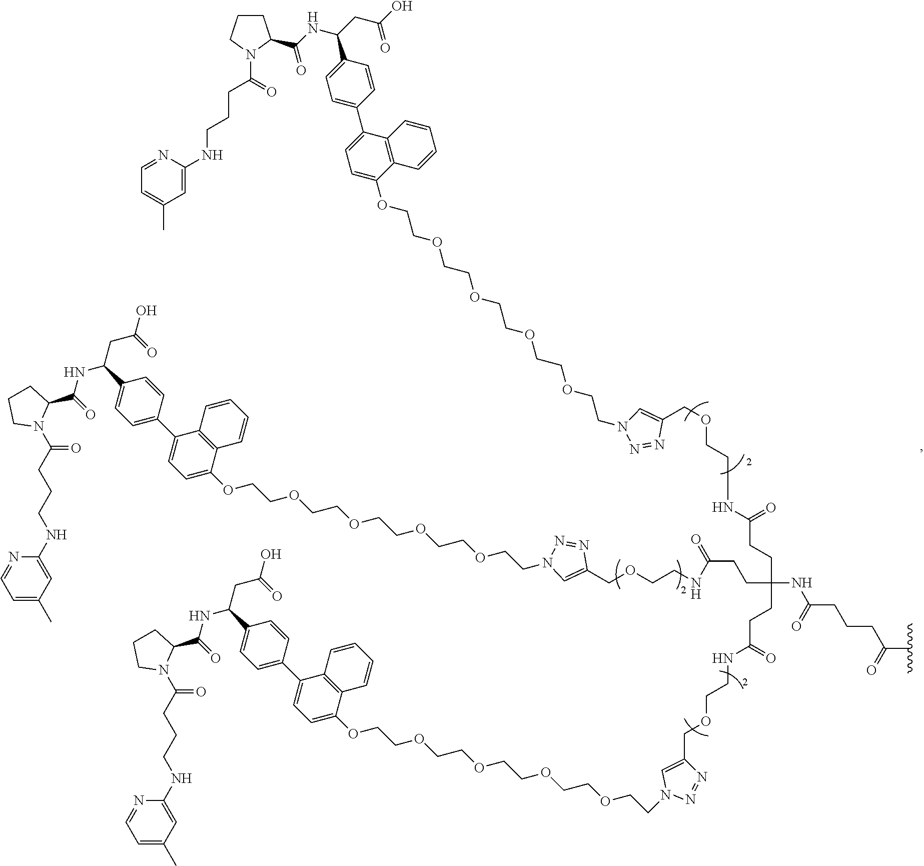

wherein [symbol] indicates the point of connection to the RNAi agent.
32. The RNAi agent of embodiment 31, wherein RNAi agent is conjugated to a targeting ligand having the following structure:

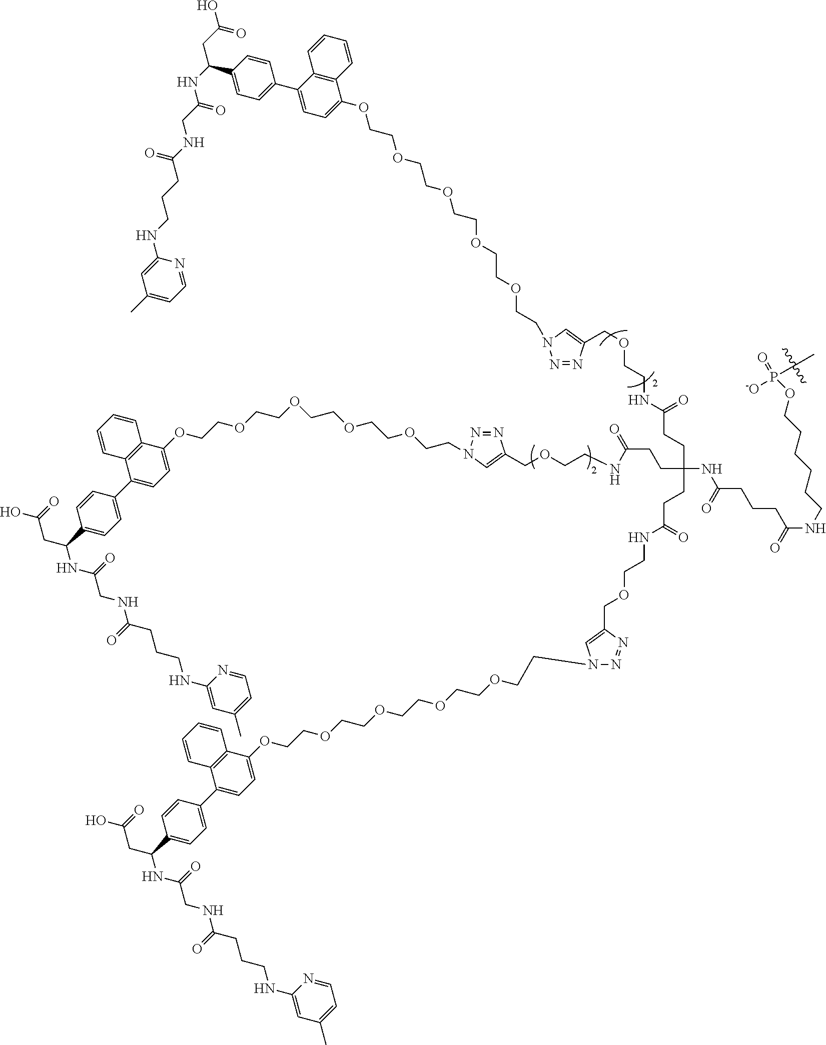

33. The RNAi agent of any one of embodiments 26-32, wherein the targeting ligand is conjugated to the sense strand.
34. The RNAi agent of embodiment 33, wherein the targeting ligand is conjugated to the 5′ terminal end of the sense strand.
35. The RNAi agent of any of embodiments 1-34, wherein the influenza A viral genome is selected from the viral genomes of the group consisting of:
   H1N1 viral genome;
   H2N2 viral genome;
   H3N2 viral genome;
   H5N1 viral genome;
   H7N9 viral genome; and
   H10N8 viral genome.
36. A composition comprising the RNAi agent of any one of embodiments 1-35, wherein the composition further comprises a pharmaceutically acceptable excipient.
37. The composition of embodiment 36, further comprising a second RNAi agent capable of inhibiting the expression of influenza A viral genome.
38. The composition of embodiment 37, wherein the influenza A viral genome is selected from the viral genomes of the group consisting of.
   H1N1 viral genome;
   H2N2 viral genome;
   H3N2 viral genome;
   H5N1 viral genome;
   H7N9 viral genome; and
   H10N8 viral genome.
39. The composition of any one of embodiments 36-38, further comprising one or more additional therapeutics.
40. The composition of any one of embodiments 36-39, wherein the composition is formulated for administration by inhalation.
41. The composition of embodiment 40, wherein the composition is delivered by a metered-dose inhaler, jet nebulizer, vibrating mesh nebulizer, or soft mist inhaler.
42. The composition of any of embodiments 36-41, wherein the RNAi agent is a sodium salt.
43. The composition of embodiment 36, wherein the pharmaceutically acceptable excipient is water for injection.
44. The composition of embodiment 36, wherein the pharmaceutically acceptable excipient is a buffered saline solution.
45. A method for inhibiting expression of an influenza A viral genome in a cell and/or treating one or more symptoms or diseases associated with influenza A viral infection, the method comprising introducing into a cell and/or administering to a subject, an effective amount of an RNAi agent wherein the RNAi agent targets the M1 influenza A viral genomic segment transcript by having an antisense strand that comprises at least 15 contiguous nucleotides differing by 0, 1, 2, or 3 nucleotides that are complementary to a stretch of at least 15 contiguous nucleotides of SEQ ID NO. 1, and wherein the RNAi agent is optionally linked to a targeting ligand, preferably wherein the targeting ligand has affinity for a cell receptor expressed on an epithelial cell, and most preferably wherein the targeting ligand is an αvβ6 integrin targeting ligand.
46. A method for inhibiting expression of an influenza A viral genome in a cell, the method comprising introducing into a cell an effective amount of an RNAi agent of any one of embodiments 1-35 or the composition of any one of embodiments 36-44.
47. The method of embodiment 45 or 46, wherein the influenza A viral genome is selected from the viral genomes of the group consisting of:
   H1N1 viral genome;
   H2N2 viral genome;
   H3N2 viral genome;
   H5N1 viral genome;
   H7N9 viral genome; and
   H10N8 viral genome.
48. The method of any of embodiments 45-47, wherein the cell is within a subject.
49. The method of embodiment 48, wherein the subject is a human subject.
50. The method of any one of embodiments 45-49, wherein following the administration of the RNAi agent the influenza A viral genome is inhibited by at least about 30%.
51. A method of treating one or more symptoms or diseases associated with influenza A viral infection, the method comprising administering to a human subject in need thereof a therapeutically effective amount of the composition of any one of embodiments 36-44.
52. The method of embodiment 45 or embodiment 51, wherein the disease is a respiratory disease.
53. The method of embodiment 52, wherein the respiratory disease is pulmonary inflammation.
54. The method of embodiment 52, wherein the disease is influenza A viral infection.
55. The method of embodiment 54, wherein the influenza A viral infection is caused by an influenza A virus subtype selected group consisting of
   H1N1;
   H2N2;
   H3N2;
   H5N1;
   H7N9; and
   H10N8.
56. The method of any one of embodiments 45-55, wherein the RNAi agent is administered at a deposited dose of about 0.01 mg/kg to about 5.0 mg/kg of body weight of the subject.
57. The method of any one of embodiments 45-56, wherein the RNAi agent is administered at a deposited dose of about 0.03 mg/kg to about 2.0 mg/kg of body weight of the subject.
58. The method of any one of embodiments 45-57, wherein the RNAi agent is administered in two or more doses.
59. Use of the RNAi agent of any one of embodiments 1-35, for the treatment of a disease, disorder, or symptom that is mediated at least in part by influenza A viral genome activity and/or influenza A viral genome expression.
60. Use of the composition according to any one of embodiments 36-44, for the treatment of a disease, disorder, or symptom that is mediated at least in part by influenza A viral genome activity and/or influenza A viral genome expression.
61. Use of the composition according to any one of embodiments 36-44, for the manufacture of a medicament for treatment of a disease, disorder, or symptom that is mediated at least in part by influenza A viral genome and/or influenza A viral genome expression.
62. The use of any one of embodiments 59-61, wherein the disease is influenza infection.
63. A method of manufacturing an RNAi agent of any one of embodiments 1-35, comprising annealing a sense strand and an antisense strand to form a double-stranded ribonucleic acid molecule.
64. The method of embodiment 63, wherein the sense strand comprises a targeting ligand.
65. The method of embodiment 64, comprising conjugating a targeting ligand to the sense strand.

The above provided embodiments and items are now illustrated with the following, non-limiting examples.

### EXAMPLES

### Example 1. Synthesis of IAV RNAi Agents

IAV RNAi agent duplexes disclosed herein were synthesized in accordance with the following:

### A. Synthesis

The sense and antisense strands of the IAV RNAi agents were synthesized according to phosphoramidite technology on solid phase used in oligonucleotide synthesis. Depending on the scale, a MerMade96E® (Bioautomation), a MerMade12® (Bioautomation), or an OP Pilot 100 (GE Healthcare) was used. Syntheses were performed on a solid support made of controlled pore glass (CPG, 500 Å or 600 Å, obtained from Prime Synthesis, Aston, PA, USA). All RNA and 2′-modified RNA phosphoramidites were purchased from Thermo Fisher Scientific (Milwaukee, WI, USA). Specifically, the 2′-O-methyl phosphoramidites that were used included the following: (5′-O-dimethoxytrityl-N⁶-(benzoyl)-2′-O-methyl-adenosine-3′-O-(2-cyanoethyl-N,N-diisopropylamino) phosphoramidite, 5′-O-dimethoxy-trityl-N⁴-(acetyl)-2′-O-methyl-cytidine-3′-O-(2-cyanoethyl-N,N-diisopropyl-amino) phosphoramidite, (5′-O-dimethoxytrityl-N²-(isobutyryl)-2′-O-methyl-guanosine-3′-O-(2-cyanoethyl-N,N-diisopropylamino) phosphoramidite, and 5′-O-dimethoxytrityl-2′-O-methyl-uridine-3′-O-(2-cyanoethyl-N,N-diisopropylamino) phosphoramidite. The 2′-deoxy-2′-fluoro-phosphoramidites carried the same protecting groups as the 2′-O-methyl RNA amidites. 5′-dimethoxytrityl-2′-O-methyl-inosine-3′-O-(2-cyanoethyl-N,N-diisopropylamino) phosphoramidites were purchased from Glen Research (Virginia). The inverted abasic (3′-O-dimethoxytrityl-2′-deoxyribose-5′-O-(2-cyanoethyl-N,N-diisopropylamino) phosphoramidites were purchased from ChemGenes (Wilmington, MA, USA). The following UNA phosphoramidites were used: 5′-(4,4′-Dimethoxytrityl)-N6-(benzoyl)-2′,3′-seco-adenosine, 2′-benzoyl-3′-[(2-cyanoethyl)-(N,N-diisopropyl)]-phosphoramidite, 5′-(4,4′-Dimethoxytrityl)-N-acetyl-2′,3′-seco-cytosine, 2′-benzoyl-3′-[(2-cyanoethyl)-(N,N-diiso-propyl)]-phosphoramidite, 5′-(4,4′-Dimethoxytrityl)-N-isobutyryl-2′,3′-seco-guanosine, 2′-benzoyl-3′-[(2-cyanoethyl)-(N,N-diisopropyl)]-phosphoramidite, and 5′-(4,4′-Dimethoxy-trityl)-2′,3′-seco-uridine, 2′-benzoyl-3′-[(2-cyanoethyl)-(N,N-diiso-propyl)]-phosphoramidite. TFA aminolink phosphoramidites were also commercially purchased (ThermoFisher). Linker L6 was purchased as propargyl-PEG5-NHS from BroadPharm (catalog #BP-20907) and coupled to the NH₂-C₆ group from an aminolink phosphoramidite to form -L6-C6-, using standard coupling conditions. The linker Alk-cyHex was similarly commercially purchased from Lumiprobe (alkyne phosphoramidite, 5′-terminal) as a propargyl-containing compound phosphoramidite compound to form the linker -Alk-cyHex-. In each case, phosphorothioate linkages were introduced as specified using the conditions set forth herein. The cyclopropyl phosphonate phosphoramidites were synthesized in accordance with International Patent Application Publication No. WO 2017/214112 (see also Altenhofer et. al., Chem. Communications (Royal Soc. Chem.), 57(55):6808-6811 (July 2021)). The (NAG37)s targeting ligand phosphoramidite compounds used in synthesizing the RNAi agents disclosed herein for performing certain SEAP studies described below were synthesized in accordance with International Patent Application Publication No. WO 2018/044350 to Arrowhead Pharmaceuticals, Inc.; the targeting ligand-containing phosphoramidite compounds were added during the solid phase oligonucleotide synthesis process described herein.

Tri-alkyne-containing phosphoramidites were dissolved in anhydrous dichloromethane or anhydrous acetonitrile (50 mM), while all other amidites were dissolved in anhydrous acetonitrile (50 mM) and molecular sieves (3 Å) were added. 5-Benzylthio-1H-tetrazole (BTT, 250 mM in acetonitrile) or 5-Ethylthio-1H-tetrazole (ETT, 250 mM in acetonitrile) was used as activator solution. Coupling times were 10 minutes (RNA), 90 seconds (2′ O-Me), and 60 seconds (2′ F). In order to introduce phosphorothioate linkages, a 100 mM solution of 3-phenyl 1,2,4-dithiazoline-5-one (POS, obtained from PolyOrg, Inc., Leominster, MA, USA) in anhydrous acetonitrile was employed.

Alternatively, tri-alkyne moieties were introduced post-synthetically (see section E, below). For this route, the sense strand was functionalized with a 5′ and/or 3′ terminal nucleotide containing a primary amine. TFA aminolink phosphoramidite was dissolved in anhydrous acetonitrile (50 mM) and molecular sieves (3 Å) were added. 5-Benzylthio-1H-tetrazole (BTT, 250 mM in acetonitrile) or 5-Ethylthio-1H-tetrazole (ETT, 250 mM in acetonitrile) was used as activator solution. Coupling times were 10 minutes (RNA), 90 seconds (2′ O-Me), and 60 seconds (2′ F). In order to introduce phosphorothioate linkages, a 100 mM solution of 3-phenyl 1,2,4-dithiazoline-5-one (POS, obtained from PolyOrg, Inc., Leominster, MA, USA) in anhydrous acetonitrile was employed.

### B. Cleavage and Deprotection of Support Bound Oligomer

After finalization of the solid phase synthesis, the dried solid support was treated with a 1:1 volume solution of 40 wt. % methylamine in water and 28% to 31% ammonium hydroxide solution (Aldrich) for 1.5 hours at 30° C. The solution was evaporated and the solid residue was reconstituted in water (see below).

### C. Purification

Crude oligomers were purified by anionic exchange HPLC using a TSKgel SuperQ-5PW 13 μm column and Shimadzu LC-8 system. Buffer A was 20 mM Tris, 5 mM EDTA, pH 9.0 and contained 20% Acetonitrile and buffer B was the same as buffer A with the addition of 1.5 M sodium chloride. UV traces at 260 nm were recorded. Appropriate fractions were pooled then run on size exclusion HPLC using a GE Healthcare XK 16/40 column packed with Sephadex G-25 fine with a running buffer of 100 mM ammonium bicarbonate, pH 6.7 and 20% Acetonitrile or filtered water. Alternatively, pooled fractions were desalted and exchanged into an appropriate buffer or solvent system via tangential flow filtration.

### D. Annealing

Complementary strands were mixed by combining equimolar RNA solutions (sense and antisense) in 1×PBS (Phosphate-Buffered Saline, 1×, Corning, Cellgro) to form the RNAi agents. Some RNAi agents were lyophilized and stored at −15 to −25° C. Duplex concentration was determined by measuring the solution absorbance on a UV-Vis spectrometer in 1×PBS. The solution absorbance at 260 nm was then multiplied by a conversion factor (0.050 mg/(mL-cm)) and the dilution factor to determine the duplex concentration.

### E. Conjugation of Tri-Alkyne Linker

In some embodiments a tri-alkyne linker is conjugated to the sense strand of the RNAi agent on resin as a phosphoramidite (see Example 1G for the synthesis of an example tri-alkyne linker phosphoramidite and Example 1A for the conjugation of the phosphoramidite.). In other embodiments, a tri-alkyne linker may be conjugated to the sense strand following cleavage from the resin, described as follows: either prior to or after annealing, in some embodiments, the 5′ or 3′ amine functionalized sense strand is conjugated to a tri-alkyne linker. An example tri-alkyne linker structure that can be used in forming the constructs disclosed herein is as follows:

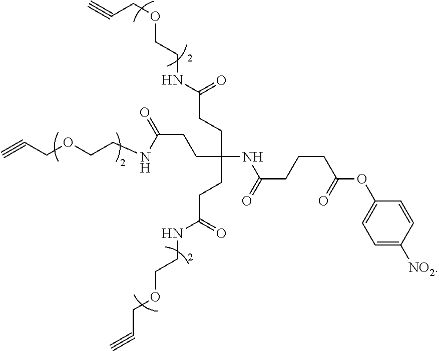

To conjugate the tri-alkyne linker to the annealed duplex, amine-functionalized duplex was dissolved in 90% DMSO/10% H₂O, at ˜50-70 mg/mL. 40 equivalents triethylamine was added, followed by 3 equivalents tri-alkyne-PNP. Once complete, the conjugate was precipitated twice in a solvent system of 1× phosphate buffered saline/acetonitrile (1:14 ratio), and dried.

### F. Synthesis of Targeting Ligand SM6.1 ((S)-3-(4-(4-((14-azido-3,6,9,12-tetraoxatetradecyl)oxy)naphthalen-1-yl)phenyl)-3-(2-(4-((4-methylpyridin-2-yl)amino)butanamido)acetamido)propanoic acid)

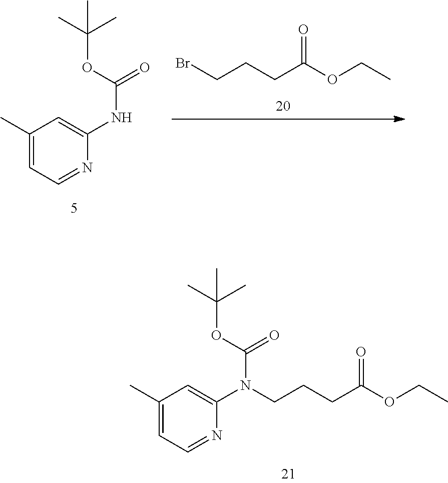

Compound 5 (tert-Butyl(4-methylpyridin-2-yl)carbamate) (0.501 g, 2.406 mmol, 1 equiv.) was dissolved in DMF (17 mL). To the mixture was added NaH (0.116 mg, 3.01 mmol, 1.25 eq, 60% dispersion in oil) The mixture stirred for 10 min before adding Compound 20 (Ethyl 4-Bromobutyrate (0.745 g, 3.82 mmol, 0.547 mL)) (Sigma 167118). After 3 hours the reaction was quenched with ethanol (18 mL) and concentrated. The concentrate was dissolved in DCM (50 mL) and washed with saturated aq. NaCl solution (1×50 mL), dried over Na₂SO₄, filtered and concentrated. The product was purified on silica column, gradient 0-5% Methanol in DCM.

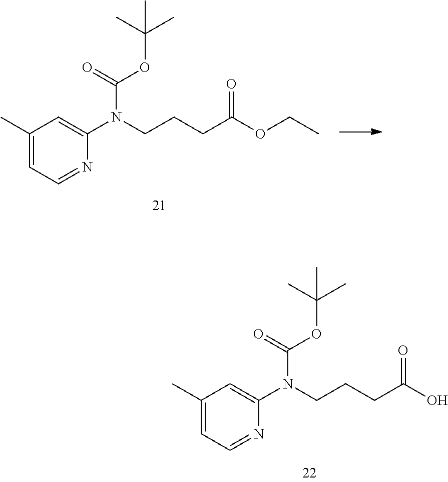

Compound 21 was dissolved (0.80 g, 2.378 mmol) in 100 mL of Acetone:0.1 M NaOH [1:1]. The reaction was monitored by TLC (5% ethyl acetate in hexane). The organics were concentrated away, and the residue was acidified to pH 3-4 with 0.3 M Citric Acid (40 mL). The product was extracted with DCM (3×75 mL). The organics were pooled, dried over Na₂SO₄, filtered and concentrated. The product was used without further purification.

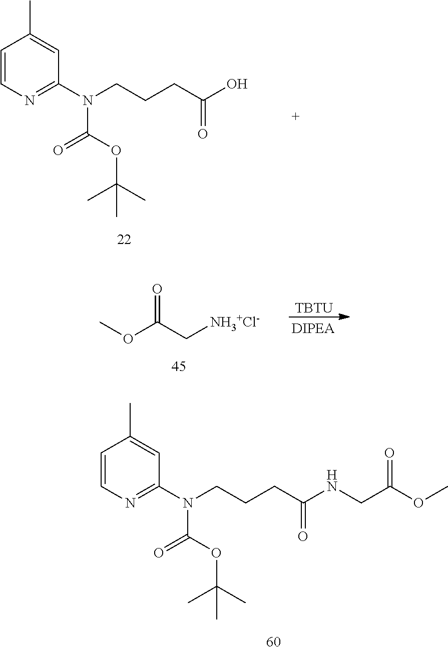

To a solution of Compound 22 (1.1 g, 3.95 mmol, 1 equiv.), Compound 45 (595 mg, 4.74 mmol, 1.2 equiv.), and TBTU (1.52 g, 4.74 mmol, 1.2 equiv.) in anhydrous DMF (10 mL) was added diisopropylethylamine (2.06 mL, 11.85 mmol, 3 equiv.) at 0° C. The reaction mixture was warmed to room temperature and stirred 3 hours. The reaction was quenched by saturated NaHCO₃ solution (10 mL). The aqueous phase was extracted with ethyl acetate (3×10 mL) and the organic phase was combined, dried over anhydrous Na₂SO₄, and concentrated. The product was separated by CombiFlash® using silica gel as the stationary phase. LC-MS: calculated [M+H]+ 366.20, found 367.

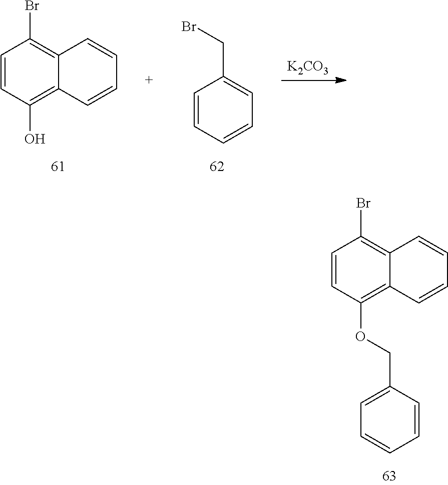

To a solution of compound 61 (2 g, 8.96 mmol, 1 equiv.), and compound 62 (2.13 mL, 17.93 mmol, 2 equiv.) in anhydrous DMF (10 mL) was added K₂CO₃ (2.48 g, 17.93 mmol, 2 equiv.) at 0° C. The reaction mixture was warmed to room temperature and stirred overnight. The reaction was quenched by water (10 mL). The aqueous phase was extracted with ethyl acetate (3×10 mL) and the organic phase was combined, dried over anhydrous Na₂SO₄, and concentrated. The product was separated by CombiFlash® using silica gel as the stationary phase.

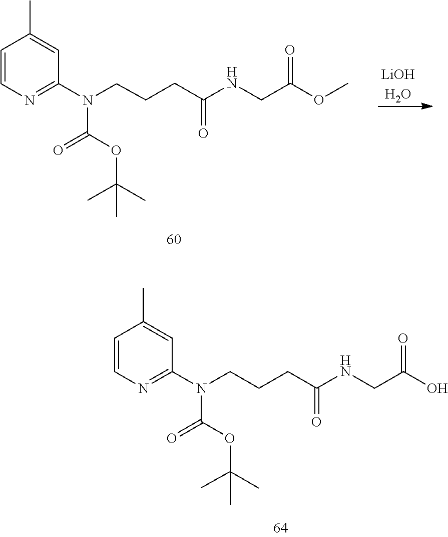

To a solution of compound 60 (1.77 g, 4.84 mmol, 1 equiv.) in THF (5 mL) and H₂O (5 mL) was added lithium hydroxide monohydrate (0.61 g, 14.53 mmol, 3 equiv.) portion-wise at 0° C. The reaction mixture was warmed to room temperature. After stirring at room temperature for 3 hours, the reaction mixture was acidified by HCl (6 N) to pH 3.0. The aqueous phase was extracted with ethyl acetate (3×20 mL) and the organic layer was combined, dried over Na₂SO₄, and concentrated. LC-MS: calculated [M+H]+ 352.18, found 352.

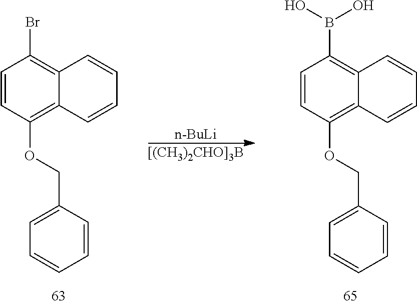

To a solution of compound 63 (1.88 g, 6.0 mmol, 1.0 equiv.) in anhydrous THF (20 mL) was added n-BuLi in hexane (3.6 mL, 9.0 mmol, 1.5 equiv.) drop-wise at −78° C. The reaction was kept at −78° C. for another 1 hour. Triisopropylborate (2.08 mL, 9.0 mmol, 1.5 equiv.) was then added into the mixture at −78° C. The reaction was then warmed up to room temperature and stirred for another 1 hour. The reaction was quenched by saturated NH₄Cl solution (20 mL) and the pH was adjusted to 3. The aqueous phase was extracted with EtOAc (3×20 mL) and the organic phase was combined, dried over Na₂SO₄, and concentrated.

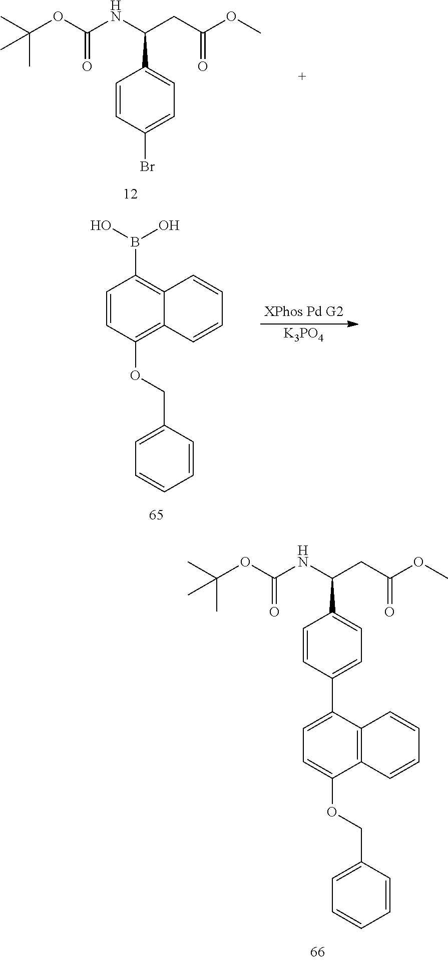

Compound 12 (300 mg, 0.837 mmol, 1.0 equiv.), Compound 65 (349 mg, 1.256 mmol, 1.5 equiv.), XPhos Pd G2 (13 mg, 0.0167 mmol, 0.02 equiv.), and K₃PO₄ (355 mg, 1.675 mmol, 2.0 equiv.) were mixed in a round-bottom flask. The flask was sealed with a screw-cap septum, and then evacuated and backfilled with nitrogen (this process was repeated a total of 3 times). Then, THF (8 mL) and water (2 mL) were added via syringe. The mixture was bubbled with nitrogen for 20 min and the reaction was kept at room temperature for overnight. The reaction was quenched with water (10 mL), and the aqueous phase was extracted with ethyl acetate (3×10 mL). The organic phase was dried over Na₂SO₄, concentrated, and purified via CombiFlash® using silica gel as the stationary phase and was eluted with 15% EtOAc in hexane. LC-MS: calculated [M+H]+ 512.24, found 512.56.

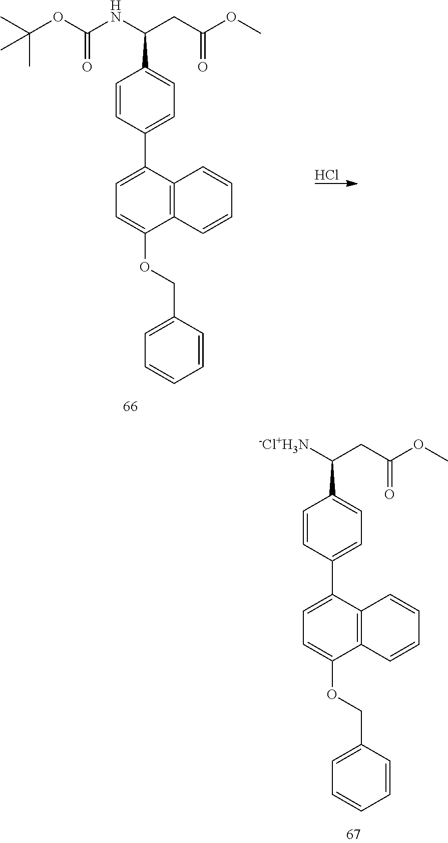

Compound 66 (858 mg, 1.677 mmol, 1.0 equiv.) was cooled by ice bath. HCl in dioxane (8.4 mL, 33.54 mmol, 20 equiv.) was added into the flask. The reaction was warmed to room temperature and stirred for another 1 hr. The solvent was removed by rotary evaporator and the product was directly used without further purification. LC-MS: calculated [M+H]+ 412.18, found 412.46.

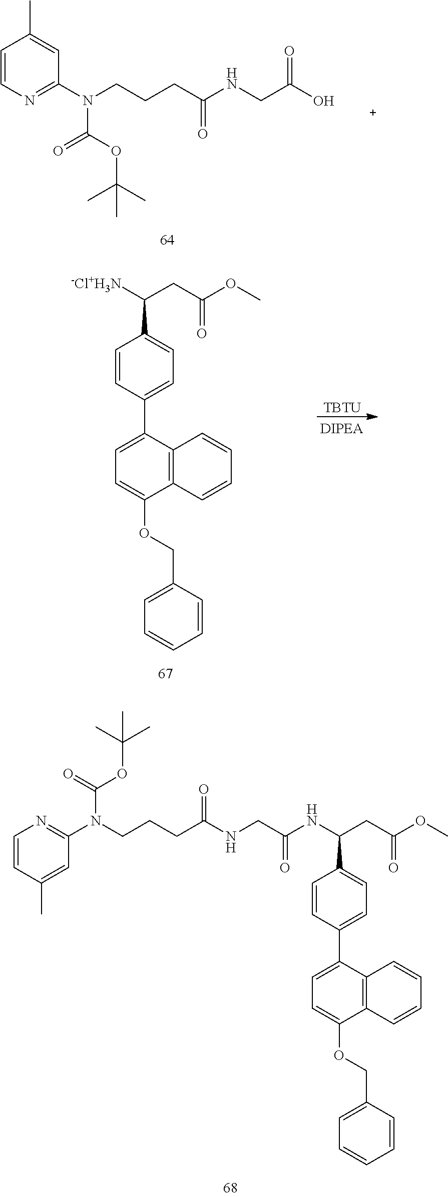

To a solution of compound 64 (500 mg, 1.423 mmol, 1 equiv.), compound 67 (669 mg, 1.494 mmol, 1.05 equiv.), and TBTU (548 mg, 0.492 mmol, 1.2 equiv.) in anhydrous DMF (15 mL) was added diisopropylethylamine (0.744 mL, 4.268 mmol, 3 equiv.) at 0° C. The reaction mixture was warmed to room temperature and stirred for another 1 hr. The reaction was quenched by saturated NaHCO₃ aqueous solution (10 mL) and the product was extracted with ethyl acetate (3×20 mL). The organic phase was combined, dried over Na₂SO₄, and concentrated. The product was purified by CombiFlash® using silica gel as the stationary phase and was eluted with 3-4% methanol in DCM. The yield was 96.23%. LC-MS: calculated [M+H]+ 745.35, found 746.08.

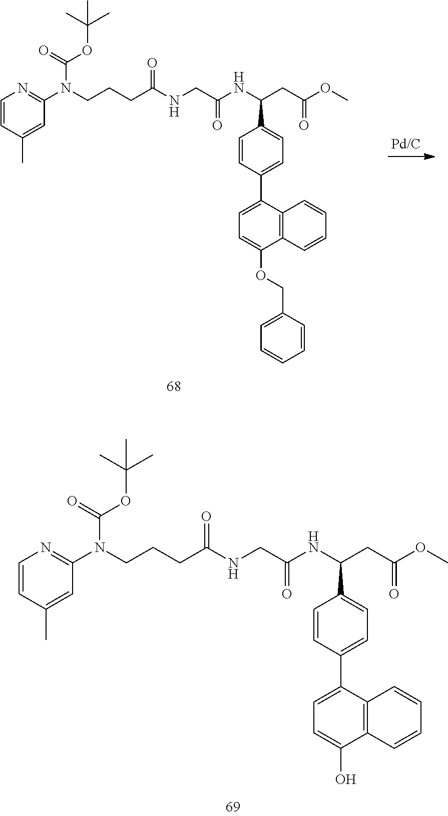 [SMILES: COC(=O)C[C@H](NC(=O)CNC(=O)CCCN(C(=O)OC(C)(C)C)c1cc(C)ccn1)c1ccc(-c2ccc(OCc3ccccc3)c3ccccc23)cc1 ; COC(=O)C[C@H](NC(=O)CNC(=O)CCCN(C(=O)OC(C)(C)C)c1cc(C)ccn1)c1ccc(-c2ccc(O)c3ccccc23)cc1]

To a solution of compound 68 (1.02 g, 1.369 mmol, 1 equiv.) in ethyl acetate (10 mL) was added 10% Pd/C (0.15 g, 50% H₂O) at room temperature. The reaction mixture was warmed to room temperature and the reaction was monitored by LC-MS. The reaction was kept at room temperature overnight. The solids were filtered through Celite® and the solvent was removed by rotary evaporator. The product was directly used without further purification. LC-MS: [M+H]+ 655.31, found 655.87.

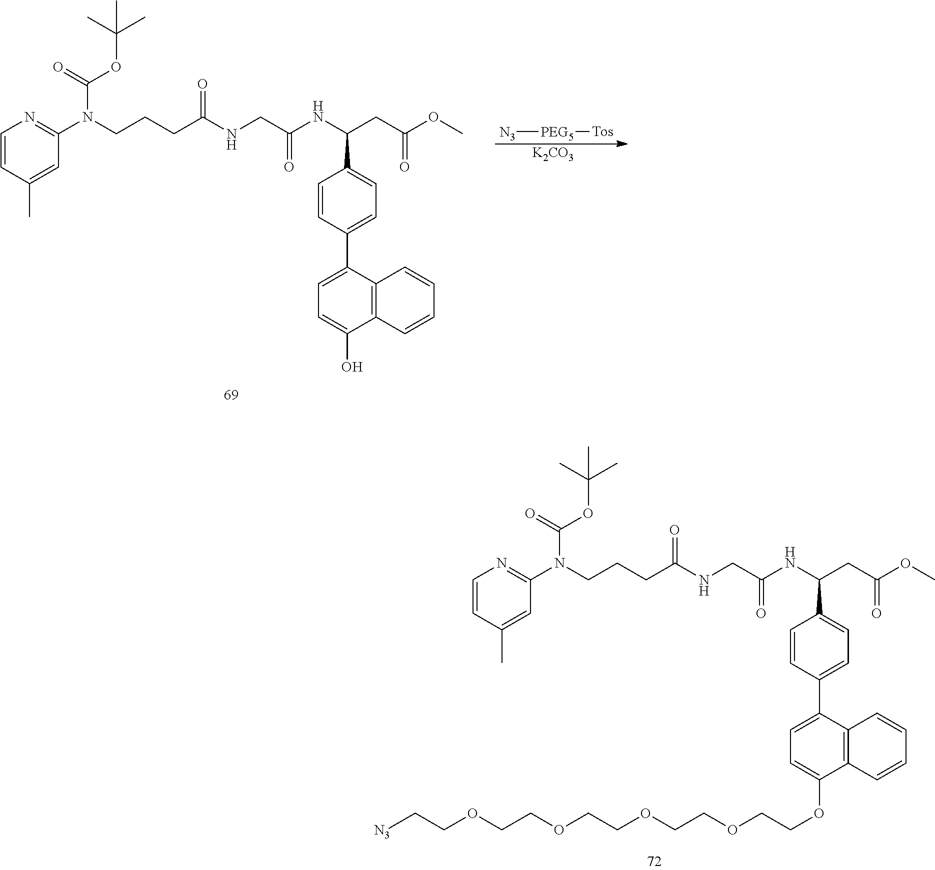

To a solution of compound 69 (100 mg, 0.152 mmol, 1 equiv.) and azido-PEG₅-OTs (128 mg, 0.305 mmol, 2 equiv.) in anhydrous DMF (2 mL) was added K₂CO₃ (42 mg, 0.305 mmol, 2 equiv.) at 0° C. The reaction mixture was stirred for 6 hours at 80° C. The reaction was quenched by saturated NaHCO₃ solution and the aqueous layer was extracted with ethyl acetate (3×10 mL). The organic phase was combined, dried over Na₂SO₄, and concentrated. LC-MS: calculated [M+H]+ 900.40, found 901.46.

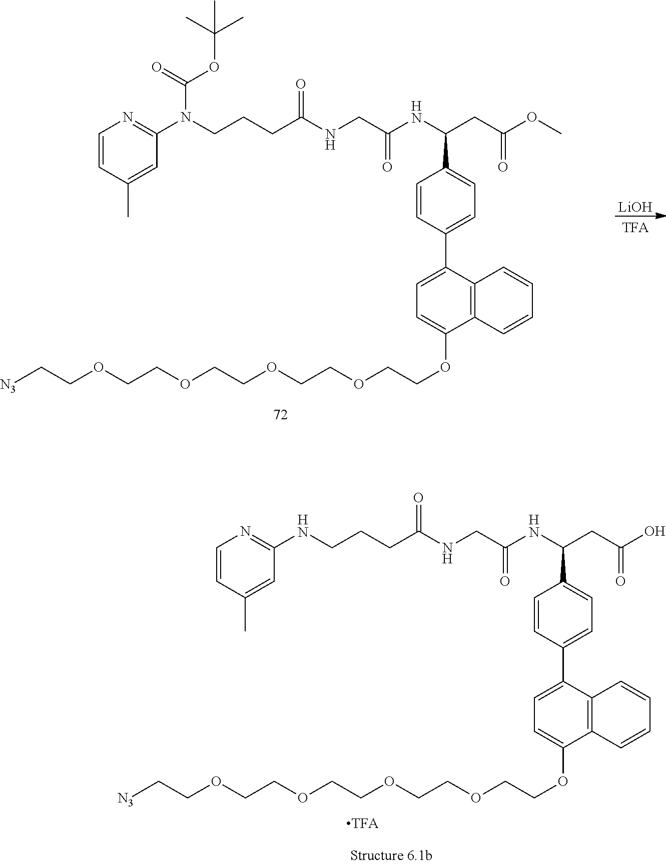 [SMILES: COC(=O)C[C@H](NC(=O)CNC(=O)CCCN(C(=O)OC(C)(C)C)c1cc(C)ccn1)c1ccc(-c2ccc(OCCOCCOCCOCCOCCN=[N+]=[N-])c3ccccc23)cc1 ; "LiOH" ; Cc1ccnc(NCCCC(=O)NCC(=O)N[C@@H](CC(=O)O)c2ccc(-c3ccc(OCCOCCOCCOCCOCCN=[N+]=[N-])c4ccccc34)cc2)c1]

To a solution of compound 72 (59 mg, 0.0656 mmol, 1.0 equiv.) in THF (2 mL) and water (2 mL) was added lithium hydroxide (5 mg, 0.197 mmol, 3.0 equiv.) at room temperature. The mixture was stirred at room temperature for another 1 hr. The pH was adjusted to 3.0 by HCl (6N) and the aqueous phase was extracted with EtOAc (3×10 mL). The organic phase was combined, dried over Na₂SO₄, and concentrated. TFA (0.5 mL) and DCM (0.5 mL) was added into the residue and the mixture was stirred at room temperature for another 3 hr. The solvent was removed by rotary evaporator. LC-MS: calculated [M+H]+ 786.37, found 786.95.

### G. Synthesis of TriAlk 14

TriAlk14 and (TriAlk14)s as shown in Table 11, above, may be synthesized using the synthetic route shown below. Compound 14 may be added to the sense strand as a phosphoramidite using standard oligonucleotide synthesis techniques, or compound 22 may be conjugated to the sense strand comprising an amine in an amide coupling reaction.

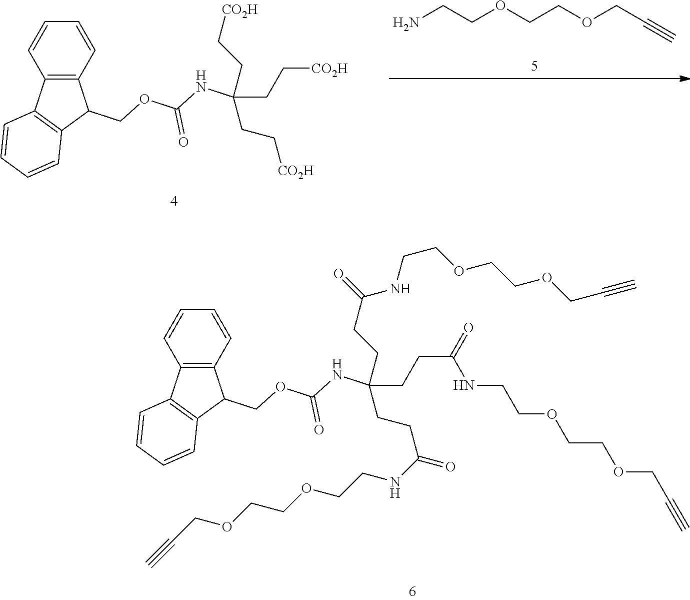

To a 3-L jacketed reactor was added 500 mL DCM and 4 (75.0 g, 0.16 mol). The internal temperature of the reaction was cooled to 0° C. and TBTU (170.0 g, 0.53 mol) was added. The suspension was then treated with the amine 5 (75.5 g, 0.53 mol) dropwise keeping the internal temperature less than 5° C. The reaction was then treated with DIPEA (72.3 g, 0.56 mol) slowly, keeping the internal temperature less than 5° C. After the addition was complete, the reaction was warmed up to 23° C. over 1 hour, and allowed to stir for 3 hours. A 10% kicker charge of all three reagents were added and allowed to stir an additional 3 hours. The reaction was deemed complete when <1% of 4 remained. The reaction mixture was washed with saturated ammonium chloride solution (2×500 mL) and once with saturated sodium bicarbonate solution (500 mL). The organic layer was then dried over sodium sulfate and concentrated to an oil. The mass of the crude oil was 188 g which contained 72% 6 by QNMR. The crude oil was carried to the next step. Calculated mass for C₄₆H₆₀N₄O₁₁=845.0 m/z. Found [M+H]=846.0.

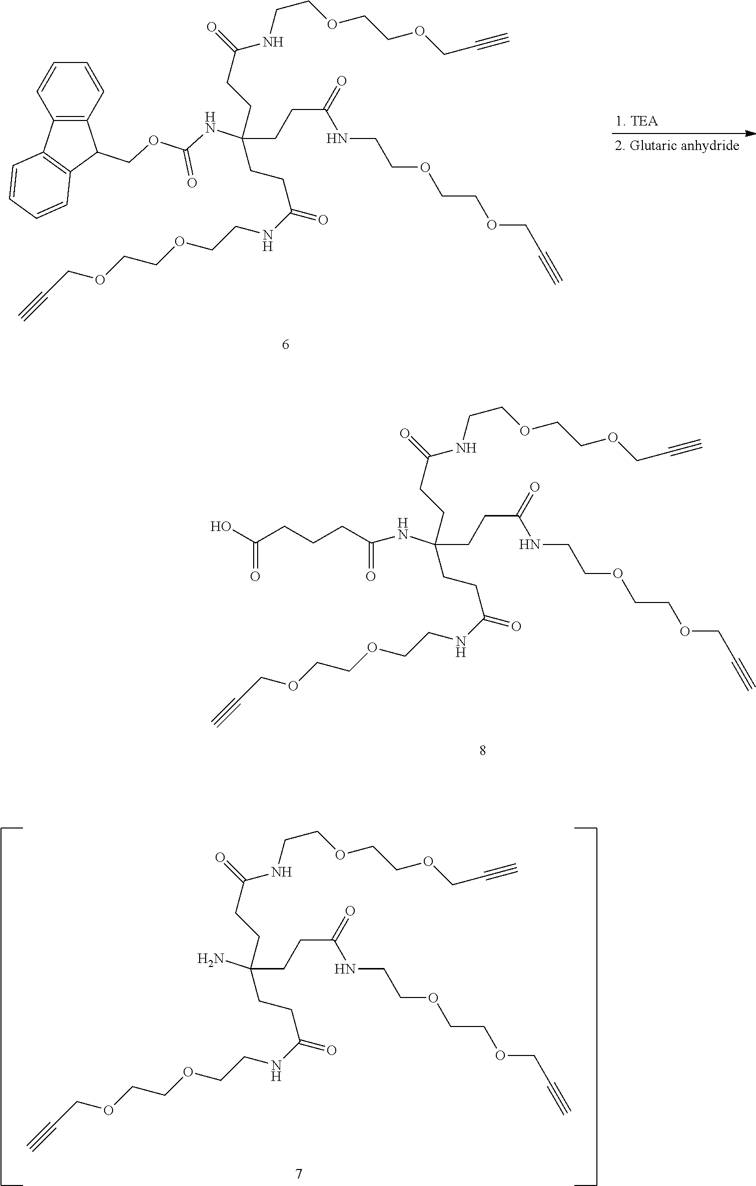

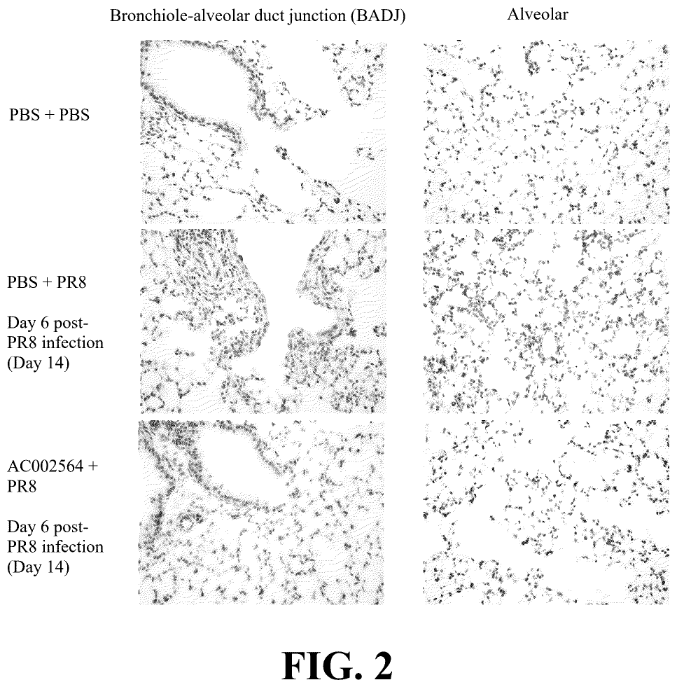

The 121.2 g of crude oil containing 72 wt % compound 6 (86.0 g, 0.10 mol) was dissolved in DMF (344 mL) and treated with TEA (86 mL, 20 v/v %), keeping the internal temperature below 23° C. The formation of dibenzofulvene (DBF) relative to the consumption of Fmoc-amine 6 was monitored via HPLC method 1 (FIG. 2) and the reaction was complete within 10 hours. To the solution was added glutaric anhydride (12.8 g, 0.11 mol) and the intermediate amine 7 was converted to compound 8 within 2 hours. Upon completion, the DMF and TEA were removed at 30° C. under reduced pressure resulting in 100 g of a crude oil. Due to the high solubility of compound 7 in water, an aqueous workup could not be used, and chromatography is the only way to remove DBF, TMU, and glutaric anhydride. The crude oil (75 g) was purified on a Teledyne ISCO Combi-Flash® purification system in three portions. The crude oil (25 g) was loaded onto a 330 g silica column and eluted from 0-20% methanol/DCM over 30 minutes resulting in 42 g of compound 8 (54% yield over 3 steps). Calculated mass for C₃₆H₅₅N₄O₁₂=736.4 m/z. Found [M+H]=737.0.

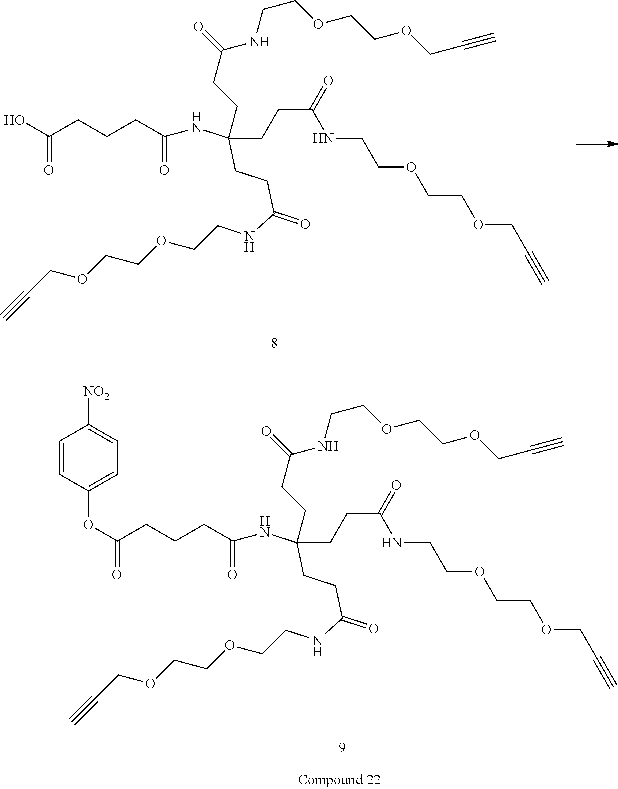 [SMILES: C#CCOCCOCCNC(=O)CCC(CCC(=O)NCCOCCOCC#C)(CCC(=O)NCCOCCOCC#C)NC(=O)CCCC(=O)O ; C#CCOCCOCCNC(=O)CCC(CCC(=O)NCCOCCOCC#C)(CCC(=O)NCCOCCOCC#C)NC(=O)CCCC(=O)Oc1ccc([N+](=O)[O-])cc1]

Compound 8 (42.0 g, 0.057 mol) was co-stripped with 10 volumes of acetonitrile prior to use to remove any residual methanol from chromatography solvents. The oil was redissolved in DMF (210 mL) and cooled to 0° C. The solution was treated with 4-nitrophenol (8.7 g, 0.063 moL) followed by EDC-hydrochloride (12.0 g, 0.063 mol) and found to reach completion within 10 hours. The solution was cooled to 0° C. and 10 volumes ethyl acetate was added followed by 10 volumes saturated ammonium chloride solution, keeping the internal temperature below 15° C. The layers were allowed to separate and the ethyl acetate layer was washed with brine. The combined aqueous layers were extracted twice with 5 volumes ethyl acetate. The combined organic layers were dried over sodium sulfate and concentrated to an oil. The crude oil (55 g) was purified on a Teledyne ISCO Combi-Flash® purification system in three portions. The crude oil (25 g) was loaded onto a 330 g silica column and eluted from 0-10% methanol/DCM over 30 minutes resulting in 22 g of pure 9 (Compound 22) (50% yield). Calculated mass for C₄₂H₅₉N₅O₁₄=857.4 m/z. Found [M+H]=858.0.

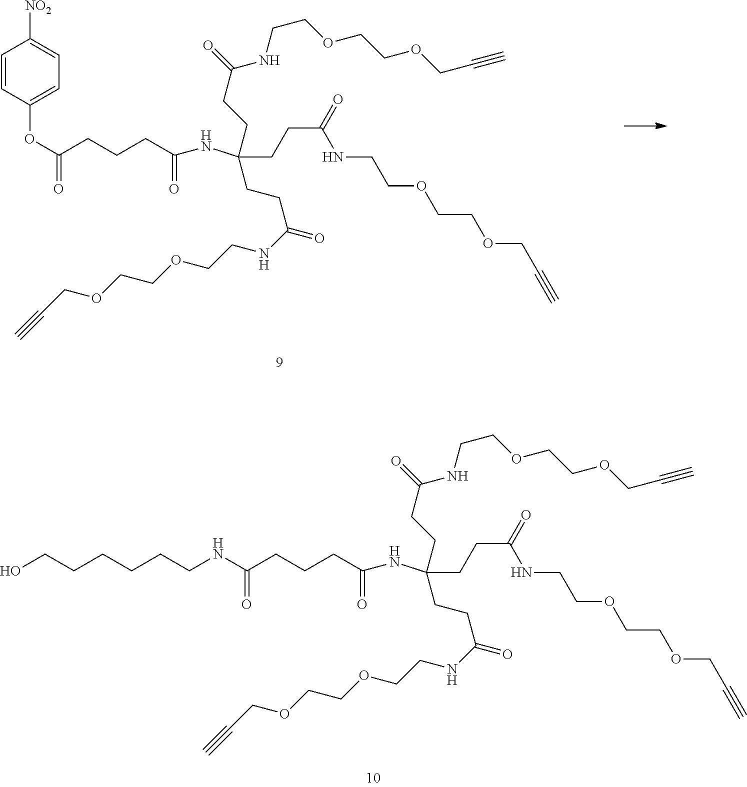 [SMILES: C#CCOCCOCCNC(=O)CCC(CCC(=O)NCCOCCOCC#C)(CCC(=O)NCCOCCOCC#C)NC(=O)CCCC(=O)Oc1ccc([N+](=O)[O-])cc1 ; C#CCOCCOCCNC(=O)CCC(CCC(=O)NCCOCCOCC#C)(CCC(=O)NCCOCCOCC#C)NC(=O)CCCC(=O)NCCCCCCO]

A solution of ester 9 (49.0 g, 57.1 mmol) and 6-amino-1-hexanol (7.36 g, 6.28 mmol) in dichloromethane (3 volumes) was treated with triethylamine (11.56 g, 111.4 mmol) dropwise. The reaction was monitored by observing the disappearance of compound 9 on HPLC Method 1 and was found to be complete in 10 minutes. The crude reaction mixture was diluted with 5 volumes dichloromethane and washed with saturated ammonium chloride (5 volumes) and brine (5 volumes). The organic layer was dried over sodium sulfate and concentrated to an oil. The crude oil was purified on a Teledyne ISCO Combi-Flash® purification system using a 330 g silica column. The 4-nitrophenol was eluted with 100% ethyl acetate and 10 was flushed from the column using 20% methanol/DCM resulting in a colorless oil (39 g, 81% yield). Calculated mass for C₄₂H₆₉N₅O₁₂=836.0 m/z. Found [M+H]=837.0.

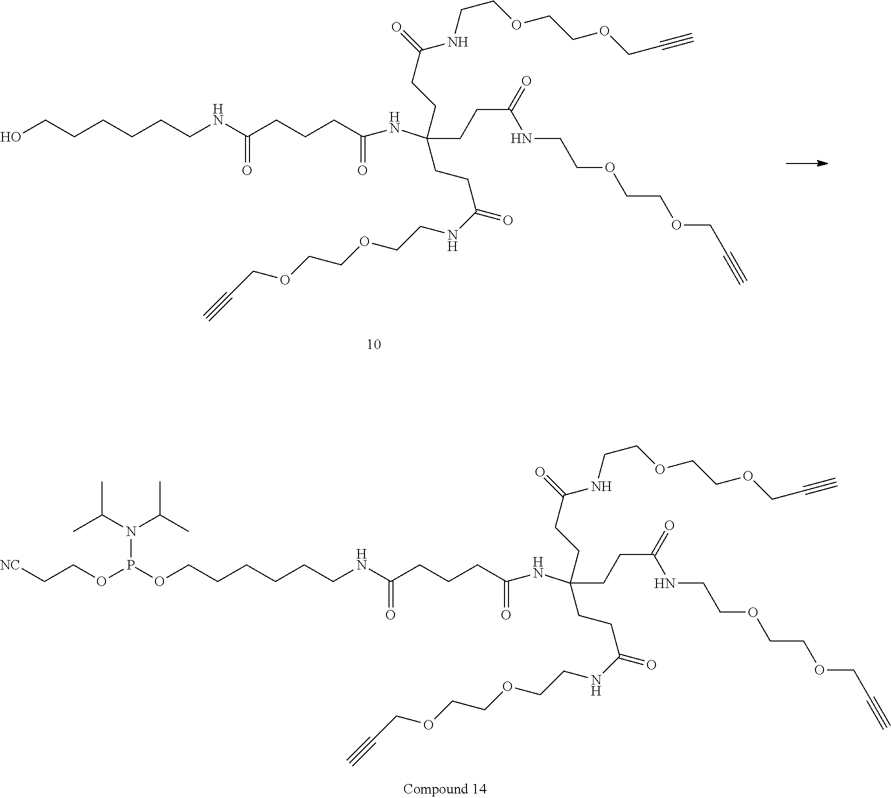 [SMILES: C#CCOCCOCCNC(=O)CCC(CCC(=O)NCCOCCOCC#C)(CCC(=O)NCCOCCOCC#C)NC(=O)CCCC(=O)NCCCCCCO ; C#CCOCCOCCNC(=O)CCC(CCC(=O)NCCOCCOCC#C)(CCC(=O)NCCOCCOCC#C)NC(=O)CCCC(=O)NCCCCCCOP(OCCC#N)N(C(C)C)C(C)C]

Alcohol 10 was co-stripped twice with 10 volumes of acetonitrile to remove any residual methanol from chromatography solvents and once more with dry dichloromethane (KF <60 ppm) to remove trace water. The alcohol 10 (2.30 g, 2.8 mmol) was dissolved in 5 volumes dry dichloromethane (KF<50 ppm) and treated with diisopropylammonium tetrazolide (188 mg, 1.1 mmol). The solution was cooled to 0° C. and treated with 2-cyanoethyl N,N,N′,N′-tetraisopropylphosphoramidite (1.00 g, 3.3 mmol) dropwise. The solution was removed from ice-bath and stirred at 20° C. The reaction was found to be complete within 3-6 hours. The reaction mixture was cooled to 0° C. and treated with 10 volumes of a 1:1 solution of saturated ammonium bicarbonate/brine and then warmed to ambient over 1 minute and allowed to stir an additional 3 minutes at 20° C. The biphasic mixture was transferred to a separatory funnel and 10 volumes of dichloromethane was added. The organic layer was separated and washed with 10 volumes of saturated sodium bicarbonate solution to hydrolyze unreacted bis-phosphorous reagent. The organic layer was dried over sodium sulfate and concentrated to an oil resulting in 3.08 g of 94 wt % Compound 14. Calculated mass for C₅₁H₈₆N₇O₁₃P=1035.6 m/z. Found [M+H]=1036.

### H. Conjugation of Targeting Ligands

Either prior to or after annealing, the 5′ or 3′ tridentate alkyne functionalized sense strand is conjugated to targeting ligands. The following example describes the conjugation of targeting ligands to the annealed duplex: Stock solutions of 0.5M Tris(3-hydroxypropyltriazolylmethyl)amine (THPTA), 0.5M of Cu(II) sulfate pentahydrate (Cu(II)SO₄·5H₂O) and 2M solution of sodium ascorbate were prepared in deionized water. A 75 mg/mL solution in DMSO of targeting ligand was made. In a 1.5 mL centrifuge tube containing tri-alkyne functionalized duplex (3 mg, 75 μL, 40 mg/mL in deionized water, ˜15,000 g/mol), 25 μL of 1M Hepes pH 8.5 buffer is added. After vortexing, 35 μL of DMSO was added and the solution is vortexed. Targeting ligand was added to the reaction (6 equivalents/duplex, 2 equivalents/alkyne, ˜15 μL) and the solution is vortexed. Using pH paper, pH was checked and confirmed to be pH ˜8. In a separate 1.5 mL centrifuge tube, 50 μL of 0.5M THPTA was mixed with 10 uL of 0.5M Cu(II)SO₄·5H₂O, vortexed, and incubated at room temp for 5 min. After 5 min, THPTA/Cu solution (7.2 μL, 6 equivalents 5:1 THPTA:Cu) was added to the reaction vial, and vortexed. Immediately afterwards, 2M ascorbate (5 μL, 50 equivalents per duplex, 16.7 per alkyne) was added to the reaction vial and vortexed. Once the reaction was complete (typically complete in 0.5-1 h), the reaction was immediately purified by non-denaturing anion exchange chromatography.

### Example 2. Influenza A/Puerto Rico/8/34 PR8 Mouse Model

To study the effects of the IAV RNAi agents, the Influenza A/Puerto Rico/8/34 PR8 mouse model (“PR8 mouse model”) was established. The influenza A/Puerto Rico/8/34 (PR8) strain is a Biosafety Level 2 (BSL2) level virus that has been widely used in the laboratory as a mouse influenza model for studying acute lung injury/inflammation. PR8 has been previously shown to cause severe pathogenicity in mice (CF Basler, et al., Sequence of the 1918 pandemic influenza virus nonstructural gene (NS) segment and characterization of recombinant viruses bearing the 1918 NS genes. Proc Natl Acad Sci USA 98, 2746-2751 (2001)). PR8 has had over 100 passages in each of mice, ferrets, and embryonated chicken eggs, resulting in complete attenuation of the virus and its inability to replicate in humans (Annex 5, WHO Technical Report Series No 941, 2007).

C57BL/6 mice were infected with PR8, and the PR8-infected mice were subsequently administered IAV RNAi agents. C57BL/6 mice were also first administered IAV RNAi agents, and then subsequently infected with PR8. The optimal sub-lethal dose of PR8 infection was determined upon examination of the body weight loss after PR8 infection. If animal weight loss exceeded 20% of the pre-dose body weight, the viral dose was determined to be lethal and not optimal. Accordingly, the dose of the PR8 was subsequently adjusted for optimal sub-lethal dose.

### Example 3. Influenza A/California/07/2009 H1N1 Mouse Model

To study the effects of the IAV RNAi agents, the Influenza A/California/07/2009 H1N1 mouse model (“CA07 H1N1 mouse model”) was established. The influenza A/California/07/2009 (H1N1) (hereinafter “CA07 H1N1”) strain is a Biosafety Level 2 (BSL2) level virus, and has emerged with rapid human-to-human spread and caused the first pandemic of the 21st century. Rockman S, Laurie K, Barr I. Pandemic Influenza Vaccines: What did We Learn from the 2009 Pandemic and are We Better Prepared Now? Vaccines (Basel). 2020 May 7; 8(2):211. doi: 10.3390/vaccines8020211. PMID: 32392812; PMCID: PMC7349738. This virus replaced the previous A(H1N1), and continues to circulate today as a seasonal virus.

C57BL/6 mice were infected with CA07 H1N1, and the CA07 H1N1-infected mice were subsequently administered IAV RNAi agents. C57BL/6 mice were also first administered IAV RNAi agents, and then subsequently infected with CA07 H1N1. The optimal sub-lethal dose of CA07 H1N1 infection was determined upon examination of the body weight loss after CA07 H1N1 infection. If animal weight loss exceeded 20% of the pre-dose body weight, the viral dose was determined to be lethal and not optimal. Accordingly, the dose of the CA07 H1N1 was subsequently adjusted for optimal sub-lethal dose.

### Example 4. In Vivo Administration of IAV RNAi Agents to Mice Subsequently Infected with PR8

Female C57Bl/6 mice animals were dosed with IAV RNAi agents and subsequently infected with PR8, in accordance with Example 3 (“PR8 model mice”). On Days 1 and 3, four (n=4) mice (for Group 1) and six (n=6) mice (for Groups 2-6) were dosed with either saline or IAV RNAi agent formulated in saline (at 3 mg/kg), via intratracheal (IT) administration. On Day 8, the animals were dosed with either PBS or PR8 (BEI) formulated in PBS, via intranasal (IN) injection. Dosing was in accordance with Table 12 below.

TABLE 12
Dosing for mice animals of Example 4

| Group | Dose (RNAi Agent) | Dose (PR8) |
|---|---|---|
| 1 | Day 1 and 3: IT 50 μL Saline | Day 8: IN 100 μL PBS |
| 2 | Day 1 and 3: IT 50 μL Saline | Day 8: IN 100 μL PR8 |
| | | (938EID50) |
| 3 | Day 1 and 3: IT 50 μL 3 mg/kg | Day 8: IN 100 μL PR8 |
| | AC002564 | (938EID50) |
| 4 | Day 1 and 3: IT 50 μL 3 mg/kg | Day 8: IN 100 μL PR8 |
| | AC002567 | (938EID50) |
| 5 | Day 1 and 3: IT 50 μL 3 mg/kg | Day 8: IN 100 μL PR8 |
| | AC002568 | (938EID50) |
| 6 | Day 1 and 3: IT 50 μL 3 mg/kg | Day 8: IN 100 μL PR8 |
| | AC002569 | (938EID50) |

PR8 dose was quantified in EID50 (50% egg infective dose). This EID50 dose was determined to be the optimal sub-lethal dose in accordance with the PR8 mouse model described in Example 2, above.

On Day 14, the animals were sacrificed. Bronchoalveolar lavage fluid (BALF) and lungs (both left and right) were collected and harvested. Expression of mouse H1N1 and M1 in right lung was determined using qPCR, with 18S rRNA as endogenous control gene, normalized to Group 2. Average H1N1 and M1 expression for each animal in lung tissue was normalized relative to Group 2 (no RNAi agent+PR8 infection). Results are shown in Table 13 below.

TABLE 13
Average relative expression of HIN1 and M1 in mice lung of Example 4.

| | Avg | | | | | |
|---|---|---|---|---|---|---|
| Group ID | H1N1 | Low | High | Avg M1 | Low | High |
| 1. Saline + PBS | N/A | N/A | N/A | N/A | N/A | N/A |
| 2. Saline + PR8 | 1.000 | 0.218 | 0.278 | 1.000 | 0.196 | 0.243 |
| 3. 3 mg/kg AC002564 + | 0.093 | 0.040 | 0.069 | 0.135 | 0.054 | 0.090 |
| PR8 | | | | | | |
| 4. 3 mg/kg AC002567 + | 0.861 | 0.269 | 0.391 | 1.267 | 0.435 | 0.663 |
| PR8 | | | | | | |
| 5. 3 mg/kg AC002568 + | 0.521 | 0.182 | 0.279 | 0.627 | 0.196 | 0.284 |
| PR8 | | | | | | |
| 6. 3 mg/kg AC002569 + | 0.379 | 0.092 | 0.121 | 0.431 | 0.087 | 0.110 |
| PR8 | | | | | | |

Groups 3, 5, and 6 showed reductions in H1N1, with Group 3 (AC02564) in particular showing approximately 90% inhibition (0.093) of H1N1. Similarly, Groups 3, 5, and 6 showed reductions in M1, again with Group 3 (AC02564) showing reductions of approximately 87% (0.135). As noted in Tables 8A, 8C, 8D, and 8E, above, The IAV RNAi agent of Group 3 (AC002564) targets the M1 vRNA segment of influenza A (Table 8A); the IAV RNAi agent of Group 4 (AC002567) targets the PB1 vRNA segment of influenza A (Table 8C); the IAV RNAi agent of Group 5 (AC002568) targets the PB2 vRNA segment of influenza A (Table 8D); and the IAV RNAi agent of Group 6 (AC002569) targets the NP vRNA segment of influenza A (Table 8E). While targeting any specific vRNA segment of the influenza A genome that is conserved across multiple viral genome variants could potentially provide for a therapeutic effect, targeting the M1 vRNA segment, such as the IAV RNAi agent of Group 3 (AC002564), is a particularly promising. M1 is the most abundant protein in the influenza viron and plays critical roles in many aspects of the virus life cycle, including Influenza A viral ribonucleoprotein (vRNP) transport between the cytoplasm and the nucleus; regulation of vRNP transcription and replication; interaction with viral envelope proteins; and recruitment of viral and host components at the assembly site and initiation of budding. Mutations are extremely rare in M1. For at least these reasons, M1 is highly conserved and while it is generally regarded as the most optimal vRNA segment target for a broad-spectrum influenza A vaccine, there is no vaccine available yet targeting M1. Further while some attempts have been undertaken to develop small molecules targeting M1, none have been successful to date.

FIG. 2 shows the immunohistochemistry (IHC) of mouse PR8-infected (or non-infected PBS) lungs at 6 days post-infection, or Day 14. The mouse lung tissues were stained for anti-hemagglutinin (anti-HA) influenza A virus H1N1 IgG in accordance with manufacturer's instructions; the anti-HA immunogen is of recombinant protein encompassing a sequence within the C-terminus region of Influenza A virus H1N1 HA (Hemagglutinin) (A/WSN/1933(H1N1)) (GeneTex, Catalog #: GTX127357). As shown in FIG. 2, at Day 14 (6 days post-PR8 infection), RNAi agent AC002564 showed significantly reduced influenza A viral replication, as evidenced by the reduced anti-HA staining of the mice administered with AC002564+PR8 in comparison with the mice administered with saline (no IAV RNAi agent)+PR8, which is consistent the reductions reported above in Table 13.

### Example 5. In Vivo Administration of IAV RNAi Agents to Mice Subsequently Infected with PR8

Female C57Bl/6 mice animals were dosed with IAV RNAi agents and subsequently infected with PR8, in accordance with Example 3 (“PR8 model mice”). On Days 1 and 3, four (n=4) mice (for Group 1) and six (n=6) mice (for Groups 2-6) were dosed with either saline or IAV RNAi agent formulated in saline (at 3 mg/kg), via intratracheal (IT) administration. On Day 7, the animals were dosed with either PBS or PR8 (BEI) formulated in PBS, via intranasal (IN) injection. Dosing was in accordance with Table 14 below.

TABLE 14
Dosing for mice animals of Example 5

| Group | Dose (RNAi Agent) | Dose (PR8) |
|---|---|---|
| 1 | Day 1 and 3: IT 50 μL Saline | Day 7: IN 100 μL PBS |
| 2 | Day 1 and 3: IT 50 μL Saline | Day 7: IN 100 μL PR8 |
| | | (938EID50) |
| 3 | Day 1 and 3: IT 50 μL 3 mg/kg | Day 7: IN 100 μL PR8 |
| | AC002322 | (938EID50) |
| 4 | Day 1 and 3: IT 50 μL 3 mg/kg | Day 7: IN 100 μL PR8 |
| | AC002317 | (938EID50) |
| 5 | Day 1 and 3: IT 50 μL 3 mg/kg | Day 7: IN 100 μL PR8 |
| | AC002565 | (938EID50) |
| 6 | Day 1 and 3: IT 50 μL 3 mg/kg | Day 7: IN 100 μL PR8 |
| | AC002566 | (938EID50) |

PR8 dose was quantified in EID50 (50% egg infective dose). This EID50 dose was determined to be the optimal sub-lethal dose in accordance with the PR8 mouse model described in Example 2, above.

On Day 14, the animals were sacrificed. Lungs (both left and right) were collected and harvested. Expression of mouse H1N1 in right lung was determined using qPCR, with 18S rRNA as endogenous control gene. Average H1N1 expression for each animal in lung tissue was normalized relative to Group 2 (no RNAi agent+PR8 infection). Results are shown in Table 15 below.

TABLE 15
Average relative expression of H1N1 in mice lung of Example 5.

| Group ID | Avg H1N1 | High | Low |
|---|---|---|---|
| 1. Saline + PBS | N/A | N/A | N/A |
| 2. Saline + PR8 | 1.000 | 0.208 | 0.262 |
| 3. 3 mg/kg AC002322 + PR8 | 1.103 | 0.277 | 0.370 |
| 4. 3 mg/kg AC002317 + PR8 | 1.049 | 0.195 | 0.239 |
| 5. 3 mg/kg AC002565 + PR8 | 1.044 | 0.296 | 0.413 |
| 6. 3 mg/kg AC002566 + PR8 | 1.130 | 0.245 | 0.312 |

No reductions in H1N1 were seen in this Example.

### Example 6. In Vivo Administration of IAV RNAi Agents to Mice Subsequently Infected with PR8

Female C57Bl/6 mice animals were dosed with IAV RNAi agents and subsequently infected with PR8, in accordance with Example 3 (“PR8 model mice”). On Days 1 and 3, four (n=4) mice (for Group 1) and six (n=6) mice (for Groups 2-6) were dosed with either saline or IAV RNAi agent formulated in saline (at 3 mg/kg), via intratracheal (IT) administration. On Day 15, the animals were dosed with either PBS or PR8 (BEI) formulated in PBS, via intranasal (IN) injection. Dosing was in accordance with Table 16 below.

TABLE 16
Dosing for mice animals of Example 6.

| Group | Dose (RNAi Agent) | Dose (PR8) |
|---|---|---|
| 1 | Day 1 and 3: IT 50 μL Saline | Day 15: IN 100 μL PBS |
| 2 | Day 1 and 3: IT 50 μL Saline | Day 15: IN 100 μL PR8 |
| | | (938EID50) |
| 3 | Day 1 and 3: IT 50 μL 3 mg/kg | Day 15: IN 100 μL PR8 |
| | AC002322 | (938EID50) |
| 4 | Day 1 and 3: IT 50 μL 3 mg/kg | Day 15: IN 100 μL PR8 |
| | AC002317 | (938EID50) |
| 5 | Day 1 and 3: IT 50 μL 3 mg/kg | Day 15: IN 100 μL PR8 |
| | AC002565 | (938EID50) |
| 6 | Day 1 and 3: IT 50 μL 3 mg/kg | Day 15: IN 100 μL PR8 |
| | AC002566 | (938EID50) |

PR8 dose was quantified in EID50 (50% egg infective dose). This EID50 dose was determined to be the optimal sub-lethal dose in accordance with the PR8 mouse model described in Example 2, above.

On Day 18, the animals were sacrificed. Lungs (both left and right) were collected and harvested. Expression of mouse H1N1 in lung was determined using qPCR, with B2M as endogenous control gene. Average H1N1 expression for each animal in lung tissue was normalized relative to Group 2 (no RNAi agent+PR8 infection). Results are shown in Table 17 below.

TABLE 17
Average relative expression of H1N1 in mice lung of Example 6.

| | Avg | Std Dev |
|---|---|---|
| Group ID | H1N1 | (+/−) |
| 1. Saline + PBS | N/A | N/A |
| 2. Saline + PR8 | 1.020 | 0.221 |
| 3. 3 mg/kg AC002322 + PR8 | 0.714 | 0.273 |
| 4. 3 mg/kg AC002317 + PR8 | 1.158 | 0.650 |
| 5. 3 mg/kg AC002565 + PR8 | 0.679 | 0.404 |
| 6. 3 mg/kg AC002566 + PR8 | 0.866 | 0.435 |

Only very limited reductions in H1N1, if any (i.e., Group 4), were seen in this Example.

### Example 7. In Vivo Administration of IAV RNAi Agents to Mice Subsequently Infected with PR8

Female C57Bl/6 mice animals were dosed with IAV RNAi agents and subsequently infected with PR8, in accordance with Example 3 (“PR8 model mice”). On Days 1 and 3, four (n=4) mice (for Group 1) and six (n=6) mice (for Groups 2-5) were dosed with either saline or IAV RNAi agent formulated in saline (at 3 mg/kg), via intratracheal (IT) administration. On Day 8, the animals were dosed with either PBS or PR8 (BEI) formulated in PBS, via intranasal (IN) injection. Dosing was in accordance with Table 18 below.

TABLE 18
Dosing for mice animals of Example 7.

| Group | Dose (RNAi Agent) | Dose (PR8) |
|---|---|---|
| 1 | Day 1 and 3: IT 50 μL Saline | Day 8: IN 100 μL PBS |
| 2 | Day 1 and 3: IT 50 μL Saline | Day 8: IN 100 μL PR8 |
| | | (938EID50) |
| 3 | Day 1 and 3: IT 50 μL 3 mg/kg | Day 8: IN 100 μL PR8 |
| | AC002564 | (938EID50) |
| 4 | Day 1 and 3: IT 50 μL 3 mg/kg | Day 8: IN 100 μL PR8 |
| | AC002567 | (938EID50) |
| 5 | Day 1 and 3: IT 50 μL 3 mg/kg | Day 8: IN 100 μL PR8 |
| | AC002568 | (938EID50) |

PR8 dose was quantified in EID50 (50% egg infective dose). This EID50 dose was determined to be the optimal sub-lethal dose in accordance with the PR8 mouse model described in Example 2, above.

On Day 13, the animals were sacrificed. Lungs (both left and right) were collected and harvested. Expression of mouse H1N1 and M1 in lung was determined using qPCR, with B2M as endogenous control gene. Average H1N1 and M1 expression for each animal in lung tissue was normalized relative to Group 2 (no RNAi agent+PR8 infection). Results are shown in Table 19 below.

TABLE 19
Average relative expression of HIN1 and M1 in mice lung of Example 7.

| | Avg | | | | | |
|---|---|---|---|---|---|---|
| Group ID | H1N1 | Low | High | Avg M1 | Low | High |
| 1. Saline + PBS | N/A | N/A | N/A | N/A | N/A | N/A |
| 2. Saline + PR8 | 1.000 | 0.341 | 0.517 | 1.000 | 0.334 | 0.502 |
| 3. 3 mg/kg AC002564 + | 0.041 | 0.024 | 0.055 | 0.054 | 0.031 | 0.070 |
| PR8 | | | | | | |
| 4. 3 mg/kg AC002567 + | 0.930 | 0.240 | 0.104 | 1.161 | 0.401 | 0.245 |
| PR8 | | | | | | |
| 5. 3 mg/kg AC002568 + | 0.496 | 0.130 | 0.177 | 0.615 | 0.210 | 0.320 |
| PR8 | | | | | | |

Groups 3 and 5 showed reductions in H1N1, with Group 3 in particular (AC002564) showing reductions of nearly 96% (0.041) in H1N1. Groups 3 and 5 similarly showed reductions in M1, again with Group 3 (AC002564) showing reductions of approximately 95% (0.054) in M1.

### Example 8. In Vivo Administration of IAV RNAi Agents to Mice Subsequently Infected with PR8

Female C57Bl/6 mice animals were dosed with IAV RNAi agents and subsequently infected with PR8, in accordance with Example 3 (“PR8 model mice”). On Days 1 and 4, four (n=4) mice (for Group 1) and six (n=6) mice (for Groups 2-9) were dosed with either saline or IAV RNAi agent formulated in saline (at 3 mg/kg), via intratracheal (IT) administration. On Day 9, 16, 23, or 30, the animals were dosed with either PBS or PR8 (BEI) formulated in PBS, via intranasal (IN) injection. Dosing was in accordance with Table 20 below.

TABLE 20
Dosing for mice animals of Example 8.

| Group | Dose (RNAi Agent) | Dose (PR8) | Sacrifice |
|---|---|---|---|
| 1 | Day 1 and 4: IT 50 μL | Day 9: IN 100 μL PBS | Day 16 |
| | Saline | | |
| 2 | Day 1 and 4: IT 50 μL | Day 9: IN 100 μL PR8 | Day 16 |
| | Saline | (938EID50) | |
| 3 | Day 1 and 4: IT 50 μL | Day 16: IN 100 μL PR8 | Day 23 |
| | Saline | (938EID50) | |
| 4 | Day 1 and 4: IT 50 μL | Day 23: IN 100 μL PR8 | Day 30 |
| | Saline | (938EID50) | |
| 5 | Day 1 and 4: IT 50 μL | Day 30: IN 100 μL PR8 | Day 37 |
| | Saline | (938EID50) | |
| 6 | Day 1 and 4: IT 50 μL | Day 9: IN 100 μL PR8 | Day 16 |
| | 3 mg/kg AC002564 | (938EID50) | |
| 7 | Day 1 and 4: IT 50 μL | Day 16: IN 100 μL PR8 | Day 23 |
| | 3 mg/kg AC002564 | (938EID50) | |
| 8 | Day 1 and 4: IT 50 μL | Day 23: IN 100 μL PR8 | Day 30 |
| | 3 mg/kg AC002564 | (938EID50) | |
| 9 | Day 1 and 4: IT 50 μL | Day 30: IN 100 μL PR8 | Day 37 |
| | 3 mg/kg AC002564 | (938EID50) | |

PR8 dose was quantified in EID50 (50% egg infective dose). This EID50 dose was determined to be the optimal sub-lethal dose in accordance with the PR8 mouse model described in Example 2, above.

On Day 16, 23, 30, or 37, the animals were sacrificed, in accordance with Table 20 above. Bronchoalveolar lavage fluid (BALF) and lungs (both left and right) were collected and harvested. Expression of mouse H1N1 and M1 in lung was determined using qPCR, with B32M as endogenous control gene. Average H1N1 and M1 expression for each animal in lung tissue was normalized relative to Group 2 (no RNAi agent+Day 9 PR8 infection). Results are shown in Table 21 below.

TABLE 21
Average relative expression of H1N1 and M1 in mice lung of Example 7.

| | Avg | | | | | |
|---|---|---|---|---|---|---|
| Group ID | H1N1 | Low | High | Avg M1 | Low | High |
| 1. Saline + D9 PBS | N/A | N/A | N/A | N/A | N/A | N/A |
| 2. Saline + D9 PR8 | 1.000 | 0.140 | 0.163 | 1.000 | 0.137 | 0.159 |
| 3. Saline + D16 PR8 | 1.517 | 0.206 | 0.238 | 1.496 | 0.177 | 0.201 |
| 4. Saline + D23 PR8 | 1.605 | 0.361 | 0.466 | 1.631 | 0.291 | 0.354 |
| 5. Saline + D30 PR8 | 1.585 | 0.601 | 0.969 | 1.585 | 0.531 | 0.798 |
| 6. 3 mg/kg AC002564 + | 0.046 | 0.031 | 0.094 | 0.050 | 0.034 | 0.102 |
| D9 PR8 | | | | | | |
| 7. 3 mg/kg AC002564 + | 0.287 | 0.147 | 0.301 | 0.297 | 0.124 | 0.213 |
| D16 PR8 | | | | | | |
| 8. 3 mg/kg AC002564 − | 0.557 | 0.264 | 0.501 | 0.516 | 0.184 | 0.286 |
| D23 PR8 | | | | | | |
| 9. 3 mg/kg AC002564 − | 0.837 | 0.328 | 0.539 | 0.821 | 0.313 | 0.505 |
| D30 PR8 | | | | | | |

Reductions in H1N1 and M1 from the IAV RNAi agent AR002564 (Groups 6-9) showed that meaningful knockdown was sustained in this PR8 mouse model in this Example at least through Day 23.

### Example 9. In Vivo Administration of IAV RNAi Agents to Mice Subsequently Infected with PR8

Female C57Bl/6 mice animals were dosed with IAV RNAi agents and subsequently infected with PR8. On Days 1 and 4, four (n=4) mice (for Group 1) and six (n=6) mice (for Groups 2-7) were dosed with either saline or IAV RNAi agent formulated in saline (at 0.5 mg/kg, 1 mg/kg, 3 mg/kg), via intratracheal (IT) administration. On Day 8, the animals were dosed with either PBS or PR8 (BEI) formulated in PBS, via intranasal (IN) injection. Dosing was in accordance with Table 22 below.

TABLE 22
Dosing for mice animals of Example 9.

| Group | Dose (RNAi Agent) | Dose (PR8) | Sacrifice |
|---|---|---|---|
| 1 | Day 1 and 4: IT 50 μL | Day 8: IN 100 μL PBS | Day 14 |
| | Saline | | |
| 2 | Day 1 and 4: IT 50 μL | Day 8: IN 100 μL PR8 | Day 14 |
| | Saline | (938EID50) | |
| 3 | Day 1 and 4: IT 50 μL | Day 8: IN 100 μL PR8 | Day 21 |
| | Saline | (938EID50) | |
| 4 | Day 1 and 4: IT 50 μL | Day 8: IN 100 μL PR8 | Day 14 |
| | 3 mg/kg AC002564 | (938EID50) | |
| 5 | Day 1 and 4: IT 50 μL | Day 8: IN 100 μL PR8 | Day 14 |
| | 1.5 mg/kg AC002564 | (938EID50) | |
| 6 | Day 1 and 4: IT 50 μL | Day 8: IN 100 μL PR8 | Day 14 |
| | 0.5 mg/kg AC002564 | (938EID50) | |
| 7 | Day 1 and 4: IT 50 μL | Day 8: IN 100 μL PR8 | Day 21 |
| | 3 mg/kg AC002564 | (938EID50) | |

PR8 dose was quantified in EID50 (50% egg infective dose). This EID50 dose was determined to be the optimal sub-lethal dose in accordance with the PR8 mouse model described in Example 2, above.

On Day 14 or 21, the animals were sacrificed, in accordance with Table 22 above. Lungs (both left and right) were collected and harvested. Expression of mouse H1N1 and M1 in lung was determined using qPCR, with B2M as endogenous control gene. Average H1N1 and M1 expression for each animal in lung tissue was normalized relative to Group 2 (no RNAi agent+Day 8 PR8 infection). Results are shown in Table 23 below.

TABLE 23
Average relative expression of H1N1 and M1 in mice lung of Example 9.

| | Avg | | | Avg | | |
|---|---|---|---|---|---|---|
| Group ID | H1N1 | Low | High | M1 | Low | High |
| 1. Saline + D8 PBS | N/A | N/A | N/A | N/A | N/A | N/A |
| 2. Saline + D8 PR8, D14 Sac | 1.000 | 0.425 | 0.739 | 1.000 | 0.434 | 0.768 |
| 3. Saline + D8 PR8, D21 Sac | 0.000 | 0.000 | 0.000 | N/A | N/A | N/A |
| 4. 3 mg/kg AC002564 + PR8, | 0.137 | 0.068 | 0.136 | 0.132 | 0.066 | 0.133 |
| D14 Sac | | | | | | |
| 5. 1.5 mg/kg AC002564 + | 0.132 | 0.048 | 0.076 | 0.138 | 0.048 | 0.074 |
| PR8, D14 Sac | | | | | | |
| 6. 0.5 mg/kg AC002564 + | 0.260 | 0.106 | 0.180 | 0.255 | 0.103 | 0.173 |
| PR8, D14 Sac | | | | | | |
| 7. 3 mg/kg AC002564 + | N/A | N/A | N/A | N/A | N/A | N/A |
| PR8, D21 Sac | | | | | | |

Groups 4-6 showed reductions in H1N1 and M1 in PR8 mouse model through Day 14. Data was unavailable for mice animals sacrificed at Day 21 (Groups 3 and 7).

### Example 10. In Vivo Administration of IAV RNAi Agents to Mice Subsequently Infected with PR8

Female C57Bl/6 mice animals were dosed with IAV RNAi agents and subsequently infected with PR8, in accordance with Example 3 (“PR8 model mice”). On Days 1 and 3, four (n=4) mice (for Group 1) and six (n=6) mice (for Groups 2-3) were dosed with either saline or IAV RNAi agent formulated in saline (at 3 mg/kg), via intratracheal (IT) administration. On Day 8, the animals were dosed with either PBS or PR8 (BEI) formulated in PBS, via intranasal (IN) injection. Dosing was in accordance with Table 24 below.

TABLE 24
Dosing for mice animals of Example 10.

| Group | Dose (RNAi Agent) | Dose (PR8) |
|---|---|---|
| 1 | Day 1 and 3: IT 50 μL Saline | Day 8: IN 100 μL PBS |
| 2 | Day 1 and 3: IT 50 μL Saline | Day 8: IN 100 μL PR8 |
| | | (938EID50) |
| 3 | Day 1 and 3: IT 50 μL 3 mg/kg | Day 8: IN 100 μL PR8 |
| | AC002564 | (938EID50) |

PR8 dose was quantified in EID50 (50% egg infective dose). This EID50 dose was determined to be the optimal sub-lethal dose in accordance with the PR8 mouse model described in Example 2, above.

On Day 15, the animals were sacrificed. Lungs (both left and right) were collected and harvested. Expression of mouse H1N1 and M1 in lung was determined using qPCR, with 18s rRNA as endogenous control gene, normalized to Group 2. Average H1N1 and M1 expression for each animal in lung tissue was normalized relative to Group 2 (no RNAi agent+PR8 infection). Results are shown in Table 25 below.

TABLE 25
Average relative expression of H1N1 and M1 in mice lung of Example 10.

| | Avg | | | | | |
|---|---|---|---|---|---|---|
| Group ID | H1N1 | Low | High | Avg M1 | Low | High |
| 1. Saline + PBS | N/A | N/A | N/A | N/A | N/A | N/A |
| 2. Saline + PR8 | 1.000 | 0.329 | 0.490 | 1.000 | 0.333 | 0.498 |
| 3. 3 mg/kg AC002564 + | 0.094 | 0.079 | 0.490 | 0.088 | 0.073 | 0.437 |
| PR8 | | | | | | |

Group 3 showed reductions in H1N1 and in M1, of nearly 90-91% reductions (0.094, 0.088) in H1N1 and M1 at Day 15, with 2×3.0 mg/kg dose before PR8 viral challenge.

### Example 11. In Vivo Administration of IAV RNAi Agents to Mice Subsequently Infected with PR8

Female C57Bl/6 mice animals were dosed with IAV RNAi agents and subsequently infected with PR8, in accordance with Example 3 (“PR8 model mice”). On Days 1 and 3, four (n=4) mice (for Group 1) and six (n=6) mice (for Groups 2-3) were dosed with either saline or IAV RNAi agent formulated in saline (at 3 mg/kg), via intratracheal (IT) administration. On Day 7, the animals were dosed with either PBS or PR8 (BEI) formulated in PBS, via intranasal (IN) injection. Dosing was in accordance with Table 26 below.

TABLE 26
Dosing for mice animals of Example 11.

| Group | Dose (RNAi Agent) | Dose (PR8) |
|---|---|---|
| 1 | Day 1 and 3: IT 50 μL Saline | Day 7: IN 100 μL PBS |
| 2 | Day 1 and 3: IT 50 μL Saline | Day 7: IN 100 μL PR8 |
| | | (938EID50) |
| 3 | Day 1 and 3: IT 50 μL 3 mg/kg | Day 7: IN 100 μL PR8 |
| | AC002564 | (938EID50) |

PR8 dose was quantified in EID50 (50% egg infective dose). This EID50 dose was determined to be the optimal sub-lethal dose in accordance with the PR8 mouse model described in Example 2, above.

On Day 15, the animals were sacrificed. Lungs (both left and right) were collected and harvested. Expression of mouse H1N1 and M1 in lung was determined using qPCR, with 18s rRNA as endogenous control gene. Average H1N1 and M1 expression for each animal in lung tissue was normalized relative to Group 2 (no RNAi agent+PR8 infection). Results are shown in Table 27 below.

TABLE 27
Average relative expression of H1N1 and M1 in mice lung of Example 9.

| | Avg | | | Avg | | |
|---|---|---|---|---|---|---|
| Group ID | H1N1 | Low | High | M1 | Low | High |
| 1. Saline + PBS | N/A | N/A | N/A | N/A | N/A | N/A |
| 2. Saline + PR8 | 1.000 | 0.532 | 1.137 | 1.000 | 0.493 | 0.973 |
| 3. 3 mg/kg AC002564 + PR8 | 0.076 | 0.040 | 0.083 | 0.083 | 0.038 | 0.069 |

Group 3 showed reductions in H1N1 and in M1, of nearly 92-93% reductions (0.076, 0.083) in H1N1 and M1 at Day 15, with 2×3.0 mg/kg dose before PR8 viral challenge.

### Example 12. In Vivo Administration of IAV RNAi Agents to Mice Subsequently Infected with PR8

Female C57Bl/6 mice animals were dosed with IAV RNAi agents and subsequently infected with PR8, in accordance with Example 3 (“PR8 model mice”). On Days 1 and 3, four (n=4) mice (for Group 1) and five (n=5) mice (for Groups 2-8) were dosed with either saline or IAV RNAi agent formulated in saline (at 3 mg/kg), via intratracheal (IT) administration. On Day 8, the animals were dosed with either PBS or PR8 (BEI) formulated in PBS, via intranasal (IN) injection. Dosing was in accordance with Table 28 below.

TABLE 28
Dosing for mice animals of Example 12.

| Group | Dose (RNAi Agent) | Dose (PR8) |
|---|---|---|
| 1 | Day 1 and 3: IT 50 μL Saline | Day 8: IN 100 μL PBS |
| 2 | Day 1 and 3: IT 50 μL Saline | Day 8: IN 100 μL PR8 |
| | | (938EID50) |
| 3 | Day 1 and 3: IT 50 μL 3 mg/kg | Day 8: IN 100 μL PR8 |
| | AC002754 | (938EID50) |
| 4 | Day 1 and 3: IT 50 μL 3 mg/kg | Day 8: IN 100 μL PR8 |
| | AC002755 | (938EID50) |
| 5 | Day 1 and 3: IT 50 μL 3 mg/kg | Day 8: IN 100 μL PR8 |
| | AC002756 | (938EID50) |
| 6 | Day 1 and 3: IT 50 μL 3 mg/kg | Day 8: IN 100 μL PR8 |
| | AC002757 | (938EID50) |
| 7 | Day 1 and 3: IT 50 μL 3 mg/kg | Day 8: IN 100 μL PR8 |
| | AC002758 | (938EID50) |
| 8 | Day 1 and 3: IT 50 μL 3 mg/kg | Day 8: IN 100 μL PR8 |
| | AC002759 | (938EID50) |

PR8 dose was quantified in EID50 (50% egg infective dose). This EID50 dose was determined to be the optimal sub-lethal dose in accordance with the PR8 mouse model described in Example 2, above.

On Day 15, the animals were sacrificed. Lungs (both left and right) were collected and harvested. Expression of mouse H1N1 and M1 in lung was determined using PCR, with 18s rRNA as endogenous control gene. Average H1N1 and M1 expression for each animal in lung tissue was normalized relative to Group 2 (no RNAi agent+PR8 infection). Results are shown in Table 29 below.

TABLE 29
Average relative expression of H1N1 and M1 in mice lung of Example 12.

| | Avg | | | Avg | | |
|---|---|---|---|---|---|---|
| Group ID | H1N1 | Low | High | M1 | Low | High |
| 1. Saline + PBS | N/A | N/A | N/A | N/A | N/A | N/A |
| 2. Saline + PR8 | 1.000 | 0.229 | 0.298 | 1.000 | 0.195 | 0.242 |
| 3. 3 mg/kg AC002754 + PR8 | 0.987 | 0.595 | 1.500 | 0.841 | 0.364 | 0.643 |
| 4. 3 mg/kg AC002755 + PR8 | 0.516 | 0.082 | 0.098 | 0.592 | 0.109 | 0.133 |
| 5. 3 mg/kg AC002756 + PR8 | 0.393 | 0.069 | 0.084 | 0.423 | 0.070 | 0.084 |
| 6. 3 mg/kg AC002757 + PR8 | 0.938 | 0.145 | 0.171 | 0.843 | 0.104 | 0.118 |
| 7. 3 mg/kg AC002758 + PR8 | 0.888 | 0.185 | 0.234 | 0.875 | 0.156 | 0.190 |
| 8. 3 mg/kg AC002759 + PR8 | 1.135 | 0.390 | 0.595 | 0.892 | 0.193 | 0.246 |

Rather modest reductions in H1N1 and M1 were seen ins Groups 4 and 5, with effectively no reductions seen from the IAV RNAi agents in Groups 3, 6, 7, and 8.

### Example 13. In Vivo Administration of IAV RNAi Agents to Mice Subsequently Infected with PR8

Female C57Bl/6 mice animals were dosed with IAV RNAi agents and subsequently infected with PR8, in accordance with Example 3 (“PR8 model mice”). On Days 1 and 3, four (n=4) mice (for Group 1) and five (n=5) mice (for Groups 2-8) were dosed with either saline or IAV RNAi agent formulated in saline (at 3 mg/kg), via intratracheal (IT) administration. On Day 8, the animals were dosed with either PBS or PR8 (BEI) formulated in PBS, via intranasal (IN) injection. Dosing was in accordance with Table 30 below.

TABLE 30
Dosing for mice animals of Example 13.

| Group | Dose (RNAi Agent) | Dose (PR8) |
|---|---|---|
| 1 | Day 1 and 3: IT 50 μL Saline | Day 8: IN 100 μL PBS |
| 2 | Day 1 and 3: IT 50 μL Saline | Day 8: IN 100 μL PR8 |
| | | (938EID50) |
| 3 | Day 1 and 3: IT 50 μL 3 mg/kg | Day 8: IN 100 μL PR8 |
| | AC002760 | (938EID50) |
| 4 | Day 1 and 3: IT 50 μL 3 mg/kg | Day 8: IN 100 μL PR8 |
| | AC002761 | (938EID50) |
| 5 | Day 1 and 3: IT 50 μL 3 mg/kg | Day 8: IN 100 μL PR8 |
| | AC002762 | (938EID50) |
| 6 | Day 1 and 3: IT 50 μL 3 mg/kg | Day 8: IN 100 μL PR8 |
| | AC002763 | (938EID50) |
| 7 | Day 1 and 3: IT 50 μL 3 mg/kg | Day 8: IN 100 μL PR8 |
| | AC002764 | (938EID50) |
| 8 | Day 1 and 3: IT 50 μL 3 mg/kg | Day 8: IN 100 μL PR8 |
| | AC002765 | (938EID50) |

PR8 dose was quantified in EID50 (50% egg infective dose). This EID50 dose was determined to be the optimal sub-lethal dose in accordance with the PR8 mouse model described in Example 2, above.

On Day 14, the animals were sacrificed. Lungs (both left and right) were collected and harvested. Expression of mouse H1N1 and M1 in lung was determined using qPCR, with 18s rRNA as endogenous control gene. Average H1N1 and M1 expression for each animal in lung tissue was normalized relative to Group 2 (no RNAi agent+PR8 infection). Results are shown in Table 31 below.

TABLE 31
Average relative expression of H1N1
and M1 in mice lung of Example 13.

| | Avg | | | Avg | | |
|---|---|---|---|---|---|---|
| Group ID | H1N1 | Low | High | M1 | Low | High |
| 1. Saline + PBS | N/A | N/A | N/A | N/A | N/A | N/A |
| 2. Saline + PR8 | 1.000 | 0.213 | 0.271 | 1.000 | 0.268 | 0.366 |
| 3. 3 mg/kg AC002760 + | 0.336 | 0.146 | 0.258 | 0.582 | 0.239 | 0.404 |
| PR8 | | | | | | |
| 4. 3 mg/kg AC002761 + | 0.334 | 0.128 | 0.208 | 0.574 | 0.207 | 0.323 |
| PR8 | | | | | | |
| 5. 3 mg/kg AC002762 + | 0.679 | 0.221 | 0.328 | 0.923 | 0.269 | 0.380 |
| PR8 | | | | | | |
| 6. 3 mg/kg AC002763 + | 0.665 | 0.096 | 0.112 | 0.907 | 0.226 | 0.300 |
| PR8 | | | | | | |
| 7. 3 mg/kg AC002764 + | 0.397 | 0.133 | 0.200 | 0.471 | 0.179 | 0.288 |
| PR8 | | | | | | |
| 8. 3 mg/kg AC002765 + | 0.670 | 0.202 | 0.290 | 0.979 | 0.304 | 0.441 |
| PR8 | | | | | | |

Groups 3-8 showed only relatively modest reductions in H1N1 and M1.

### Example 14. In Vivo Administration of IAV RNAi Agents to Mice Subsequently Infected with PR8

Female C57Bl/6 mice animals were dosed with IAV RNAi agents and subsequently infected with PR8, in accordance with Example 3 (“PR8 model mice”). On Days 1 and 3, four (n=4) mice (for Group 1) and five (n=5) mice (for Groups 2-8) were dosed with either saline or IAV RNAi agent formulated in saline (at 3 mg/kg), via intratracheal (IT) administration. On Day 8, the animals were dosed with either PBS or PR8 (BEI) formulated in PBS, via intranasal (IN) injection. Dosing was in accordance with Table 32 below.

TABLE 32
Dosing for mice animals of Example 14.

| Group | Dose (RNAi Agent) | Dose (PR8) |
|---|---|---|
| 1 | Day 1 and 3: IT 50 μL Saline | Day 8: IN 100 μL PBS |
| 2 | Day 1 and 3: IT 50 μL Saline | Day 8: IN 100 μL PR8 |
| | | (938EID50) |
| 3 | Day 1 and 3: IT 50 μL 3 mg/kg | Day 8: IN 100 μL PR8 |
| | AC002766 | (938EID50) |
| 4 | Day 1 and 3: IT 50 μL 3 mg/kg | Day 8: IN 100 μL PR8 |
| | AC002767 | (938EID50) |
| 5 | Day 1 and 3: IT 50 μL 3 mg/kg | Day 8: IN 100 μL PR8 |
| | AC002768 | (938EID50) |
| 6 | Day 1 and 3: IT 50 μL 3 mg/kg | Day 8: IN 100 μL PR8 |
| | AC002769 | (938EID50) |
| 7 | Day 1 and 3: IT 50 μL 3 mg/kg | Day 8: IN 100 μL PR8 |
| | AC002770 | (938EID50) |
| 8 | Day 1 and 3: IT 50 μL 3 mg/kg | Day 8: IN 100 μL PR8 |
| | AC002771 | (938EID50) |

PR8 dose was quantified in EID50 (50% egg infective dose). This EID50 dose was determined to be the optimal sub-lethal dose in accordance with the PR8 mouse model described in Example 2, above.

On Day 15, the animals were sacrificed. Lungs (both left and right) were collected and harvested. Expression of mouse H1N1 and M1 in lung was determined using qPCR, with 18s rRNA as endogenous control gene. Average H1N1 and M1 expression for each animal in lung tissue was normalized relative to Group 2 (no RNAi agent+PR8 infection). Results are shown in Table 33 below.

TABLE 33
Average relative expression of H1N1 and M1 in mice lung
of Example 14.

| | Avg | | | Avg | | |
|---|---|---|---|---|---|---|
| Group ID | H1N1 | Low | High | M1 | Low | High |
| 1. Saline + PBS | N/A | N/A | N/A | N/A | N/A | N/A |
| 2. Saline + PR8 | 1.000 | 0.197 | 0.245 | 1.000 | 0.186 | 0.229 |
| 3. 3 mg/kg AC002766 + PR8 | 0.841 | 0.256 | 0.368 | 0.971 | 0.317 | 0.470 |
| 4. 3 mg/kg AC002767 + PR8 | 0.622 | 0.232 | 0.370 | 0.767 | 0.296 | 0.483 |
| 5. 3 mg/kg AC002768 + PR8 | 1.011 | 0.262 | 0.354 | 1.250 | 0.359 | 0.503 |
| 6. 3 mg/kg AC002769 + PR8 | 0.589 | 0.259 | 0.462 | 0.831 | 0.316 | 0.511 |
| 7. 3 mg/kg AC002770 + PR8 | 0.665 | 0.227 | 0.346 | 0.783 | 0.226 | 0.318 |
| 8. 3 mg/kg AC002771 + PR8 | 1.291 | 0.423 | 0.630 | 1.456 | 0.410 | 0.570 |

Groups 3, 4, 6, and 7 showed only relatively modest reductions in H1N1 and M1.

### Example 15. In Vivo Administration of IAV RNAi Agents to Mice Previously Infected with PR8

Female C57Bl/6 mice animals were infected with PR8, and subsequently dosed with IAV RNAi agents, in accordance with Example 3 (“PR8 model mice”). On Day 1, four (n=4) mice (for Group 1) and five (n=5) mice (for Groups 2-11) animals were dosed with either PBS or PR8 (BEI) formulated in PBS, via intranasal (IN) injection. On Day 5, the animals were dosed with either saline or IAV RNAi agent formulated in saline (at 3 mg/kg), via intratracheal (IT) administration. Dosing was in accordance with Table 34 below.

TABLE 34
Dosing for mice animals of Example 15.

| Group | Dose (RNAi Agent) | Dose (PR8) | Sacrifice |
|---|---|---|---|
| 1 | Day 5: IT 50 μL Saline | Day 1: IN 100 μL PBS | Day 10 |
| 2 | Day 5: IT 50 μL Saline | Day 1: IN 100 μL PR8 | Day 5, |
| | | (469 EID50) | 6 hours |
| | | | post PR8 |
| | | | infection |
| 3 | Day 5: IT 50 μL 3 mg/kg | Day 1: IN 100 μL PR8 | Day 5, |
| | AC002564 | (469 EID50) | 6 hours |
| | | | post PR8 |
| | | | infection |
| 4 | Day 5: IT 50 μL Saline | Day 1: IN 100 μL PR8 | Day 6 |
| | | (469 EID50) | |
| 5 | Day 5: IT 50 μL 3 mg/kg | Day 1: IN 100 μL PR8 | Day 6 |
| | AC002564 | (469 EID50) | |
| 6 | Day 5: IT 50 μL Saline | Day 1: IN 100 μL PR8 | Day 7 |
| | | (469 EID50) | |
| 7 | Day 5: IT 50 μL 3 mg/kg | Day 1: IN 100 μL PR8 | Day 7 |
| | AC002564 | (469 EID50) | |
| 8 | Day 5: IT 50 μL Saline | Day 1: IN 100 μL PR8 | Day 8 |
| | | (469 EID50) | |
| 9 | Day 5: IT 50 μL 3 mg/kg | Day 1: IN 100 μL PR8 | Day 8 |
| | AC002564 | (469 EID50) | |
| 10 | Day 5: IT 50 μL Saline | Day 1: IN 100 μL PR8 | Day 9 |
| | | (469 EID50) | |
| 11 | Day 5: IT 50 μL 3 mg/kg | Day 1: IN 100 μL PR8 | Day 9 |
| | AC002564 | (469 EID50) | |

PR8 dose was quantified in EID50 (50% egg infective dose). This EID50 dose was determined to be the optimal sub-lethal dose in accordance with the PR8 mouse model described in Example 2, above.

On Day 5, 6, 7, 8, 9, or 10, the animals were sacrificed, in accordance with Table 34 above. Lungs (both left and right) were collected and harvested. Expression of mouse H1N1 and M1 in lung was determined using qPCR, with 18s rRNA as endogenous control gene. Average H1N1 and M1 expression for each animal in lung tissue was normalized relative to each control group dosed with saline; Group 3 is normalized to Group 2, Group 5 is normalized to Group 4, Group 7 is normalized to Group 6, Group 9 is normalized to Group 8, and Group 11 is normalized to Group 10. Results are shown in Table 35 below.

TABLE 35
Average relative expression of H1N1 and M1 in mice lung of Example 15.

| | Avg | | | Avg | | |
|---|---|---|---|---|---|---|
| Group ID | H1N1 | Low | High | M1 | Low | High |
| 1. Saline + PBS, D10 Sac | N/A | N/A | N/A | N/A | N/A | N/A |
| 2. Saline + PR8, D5 Sac | 1.000 | 0.371 | 0.589 | 1.000 | 0.317 | 0.463 |
| 3. 3 mg/kg AC002564 + | 0.596 | 0.198 | 0.295 | 0.585 | 0.119 | 0.150 |
| PBS, D5 Sac | | | | | | |
| 4. Saline + PR8, D6 Sac | 1.000 | 0.172 | 0.208 | 1.000 | 0.175 | 0.212 |
| 5. 3 mg/kg AC002564 + | 0.611 | 0.236 | 0.384 | 0.507 | 0.086 | 0.103 |
| PR8, D6 Sac | | | | | | |
| 6. Saline + PR8, D7 Sac | 1.000 | 0.335 | 0.504 | 1.000 | 0.168 | 0.202 |
| 7. 3 mg/kg AC002564 + | 0.423 | 0.105 | 0.140 | 0.494 | 0.116 | 0.151 |
| PR8, D7 Sac | | | | | | |
| 8. Saline + PR8, D8 Sac | 1.000 | 0.207 | 0.262 | 1.000 | 0.122 | 0.139 |
| 9. 3 mg/kg AC002564 + | 0.236 | 0.075 | 0.109 | 0.317 | 0.081 | 0.109 |
| PR8, D8 Sac | | | | | | |
| 10. Saline + PR8, D9 Sac | 1.000 | 0.325 | 0.482 | 1.000 | 0.272 | 0.374 |
| 11. 3 mg/kg AC002564 + | 0.074 | 0.021 | 0.030 | 0.065 | 0.018 | 0.024 |
| PR8, D9 Sac | | | | | | |

Groups 3, 5, 7, 9, and 11 showed reduction in H1N1 and M1. Specifically, Group 11 showed ˜92% reduction (0.074) of H1N1 and 93% reduction (0.065) of M1 at Day 9.

### Example 16. In Vivo Administration of IAV RNAi Agents to Mice Subsequently Infected with CA07 H1N1

Female C57Bl/6 mice animals were dosed with IAV RNAi agents and subsequently infected with CA07, in accordance with Example 4 (“CA07 H1N1 model mice”). On Days 1 and 3, four (n=4) mice (for Group 1) and five (n=5) mice (for Groups 2-8) were dosed with either saline or IAV RNAi agent formulated in saline (at 3 mg/kg), via intratracheal (IT) administration. On Day 8, the animals were dosed with either PBS or CA07 H1N1 formulated in PBS, via intranasal (N) injection. Dosing was in accordance with Table 36 below.

TABLE 36
Dosing for mice animals of Example 16.

| Group | Dose (RNAi Agent) | Dose (CA07) |
|---|---|---|
| 1 | Day 1 and 3: IT 50 μL Saline | Day 8: IN 100 μL PBS |
| 2 | Day 1 and 3: IT 50 μL Saline | Day 8: IN 100 μL CA07 |
| | | (4240 TCID50) |
| 3 | Day 1 and 3: IT 50 μL 3 mg/kg | Day 8: IN 100 μL CA07 |
| | AC002564 | (4240 TCID50) |
| 4 | Day 1 and 3: IT 50 μL 3 mg/kg | Day 8: IN 100 μL CA07 |
| | AC002865 | (4240 TCID50) |
| 5 | Day 1 and 3: IT 50 μL 3 mg/kg | Day 8: IN 100 μL CA07 |
| | AC002866 | (4240 TCID50) |
| 6 | Day 1 and 3: IT 50 μL 3 mg/kg | Day 8: IN 100 μL CA07 |
| | AC002869 | (4240 TCID50) |
| 7 | Day 1 and 3: IT 50 μL 3 mg/kg | Day 8: IN 100 μL CA07 |
| | AC002870 | (4240 TCID50) |
| 8 | Day 1 and 3: IT 50 μL 3 mg/kg | Day 8: IN 100 μL CA07 |
| | AC002872 | (4240 TCID50) |

CA07 dose was quantified in TCID50 (50% tissue culture infective dose). This TCID50 dose was determined to be the optimal sub-lethal dose in accordance with the CA07 mouse model described in Example 3, above.

On Day 14, the animals were sacrificed. Lungs (both left and right) were collected and harvested. Expression of mouse H1N1 and M1 in lung was determined using qPCR, with 18s rRNA as endogenous control gene. Average H1N1 and M1 expression for each animal in lung tissue was normalized relative to Group 2 (no RNAi agent+CA07 infection). Results are shown in Table 37 below.

TABLE 37
Average relative expression of HIN1 and M1 in mice lung of Example 16.

| | Avg | | | Avg | | |
|---|---|---|---|---|---|---|
| Group ID | H1N1 | Low | High | M1 | Low | High |
| 1. Saline + PBS | N/A | N/A | N/A | N/A | N/A | N/A |
| 2. Saline + CA07 | 1.000 | 0.223 | 0.286 | 1.000 | 0.225 | 0.290 |
| 3. 3 mg/kg AC002564 + CA07 | 0.192 | 0.084 | 0.151 | 0.309 | 0.146 | 0.277 |
| 4. 3 mg/kg AC002865 + CA07 | 0.320 | 0.140 | 0.248 | 0.471 | 0.161 | 0.245 |
| 5. 3 mg/kg AC002866 + CA07 | 0.169 | 0.055 | 0.082 | 0.241 | 0.068 | 0.094 |
| 6. 3 mg/kg AC002869 + CA07 | 0.109 | 0.055 | 0.113 | 0.158 | 0.077 | 0.151 |
| 7. 3 mg/kg AC002870 + CA07 | 0.232 | 0.093 | 0.155 | 0.304 | 0.119 | 0.194 |
| 8. 3 mg/kg AC002872 + CA07 | 0.230 | 0.035 | 0.042 | 0.324 | 0.044 | 0.050 |

Each of Groups 3-8 showed reductions in H1N1 and in M1.

### Example 17. In Vivo Administration of IAV RNAi Agents to Mice Subsequently Infected with CA07 H1N1

Female C57Bl/6 mice animals were dosed with IAV RNAi agents and subsequently infected with CA07, in accordance with Example 4 (“CA07 H1N1 model mice”). On Days 1 and 3, four (n=4) mice (for Group 1) and five (n=5) mice (for Groups 2-8) were dosed with either saline or IAV RNAi agent formulated in saline (at 3 mg/kg), via intratracheal (IT) administration. On Day 8, the animals were dosed with either PBS or CA07 H1N1 formulated in PBS, via intranasal (IN) injection. Dosing was in accordance with Table 38 below.

TABLE 38
Dosing for mice animals of Example 17.

| Group | Dose (RNAi Agent) | Dose (CA07) |
|---|---|---|
| 1 | Day 1 and 3: IT 50 μL Saline | Day 8: IN 100 μL PBS |
| 2 | Day 1 and 3: IT 50 μL Saline | Day 8: IN 100 μL CA07 |
| | | (4240 TCID50) |
| 3 | Day 1 and 3: IT 50 μL 3 mg/kg | Day 8: IN 100 μL CA07 |
| | AC002601 | (4240 TCID50) |
| 4 | Day 1 and 3: IT 50 μL 3 mg/kg | Day 8: IN 100 μL CA07 |
| | AC002929 | (4240 TCID50) |
| 5 | Day 1 and 3: IT 50 μL 3 mg/kg | Day 8: IN 100 μL CA07 |
| | AC002930 | (4240 TCID50) |
| 6 | Day 1 and 3: IT 50 μL 3 mg/kg | Day 8: IN 100 μL CA07 |
| | AC002932 | (4240 TCID50) |
| 7 | Day 1 and 3: IT 50 μL 3 mg/kg | Day 8: IN 100 μL CA07 |
| | AC002933 | (4240 TCID50) |
| 8 | Day 1 and 3: IT 50 μL 3 mg/kg | Day 8: IN 100 μL CA07 |
| | AC002927 | (4240 TCID50) |

CA07 dose was quantified in TCID50 (50% tissue culture infective dose). This TCID50 dose was determined to be the optimal sub-lethal dose in accordance with the CA07 mouse model described in Example 3, above.

On Day 14, the animals were sacrificed. Lungs (both left and right) were collected and harvested. Expression of mouse H1N1 and M1 in lung was determined using qPCR, with 18s rRNA as endogenous control gene. Average H1N1 and M1 expression for each animal in lung tissue was normalized relative to Group 2 (no RNAi agent+CA07 infection). Results are shown in Table 39 below.

TABLE 39
Average relative expression of H1N1 and M1 in mice lung of Example 17.

| | Avg | | | Avg | | |
|---|---|---|---|---|---|---|
| Group ID | H1N1 | Low | High | M1 | Low | High |
| 1. Saline + PBS | N/A | N/A | N/A | N/A | N/A | N/A |
| 2. Saline + CA07 | 1.000 | 0.330 | 0.493 | 1.000 | 0.334 | 0.501 |
| 3. 3 mg/kg AC002601 + CA07 | 0.196 | 0.090 | 0.166 | 0.250 | 0.121 | 0.235 |
| 4. 3 mg/kg AC002929 + CA07 | 0.113 | 0.061 | 0.134 | 0.132 | 0.072 | 0.157 |
| 5. 3 mg/kg AC002930 + CA07 | 0.096 | 0.036 | 0.058 | 0.121 | 0.039 | 0.057 |
| 6. 3 mg/kg AC002932 + CA07 | 0.096 | 0.038 | 0.064 | 0.131 | 0.045 | 0.069 |
| 7. 3 mg/kg AC002933 + CA07 | 0.129 | 0.080 | 0.209 | 0.169 | 0.097 | 0.226 |
| 8. 3 mg/kg AC002927 + CA07 | 0.083 | 0.031 | 0.049 | 0.111 | 0.047 | 0.081 |

Each of Groups 3-8 showed reductions in H1N1 and in M1.

### Example 18. In Vivo Administration of IAV RNAi Agents to Mice Subsequently Infected with CA07H1N1

Female C57Bl/6 mice animals were dosed with IAV RNAi agents and subsequently infected with CA07, in accordance with Example 4 (“CA07 H1N1 model mice”). On Days 1 and 3, four (n=4) mice (for Group 1) and five (n=5) mice (for Groups 2-8) were dosed with either saline or IAV RNAi agent formulated in saline (at 3 mg/kg), via intratracheal (IT) administration. On Day 8, the animals were infected with either PBS or 4240 TCID50 of CA07 H1N1 formulated in PBS, via intranasal (IN) injection. Dosing was in accordance with Table 40 below.

TABLE 40
Dosing for mice animals of Example 18.

| Group | Dose (RNAi Agent) | Dose (CA07) |
|---|---|---|
| 1 | Day 1: IT 50 μL Saline | Day 8: IN 100 μL PBS |
| | Day 3: IT 100 μL Saline | |
| 2 | Day 1: IT 50 μL Saline | Day 8: IN 100 μL CA07 |
| | Day 3: IT 100 μL Saline | (4240 TCID50) |
| 3 | Day 1: IT 50 μL 3 mg/kg | Day 8: IN 100 μL CA07 |
| | AC002601 | (4240 TCID50) |
| | Day 3: IT 100 μL 3 mg/kg | |
| | AC002601 | |
| 4 | Day 1: IT 50 μL 3 mg/kg | Day 8: IN 100 μL CA07 |
| | AC002686 | (4240 TCID50) |
| | Day 3: IT 100 μL 3 mg/kg | |
| | AC002686 | |
| 5 | Day 1: IT 50 μL 3 mg/kg | Day 8: IN 100 μL CA07 |
| | AC002685 | (4240 TCID50) |
| | Day 3: IT 100 μL 3 mg/kg | |
| | AC002685 | |
| 6 | Day 1: IT 50 μL 3 mg/kg | Day 8: IN 100 μL CA07 |
| | AC002905 | (4240 TCID50) |
| | Day 1: IT 100 μL 3 mg/kg | |
| | AC002905 | |
| 7 | Day 1: IT 50 μL 3 mg/kg | Day 8: IN 100 μL CA07 |
| | AC002906 | (4240 TCID50) |
| | Day 3: IT 100 μL 3 mg/kg | |
| | AC002906 | |
| 8 | Day 1: IT 50 μL 3 mg/kg | Day 8: IN 100 μL CA07 |
| | AC002907 | (4240 TCID50) |
| | Day 3: IT 100 μL 3 mg/kg | |
| | AC002907 | |

CA07 dose was quantified in TCID50 (50% tissue culture infective dose). This TCID50 dose was determined to be the optimal sub-lethal dose in accordance with the CA07 mouse model described in Example 3, above.

On Day 14, the animals were sacrificed. Lungs (both left and right) were collected and harvested. Expression of mouse H1N1 and M1 in lung was determined using qPCR, with 18s rRNA as endogenous control gene. Average H1N1 and M1 expression for each animal in lung tissue was normalized relative to Group 2 (no RNAi agent+CA07 infection). Results are shown in Table 41 below.

TABLE 41
Average relative expression of H1N1 and M1 in mice lung of Example 18.

| | Avg | | | Avg | | |
|---|---|---|---|---|---|---|
| Group ID | H1N1 | Low | High | M1 | Low | High |
| 1. Saline + PBS | N/A | N/A | N/A | N/A | N/A | N/A |
| 2. Saline + CA07 | 1.000 | 0.465 | 0.869 | 1.000 | 0.428 | 0.749 |
| 3. 3 mg/kg AC002601+ CA07 | 0.122 | 0.026 | 0.033 | 0.138 | 0.029 | 0.037 |
| 4. 3 mg/kg AC002686+ CA07 | 0.623 | 0.193 | 0.279 | 0.659 | 0.199 | 0.285 |
| 5. 3 mg/kg AC002685+ CA07 | 0.415 | 0.078 | 0.096 | 0.402 | 0.047 | 0.054 |
| 6. 3 mg/kg AC002905+ CA07 | 0.686 | 0.192 | 0.267 | 0.553 | 0.149 | 0.204 |
| 7. 3 mg/kg AC002906+ CA07 | 1.077 | 0.287 | 0.392 | 0.788 | 0.158 | 0.197 |
| 8. 3 mg/kg AC002907+ CA07 | 0.951 | 0.223 | 0.291 | 0.652 | 0.132 | 0.165 |

The IAV RNAi agent of Group 3 (AC002601) was particularly potent, showing reductions over greater than 86% in both H1N1 and M1, while the IAV RNAi agents of the remaining Groups (i.e., Groups 4-8) showed only modest or in some cases no inhibition of H1N1 and M1.

### Example 19. In Vivo Administration of IAV RNAi Agents to Mice Subsequently Infected with CA07 H1N1

Female C57Bl/6 mice animals were dosed with IAV RNAi agents and subsequently infected with CA07, in accordance with Example 4 (“CA07 H1N1 model mice”). On Days 1 and 3, four (n=4) mice (for Group 1) and five (n=5) mice (for Groups 2-8) were dosed with either saline or IAV RNAi agent formulated in saline (at 3 mg/kg), via intratracheal (IT) administration. On Day 8, the animals were dosed with either PBS or CA07 H1N1 formulated in PBS, via intranasal (IN) injection. Dosing was in accordance with Table 42 below.

TABLE 42
Dosing for mice animals of Example 19.

| Group | Dose (RNAi Agent) | Dose (CA07) |
|---|---|---|
| 1 | Day 1 and 3: IT 50 μL Saline | Day 8: IN 100 μL PBS |
| 2 | Day 1 and 3: IT 50 μL Saline | Day 8: IN 100 μL CA07 |
| | | (4240 TCID50) |
| 3 | Day 1 and 3: IT 50 μL 3 mg/kg | Day 8: IN 100 μL CA07 |
| | AC002564 | (4240 TCID50) |
| 4 | Day 1 and 3: IT 50 μL 3 mg/kg | Day 8: IN 100 μL CA07 |
| | AC002569 | (4240 TCID50) |
| 5 | Day 1 and 3: IT 50 μL 3 mg/kg | Day 8: IN 100 μL CA07 |
| | AC002566 | (4240 TCID50) |
| 6 | Day 1 and 3: IT 50 μL 3 mg/kg | Day 8: IN 100 μL CA07 |
| | AC002567 | (4240 TCID50) |
| 7 | Day 1 and 3: IT 50 μL 3 mg/kg | Day 8: IN 100 μL CA07 |
| | AC002568 | (4240 TCID50) |
| 8 | Day 1 and 3: IT 50 μL 3 mg/kg | Day 8: IN 100 μL CA07 |
| | AC002601 | (4240 TCID50) |

CA07 dose was quantified in TCID50 (50% tissue culture infective dose). This TCID50 dose was determined to be the optimal sub-lethal dose in accordance with the CA07 mouse model described in Example 3, above.

On Day 14, the animals were sacrificed. Lungs (both left and right) were collected and harvested. Expression of mouse H1N1 and M1 in lung was determined using qPCR, with 18s rRNA as endogenous control gene. Average H1N1 and M1 expression for each animal in lung tissue was normalized relative to Group 2 (no RNAi agent+CA07 infection). Results are shown in Table 43 below.

TABLE 43
Average relative expression of HIN1 and M1 in mice lung of Example 19.

| | Avg | | | Avg | | |
|---|---|---|---|---|---|---|
| Group ID | H1N1 | Low | High | M1 | Low | High |
| 1. Saline + PBS | N/A | N/A | N/A | N/A | N/A | N/A |
| 2. Saline + CA07 | 1.000 | 0.281 | 0.392 | 1.000 | 0.306 | 0.441 |
| 3. 3 mg/kg AC002564 + CA07 | 0.282 | 0.115 | 0.195 | 0.233 | 0.083 | 0.128 |
| 4. 3 mg/kg AC002569 + CA07 | 0.560 | 0.232 | 0.397 | 0.579 | 0.179 | 0.259 |
| 5. 3 mg/kg AC002566 + CA07 | 0.344 | 0.106 | 0.153 | 0.300 | 0.079 | 0.108 |
| 6. 3 mg/kg AC002567 + CA07 | 1.193 | 0.513 | 0.899 | 0.782 | 0.243 | 0.352 |
| 7. 3 mg/kg AC002568 + CA07 | 1.055 | 0.359 | 0.545 | 0.747 | 0.286 | 0.464 |
| 8. 3 mg/kg AC002601 + CA07 | 0.126 | 0.034 | 0.046 | 0.123 | 0.041 | 0.062 |

Groups 3 and 8 showed substantial reductions in H1N1 and M1; Groups 4 and 5 showed more modest reductions in H1N1 and M1; and Groups 6 and 7 showed no reductions in H1N1 and only very limited reductions in M1.

### Example 20. In Vivo Administration of IAV RNAi Agents to Mice Subsequently Infected with CA07 H1N1

Female C57Bl/6 mice animals were dosed with IAV RNAi agents and subsequently infected with CA07, in accordance with Example 4 (“CA07 H1N1 model mice”). On Days 1 and 3, four (n=4) mice (for Group 1) and five (n=5) mice (for Groups 2-8) were dosed with either saline or IAV RNAi agent formulated in saline (at 1.5 mg/kg), via intratracheal (IT) administration. On Day 8, the animals were dosed with either PBS or CA07 H1N1 formulated in PBS, via intranasal (IN) injection. Dosing was in accordance with Table 44 below.

TABLE 44
Dosing for mice animals of Example 20.

| Group | Dose (RNAi Agent) | Dose (CA07) |
|---|---|---|
| 1 | Day 1 and 3: IT 50 μL Saline | Day 8: IN 100 μL PBS |
| 2 | Day 1 and 3: IT 50 μL Saline | Day 8: IN 100 μL CA07 |
| | | (4240 TCID50) |
| 3 | Day 1 and 3: IT 50 μL 1.5 mg/kg | Day 8: IN 100 μL CA07 |
| | AC002564 | (4240 TCID50) |
| 4 | Day 1 and 3: IT 50 μL 1.5 mg/kg | Day 8: IN 100 μL CA07 |
| | AC002865 | (4240 TCID50) |
| 5 | Day 1 and 3: IT 50 μL 1.5 mg/kg | Day 8: IN 100 μL CA07 |
| | AC002866 | (4240 TCID50) |
| 6 | Day 1 and 3: IT 50 μL 1.5 mg/kg | Day 8: IN 100 μL CA07 |
| | AC002869 | (4240 TCID50) |
| 7 | Day 1 and 3: IT 50 μL 1.5 mg/kg | Day 8: IN 100 μL CA07 |
| | AC002870 | (4240 TCID50) |
| 8 | Day 1 and 3: IT 50 μL 1.5 mg/kg | Day 8: IN 100 μL CA07 |
| | AC002872 | (4240 TCID50) |

CA07 dose was quantified in TCID50 (50% tissue culture infective dose). This TCID50 dose was determined to be the optimal sub-lethal dose in accordance with the CA07 mouse model described in Example 3, above.

On Day 14, the animals were sacrificed. Lungs (both left and right) were collected and harvested. Expression of mouse H1N1 and M1 in lung was determined using qPCR, with 18s rRNA as endogenous control gene. Average H1N1 and M1 expression for each animal in lung tissue was normalized relative to Group 2 (no RNAi agent+CA07 infection). Results are shown in Table 45 below.

TABLE 45
Average relative expression of H1N1 and M1 in mice lung of Example 20.

| | Avg | | | Avg | | |
|---|---|---|---|---|---|---|
| Group ID | H1N1 | Low | High | M1 | Low | High |
| 1. Saline + PBS | N/A | N/A | N/A | N/A | N/A | N/A |
| 2. Saline + CA07 | 1.000 | 0.309 | 0.448 | 1.000 | 0.221 | 0.283 |
| 3. 1.5 mg/kg AC002564 + CA07 | 0.208 | 0.099 | 0.189 | 0.252 | 0.091 | 0.143 |
| 4. 1.5 mg/kg AC002865 + CA07 | 0.140 | 0.064 | 0.118 | 0.221 | 0.090 | 0.152 |
| 5. 1.5 mg/kg AC002866 + CA07 | 0.165 | 0.069 | 0.120 | 0.215 | 0.069 | 0.103 |
| 6. 1.5 mg/kg AC002869 + CA07 | 0.155 | 0.046 | 0.066 | 0.245 | 0.087 | 0.135 |
| 7. 1.5 mg/kg AC002870 + CA07 | 0.203 | 0.066 | 0.099 | 0.354 | 0.148 | 0.255 |
| 8. 1.5 mg/kg AC002872 + CA07 | 0.237 | 0.023 | 0.026 | 0.366 | 0.085 | 0.111 |

Each of Groups 3-8 showed meaningful reductions in both H1N1 and in M1.

### Example 21. In Vivo Administration of IAV RNAi Agents to Mice Subsequently Infected with CA07 H1N1

Female C57Bl/6 mice animals were dosed with IAV RNAi agents and subsequently infected with CA07, in accordance with Example 4 (“CA07 H1N1 model mice”). On Days 1 and 3, eight (n=8) mice (for Groups 1-5) were dosed with either saline or IAV RNAi agent formulated in saline (at 3 mg/kg), via intratracheal (IT) administration. On Day 8, the animals were dosed with either PBS or CA07 H1N1 formulated in PBS, via intranasal (IN) injection. Dosing was in accordance with Table 46 below.

TABLE 46
Dosing for mice animals of Example 21.

| Group | Dose (RNAi Agent) | Dose (CA07) |
|---|---|---|
| 1 | Day 1 and 3: IT 50 μL Saline | Day 8: IN 100 μL PBS |
| 2 | Day 1 and 3: IT 50 μL Saline | Day 8: IN 100 μL CA07 |
| | | (12000 TCID50) |
| 3 | Day 1 and 3: IT 50 μL 3 mg/kg | Day 8: IN 100 μL CA07 |
| | AC002601 | (12000 TCID50) |
| 4 | Day 1 and 3: IT 50 μL Saline | Day 8: IN 100 μL CA07 |
| | | (24000 TCID50) |
| 5 | Day 1 and 3: IT 50 μL 3 mg/kg | Day 8: IN 100 μL CA07 |
| | AC002601 | (24000 TCID50) |

CA07 dose was quantified in TCID50 (50% tissue culture infective dose).

On Day 22, the animals were sacrificed. Mice were observed for body weight and survival rates post CA07 infection. The survival rates are shown in Table 47 below.

TABLE 47
Survival rates of test animals post CA07 infection of Example 21.

| Group ID | % Survival |
|---|---|
| 1. Saline + PBS | 100% 11 days post infection |
| 2. Saline + CA07 (12000 TCID50) | 62.5% 5 days post infection |
| | 50% 6 days post infection |
| | 25% 7 days post infection |
| | 12.5% 10 days post infection |
| | 12.5% 11 days post infection |
| 3. AC002601 + CA07 (12000 TCID50) | 100% 11 days post infection |
| 4. Saline + CA07 (24000 TCID50) | 75% 4 days post infection |
| | 12.5% 5 days post infection |
| | 0% 7 days post infection |
| 5. AC002601 + CA07 (24000 TCID50) | 87.5% 7 days post infection |
| | 87.5% 11 days post infection |

Dosing with AC002601, followed by subsequent infection with CA07 (12000 TCID50), achieved 100% survival rate at Day 11 post infection. Dosing with AC002601, followed by subsequent infection with CA07 (24000 TCID50), achieved 87.5% survival rate at 11 days post infection. This is contrasted with only a 12.5% survival rate 11 days post infection in Group 2 and a 0% survival rate 7 days post infection in Group 4, where in both cases the same infection was administered to the mice but no IAV RNAi agent was dosed.

### Example 22. In Vivo Administration of IAV RNAi Agents to Mice Subsequently Infected with CA07 H1N1

Female C57Bl/6 mice animals were dosed with IAV RNAi agents and subsequently infected with CA07, in accordance with Example 4 (“CA07 H1N1 model mice”). On Days 1 and 3, four (n=4) mice (for Group 1) and five (n=5) mice (for Groups 2-8) were dosed with either saline or IAV RNAi agent formulated in saline (at 3 mg/kg), via intratracheal (IT) administration. On Day 8, the animals were dosed with either PBS or CA07 H1N1 formulated in PBS, via intranasal (IN) injection. Dosing was in accordance with Table 48 below.

TABLE 48
Dosing for mice animals of Example 22.

| Group | Dose (RNAi Agent) | Dose (CA07) |
|---|---|---|
| 1 | Day 1 and 3: IT 50 μL Saline | Day 8: IN 100 μL PBS |
| 2 | Day 1 and 3: IT 50 μL Saline | Day 8: IN 100 μL CA07 |
| | | (4240 TCID50) |
| 3 | Day 1 and 3: IT 50 μL 3 mg/kg | Day 8: IN 100 μL CA07 |
| | AC002601 | (4240 TCID50) |
| 4 | Day 1 and 3: IT 50 μL 3 mg/kg | Day 8: IN 100 μL CA07 |
| | AC002908 | (4240 TCID50) |
| 5 | Day 1 and 3: IT 50 μL 3 mg/kg | Day 8: IN 100 μL CA07 |
| | AC002599 | (4240 TCID50) |
| 6 | Day 1 and 3: IT 50 μL 3 mg/kg | Day 8: IN 100 μL CA07 |
| | AC002600 | (4240 TCID50) |
| 7 | Day 1 and 3: IT 50 μL 3 mg/kg | Day 8: IN 100 μL CA07 |
| | AC002909 | (4240 TCID50) |
| 8 | Day 1 and 3: IT 50 μL 3 mg/kg | Day 8: IN 100 μL CA07 |
| | AC002367 | (4240 TCID50) |

CA07 dose was quantified in TCID50 (50% tissue culture infective dose). This TCID50 dose was determined to be the optimal sub-lethal dose in accordance with the CA07 mouse model described in Example 3, above.

On Day 14, the animals were sacrificed. Lungs (both left and right) were collected and harvested. Expression of mouse H1N1 and M1 in lung was determined using qPCR, with 18s rRNA as endogenous control gene. Average H1N1 and M1 expression for each animal in lung tissue was normalized relative to Group 2 (no RNAi agent+CA07 infection). Results are shown in Table 49 below.

TABLE 49
Average relative expression of HIN1 and M1 in mice lung of Example 22.

| | Avg | | | Avg | | |
|---|---|---|---|---|---|---|
| Group ID | H1N1 | Low | High | M1 | Low | High |
| 1. Saline + PBS | N/A | N/A | N/A | N/A | N/A | N/A |
| 2. Saline + CA07 | 1.000 | 0.577 | 1.366 | 1.000 | 0.578 | 1.369 |
| 3. 3 mg/kg AC002601 + CA07 | 0.073 | 0.040 | 0.087 | 0.062 | 0.026 | 0.044 |
| 4. 3 mg/kg AC002908 + CA07 | 0.762 | 0.414 | 0.904 | 0.513 | 0.288 | 0.659 |
| 5. 3 mg/kg AC002599 + CA07 | 0.498 | 0.255 | 0.524 | 0.376 | 0.194 | 0.399 |
| 6. 3 mg/kg AC002600 + CA07 | 0.549 | 0.223 | 0.374 | 0.272 | 0.086 | 0.126 |
| 7. 3 mg/kg AC002909 + CA07 | 1.084 | 0.468 | 0.824 | 0.463 | 0.273 | 0.663 |
| 8. 3 mg/kg AC002367 + CA07 | 0.717 | 0.377 | 0.795 | 0.312 | 0.106 | 0.161 |

Group 3 showed substantial reductions in H1N1 and M1.

### Example 23. In Vivo Administration of IAV RNAi Agents to Mice Subsequently Infected with CA07 H1N1

Female C57Bl/6 mice animals were dosed with IAV RNAi agents and subsequently infected with CA07, in accordance with Example 4 (“CA07 H1N1 model mice”). On Days 1 and 3, four (n=4) mice (for Group 1) and five (n=5) mice (for Groups 2-8) were dosed with either saline or IAV RNAi agent formulated in saline (at 3 mg/kg), via intratracheal (IT) administration. On Day 8, the animals were dosed with either PBS or CA07 H1N1 formulated in PBS, via intranasal (IN) injection. Dosing was in accordance with Table 50 below.

TABLE 50
Dosing for mice animals of Example 23.

| Group | Dose (RNAi Agent) | Dose (CA07) |
|---|---|---|
| 1 | Day 1 and 3: IT 50 μL Saline | Day 8: IN 100 μL PBS |
| 2 | Day 1 and 3: IT 50 μL Saline | Day 8: IN 100 μL CA07 |
| | | (4240 TCID50) |
| 3 | Day 1 and 3: IT 50 μL 3 mg/kg | Day 8: IN 100 μL CA07 |
| | AC002601 | (4240 TCID50) |
| 4 | Day 1 and 3: IT 50 μL 3 mg/kg | Day 8: IN 100 μL CA07 |
| | AC002564 | (4240 TCID50) |
| 5 | Day 1 and 3: IT 50 μL 3 mg/kg | Day 8: IN 100 μL CA07 |
| | AC002369 | (4240 TCID50) |
| 6 | Day 1 and 3: IT 50 μL 3 mg/kg | Day 8: IN 100 μL CA07 |
| | AC002682 | (4240 TCID50) |
| 7 | Day 1 and 3: IT 50 μL 3 mg/kg | Day 8: IN 100 μL CA07 |
| | AC002683 | (4240 TCID50) |
| 8 | Day 1 and 3: IT 50 μL 3 mg/kg | Day 8: IN 100 μL CA07 |
| | AC002910 | (4240 TCID50) |

CA07 dose was quantified in TCID50 (50% tissue culture infective dose). This TCID50 dose was determined to be the optimal sub-lethal dose in accordance with the CA07 mouse model described in Example 3, above.

On Day 14, the animals were sacrificed. Lungs (both left and right) were collected and harvested. Expression of mouse H1N1 and M1 in lung was determined using qPCR, with 18s rRNA as endogenous control gene. Average H1N1 and M1 expression for each animal in lung tissue was normalized relative to Group 2 (no RNAi agent+CA07 infection). Results are shown in Table 51 below.

TABLE 51
Average relative expression of H1N1 and M1 in mice lung of Example 23.

| | Avg | | | Avg | | |
|---|---|---|---|---|---|---|
| Group ID | H1N1 | Low | High | M1 | Low | High |
| 1. Saline + PBS | N/A | N/A | N/A | N/A | N/A | N/A |
| 2. Saline + CA07 | 1.000 | 0.161 | 0.192 | 1.000 | 0.200 | 0.250 |
| 3. 3 mg/kg AC002601 + CA07 | 0.138 | 0.034 | 0.045 | 0.169 | 0.046 | 0.062 |
| 4. 3 mg/kg AC002564 + CA07 | 0.343 | 0.047 | 0.055 | 0.349 | 0.059 | 0.071 |
| 5. 3 mg/kg AC002369 + CA07 | 0.751 | 0.259 | 0.397 | 0.869 | 0.261 | 0.373 |
| 6. 3 mg/kg AC002682 + CA07 | 0.937 | 0.179 | 0.222 | 0.971 | 0.202 | 0.256 |
| 7. 3 mg/kg AC002683 + CA07 | 1.132 | 0.204 | 0.250 | 1.152 | 0.201 | 0.243 |
| 8. 3 mg/kg AC002910 + CA07 | 1.351 | 0.373 | 0.516 | 1.267 | 0.359 | 0.501 |

Group 3 (AC002601) showed substantial reductions in H1N1 and M1. Group 4 (AC002564) showed more modest reductions in H1N1 and M1. The remaining Groups (Groups 5-8) showed limited to no inhibition of H1N1 and M1.

### Example 24. In Vivo Administration of IAV RNAi Agents to Mice Subsequently Infected with CA07 H1N1

Female C57Bl/6 mice animals were dosed with IAV RNAi agents and subsequently infected with CA07, in accordance with Example 4 (“CA07 H1N1 model mice”). On Days 1 and 3, eight (n=8) mice (for Groups 1-5) were dosed with either saline or IAV RNAi agent formulated in saline (at 3 mg/kg), via intratracheal (IT) administration. On Day 8, the animals were dosed with either PBS or CA07 H1N1 formulated in PBS, via intranasal (IN) injection. Dosing was in accordance with Table 52 below.

TABLE 52
Dosing for mice animals of Example 24.

| Group | Dose (RNAi Agent) | Dose (CA07) |
|---|---|---|
| 1 | Day 1 and 3: IT 50 μL Saline | Day 8: IN 100 μL PBS |
| 2 | Day 1 and 3: IT 50 μL Saline | Day 8: IN 100 μL CA07 |
| | | (12000 TCID50) |
| 3 | Day 1 and 3: IT 50 μL 3 mg/kg | Day 8: IN 100 μL CA07 |
| | AC002564 | (12000 TCID50) |
| 4 | Day 1 and 3: IT 50 μL Saline | Day 8: IN 100 μL CA07 |
| | | (24000 TCID50) |
| 5 | Day 1 and 3: IT 50 μL 3 mg/kg | Day 8: IN 100 μL CA07 |
| | AC002564 | (24000 TCID50) |

CA07 dose was quantified in TCID50 (50% tissue culture infective dose).

On Day 22, the animals were sacrificed. Mice were observed for body weight and survival rates post CA07 infection. The survival rates are shown in Table 53 below.

TABLE 53
Survival rates of test animals post CA07 infection of Example 24.

| Group ID | % Survival |
|---|---|
| 1. Saline + PBS | 100% 15 days post infection |
| 2. Saline + CA07 | 66% 7 days post infection |
| (12000 TCID50) | 33% 8 days post infection |
| | 33% 15 days post infection |
| 3. AC002564 + CA07 | 83.3% 8 days post infection |
| (12000 TCID50) | 83.3% 15 days post infection |
| 4. Saline + CA07 | 87.5% 5 days post infection |
| (24000 TCID50) | 75% 6 days post infection |
| | 62.5% 7 days post infection |
| | 25% 8 days post infection |
| | 25% 15 days post infection |
| 5. AC002564 + CA07 | 87.5% 7 days post infection |
| (24000 TCID50) | 75% 8 days post infection |
| | 75% 15 days post infection |

Dosing with AC002564, followed by subsequent infection with CA07 (12000 TCID50), achieved 83.3% survival rate at 15 days post infection. Dosing with AC002564, followed by subsequent infection with CA07 (24000 TCID50), achieved 75% survival rate at 15 days post infection. This is in contrast to Group 2 (only a 33% survival rate at 15 days post infection) and Group 4 (only 25% survival rate at 15 days post infection), where no RNAi agent was administered.

### Example 25. In Vivo Administration of IAV RNAi Agents to Mice Subsequently Infected with CA07 H1N1

Female C57Bl/6 mice animals were dosed with IAV RNAi agents and subsequently infected with CA07, in accordance with Example 4 (“CA07 H1N1 model mice”). On Days 1 and 3, four (n=4) mice (for Group 1) and five (n=5) mice (for Groups 2-8) were dosed with either saline or IAV RNAi agent formulated in saline (at 3 mg/kg), via intratracheal (IT) administration. On Day 8, the animals were dosed with either PBS or CA07 H1N1 formulated in PBS, via intranasal (IN) injection. Dosing was in accordance with Table 54 below.

TABLE 54
Dosing for mice animals of Example 25.

| Group | Dose (RNAi Agent) | Dose (CA07) |
|---|---|---|
| 1 | Day 1 and 3: IT 50 μL Saline | Day 8: IN 100 μL PBS |
| 2 | Day 1 and 3: IT 50 μL Saline | Day 8: IN 100 μL CA07 |
| | | (4240 TCID50) |
| 3 | Day 1 and 3: IT 50 μL 3 mg/kg | Day 8: IN 100 μL CA07 |
| | AC002601 | (4240 TCID50) |
| 4 | Day 1 and 3: IT 50 μL 3 mg/kg | Day 8: IN 100 μL CA07 |
| | AC002925 | (4240 TCID50) |
| 5 | Day 1 and 3: IT 50 μL 3 mg/kg | Day 8: IN 100 μL CA07 |
| | AC002926 | (4240 TCID50) |
| 6 | Day 1 and 3: IT 50 μL 3 mg/kg | Day 8: IN 100 μL CA07 |
| | AC002927 | (4240 TCID50) |
| 7 | Day 1 and 3: IT 50 μL 3 mg/kg | Day 8: IN 100 μL CA07 |
| | AC002928 | (4240 TCID50) |
| 8 | Day 1 and 3: IT 50 μL 3 mg/kg | Day 8: IN 100 μL CA07 |
| | AC002929 | (4240 TCID50) |

CA07 dose was quantified in TCID50 (50% tissue culture infective dose). This TCID50 dose was determined to be the optimal sub-lethal dose in accordance with the CA07 mouse model described in Example 3, above.

On Day 14, the animals were sacrificed. Lungs (both left and right) were collected and harvested. Expression of mouse H1N1 and M1 in lung was determined using qPCR, with 18s rRNA as endogenous control gene. Average H1N1 and M1 expression for each animal in lung tissue was normalized relative to Group 2 (no RNAi agent+CA07 infection). Results are shown in Table 55 below.

TABLE 55
Average relative expression of H1N1 and M1 in mice lung of Example 25.

| | Avg | | | Avg | | |
|---|---|---|---|---|---|---|
| Group ID | H1N1 | Low | High | M1 | Low | High |
| 1. Saline + PBS | N/A | N/A | N/A | N/A | N/A | N/A |
| 2. Saline + CA07 | 1.000 | 0.301 | 0.430 | 1.000 | 0.369 | 0.586 |
| 3. 3 mg/kg AC002601 + CA07 | 0.088 | 0.041 | 0.077 | 0.095 | 0.034 | 0.053 |
| 4. 3 mg/kg AC002925 + CA07 | 0.183 | 0.093 | 0.191 | 0.145 | 0.085 | 0.207 |
| 5. 3 mg/kg AC002926 + CA07 | 0.142 | 0.042 | 0.059 | 0.097 | 0.033 | 0.049 |
| 6. 3 mg/kg AC002927 + CA07 | 0.097 | 0.053 | 0.118 | 0.042 | 0.017 | 0.030 |
| 7. 3 mg/kg AC002928 + CA07 | 0.170 | 0.077 | 0.139 | 0.064 | 0.026 | 0.045 |
| 8. 3 mg/kg AC002929 + CA07 | 0.125 | 0.064 | 0.132 | 0.093 | 0.040 | 0.069 |

Each of Groups 3-8 showed reduction in H1N1 and in M1.

### Example 26. In Vivo Administration of IAV RNAi Agents to Mice Subsequently Infected with CA07 H1N1

Female C57Bl/6 mice animals were dosed with IAV RNAi agents and subsequently infected with CA07, in accordance with Example 4 (“CA07 H1N1 model mice”). On Days 1 and 3, four (n=4) mice (for Group 1) and five (n=5) mice (for Groups 2-8) were dosed with either saline or IAV RNAi agent formulated in saline (at 3 mg/kg), via intratracheal (IT) administration. On Day 8, the animals were dosed with either PBS or CA07 H1N1 formulated in PBS, via intranasal (IN) injection. Dosing was in accordance with Table 56 below.

TABLE 56
Dosing for mice animals of Example 26.

| Group | Dose (RNAi Agent) | Dose (CA07) |
|---|---|---|
| 1 | Day 1 and 3: IT 50 μL | Day 8: IN 100 μL PBS |
| | Saline | |
| 2 | Day 1 and 3: IT 50 μL | Day 8: IN 100 μL CA07 |
| | Saline | (4240 TCID50) |
| 3 | Day 1 and 3: IT 50 μL | Day 8: IN 100 μL CA07 |
| | 3 mg/kg AC002601 | (4240 TCID50) |
| 4 | Day 1 and 3: IT 50 μL | Day 8: IN 100 μL CA07 |
| | 3 mg/kg AC002930 | (4240 TCID50) |
| 5 | Day 1 and 3: IT 50 μL | Day 8: IN 100 μL CA07 |
| | 3 mg/kg AC002931 | (4240 TCID50) |
| 6 | Day 1 and 3: IT 50 μL | Day 8: IN 100 μL CA07 |
| | 3 mg/kg AC002932 | (4240 TCID50) |
| 7 | Day 1 and 3: IT 50 μL | Day 8: IN 100 μL CA07 |
| | 3 mg/kg AC002933 | (4240 TCID50) |
| 8 | Day 1 and 3: IT 50 μL | Day 8: IN 100 μL CA07 |
| | 3 mg/kg AC002934 | (4240 TCID50) |

CA07 dose was quantified in TCID50 (50% tissue culture infective dose). This TCID50 dose was determined to be the optimal sub-lethal dose in accordance with the CA07 mouse model described in Example 3, above.

On Day 14, the animals were sacrificed. Lungs (both left and right) were collected and harvested. Expression of mouse H1N1 and M1 in lung was determined using qPCR, with 18s rRNA as endogenous control gene. Average H1N1 and M1 expression for each animal in lung tissue was normalized relative to Group 2 (no RNAi agent+CA07 infection). Results are shown in Table 57 below.

TABLE 57
Average relative expression of H1N1 and M1 in mice lung of Example 26.

| | Avg | | | Avg | | |
|---|---|---|---|---|---|---|
| Group ID | H1N1 | Low | High | M1 | Low | High |
| 1. Saline + PBS | N/A | N/A | N/A | N/A | N/A | N/A |
| 2. Saline + CA07 | 1.000 | 0.590 | 1.438 | 1.000 | 0.192 | 0.238 |
| 3. 3 mg/kg AC002601 + CA07 | 0.157 | 0.050 | 0.074 | 0.478 | 0.254 | 0.541 |
| 4. 3 mg/kg AC002930 + CA07 | 0.183 | 0.090 | 0.177 | 0.441 | 0.271 | 0.701 |
| 5. 3 mg/kg AC002931 + CA07 | 0.125 | 0.056 | 0.103 | 0.282 | 0.146 | 0.305 |
| 6. 3 mg/kg AC002932 + CA07 | 0.173 | 0.110 | 0.299 | 0.244 | 0.151 | 0.392 |
| 7. 3 mg/kg AC002933 + CA07 | 0.136 | 0.065 | 0.125 | 0.178 | 0.064 | 0.100 |
| 8. 3 mg/kg AC002934 + CA07 | 0.284 | 0.112 | 0.184 | 0.306 | 0.075 | 0.099 |

Groups 3-8 showed certain reductions in H1N1 and in M1.

### Example 27. In Vivo Administration of IAV RNAi Agents to Mice Previously and Subsequently Infected with CA07 H1N1

Female C57Bl/6 Mice animals were dosed with IAV RNAi agents and subsequently infected with CA07, in accordance with Example 4 (“CA07 H1N1 model Mice”). On Days 1, 2, and/or 3, five (n=5) Mice (for Groups 1-8) were dosed with either saline or IAV RNAi agent formulated in saline (at 3 mg/kg or 6 mg/kg), via intratracheal (IT) or intranasal (IN) administration. On Day 1, the animals were dosed with either PBS or CA07 H1N1 formulated in PBS, via intranasal (IN) injection. Dosing was in accordance with Table 58 below.

TABLE 58
Dosing for mice animals of Example 27.

| Group | Dose (RNAi Agent) | Dose (CA07) |
|---|---|---|
| 1 | Day 1 (4 hours | Day 8: IN |
| | post infection): | 100 μL PBS |
| | IT 50 μL Saline | |
| | Day 2: IT 50 μL Saline | |
| 2 | Day 1 (4 hours | Day 8: IN |
| | post infection): | 100 μL CA07 |
| | IN 50 μL Saline | (4240 TCID50) |
| 3 | Day 1 (2 hours | Day 8: IN |
| | before infection): | 100 μL CA07 |
| | IN 50 μL 3 mg/kg | (4240 TCID50) |
| | AC002601 | |
| 4 | Day 1 (2 hours | Day 8: IN |
| | before infection): | 100 μL CA07 |
| | IN 50 μL 6 mg/kg | (4240 TCID50) |
| | AC002601 | |
| 5 | Day 1 (4 hours | Day 8: IN |
| | post infection): | 100 μL CA07 |
| | IN 50 μL 3 mg/kg | (4240 TCID50) |
| | AC002601 | |
| 6 | Day 1 (4 hours | Day 8: IN |
| | post infection): | 100 μL CA07 |
| | IN 50 μL 3 mg/kg | (4240 TCID50) |
| | AC002601 | |
| | Day 2: IN 50 μL | |
| | 3 mg/kg AC002601 | |
| 7 | Day 1 (4 hours | Day 8: IN |
| | post infection): | 100 μL CA07 |
| | IN 50 μL 6 mg/kg | (4240 TCID50) |
| | AC002601 | |
| | Day 2: IN 50 μL | |
| | 6 mg/kg AC002601 | |
| 8 | Day 1 (4 hours | Day 8: IN |
| | post infection): | 100 μL CA07 |
| | IN 50 μL 3 mg/kg | (4240 TCID50) |
| | AC002601 | |
| | Day 2: IN 50 μL | |
| | 3 mg/kg AC002601 | |
| | Day 3: IN 50 μL | |
| | 3 mg/kg AC002601 | |

CA07 dose was quantified in TCID50 (50% tissue culture infective dose). This TCID50 dose was determined to be the optimal sub-lethal dose in accordance with the CA07 mouse model described in Example 3, above.

On Day 7, the animals were sacrificed. Lungs (both left and right) were collected and harvested. Expression of mouse H1N1 in lung was determined using qPCR, with 18s rRNA as endogenous control gene. Average H1N1 expression for each animal in lung tissue was normalized relative to Group 2 (no RNAi agent+CA07 infection). Results are shown in Table 59 below.

TABLE 59
Average relative expression of H1N1 in mice lung of Example 27.

| | Avg | | |
|---|---|---|---|
| Group ID | H1N1 | Low | High |
| 1. Saline + PBS | N/A | N/A | N/A |
| 2. Saline + CA07 | 1.000 | 0.388 | 0.392 |
| 3. D 1 3 mg/kg AC002601 | 0.092 | 0.036 | 0.059 |
| (2 h before CA07) + CA07 | | | |
| 4. D 1 6 mg/kg AC002601 | 0.060 | 0.028 | 0.052 |
| (2 h before CA07) + CA07 | | | |
| 5. D 1 3 mg/kg AC002601 | 0.240 | 0.071 | 0.100 |
| (4 h post CA07) + CA07 | | | |
| 6. D 1 3 mg/kg AC002601 | 0.071 | 0.029 | 0.049 |
| (4 h post CA07) + D 2 3 | | | |
| mg/kg AC002601 + CA07 | | | |
| 7. D 1 6 mg/kg AC002601 | 0.084 | 0.055 | 0.154 |
| (4 h post CA07) + D2 6 | | | |
| mg/kg AC002601 + CA07 | | | |
| 8. D1 3 mg/kg AC002601 | 0.396 | 0.110 | 0.153 |
| (4 h post CA07) + D2 3 | | | |
| mg/kg AC002601 + D3 3 | | | |
| mg/kg AC002601 + CA07 | | | |

Groups 3, 4, 6, and 7 showed substantial reduction of H1N1, and Groups 5 and 8 showed more modest reduction of H1N1.

### Example 28. In Vivo Administration of IAV RNAi Agents to Mice Subsequently Infected with CA07 H1N1

Female C57Bl/6 mice animals were dosed with IAV RNAi agents and subsequently infected with CA07, in accordance with Example 4 (“CA07 H1N1 model mice”). On Days 1 and 3, five (n=5) mice (for Groups 1-8) were dosed with either saline or IAV RNAi agent formulated in saline (at 0.75 mg/kg), via intratracheal (IT) administration. On Day 8, the animals were dosed with either PBS or CA07 H1N1 formulated in PBS, via intranasal (IN) injection. Dosing was in accordance with Table 60 below.

TABLE 60
Dosing for mice animals of Example 28.

| Group | Dose (RNAi Agent) | Dose (CA07) |
|---|---|---|
| 1 | Day 1 and 3: IT 50 μL | Day 8: IN 100 μL PBS |
| | Saline | |
| 2 | Day 1 and 3: IT 50 μL | Day 8: IN 100 μL CA07 |
| | Saline | (4240 TCID50) |
| 3 | Day 1 and 3: IT 50 μL | Day 8: IN 100 μL CA07 |
| | 0.75 mg/kg AC002564 | (4240 TCID50) |
| 4 | Day 1 and 3: IT 50 μL | Day 8: IN 100 μL CA07 |
| | 0.75 mg/kg AC002863 | (4240 TCID50) |
| 5 | Day 1 and 3: IT 50 μL | Day 8: IN 100 μL CA07 |
| | 0.75 mg/kg AC002864 | (4240 TCID50) |
| 6 | Day 1 and 3: IT 50 μL | Day 8: IN 100 μL CA07 |
| | 0.75 mg/kg AC002865 | (4240 TCID50) |
| 7 | Day 1 and 3: IT 50 μL | Day 8: IN 100 μL CA07 |
| | 0.75 mg/kg AC002866 | (4240 TCID50) |
| 8 | Day 1 and 3: IT 50 μL | Day 8: IN 100 μL CA07 |
| | 0.75 mg/kg AC002869 | (4240 TCID50) |

CA07 dose was quantified in TCID50 (50% tissue culture infective dose). This TCID50 dose was determined to be the optimal sub-lethal dose in accordance with the CA07 mouse model described in Example 3, above.

On Day 14, the animals were sacrificed. Lungs (both left and right) were collected and harvested. Expression of mouse H1N1 and M1 in lung was determined using qPCR, with 18s rRNA as endogenous control gene. Average H1N1 and M1 expression for each animal in lung tissue was normalized relative to Group 2 (no RNAi agent+CA07 infection). Results are shown in Table 61 below.

TABLE 61
Average relative expression of H1N1 and M1 in mice lung of Example 28.

| | Avg | | | Avg | | |
|---|---|---|---|---|---|---|
| Group ID | H1N1 | Low | High | M1 | Low | High |
| 1. Saline + PBS | N/A | N/A | N/A | N/A | N/A | N/A |
| 2. Saline + CA07 | 1.000 | 0.472 | 0.446 | 1.000 | 0.536 | 0.611 |
| 3. 0.75 mg/kg AC002564+ CA07 | 0.301 | 0.105 | 0.162 | 0.297 | 0.090 | 0.130 |
| 4. 0.75 mg/kg AC002863+ CA07 | 0.213 | 0.067 | 0.098 | 0.213 | 0.070 | 0.104 |
| 5. 0.75 mg/kg AC002864+ CA07 | 0.159 | 0.073 | 0.136 | 0.147 | 0.063 | 0.111 |
| 6. 0.75 mg/kg AC002865+ CA07 | 0.123 | 0.047 | 0.077 | 0.123 | 0.046 | 0.072 |
| 7. 0.75 mg/kg AC002866+ CA07 | 0.189 | 0.063 | 0.095 | 0.184 | 0.086 | 0.162 |
| 8. 0.75 mg/kg AC002869+ CA07 | 0.148 | 0.055 | 0.088 | 0.114 | 0.042 | 0.066 |

Groups 3-8 each showed reductions in H1N1 and in M1.

### Example 29. In Vivo Administration of IAV RNAi Agents to Mice Subsequently Infected with CA07 H1N1

Female C57Bl/6 mice animals were dosed with IAV RNAi agents and subsequently infected with CA07, in accordance with Example 4 (“CA07 H1N1 model mice”). On Days 1 and 3, four (n=4) mice (for Group 1) and five (n=5) mice (for Groups 2-8) were dosed with either saline or IAV RNAi agent formulated in saline (at 1 mg/kg or 2 mg/kg), via intratracheal (IT) administration. On Day 8, the animals were dosed with either PBS or CA07 H1N1 formulated in PBS, via intranasal (IN) injection. Dosing was in accordance with Table 62 below.

TABLE 62
Dosing for mice animals of Example 29.

| Group | Dose (RNAi Agent) | Dose (CA07) |
|---|---|---|
| 1 | Day 1 and 3: IT 50 μL | Day 8: IN 100 μL PBS |
| | Saline | |
| 2 | Day 1 and 3: IT 50 μL | Day 8: IN 100 μL CA07 |
| | Saline | (4240 TCID50) |
| 3 | Day 1 and 3: IT 50 μL | Day 8: IN 100 μL CA07 |
| | 2 mg/kg AC002601 | (4240 TCID50) |
| 4 | Day 1 and 3: IT 50 μL | Day 8: IN 100 μL CA07 |
| | 2 mg/kg AC002927 | (4240 TCID50) |
| 5 | Day 1 and 3: IT 50 μL | Day 8: IN 100 μL CA07 |
| | 2 mg/kg AC002929 | (4240 TCID50) |
| 6 | Day 1 and 3: IT 50 μL | Day 8: IN 100 μL CA07 |
| | 1 mg/kg AC002601 | (4240 TCID50) |
| 7 | Day 1 and 3: IT 50 μL | Day 8: IN 100 μL CA07 |
| | 1 mg/kg AC002927 | (4240 TCID50) |
| 8 | Day 1 and 3: IT 50 μL | Day 8: IN 100 μL CA07 |
| | 1 mg/kg AC002929 | (4240 TCID50) |

CA07 dose was quantified in TCID50 (50 tissue culture infective dose). This TCID50 dose was determined to be the optimal sub-lethal dose in accordance with the CA07 mouse model described in Example 3, above.

On Day 14, the animals were sacrificed. Lungs (both left and right) were collected and harvested. Expression of mouse H1N1 and M1 in lung was determined using qPCR, with 18s rRNA as endogenous control gene. Average H1N1 and M1 expression for each animal in lung tissue was normalized relative to Group 2 (no RNAi agent+CA07 infection). Results are shown in Table 63 below.

TABLE 63
Average relative expression of H1N1 and M1 in mice lung of Example 29.

| | Avg | | | Avg | | |
|---|---|---|---|---|---|---|
| Group ID | H1N1 | Low | High | M1 | Low | High |
| 1. Saline + PBS | N/A | N/A | N/A | N/A | N/A | N/A |
| 2. Saline + CA07 | 1.000 | 0.171 | 0.206 | 1.000 | 0.219 | 0.280 |
| 3. 2 mg/kg AC002601+ CA07 | 0.212 | 0.095 | 0.172 | 0.226 | 0.091 | 0.152 |
| 4. 2 mg/kg AC002927+ CA07 | 0.130 | 0.046 | 0.071 | 0.135 | 0.049 | 0.076 |
| 5. 2 mg/kg AC002929+ CA07 | 0.293 | 0.168 | 0.394 | 0.309 | 0.162 | 0.340 |
| 6. 1 mg/kg AC002601+ CA07 | 0.398 | 0.199 | 0.398 | 0.450 | 0.221 | 0.433 |
| 7. 1 mg/kg AC002927+ CA07 | 0.211 | 0.096 | 0.175 | 0.257 | 0.134 | 0.278 |
| 8. 1 mg/kg AC002929+ CA07 | 0.159 | 0.063 | 0.105 | 0.225 | 0.080 | 0.125 |

Groups 3-8 each showed reductions in H1N1 and in M1.

### Example 30. In Vivo Administration of IAV RNAi Agents to Mice Previously Infected with CA07 H1N1

Female C57Bl/6 mice animals were first infected with CA07 and then subsequently dosed with IAV RNAi agents, in accordance with Example 4 (“CA07 H1N1 model mice”). On Day 1, five (n=5) mice (for Groups 1-7) were dosed with either saline or IAV RNAi agent formulated in saline (at 3 mg/kg or 6 mg/kg), via intratracheal (IT) administration. On Day 1, the animals were dosed with either PBS or CA07 H1N1 formulated in PBS, via intranasal (IN) injection. Dosing was in accordance with Table 64 below.

TABLE 64
Dosing for mice animals of Example 30.

| Group | Dose (RNAi Agent) | Dose (CA07) |
|---|---|---|
| 1 | Day 1: IT 50 μL Saline | Day 1: IN 100 μL PBS |
| | (4 hours post-infection) | |
| 2 | Day 1: IT 50 μL Saline | Day 1: IN 100 μL CA07 |
| | (4 hours post-infection) | (4240 TCID50) |
| 3 | Day 1: IT 50 μL Saline | Day 1: IN 100 μL CA07 |
| | (8 hours post-infection) | (4240 TCID50) |
| 4 | Day 1: IT 50 μL 3 mg/kg | Day 1: IN 100 μL CA07 |
| | AC002564 (4 hours post- | (4240 TCID50) |
| | infection) | |
| 5 | Day 1: IT 100 μL 6 mg/kg | Day 1: IN 100 μL CA07 |
| | AC002564 (4 hours post- | (4240 TCID50) |
| | infection) | |
| 6 | Day 1: IT 50 μL 3 mg/kg | Day 1: IN 100 μL CA07 |
| | AC002564 (8 hours post- | (4240 TCID50) |
| | infection) | |
| 7 | Day 1: IT 100 μL 6 mg/ | Day 1: IN 100 μL CA07 |
| | kgAC002564 (8 hours | (4240 TCID50) |
| | post-infection) | |

CA07 dose was quantified in TCID50 (50% tissue culture infective dose). This TCID50 dose was determined to be the optimal sub-lethal dose in accordance with the CA07 mouse model described in Example 3, above.

On Day 7, the animals were sacrificed. Lungs (both left and right) were collected and harvested. Expression of mouse H1N1 and M1 in lung was determined using qPCR, with 18s rRNA as endogenous control gene. Average H1N1 and M1 expression for each animal in lung tissue was normalized relative to Group 2 (no RNAi agent+CA07 infection). Results are shown in Table 65 below.

TABLE 65
Average relative expression of H1N1 and M1 in mice lung of Example 30.

| | Avg | | | Avg | | |
|---|---|---|---|---|---|---|
| Group ID | H1N1 | Low | High | M1 | Low | High |
| 1. Saline + PBS | N/A | N/A | N/A | N/A | N/A | N/A |
| 2. Saline (4 h post CA07) + | 1.000 | 0.180 | 0.425 | 1.000 | 0.114 | 0.288 |
| CA07 | | | | | | |
| 3. Saline (8 h post CA07) + | 0.903 | 0.285 | 0.416 | 1.101 | 0.304 | 0.419 |
| CA07 | | | | | | |
| 4. 3 mg/kg AC002564 (4 h | 0.308 | 0.113 | 0.178 | 0.476 | 0.173 | 0.272 |
| post CA07) + CA07 | | | | | | |
| 5. 6 mg/kg AC002564 (4 h | 0.219 | 0.096 | 0.172 | 0.311 | 0.111 | 0.172 |
| post CA07) + CA07 | | | | | | |
| 6. 3 mg/kg AC002564 (8 h | 0.214 | 0.054 | 0.073 | 0.320 | 0.117 | 0.185 |
| post CA07) + CA07 | | | | | | |
| 7. 6 mg/kg AC002564 (8 h | 0.283 | 0.123 | 0.218 | 0.362 | 0.161 | 0.289 |
| post CA07) + CA07 | | | | | | |

Each of Groups 4-7 showed reductions in H1N1 and in M1.

### Example 31. In Vivo Administration of IAV RNAi Agents to Mice Subsequently Infected with CA07 H1N1

Female C57Bl/6 mice animals were dosed with IAV RNAi agents and subsequently infected with CA07, in accordance with Example 4 (“CA07 H1N1 model mice”). On Day 1, four (n=4) mice (for Group 1) and five (n=5) mice (for Groups 2-8) were dosed with either saline or IAV RNAi agent formulated in saline (at 0.75 mg/kg), via intratracheal (IT) administration. On Day 8, the animals were dosed with either PBS or CA07 H1N1 formulated in PBS, via intranasal (IN) injection. Dosing was in accordance with Table 66 below.

TABLE 66
Dosing for mice animals of Example 31.

| Group | Dose (RNAi Agent) | Dose (CA07) |
|---|---|---|
| 1 | Day 1 and 3: IT 50 μL | Day 1: IN 100 μL PBS |
| | Saline | |
| 2 | Day 1 and 3: IT 50 μL | Day 1: IN 100 μL CA07 |
| | Saline | (4240 TCID50) |
| 3 | Day 1 and 3: IT 50 μL | Day 1: IN 100 μL CA07 |
| | 0.75 mg/kg AC002601 | (4240 TCID50) |
| 4 | Day 1 and 3: IT 50 μL | Day 1: IN 100 μL CA07 |
| | 0.75 mg/kg AC002928 | (4240 TCID50) |
| 5 | Day 1 and 3: IT 50 μL | Day 1: IN 100 μL CA07 |
| | 0.75 mg/kg AC002929 | (4240 TCID50) |
| 6 | Day 1 and 3: IT 50 μL | Day 1: IN 100 μL CA07 |
| | 0.75 mg/kg AC002930 | (4240 TCID50) |
| 7 | Day 1 and 3: IT 50 μL | Day 1: IN 100 μL CA07 |
| | 0.75 mg/kg AC002931 | (4240 TCID50) |
| 8 | Day 1 and 3: IT 50 μL | Day 1: IN 100 μL CA07 |
| | 0.75 mg/kg AC002927 | (4240 TCID50) |

CA07 dose was quantified in TCID50 (50% tissue culture infective dose). This TCID50 dose was determined to be the optimal sub-lethal dose in accordance with the CA07 mouse model described in Example 3, above.

On Day 14, the animals were sacrificed. Lungs (both left and right) were collected and harvested. Expression of mouse H1N1 and M1 in lung was determined using qPCR, with 18s rRNA as endogenous control gene. Average H1N1 and M1 expression for each animal in lung tissue was normalized relative to Group 2 (no RNAi agent+CA07 infection). Results are shown in Table 67 below.

TABLE 67
Average relative expression of H1N1 and M1 in mice lung of Example 31.

| | Avg | | | Avg | | |
|---|---|---|---|---|---|---|
| Group ID | H1N1 | Low | High | M1 | Low | High |
| 1. Saline + PBS | N/A | N/A | N/A | N/A | N/A | N/A |
| 2. Saline + CA07 | 1.000 | 0.297 | 0.422 | 1.000 | 0.278 | 0.384 |
| 3. 0.75 mg/kg AC002601 + | 0.176 | 0.088 | 0.175 | 0.246 | 0.128 | 0.266 |
| CA07 | | | | | | |
| 4. 0.75 mg/kg AC002928 + | 0.398 | 0.249 | 0.669 | 0.390 | 0.217 | 0.491 |
| CA07 | | | | | | |
| 5. 0.75 mg/kg AC002929 + | 0.334 | 0.130 | 0.213 | 0.316 | 0.130 | 0.222 |
| CA07 | | | | | | |
| 6. 0.75 mg/kg AC002930 + | 0.373 | 0.125 | 0.189 | 0.260 | 0.071 | 0.098 |
| CA07 | | | | | | |
| 7. 0.75 mg/kg AC002931 + | 0.564 | 0.201 | 0.312 | 0.372 | 0.186 | 0.372 |
| CA07 | | | | | | |
| 8. 0.75 mg/kg AC002927 + | 0.235 | 0.085 | 0.134 | 0.220 | 0.078 | 0.121 |
| CA07 | | | | | | |

Groups 3-8 each showed reductions in H1N1 and in M1.

### Example 32. In Vivo Administration of IAV RNAi Agents to Mice Previously Infected with CA07 H1N1

Female C57Bl/6 mice animals were infected with CA07 and then subsequently dosed with IAV RNAi agents, in accordance with Example 4 (“CA07 H1N1 model mice”). On Day 1, five (n=5) mice (for Group 1) and ten (n=10) mice (for Groups 2-5) were dosed with either saline or IAV RNAi agent formulated in saline (at 3 mg/kg), or Oseltamivir (Tamiflu®; at 20 mg/kg) (Group 5 dosed on Day 1, 2, 3, 4, 5), via intranasal (IN) administration or oral gavage. Prior to dosing with IAV RNAi agents, on Day 1, the animals were dosed with either PBS or CA07 H1N1 formulated in PBS, via intranasal (IN) injection. Dosing was in accordance with Table 68 below.

TABLE 68
Dosing for mice animals of Example 32.

| Group | Dose (RNAi Agent) | Dose (CA07) |
|---|---|---|
| 1 | Day 1 (4 hr & 8 hr post | Day 1: IN |
| | PBS infection): IN 50 μL | 100 μL PBS |
| | Saline | |
| 2 | Day 1 (4 hr & 8 hr post | Day 1: IN |
| | CA07 infection): IN 50 μL | 100 μL CA07 |
| | Saline | (4240 TCID50) |
| 3 | Day 1 (4 hr & 8 hr post | Day 1: IN |
| | CA07 infection): IN 50 μL | 100 μL CA07 |
| | 3 mg/kg AC002866 | (4240 TCID50) |
| 4 | Day 1 (4 hr & 8 hr post | Day 1: IN |
| | CA07 infection): IN 50 μL | 100 μL CA07 |
| | 3 mg/kg AC002869 | (4240 TCID50) |
| 5 | Day 1 (4 hr post CA07 | Day 1: IN |
| | infection), 2, 3, 4, 5: | 100 μL CA07 |
| | oral gavage 20 mg/kg | (4240 TCID50) |
| | Oseltamivir (Tamiflu ®) | |

CA07 dose was quantified in TCID50 (50% tissue culture infective dose). This TCID50 dose was determined to be the optimal sub-lethal dose in accordance with the CA07 mouse model described in Example 3, above.

On Day 7, the animals were sacrificed. Lungs (both left and right) were collected and harvested. Expression of mouse H1N1 (measuring genomic RNA) and M1 (measuring mRNA reductions) in lung was determined using qPCR, with 18s rRNA as endogenous control gene. Average H1N1 and M1 expression for each animal in lung tissue was normalized relative to Group 2 (no RNAi agent+CA07 infection). Results are shown in Table 69 below.

TABLE 69
Average relative expression of H1N1 (genomic RNA) and M1
(mRNA) in mice lung of Example 32.

| | Avg | | | Avg | | |
|---|---|---|---|---|---|---|
| Group ID | H1N1 | Low | High | M1 | Low | High |
| 1. Saline + PBS | N/A | N/A | N/A | N/A | N/A | N/A |
| 2. Saline + CA07 | 1.000 | 0.511 | 1.044 | 1.000 | 0.425 | 0.739 |
| 3. 3 mg/kg AC002866 + CA07 | 0.185 | 0.037 | 0.046 | 0.224 | 0.047 | 0.059 |
| 4. 3 mg/kg AC002869 + CA07 | 0.179 | 0.047 | 0.064 | 0.205 | 0.055 | 0.075 |
| 5. 20 mg/kg oseltamivir | 1.147 | 0.381 | 0.571 | 1.185 | 0.398 | 0.600 |
| (Tamiflu ®) + CA07 | | | | | | |

Groups 3 and 4 with an IAV RNAi agent dosed showed substantial reductions in H1N1 and in M1 of greater than or equal to approximately 80%, particularly when compared to commercially available oseltamivir (Tamiflu®) which showed no reductions in either H1N1 or M1 and was generally comparable to model mice administered saline.

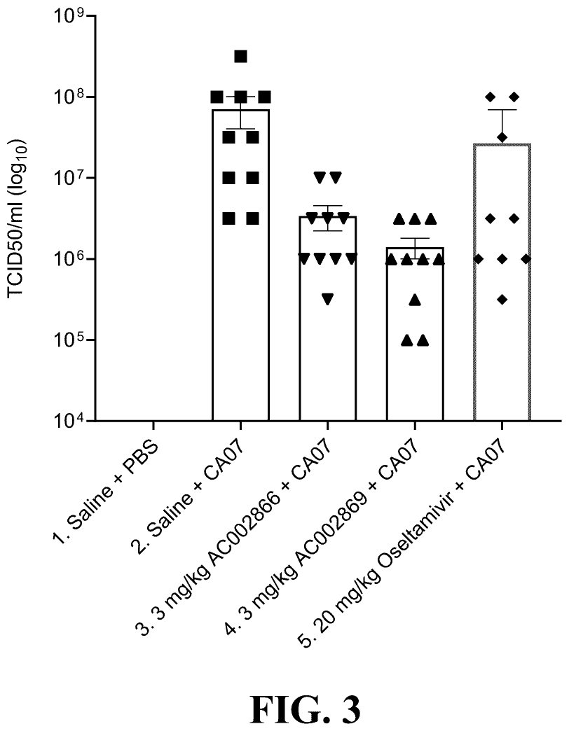

CA07 H1N1 viral load was quantified in the mice animal lungs. FIG. 3 shows the viral load of the test animals' lung at Day 7 sacrifice. IAV RNAi agent AC002869 reduced lung viral load by approximately 2 log₁₀ TCID50/mL. In comparison, the Group dosed only with oseltamivir reduced lung viral load by less than 1 log₁₀ TCID50/mL.

Mice test animal lungs were prepared for H&E staining, IHC staining, and RNA scope. Mice test animal lung samples were processed, and paraffin embedded (formalin-fixed paraffin-embedded FFPE). Sections of the lung were collected using a microtome, and mounted on slides. Histological inflammation assay was performed on the slides using an automated IHC stainer (Ventana Discovery Ultra), detected by DAB-chromogenic; the inflammation assay utilized an antibody pan-inflammation marker to detect all inflamed cells, Iba1 (Wako, Cat #: 019-19741, 1:400), secondary from Roche (DISC. OmniMap anti-Rb HRP, Cat #: 05269679001), and visual detection by chromogenic from Roche (DISC. ChromoMap DAB, Cat #: 05266645001) to detect all the inflammation cells. The lung samples were scanned using Olympus VS2000 auto scanner for image analysis. The images were then loaded onto image analysis software, Halo-Indica Lab, using the cell detection module to detect the inflamed cells (Iba1+cells).

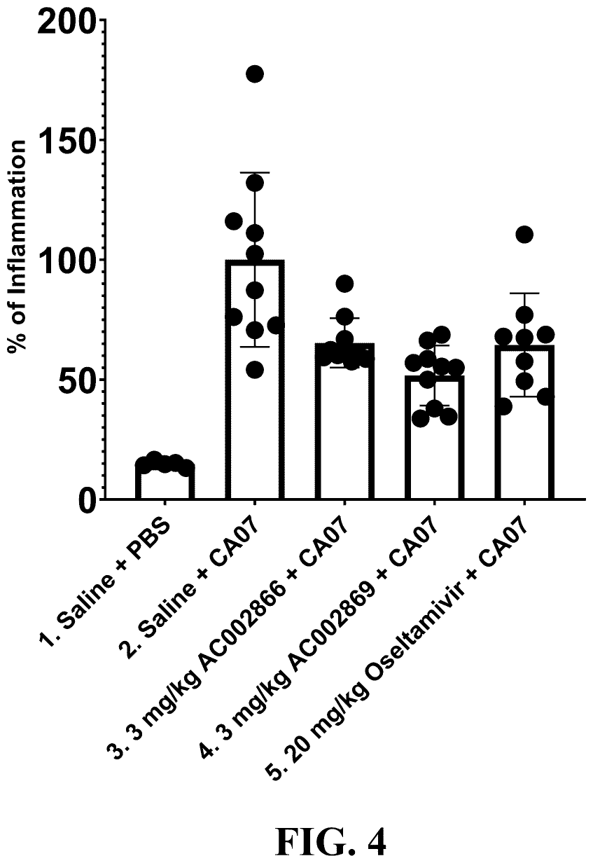

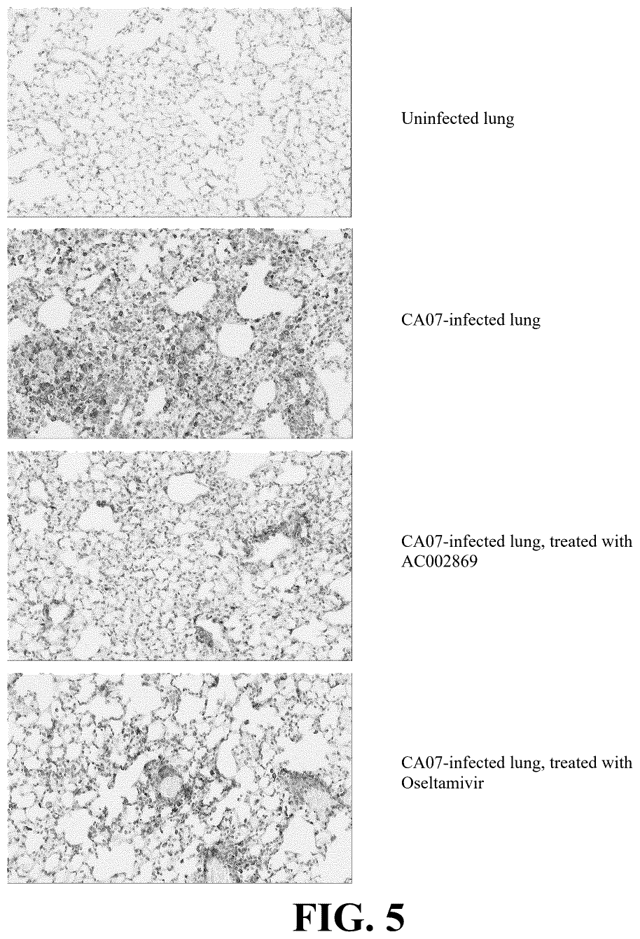

Inflammation of the test animal lungs was quantified, and the % of inflammation of the test groups was normalized to Group 2 Saline+CA07, the results are shown in FIG. 4. As shown in FIG. 4, RNAi agent AC002869 reduced lung inflammation by approximately 50%, compared to an approximately 36% reduction with oseltamivir treatment. FIG. 5 further shows histology of the mice test animal lungs, showing IAV RNAi agent AC002869 achieved reduction of inflammation in the mice test animal lungs.

### Example 33. In Vivo Administration of IAV RNAi Agents to Mice Subsequently Infected with CA07 H1N1

Female C57Bl/6 mice animals were dosed with IAV RNAi agents and subsequently infected with CA07, in accordance with Example 4 (“CA07 H1N1 model mice”). On Day 1 and 3 (for Groups 1-4), Day 3+4+5+6+7 (for Group 5), ten (n=10) mice (for Groups 1-5) were dosed with either saline or IAV RNAi agent formulated in saline (at 3 mg/kg), or Oseltamivir (Tamiflu®; at 40 mg/kg), via intranasal (IN) administration or oral gavage. On Day 8, the animals were dosed with either PBS or CA07 H1N1 formulated in PBS, via intranasal (IN) injection. Dosing was in accordance with Table 70 below.

TABLE 70
Dosing for mice animals of Example 33.

| Group | Dose (RNAi Agent) | Dose (CA07) |
|---|---|---|
| 1 | Day 1 and 3: IN 50 μL | Day 8: IN |
| | Saline | 100 μL PBS |
| 2 | Day 1 and 3: IN 50 μL | Day 8: IN |
| | Saline | 100 μL CA07 |
| | | (12000 TCID50) |
| 3 | Day 1 and 3: IN 50 μL | Day 8: IN |
| | 3 mg/kg AC002866 | 100 μL CA07 |
| | | (12000 TCID50) |
| 4 | Day 1 and 3: IN 50 μL | Day 8: IN |
| | 3 mg/kg AC002869 | 100 μL CA07 |
| | | (12000 TCID50) |
| 5 | Day 3 (2x), 4 (2x), 5 (2x), | Day 8: IN |
| | 6 (2x), 7 (2x): oral gavage | 100 μL CA07 |
| | 20 mg/kg Oseltamivir (Tamiflu ®) | (12000 TCID50) |

CA07 dose was quantified in TCID50 (50% tissue culture infective dose).

On Day 15, the animals were sacrificed. Mice were observed for body weight and survival rates post CA07 infection. Survival rate is determined upon sacrificing any test animal that loses more than 20% of initial body weight (>20% body weight loss=test animal death). The survival rates are shown in Table 71 below.

TABLE 71
Survival rates of test animals post CA07 infection of Example 33.

| Group ID | % Survival |
|---|---|
| 1. Saline + PBS | 100% 15 days post infection |
| 2. Saline + CA07 (12000 TCID50) | 90% 6 days post infection |
| | 40% 7 days post infection |
| | 10% 8 days post infection |
| | 10% 15 days post infection |
| 3. AC002866 + CA07 (12000 TCID50) | 100% 15 days post infection |
| 4. AC002869 + CA07 (12000 TCID50) | 100% 15 days post infection |
| 5. Oseltamivir (Tamiflu ®) + CA07 | 90% 6 days post infection |
| (12000 TCID50) | 80% 7 days post infection |
| | 30% 8 days post infection |
| | 20% 9 days post infection |
| | 20% 15 days post infection |

Dosing with IAV RNAi agents AC002866 or AC002869, followed by subsequent infection with CA07 (12000 TCID50), achieved 100% survival rate at 15 days post infection (Groups 3 and 4). Test animals dosed with saline followed by subsequent infection with CA07 (12000 TCID50) showed only a 10% survival rate at Day 8 post infection (Group 2). Similarly, test animals dosed with oseltamivir (Tamiflu®) showed only a 20% survival rate at 15 days post infection (Group 5). IAV RNAi agents AC002866 and AC002869 thus achieved reductions of mortality by ˜90% compared to the saline-treated Group infected with CA07 and reductions of mortality by ˜80% compared to the oseltamivir (Tamiflu®) treated Group.

### Example 34. In Vivo Administration of IAV RNAi Agents to Infected Mice with CA07 H1N1

Female C57Bl/6 mice animals were infected with CA07 and then subsequently dosed IAV RNAi agents, in accordance with Example 4 (“CA07 H1N1 model mice”). On Day 1, ten (n=10) mice in each Group were dosed with either saline or IAV RNAi agent formulated in saline (at 3 mg/kg), or Oseltamivir (Tamiflu®; at 40 mg/kg), via intranasal (IN) administration or oral gavage, in accordance with the dosing in Table 72 below.

TABLE 72
Dosing for mice animals of Example 34.

| Group | Dose (RNAi Agent) | Dose (CA07) |
|---|---|---|
| 1 | Day 1 (4 hours post infection | Day 1: IN |
| | & 8 hours post infection): | 100 μL PBS |
| | IN 50 μL Saline | |
| 2 | Day 1 (4 hours post infection | Day 1: IN |
| | & 8 hours post infection): | 100 μL CA07 |
| | IN 50 μL Saline | (12000 TCID50) |
| 3 | Day 1 (4 hours post infection | Day 1: IN |
| | & 8 hours post infection): | 100 μL CA07 |
| | IN 50 μL 3 mg/kg AC002866 | (12000 TCID50) |
| 4 | Day 1 (4 hours post infection | Day 1: IN |
| | & 8 hours post infection): | 100 μL CA07 |
| | IN 50 μL 3 mg/kg AC002869 | (12000 TCID50) |
| 5 | Day 1 (2x), Day 2 (2x), Day | Day 1: IN |
| | 3 (2x), Day 4 (2x), Day 5 (2x): | 100 μL CA07 |
| | oral gavage 40 mg/kg Oseltamivir | (12000 TCID50) |
| | (Tamiflu ®) (2 doses daily; 400 | |
| | mg/kg total dosed over 5 days) | |

CA07 dose was quantified in TCID5 (500% tissue culture infective dose).

On Day 15, the animals were sacrificed. Mice were observed for body weight and survival rates post CA07 infection. Survival rate is determined upon sacrificing any test animal that loses more than 200% of initial body weight (>20% body weight loss=test animal death). The survival rates are shown in Table 73 below.

TABLE 73
Survival rates of test animals post CA07 infection of Example 34.

| Group ID | % Survival |
|---|---|
| 1. Saline + PBS | 100% 14 days post infection |
| | (i.e., Day 15) |
| 2. Saline + CA07 | 90% 6 days post infection |
| (12000 TCID50) | 50% 7 days post infection |
| | 10% 8 days post infection |
| | 10% 14 days post infection |
| 3. AC002866 + CA07 | 90% 6 days post infection |
| (12000 TCID50) | 80% 7 days post infection |
| | 40% 8 days post infection |
| | 30% 9 days post infection |
| | 20% 10 days post infection |
| | 20% 14 days post infection |
| 4. AC002869 + CA07 | 70% 7 days post infection |
| (12000 TCID50) | 60% 8 days post infection |
| | 60% 14 days post infection |
| 5. Oseltamivir | 70% 7 days post infection |
| (Tamiflu ®) + CA07 | 20% 8 days post infection |
| (12000 TCID50) | 10% 9 days post infection |
| | 10% 14 days post infection |

Dosing with therapeutics after first infecting the animals presents a more severe model of IAV infection. As described in Table 73 above, infecting with CA07 (12000 TCID50) and subsequently dosing with IAV RNAi agents AC002866 or AC002869 achieved improved survival rates over the groups dosed with either oseltamivir or with no treatment. Test animals infected with CA07 (12000 TCID50) and subsequently administered with saline showed only a 10% survival rate at Day 8 and Day 14 post infection (Group 2). Similarly, test animals dosed with oseltamivir (Tamiflu®) showed only a 20% survival rate at Day 8 post infection (Group 5) and a 10% survival rate at Day 14 post infection. Comparatively, the Group dosed with RNAi agent AD002869 after infection with CA07, which targets the M1 influenza A genomic segment, showed 60% survival rate at 2 weeks post-infection.

### Example 35. Identification of Conserved RNAi Agent Sequences Across Influenza A Subtypes and Assessment of Viral Vulnerability

In order to identify the RNAi agent sequences disclosed in the various Examples described herein, approximately 10,000 influenza genomes of different subtypes, including H1N1, H3N2, H5N1, H5N6, H5N8, H7N2, H7N3, H7N4, H7N7, H7N9 and H9N2, were bioinformatically evaluated to locate the most highly conserved regions. By utilizing RNAi agents with sequences able to target conserved regions across different influenza genome subtypes, an RNAi agent therapeutic would be able to provide a therapeutic benefit to patients suffering from various influenza subtypes and therefore be able to address a larger set of patients.

For the RNAi agents described herein, as described elsewhere herein candidate sequences that target these highly conserved regions were identified in six genomic segments: PB1, PB2, PA, NP, NS, and M (which is referred to herein as M1, but includes the transcripts for M1 and M2). Genomic segments HA and NA were not evaluated due to the high genetic variation in these regions across different subtypes.

As described in the various Examples herein, throughout various evaluations of RNAi agents, the IAV RNAi agents that targeted the influenza A M1 genomic segments (i.e., targeting the influenza A genomic segment transcript of SEQ ID NO:1) consistently exhibited the most significant antiviral activity as compared to IAV RNAi agents that targeted other genomic segments.

### Example 36. In Vivo Administration of IAV RNAi Agents to Mice Infected with H5N1

Female C57Bl/6 mice animals first dosed with IAV RNAi agents, and were subsequently then infected with H5N1 IAV virus. Different groups of female C57Bl/6 mice animals were also dosed with IAV RNAi agents after H5N1 virus infection. On Day −7 and −5, ten (n=10) mice in Groups 3-4 were dosed with IAV RNAi agent formulated in saline (at 3 mg/kg), via intranasal (IN) or intratracheal (IT) administration; five (n=5) mice in Group 1 and ten (n=10) animals in Group 2 were dosed IN with saline. On Day 0, Groups 2-5 were dosed with H5N1 virus via intranasal (IN) administration. On Day 0, at 4 hours and 8 hours post H5N1 administration, ten (n=10) mice in Group 5 were dosed with IAV RNAi agent formulated in saline (at 3 mg/kg) via intranasal (IN) administration. Group 1 test animals were dosed IN with PBS and no H5N1. H5N1 dose was quantified in PFU (plaque forming unit). The dosing was in accordance with Table 74 below.

TABLE 74
Dosing for mice animals of Example 36.

| | | | Animals |
|---|---|---|---|
| Group | Dose (RNAi Agent) | Dose (H5N1) | (n=) |
| 1 | Day −7, Day −5: IN Saline | Day 0: IN 100 μL PBS | n = 5 |
| 2 | Day −7, Day −5: IN Saline | Day 0: IN H5N1 | n = 10 |
| | | (500 PFU in 25 μl) | |
| 3 | Day −7, Day −5: IT 25 μL | Day 0: IN H5N1 | n = 10 |
| | 3 mg/kg AC002869 | (500 PFU in 25 μl) | |
| 4 | Day −7, Day −5: IN 25 μL | Day 0: IN H5N1 | n = 10 |
| | 3 mg/kg AC002869 | (500 PFU in 25 μl) | |
| 5 | Day 0 4 hr and 8 hr after | Day 0: IN H5N1 | n = 10 |
| | H5N1 dose: IN 25 μL | (500 PFU in 25 μl) | |
| | 3 mg/kg AC002869 | | |

The administered H5N1 virus is of Influenza A Virus (A/Viet Nam/1203/2004(H5N1)).

All mice test animals were sacrificed on Day 5 (5 days post infection). Daily body weight measurement was collected. Lungs were collected, to quantify and analyze for lung viral load through PFU/TCID50, and lung histopathology.

TABLE 75
Lung viral load in mice test animals, of Example 36.

| | Mean | Mean log10 |
|---|---|---|
| Groups | PFU/lobe | PFU/lobe |
| Group 1 Saline + PBS | N/A | N/A |
| Group 2 Saline + H5N1 | 5.1E+06 | 6.4 |
| Group 3 AC002869 IT + H5N1 | 1.3E+04 | 3.9 |
| Group 4 AC002869 IN + H5N1 | 1.7E+04 | 3.3 |
| Group 5 AC002869 IN + H5N1 | 9.7E+03 | 3.8 |

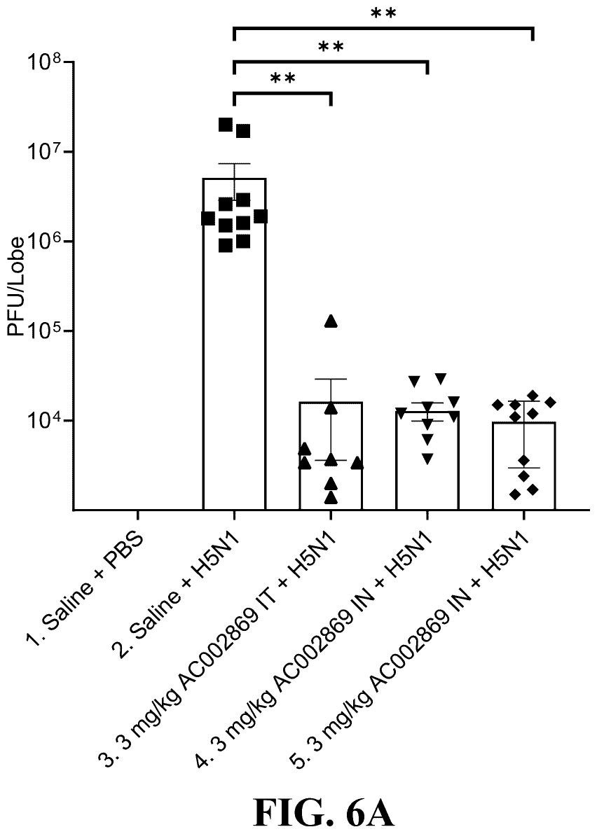

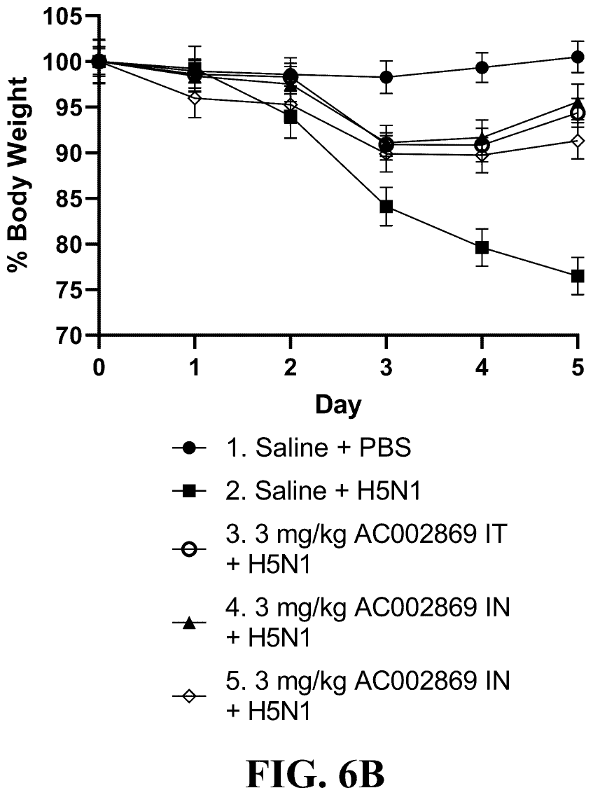

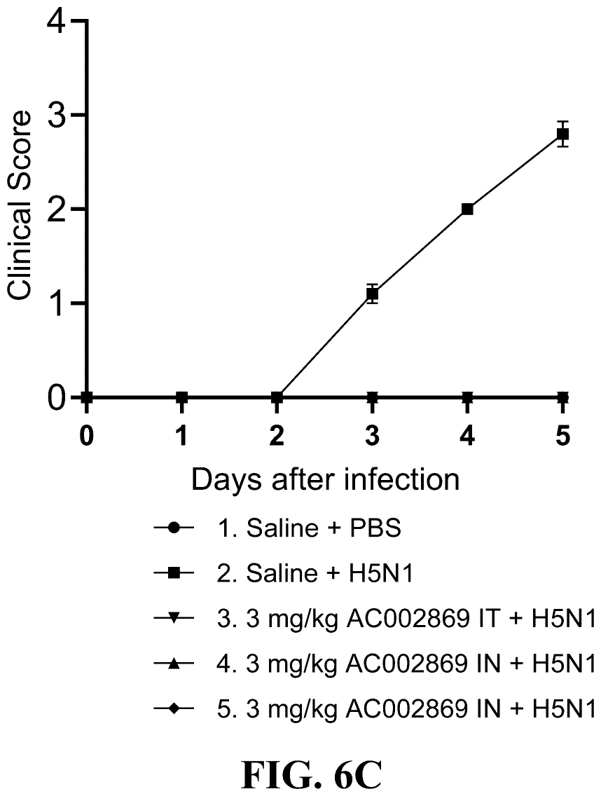

Lung viral load, quantified at PFU/lobe, is shown in Table 75 and FIG. 6A. In the mice test animals, prophylactic treatment using IAV RNAi agent AC002869, followed by H5N1 infection, showed significant reduction in lung viral load. Prophylactic treatment using AC002869, via IT (Group 3) and IN (Group 4), both achieved significant reduction in lung viral load in comparison with test animals dosed with saline (Group 2). Additionally, therapeutic treatment using AC002869 following H5N1 infection, Group 5, also showed significant reduction in lung viral load in comparison with test animals dosed with saline (Group 2). Groups 3-5 all showed approximately a 10²-fold or 2 log 10-fold reduction in lung viral load, in comparison with test animals dosed with saline (Group 2). Following analysis of variance, Tukey's honest significant difference HSD test showed p values of p<0.01 for each Group 2 vs. Group 3, Group 2 vs. Group 4, and Group 2 vs. Group 5 (denoted as ** p<0.01).

Mice test animals' weight was collected and shown in the following Table 76, as well as FIG. 6B.

TABLE 76
% body weight test animals dosed with IAV RNAi agents, of Example 36.

| | Group 1 | | Group 2 | | Group 3 | | Group 4 | | Group 5 | |
|---|---|---|---|---|---|---|---|---|---|---|
| | Saline + | | Saline + | | AC002869 | | AC002869 | | AC002869 | |
| | PBS | | H5N1 | | IT + H5N1 | | IN + H5N1 | | IN + H5N1 | |
| Day | Mean | SEM | Mean | SEM | Mean | SEM | Mean | SEM | Mean | SEM |
| 0 | 100.000 | 1.410 | 100.000 | 2.430 | 100.000 | 1.627 | 100.000 | 1.593 | 100.000 | 2.344 |
| 1 | 98.942 | 1.321 | 99.204 | 2.444 | 98.602 | 1.525 | 98.380 | 1.751 | 95.976 | 2.122 |
| 2 | 98.558 | 1.836 | 94.033 | 2.432 | 98.312 | 1.487 | 97.523 | 1.936 | 95.282 | 2.041 |
| 3 | 98.269 | 1.793 | 84.088 | 2.112 | 90.887 | 1.296 | 91.091 | 1.909 | 89.870 | 1.993 |
| 4 | 99.327 | 1.638 | 79.612 | 2.056 | 90.839 | 1.839 | 91.663 | 1.918 | 89.732 | 1.930 |
| 5 | 100.481 | 1.723 | 76.479 | 2.044 | 94.359 | 1.580 | 95.569 | 1.963 | 91.304 | 1.992 |

As shown in Table 76 and FIG. 6B, following H5N1 infection, mice test animals treated with AC002869 showed improved weight change in comparison with test animals treated with saline (Group 2). Prophylactic treatment using AC002869, via IT (Group 3) and IN (Group 4), both achieved significantly improved weight change in comparison with test animals dosed with saline (Group 2). Group 3 and Group 4 both achieved approximately 94-95% weight retention in comparison with test animals dosed with saline (Group 2, approximately 76%). Additionally, therapeutic treatment using AC002869 following H5N1 infection, Group 5, also showed significant improvement in weight retention of approximately 91% in comparison with test animals dosed with saline (Group 2, approximately 76%).

“Clinical Score” was also observed for the mice test animals. For this study, Clinical Scores are defined as:
0=normal;
1=questionable illness;
2=mild but definitive illness;
3=moderate to severe illness;
4=distinctly severe illness, moribund—euthanized; and
5=found dead.

The Clinical Scores of the mice test animals are shown in FIG. 6C. All test Groups 3-5 treated with IAV RNAi agent AC002869, either before (Groups 3 and 4, prophylactic) or after (Group 5, therapeutic) H5N1 infection, via either intranasal (Groups 4 and 5) or intratracheal administration (Group 3), showed significantly improved clinical scores in comparison with test animals dosed with saline (Group 2). After H5N1 infection, Groups 3-5 test animals showed similar clinical scores as that of Group 1 test animals not dosed with H5N1 virus, maintaining 0 clinical score out to 5 days post H5N1 infection.

The above experimental data and results demonstrate the IAV RNAi agent AC002869 described herein also has potent antiviral activity against the H5N1 variant of the influenza A virus, as AC002869 is designed to exhibit antiviral activity targeted at highly conserved regions of the influenza A virus (specifically, AC002869 targets M1). Additionally, IAV RNAi agent AC002869 shows both prophylactic as well as therapeutic antiviral activity against H5N1. Accordingly, IAV RNAi agent AC002869, as well as other IAV RNAi agents described herein, is capable of potent antiviral activity across different influenza subtypes and therefore offer patients more therapeutic benefit.

### Example 37. In Vivo Administration of IAV RNAi Agents to Mice Infected with H5N1

Female Balb/c mice animals first dosed with IAV RNAi agents, and were subsequently then infected with H5N1 IAV virus. Different groups of female Balb/c mice animals were also dosed with IAV RNAi agents after H5N1 virus infection. On Day −7 and −5, ten (n=10) mice in Group 3 were dosed with IAV RNAi agent AC002869 formulated in saline (at 3 mg/kg), via intranasal (IN) administration; ten (n=10) mice in Group 1 and Group 2 were dosed IN with saline. On Day 0, Groups 2-4 were dosed with H5N1 virus via intranasal (IN) administration. On Day 0, at 4 hours and 8 hours post H5N1 administration, ten (n=10) mice in Group 4 were dosed with IAV RNAi agent formulated in saline (at 3 mg/kg) via intranasal (IN) administration. Group 1 test animals were dosed IN with PBS and no H5N1. H5N1 dose was quantified in TCID50 (50% tissue culture infective dose). The dosing was in accordance with Table 77 below.

TABLE 77
Dosing for mice animals of Example 37.

| | | | Animals |
|---|---|---|---|
| Group | Dose (RNAi Agent) | Dose (H5N1) | (n=) |
| 1 | Day −7, Day −5: IN Saline | Day 0: IN PBS | n = 10 |
| 2 | Day −7, Day −5: IN 25 μl | Day 0: IN H5N1 | n = 10 |
| | Saline | (500 TCID50) | |
| 3 | Day −7, Day −5: IN 25 μl | Day 0: IN H5N1 | n = 10 |
| | 3 mg/kg AC002869 | (500 TCID50) | |
| 4 | Day 0 4 hr and 8 hr after | Day 0: IN H5N1 | n = 10 |
| | H5N1 dose: IN 25 μl | (500 TCID50) | |
| | 3 mg/kg AC002869 | | |

The administered H5N1 virus is of Influenza A Virus (A/whooper swan/Mongolia/244/2005(H5N1)).

All mice test animals were sacrificed on Day 14 (14 days post H5N1 infection). Daily body weight measurement was collected. Lungs were collected, to quantify and analyze for lung viral load through PFU/TCID50, and lung histopathology.

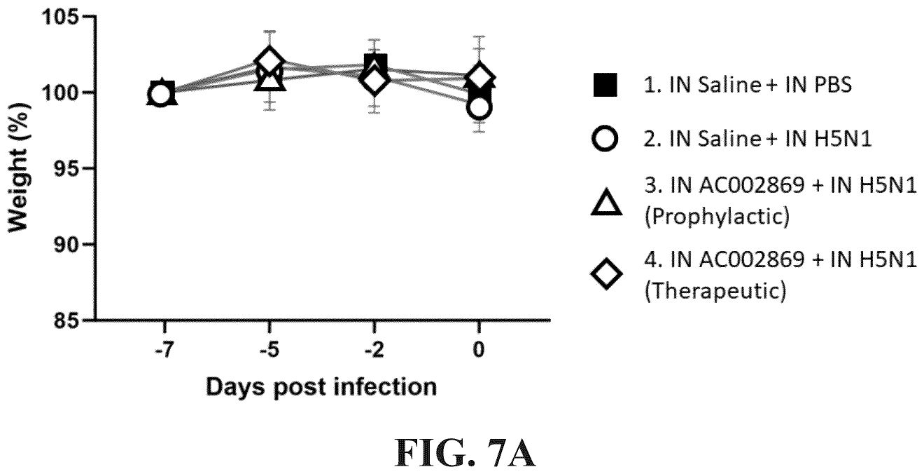

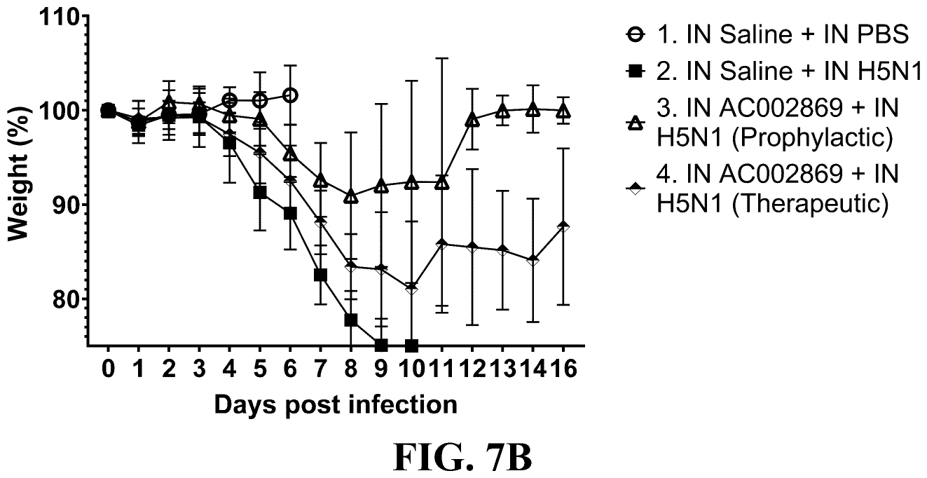

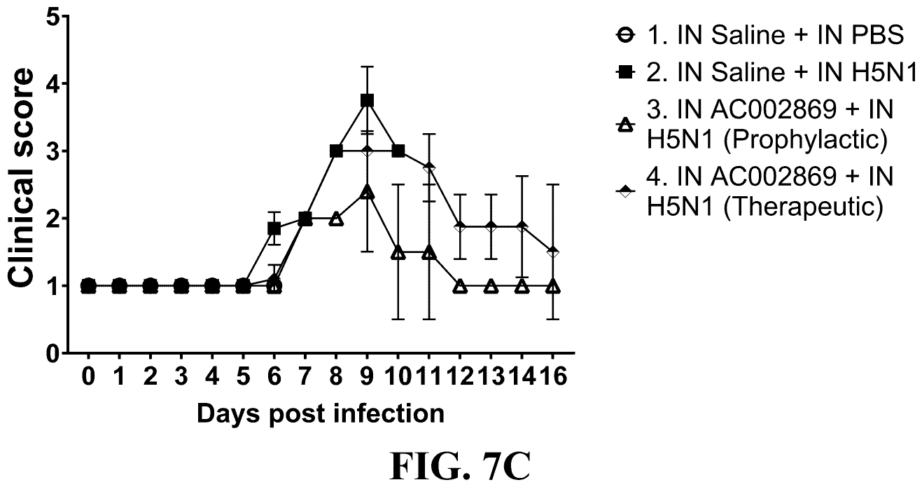

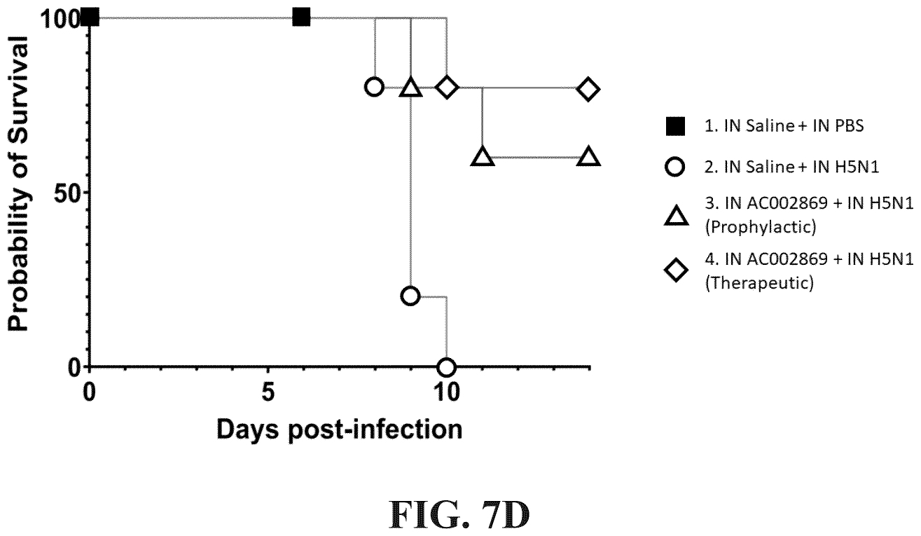

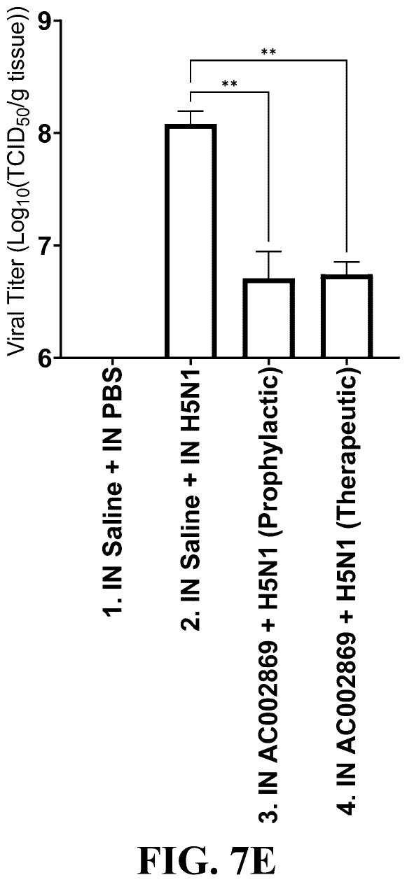

FIG. 7A shows the mice test animals weight at time points before H5N1 infection. The IAV RNAi agent AC002869 does not appear to cause significant side effects that affect animal weight.

FIG. 7B shows the mice test animals weight at time points after H5N1 infection. Test animals of Group 2, administered saline (no IAV RNAi agent) and infected with H5N1, all succumbed to H5N1 infection. Test animals of Group 3, treated on Day −7 and −5 with IAV RNAi agent AC002869 and infected with H5N1, showed weight loss at Day 5-10, but recovered to ˜100% of starting body weight by Day 12-14. Test animals of Group 4, treated with IAV RNAi agent AC002869 at 4 and 8 hours post H5N1 infection, showed greater body weight loss compared to Group 3, after a brief period of recovery between Day 10 and 11, but still improved body weight loss in comparison to Group 2.

Clinical score is also observed for the mice test animals. For this study, Clinical Scores are defined as:
1=healthy;
2=ruffled fur, panting, lethargic (triggers 2nd observation);
3=a score of 2 plus 1 additional clinical sign such as, hunched posture, orbital tightening, increased respiratory rate, or >15% weight loss (trigger 3rd observation);
4=dyspnea and/or cyanosis, reluctance to move when stimulated or >25% weight loss−immediate euthanasia

FIG. 7C shows the clinical scores of the mice test animals. Test animals of Group 3 (treated with IAV RNAi agent AC002869 before H5N1) and Group 4 (treated with IAV RNAi agent AC002869 after H5N1) both showed improved clinical scores in comparison to Group 2 test animals (treated with no IAV RNAi agent and infected with H5N1). Group 4 showed improved clinical scores in comparison with Group 3. This shows both prophylactic as well as therapeutic treatment with AC002869 show improved clinical scores in comparison to Group 2 (treated with saline and no IAV RNAi agent), while AC002869 prophylactic treatment show better clinical scores than that of therapeutic treatment. Group 3 test animals showed clinical scores of healthy (score=1) starting at Day 12.

FIG. 7D shows the survival index of the test animals. All animals of Group 2, treated with no IAV RNAi agent and infected with H5N1, showed 100% mortality by Day 10. At Day 14, Group 3 (prophylactic treatment AC002869) showed 60% survival, while Group 4 (therapeutic treatment AC002869) showed 80% survival. Both prophylactic treatment (Group 3), as well as therapeutic treatment (Group 4) via IAV RNAi agent AC002869 showed significant improvement in mortality, at 60% and 80%, respectively, in comparison to Group 2 treated with no IAV RNAi agent.

FIG. 7E shows the lung viral load in TCID50. In the mice test animals, prophylactic and therapeutic treatment of H5N1 using IAV RNAi agent AC002869, showed significant reduction in lung viral load. Prophylactic treatment Group 3 and therapeutic treatment Group 4 both achieved significant antiviral activity in lung, reducing H5N1 lung viral load by ˜1.5 log 10, in comparison to test animals dosed with no IAV RNAi agent (Group 2). Significance is denoted as ** p<0.01.

The above experimental data and results demonstrate the IAV RNAi agent AC002869 described herein also has potent antiviral activity against the H5N1 variant of the influenza A virus, as AC002869 is designed to exhibit antiviral activity targeted at highly conserved regions of the influenza A virus (specifically, AC002869 targets M1). Additionally, IAV RNAi agent AC002869 shows both prophylactic as well as therapeutic antiviral activity against H5N1. Accordingly, IAV RNAi agent AC002869, as well as other IAV RNAi agents described herein, is capable of potent antiviral activity across different influenza subtypes and therefore offer patients more therapeutic benefit.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An RNAi agent for inhibiting expression of an influenza A viral genome, comprising:
an antisense strand comprising at least 17 contiguous nucleotides differing by 0 or 1 nucleotides from any one of the sequences provided in Table 2A, 2B, 2C, 2D, 2E, 2F, 3A, 3B, 3C, 3D, 3E, or 3F; and
a sense strand comprising a nucleotide sequence that is at least partially complementary to the antisense strand.

2-5. (canceled)

6. The RNAi agent of claim 1, wherein at least one nucleotide of the RNAi agent is a modified nucleotide or includes a modified internucleoside linkage, wherein the at least one modified nucleotide of the RNAi agent is selected from the group consisting of: 2′-O-methyl nucleotide, 2′-fluoro nucleotide, 2′-deoxy nucleotide, 2′,3′-seco nucleotide mimic, locked nucleotide, 2′-F-arabino nucleotide, 2′-methoxyethyl nucleotide, abasic nucleotide, ribitol, inverted nucleotide, inverted 2′-O-methyl nucleotide, inverted 2′-deoxy nucleotide, 2′-amino-modified nucleotide, 2′-alkyl-modified nucleotide, morpholino nucleotide, vinyl phosphonate-containing nucleotide, cyclopropyl phosphonate-containing nucleotide, and 3′-O-methyl nucleotide.

7. (canceled)

8. The RNAi agent of claim 1, wherein the antisense strand comprises the nucleotide sequence of any one of the modified sequences provided in Table 3A, 3B, 3C, 3D, 3E, and 3F.

9. The RNAi agent of claim 1, wherein the sense strand comprises the nucleotide sequence of any one of the modified sequences provided in Table 4A, 4B, 4C, 4D, 4E, and 4F.

10. (canceled)

11. The RNAi agent of claim 1, wherein the sense strand is between 18 and 30 nucleotides in length, and the antisense strand is between 18 and 30 nucleotides in length.

12-16. (canceled)

17. The RNAi agent of claim 1, wherein the sense strand comprises one or two inverted abasic residues.

18. The RNAi agent of claim 1, wherein the RNAi agent comprises a sense strand and an antisense strand that form a duplex having the structure of any one of the duplexes in Table 7A-1, 7A-2, 7A-3, 7A-4, 7A-5, 7A-6, 7B-1, 7B-2, 7B-3, 7B-4, 7B-5, 7B-6, 8A, 8B, 8C, 8D, 8E, 8F, 9A, 9B, 9C, 9D, 9E, 9F, 10A, 10B, 10C, 10D, 10E, or 10F.

19. (canceled)

20. The RNAi agent of claim 1, comprising an antisense strand that consists of, consists essentially of, or comprises a nucleotide sequence that differs by 0 or 1 nucleotides from the following nucleotide sequence (5′→3′):
(SEQ ID NO: 1590)
UUACGUUUCGACCUCGGUUAG.

21. The RNAi agent of claim 20, wherein the sense strand consists of, consists essentially of, or comprises a nucleotide sequence that differs by 0 or 1 nucleotides from the following nucleotide sequence (5′→3′):
(SEQ ID NO: 1706)
CUAACCGAGGUCGAAACGUAA.

22. (canceled)

23. The RNAi agent of claim 1, comprising an antisense strand that comprises, consists of, or consists essentially of a modified nucleotide sequence that differs by 0 or 1 nucleotides from one of the following nucleotide sequences (5′→3′):
(SEQ ID NO: 1176)
cPrpusUfsascguUfucgaCfcUfcGfguuasg;
or
(SEQ ID NO: 1175)
cPrpusUfsascGfuuucgaCfcUfcGfguuasg;
wherein a represents 2′-O-methyl adenosine, c represents 2′-O-methyl cytidine, g represents 2′-O-methyl guanosine, and u represents 2′-O-methyl uridine; Af represents 2′-fluoro adenosine, Cf represents 2′-fluoro cytidine, Gf represents 2′-fluoro guanosine, and Uf represents 2′-fluoro uridine; cPrpu represents a 5′-cyclopropyl phosphonate-2′-O-methyl uridine; s represents a phosphorothioate linkage; and wherein all or substantially all of the nucleotides on the sense strand are modified nucleotides.

24. The RNAi agent of claim 1, wherein the sense strand comprises, consists of, or consists essentially of a modified nucleotide sequence that differs by 0 or 1 nucleotides from one of the following nucleotide sequences (5′→3′):
(SEQ ID NO: 1373)
csuaaccgaGfgUfcGfaaacguaa;
or
(SEQ ID NO: 1374)
csuaaccgaGfgUfcgaaacguaa;
wherein a represents 2′-O-methyl adenosine, c represents 2′-O-methyl cytidine, g represents 2′-O-methyl guanosine, and u represents 2′-O-methyl uridine; Af represents 2′-fluoro adenosine, Cf represents 2′-fluoro cytidine, Gf represents 2′-fluoro guanosine, and Uf represents 2′-fluoro uridine; cPrpu represents a 5′-cyclopropyl phosphonate-2′-O-methyl uridine; s represents a phosphorothioate linkage; and wherein all or substantially all of the nucleotides on the antisense strand are modified nucleotides.

25. (canceled)

26. The RNAi agent of claim 1, wherein the RNAi agent is linked to a targeting ligand.

27-29. (canceled)

30. The RNAi agent of claim 26, wherein the targeting ligand comprises the structure: 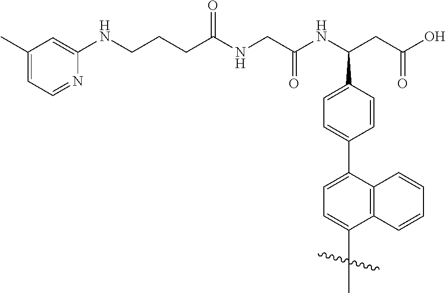 [SMILES: *c1ccc(-c2ccc([C@H](CC(=O)O)NC(=O)CNC(=O)CCCNc3cc(C)ccn3)cc2)c2ccccc12]
or a pharmaceutically acceptable salt thereof, or 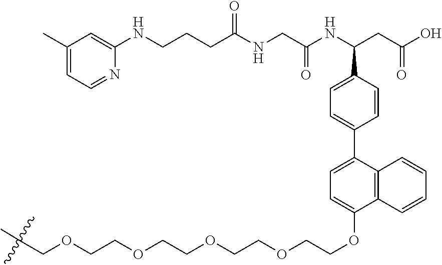 [SMILES: *COCCOCCOCCOCCOc1ccc(-c2ccc([C@H](CC(=O)O)NC(=O)CNC(=O)CCCNc3cc(C)ccn3)cc2)c2ccccc12]
or a pharmaceutically acceptable salt thereof,
wherein [symbol] indicates the point of connection to the RNAi agent.

31. The RNAi agent of claim 26, wherein the targeting ligand has a structure selected from the group consisting of: 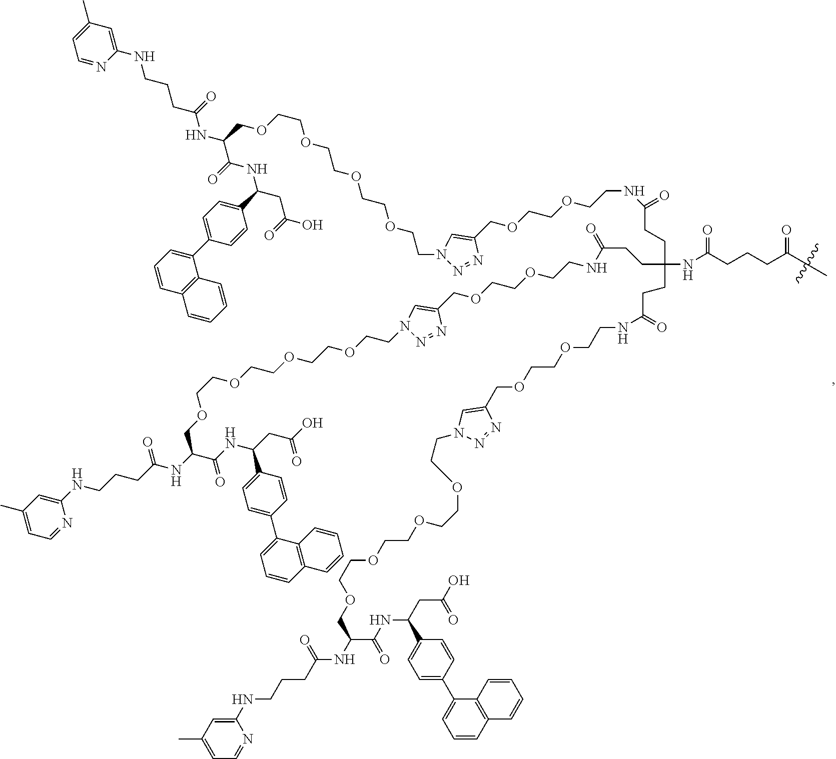 [SMILES: *C(=O)CCCC(=O)NC(CCC(=O)NCCOCCOCc1cn(CCOCCOCCOCCOC[C@H](NC(=O)CCCNc2cc(C)ccn2)C(=O)N[C@@H](CC(=O)O)c2ccc(-c3cccc4ccccc34)cc2)nn1)(CCC(=O)NCCOCCOCc1cn(CCOCCOCCOCCOC[C@H](NC(=O)CCCNc2cc(C)ccn2)C(=O)N[C@@H](CC(=O)O)c2ccc(-c3cccc4ccccc34)cc2)nn1)CCC(=O)NCCOCCOCc1cn(CCOCCOCCOCCOC[C@H](NC(=O)CCCNc2cc(C)ccn2)C(=O)N[C@@H](CC(=O)O)c2ccc(-c3cccc4ccccc34)cc2)nn1] 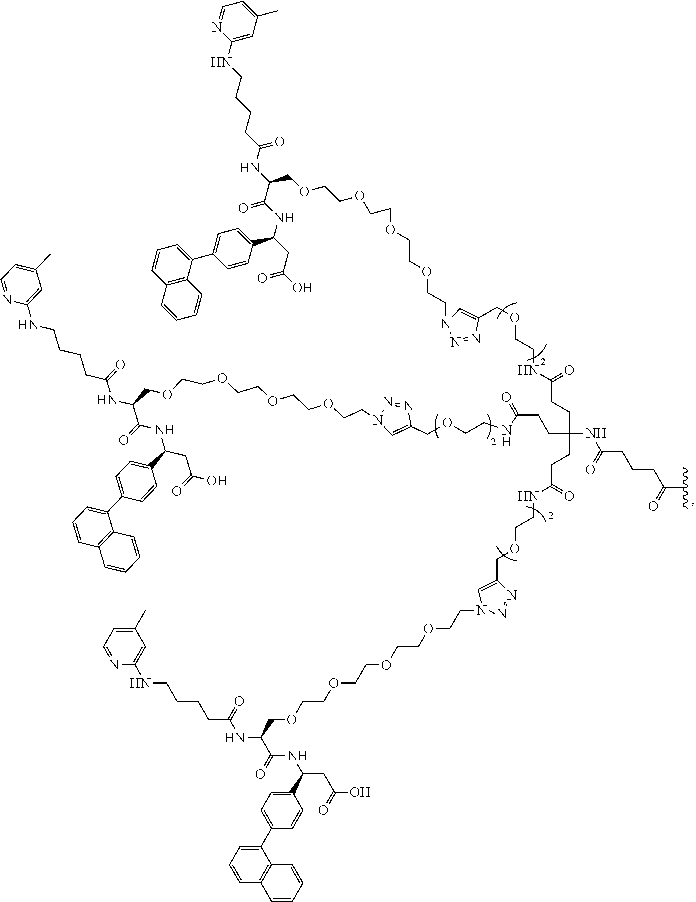 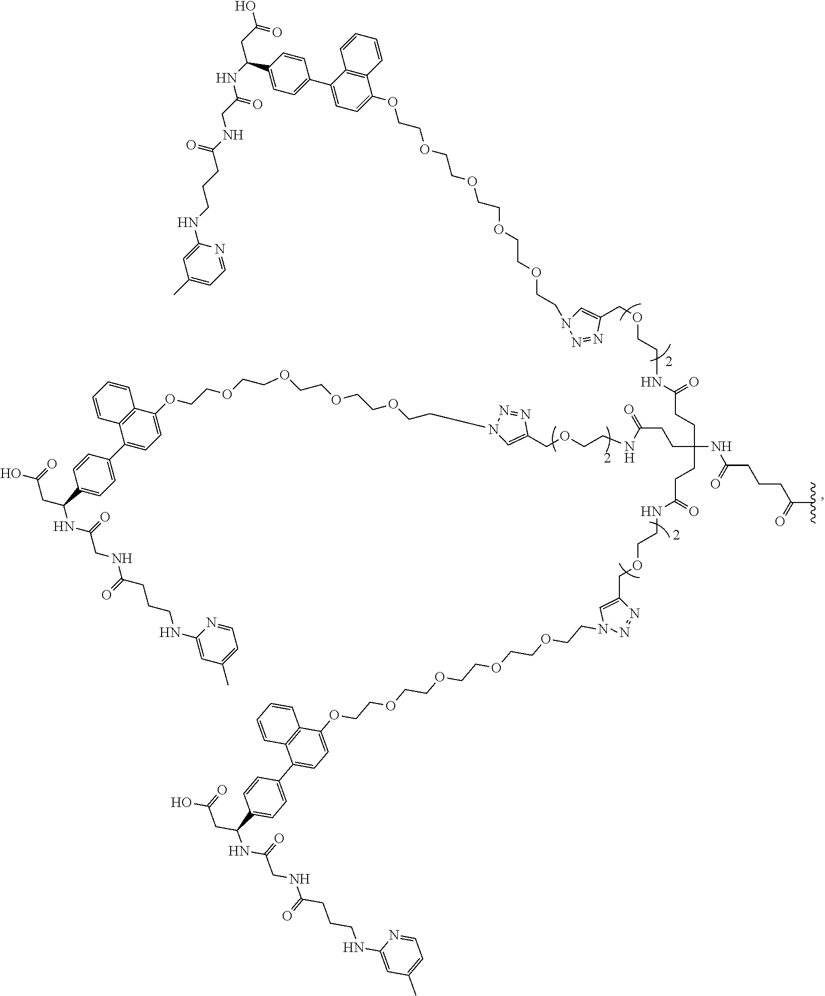 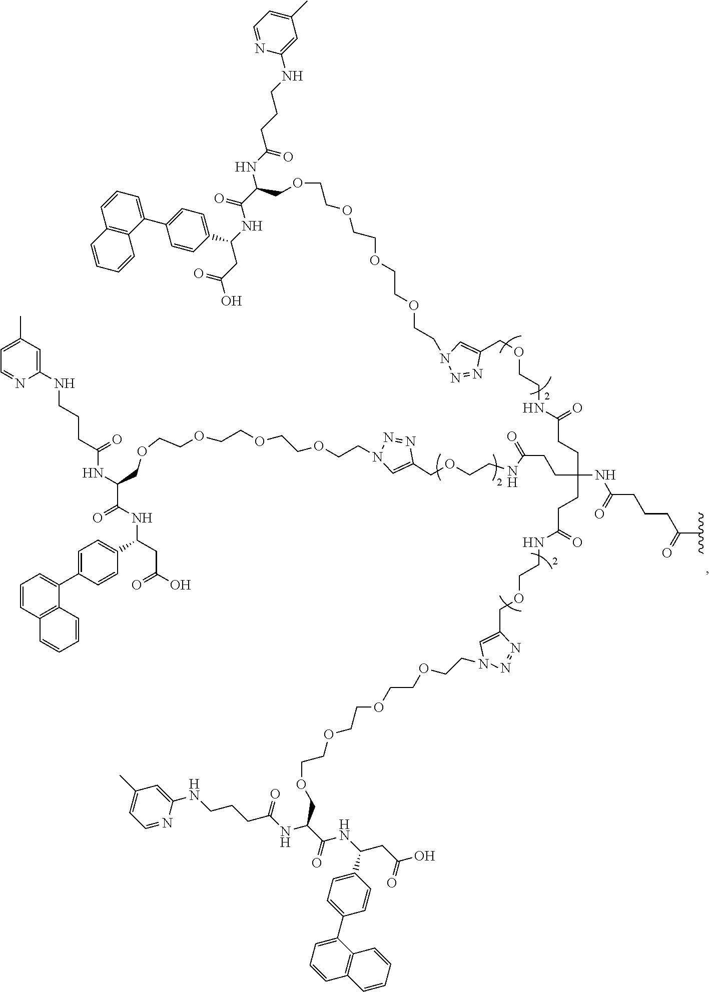 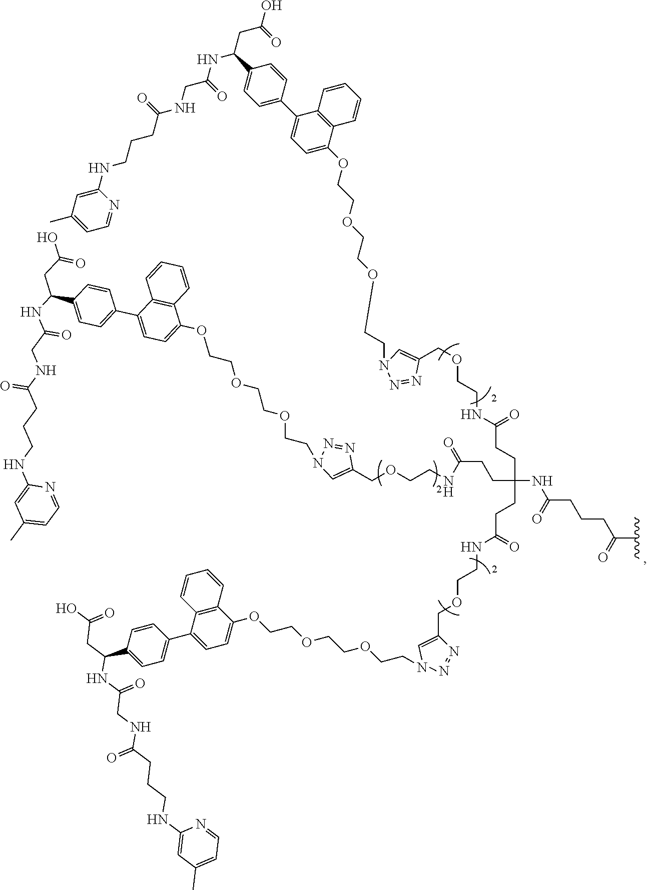 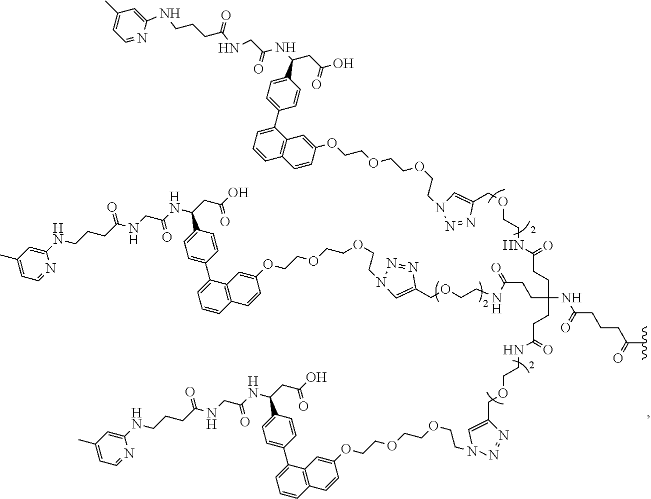 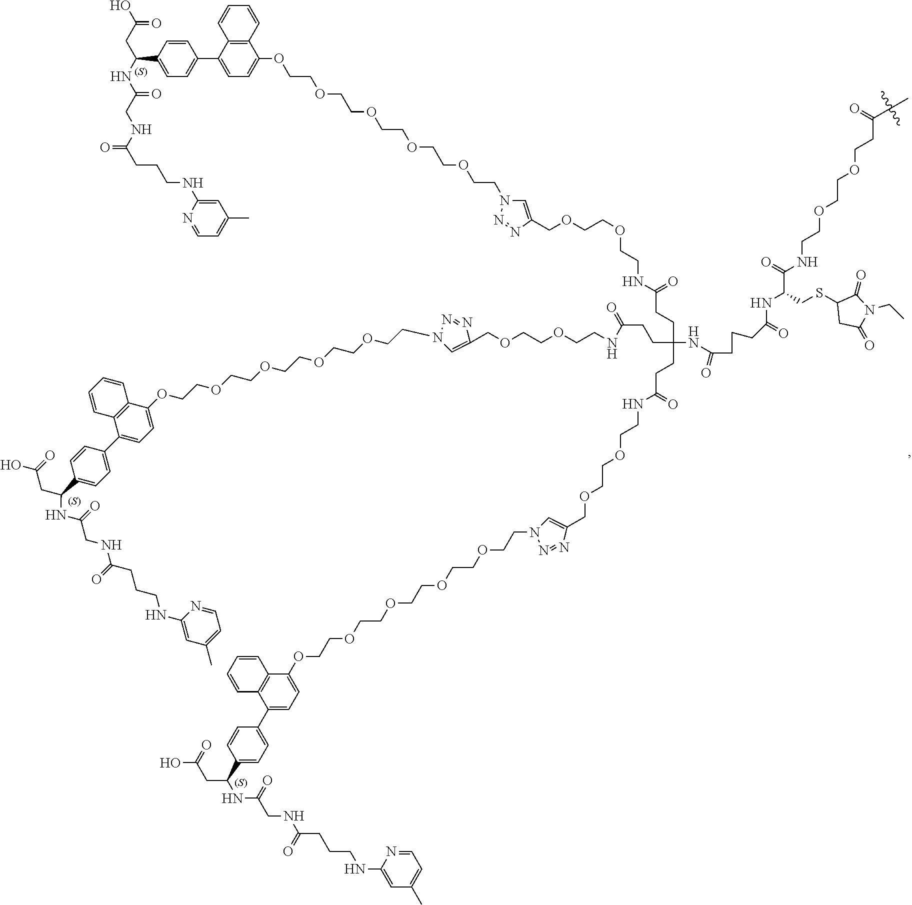 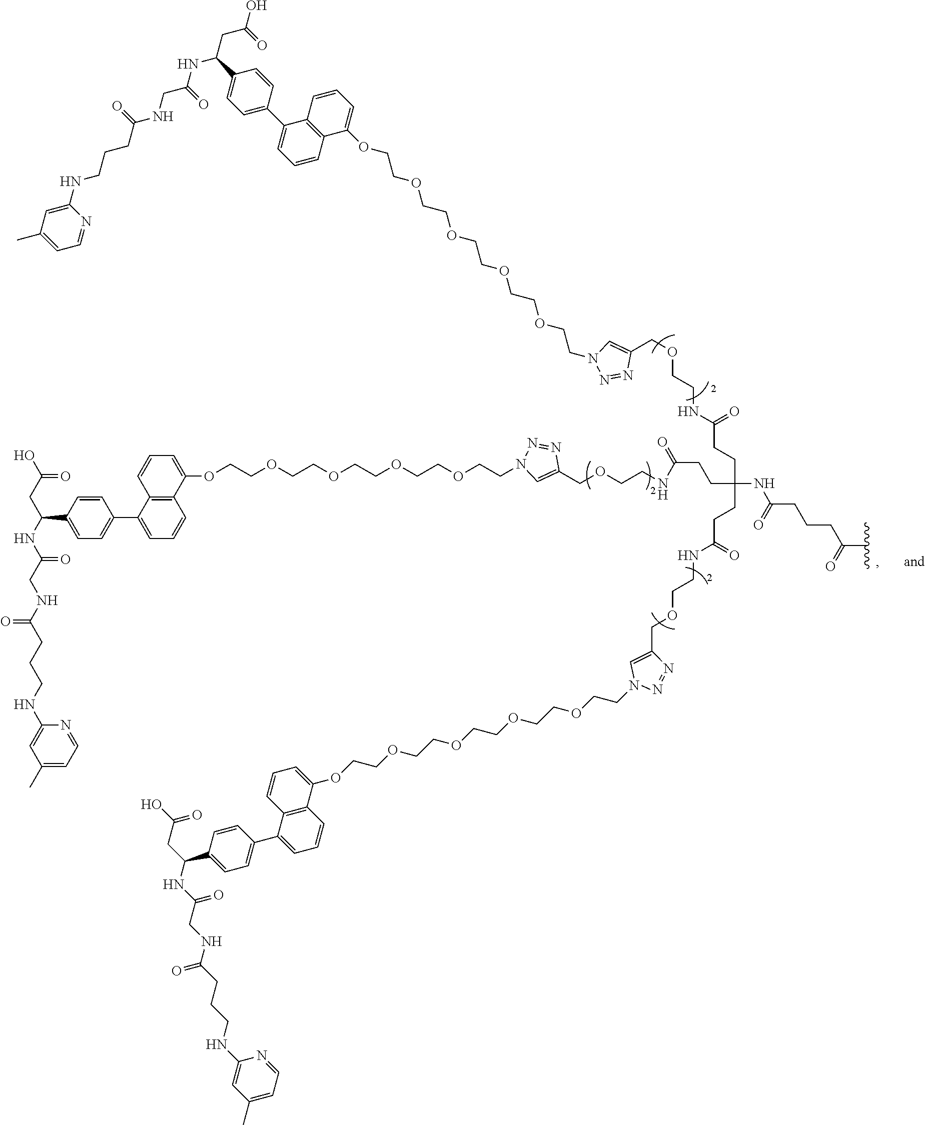 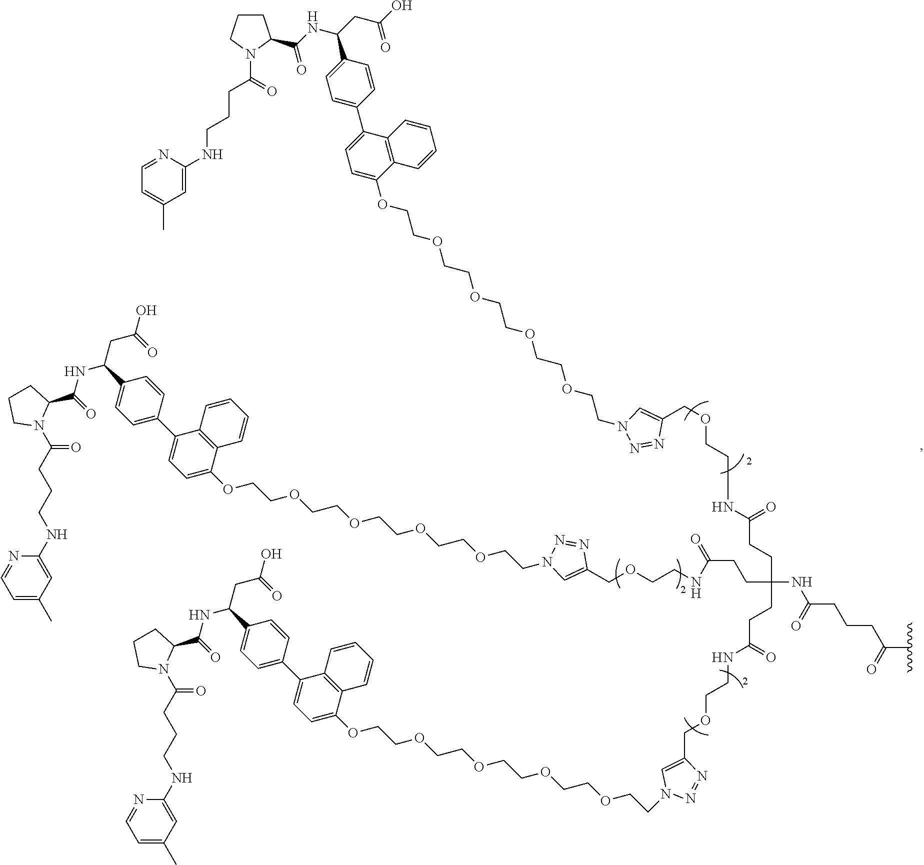
wherein [symbol] indicates the point of connection to the RNAi agent.

32-33. (canceled)

34. The RNAi agent of claim 26, wherein the targeting ligand is conjugated to the 5′ terminal end of the sense strand.

35. The RNAi agent of claim 1, wherein the influenza A viral genome is selected from the viral genomes of the group consisting of:
H1N1 viral genome;
H2N2 viral genome;
H3N2 viral genome;
H5N1 viral genome;
H7N9 viral genome; and
H10N8 viral genome.

36. A composition comprising the RNAi agent of claim 1, wherein the composition further comprises a pharmaceutically acceptable excipient.

37-39. (canceled)

40. The composition of claim 36, wherein the composition is formulated for administration by inhalation.

41. (canceled)

42. The composition of claim 36, wherein the RNAi agent is a sodium salt.

43-45. (canceled)

46. A method for inhibiting expression of an influenza A viral genome in a cell, the method comprising introducing into a cell an effective amount of an RNAi agent of claim 1.

47-65. (canceled)
